(19) United States
(12) Patent Application Publication
(10) Pub. No.: US 2023/0278959 A1
(43) Pub. Date: Sep. 7, 2023

(54) Synthesis of Optically Active Indoline Derivatives Via Ruthenium(II)-Catalyzed Enantioselective C-H Functionalization

(71) Applicant: Mississippi State University, Starkville, MS (US)

(72) Inventors: Xin Cui, Starkville, MS (US); Zhong-Yuan Li, Starkville, MS (US); Hetti Handi Chaminda Lakmal, Starkville, MS (US)

(21) Appl. No.: 17/923,559

(22) PCT Filed: May 4, 2021
(86) PCT No.: PCT/US2021/030741

§ 371 Date: Nov. 4, 2022

Related U.S. Application Data
(60) Provisional application No. 63/019,812, filed on May 4, 2020.

(51) Int. Cl. C07D 209/92; B01J 31/22

(52) U.S. Cl. CPC C07D 209/92; B01J 31/2295; B01J 2531/821; B01J 2540/68

(57) ABSTRACT

Provided herein are a method of Ru(II)-catalyzed enantioselective synthesis of a cyclic compound and cyclic compounds formed therefrom. The method includes providing a precursor compound having an unfunctionalized C—H bond and activating the unfunctionalized C—H bond by reacting the precursor compound in the presence of co-catalysts including a Ru(II) arene complex and a chiral transient directing group (CTDG).

### RELATED APPLICATIONS

This application claims the benefit of U.S. Provisional Application Ser. No. 63/019,812, filed May 4, 2020, the entire disclosure of which is incorporated herein by this reference.

### TECHNICAL FIELD

The present invention relates to indoline derivatives and methods of synthesizing the same. In particular, the presently-disclosed subject matter relates to optically active indolines and a Ru(II)-catalyzed method for enantioselective synthesis thereof.

### BACKGROUND

Development of new catalytic systems for enantioselective C—H functionalization has been growing rapidly with multidisciplinary impacts. Among different approaches, directed C—H bond activation has emerged as a general and effective tool. Beyond the C—H oxidative addition-based pathways, mechanistically new reactivities and selectivities by high-valent metals, including Pd(II), Ru(II), Rh(III), and others, have emerged via metalation/deprotonation pathways. Unlike low-valent metal-catalyzed systems, their enantioselective versions encounter mechanistic complication and intrinsic challenges that make many “privileged” ligands incompatible.

Research over the past decade has enabled successful application of monoprotected amino acids (MPAA) and related ligands in Pd(II)-catalyzed enantioselective C—H activation (FIG. 1A). More recently, bi-dentate chiral transient directing groups (CTDGs) and chiral transient mediators have been developed to address major challenges. The conformationally organized intermediates resulting from the chelation of the MPAA ligands and CTDGs at the square planar Pd center are key to the superior enantiocontrol. Moreover, Rh(III)-catalyzed enantioselective processes have been accomplished with both chiral Cp* ligands and engineered enzymes (FIG. 1B). While Rh(III) serves as the major focus of d⁶ metal catalysts with continued development, the scope was also extended to Ir(III) and very recently, Co(III). Sharing the general C—H metalation step, each metal species has shown distinct stereoselectivity and reactivity profiles, which have brought in new opportunities for the enantioselective access to various target molecules.

During the past two decades, Ru(II) arene complexes have emerged as effective and favorable catalysts for C—H activation owing to their cost-effectiveness, easy preparation, versatile and distinct reactivity and selectivity. Nevertheless, enantioselective C—H activation with Ru(II) remains unknown (FIG. 1C). With only three coordination sites, Ru(II) arene catalysts are not readily compatible with the design of ligands or bidentate CTDGs for Pd. Meanwhile, the inactivity of the Ru(II)Cp complexes in C—H activation has limited the application of chiral Cp* ligands. While Ru(II) continues to advance as an active metal catalyst for C—H activation, developing enantioselective versions as new synthetic tools is highly desirable as this would unlock practical and inexpensive access to meet the increasing need for new optically pure structures.

Accordingly, there remains a need for enantioselective catalysts and optically pure structures produced therefrom.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments, the presently-disclosed subject matter includes a method of Ru(II)-catalyzed enantioselective synthesis of a cyclic compound, the method comprising providing a precursor compound having an unfunctionalized C—H bond; and activating the unfunctionalized C—H bond by reacting the precursor compound in the presence of co-catalysts including a Ru(II) arene complex and a chiral transient directing group (CTDG). In some embodiments, the Ru(II) arene complex comprises a structure according to Formula I:

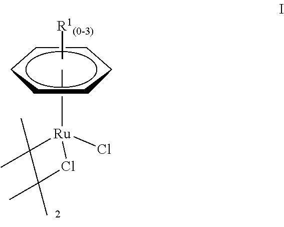

wherein R¹ includes a branched or unbranched alkyl. In some embodiments, the Ru(II) arene complex includes:

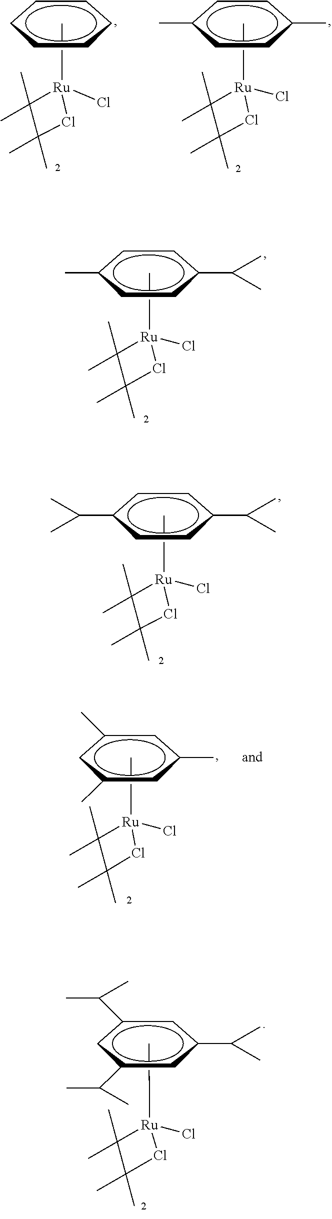

In some embodiments, the Ru(II) arene complex is:

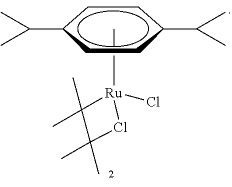

In some embodiments, the CTDG is an α-branched chiral amine. In some embodiments, the CTDG includes:

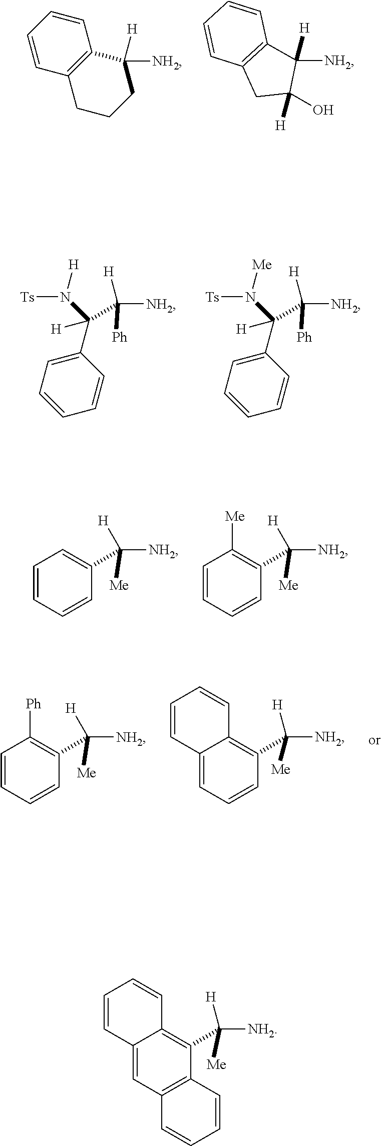

In some embodiments, the CTDG is:

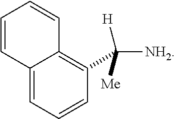

In some embodiments, the Ru(II) arene complex comprises a structure according to Formula I:

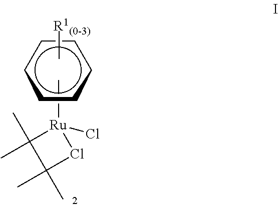

wherein R¹ includes a branched or unbranched alkyl and the CTDG is an α-branched chiral amine. In some embodiments, the Ru(II) arene complex includes:

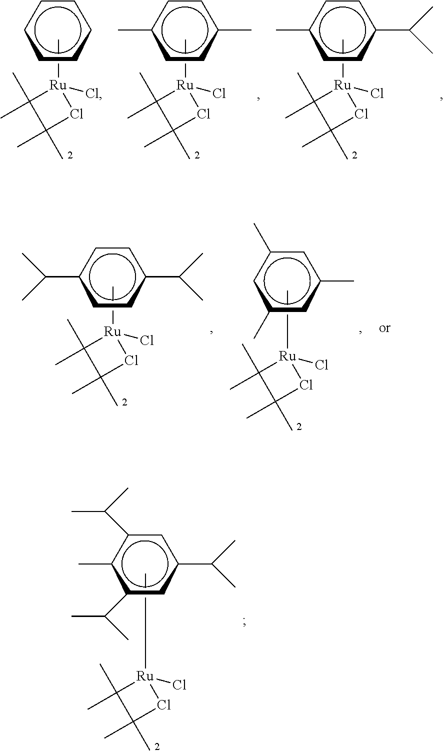

and the CTDG includes

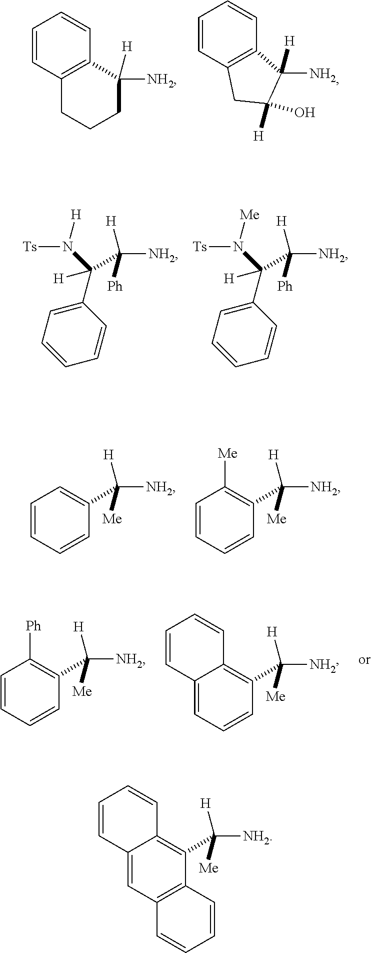

In some embodiments, the Ru(II) arene complex is:

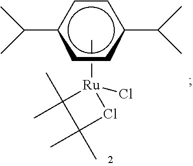

### and the CTDG is:

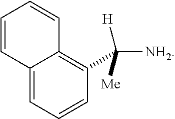

In some embodiments, the precursor compound comprises a compound according to Formula II:

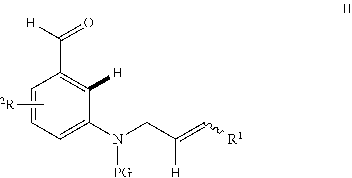

wherein R¹ is selected from the group consisting of alkyl, substituted alkyl, aryl, substituted aryl, electron withdrawing group, and combinations thereof; wherein R² is selected from the group consisting of H, alkyl, alkoxy, CF₃, halogen, and combinations thereof; and wherein PG is a protecting group. In some embodiments, the cyclic compound is an indoline derivative.

In some embodiments, the precursor compound comprises a compound according to Formula III:

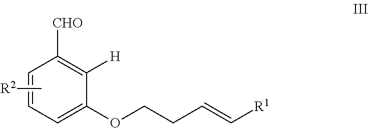

wherein R¹ is selected from the group consisting of alkyl, substituted alkyl, aryl, substituted aryl, electron withdrawing group, and combinations thereof; and wherein R² is selected from the group consisting of H, alkyl, alkoxy, CF₃, halogen, and combinations thereof. In some embodiments, the cyclic compound is a chromane derivative.

Also provided herein, in some embodiments, is a cyclic compound having a structure according to Formula IV:

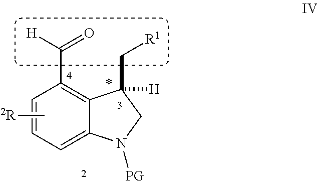

wherein R¹ is selected from the group consisting of alkyl, substituted alkyl, aryl, substituted aryl, electron withdrawing group, and combinations thereof; wherein R² is selected from the group consisting of H, alkyl, alkoxy, CF₃, halogen, and combinations thereof, and wherein PG is a protecting group. In some embodiments, the cyclic compound includes:

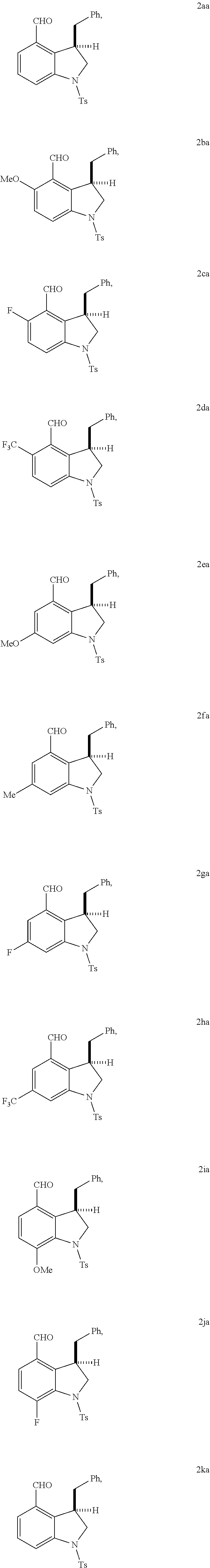

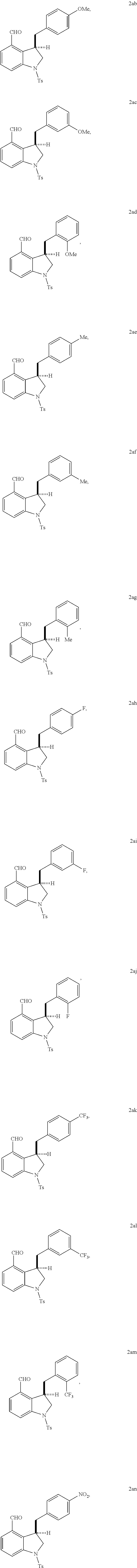

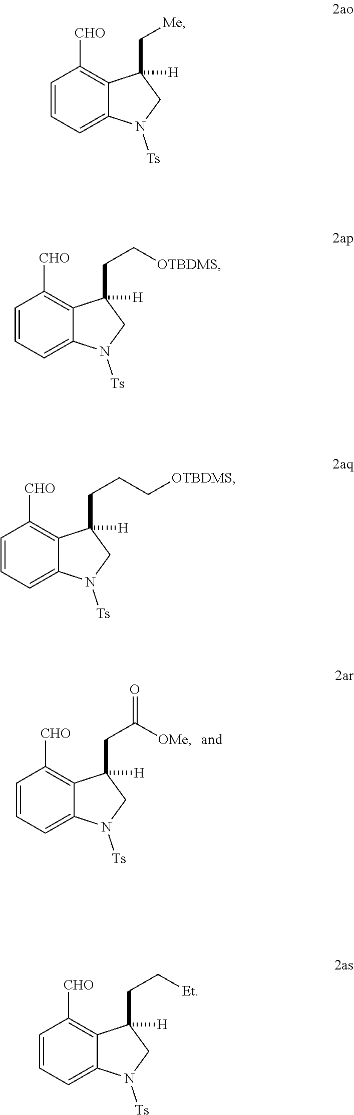 [SMILES: CC[C@H]1CN(S(=O)(=O)c2ccc(C)cc2)c2cccc(C=O)c21 ; Cc1ccc(S(=O)(=O)N2C[C@H](CCO[Si](C)(C)C(C)(C)C)c3c(C=O)cccc32)cc1 ; Cc1ccc(S(=O)(=O)N2C[C@H](CCCO[Si](C)(C)C(C)(C)C)c3c(C=O)cccc32)cc1 ; COC(=O)C[C@H]1CN(S(=O)(=O)c2ccc(C)cc2)c2cccc(C=O)c21 ; CCCC[C@H]1CN(S(=O)(=O)c2ccc(C)cc2)c2cccc(C=O)c21]

Further provided herein, in some embodiments, is a tricyclic compound having a structure according to Formula V.

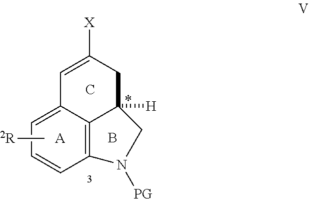

wherein X is CHO; wherein R² is selected from the group consisting of H, alkyl, alkoxy, CF₃, halogen, and combinations thereof, and wherein PG is a protecting group. In some embodiments, the compound is:

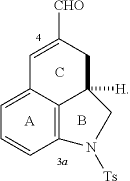

Further features and advantages of the presently-disclosed subject matter will become evident to those of ordinary skill in the art after a study of the description, figures, and non-limiting examples in this document.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIGS. 1A-C show images illustrating various transition metal catalysts for enantioselective C—H activation via directed metalation/deprotonation. (A) Pd(II)-catalyzed enantioselective C—H activation. (B) Rh(III), Ir(III), Co(III)-catalyzed enantioselective C—H activation. (C) A missing tool including Ru(II)-catalyzed enantioselective C—H activation.

FIGS. 2A-B show images illustrating indoline formation via C—H activation and functionalization. (A) Schematic showing various approaches capable of constructing any of the four non-aromatic bonds through C—H activation and functionalization. (B) Schematic showing a proposed CTDG strategy using Ru(II).

FIG. 3 shows a schematic illustrating a postulated asymmetric induction model.

FIGS. 4A-D show images and tables illustrating Ru(II)-catalyzed enantioselective hydroarylation under various conditions. (A) Schematic showing the hydroarylation reaction. Unless stated otherwise, the reaction conditions in A were 1aa (0.05 mmol), [Ru(p-cymene)Cl₂]₂ (5 mol %), AgBF₄ (20 mol %), acid (30 mol %), chiral amine (50 mol %), KH₂PO₄ (2.0 equiv), solvent 0.4 mL, 24 h. (B) Structures of various α-branched chiral amines and their effect as CTDGs in the reaction. Standard condition for B include: additive—AcOH (5 equivalents); solvent —ClCH₂CH₂Cl. (C) Effects of various additives and solvents in the reaction. Standard conditions for C include: CTDG—CA8; additive—A1-9 (30 mol %) and KH₂PO₄ (2 equivalents). Yield percent was determined by ¹H NMR with PhNO₂ as internal standard. In entry 22, (S)-1-(1-Naphthyl)ethylamine (ent-CA8) was used. (D) Effects of various arene ligands as the Ru catalyst in the reaction of A. Standard conditions for D include: CTDG—CA8; additive —A7 (30 mol %) and KH₂PO₄ (2 equivalents); solvent—PhCl:HFIP.

FIGS. 5A-B show images illustrating Ru(II)-catalyzed enantioselective hydroarylation with various arene moieties. (A) Schematic of Ru(II)-catalyzed enantioselective hydroarylation reaction with various arene moieties represented by R². Unless stated otherwise, the reaction conditions in A were 1 (0.1 mmol), [Ru(p-cymene)Cl₂]₂ (5 mol %), AgBF₄ (20 mol %), acid (30 mol %), chiral amine (50 mol %), KH₂PO₄ (2.0 equiv), solvent 0.8 mL, 60° C., 24 h. Isolated yield. (B) Resulting structures 2 from various starting arene moieties and associated yields. ^{b}80° C.; C70° C.; ^{d}90 C; ^{e}48 h.

FIGS. 6A-C show images and tables illustrating Ru(II)-catalyzed enantioselective hydroarylation to various internal alkene units. (A) Schematic of Ru(II)-catalyzed enantioselective hydroarylation reaction with various internal alkene units represented by R¹. Unless stated otherwise, the reaction conditions in A were 1 (0.1 mmol), [Ru(p-cymene)Cl₂]₂ (5 mol %), AgBF₄ (20 mol %), acid (30 mol %), chiral amine (50 mol %), KH₂PO₄ (2.0 equiv), solvent 0.8 mL, 60° C., 24 h. Isolated yield. (B) Resulting structures 2 from various internal alkene units and associated yields. ^{b}70° C.; ^{c}48 h. (C) Schematic comparing the performances of the E and Z isomers.

FIG. 7 shows schematics illustrating H/D exchange reactions carried out with a racemic form of amine CA5 in DCE at 30° C. (1), a racemic form of amine CA5 in DCE at 40° C. (2), and a control without amines at 40° C. (3).

FIGS. 8A-B show schematics illustrating a postulated mechanism and enantiocontrol for Ru(II)-catalyzed enantioselective hydroarylation. (A) Schematic of postulated mechanism. (B) Schematic of postulated enantiocontrol.

FIGS. 9A-C show images illustrating synthesis of intermediate-related ruthenacycles. (A) Schematic showing the reaction that forms 6a and 6b from 5, where R=4-ClC₆H₄. (B) Structures of 6a and 6b. (C) X-ray showing the structure of 6b. (D) Schematic showing H/D exchange (4) and catalytic reactions (5) employing tert-butylamine. The H/D exchange reaction (4) used the conditions of FIG. 7 (2) and the catalytic reaction used the conditions of FIG. 5A.

FIG. 10 shows a schematic illustrating synthetic applications of the chiral indolines.

FIG. 11 shows a schematic illustrating Ru(II)-catalyzed enantioselective access to various indoline-based bicyclic and polycyclic structures through a chiral transient directing group.

FIGS. 12A-B show images illustrating Ru(II)-catalyzed enantioselective C—H activation/hydroarylation for synthesis of chromane derivatives. (A) Schematic showing the synthesis of various chromane derivatives. (B) Structures of various chromane derivatives.

FIG. 13 shows a schematic illustrating synthesis of compound S1 through intermediate SS1.

FIG. 14 shows a schematic illustrating synthesis of compound S2.

FIG. 15 shows a schematic illustrating synthesis of compound S2.

FIG. 16 shows a schematic illustrating synthesis of compound S1.

FIG. 17 shows a schematic illustrating synthesis of compound 1.

FIG. 18 shows a schematic illustrating synthesis of compound 2aa.

FIG. 19 shows a schematic illustrating synthesis of compounds 2as and E-1as.

FIG. 20 shows a schematic illustrating synthesis of compounds 2as and Z-1as.

FIG. 21 shows a schematic illustrating synthesis of compound 3a.

FIG. 22 shows schematics illustrating synthesis of compound 2aa under different reaction conditions.

FIG. 23 shows an image illustrating ¹H NMR of starting material after H/D exchange experiment at 30° C. (1).

FIG. 24 shows am image illustrating ¹H NMR of starting material after H/D exchange experiment at 40° C. (2).

FIG. 25 shows an image illustrating ¹H NMR of the product after H/D exchange experiment at 40° C. (2).

FIG. 26 shows an image illustrating ¹H NMR of starting material after H/D exchange experiment at 40° C. without amine (3).

FIG. 27 shows a schematic illustrating synthesis of compound 1aa under different reaction conditions.

FIG. 28 shows an image illustrating ¹H NMR of starting material after H/D exchange experiment at 40° C. with t-Butylamine (4).

FIG. 29 shows an image illustrating characterization of a reaction intermediate.

FIG. 30 shows an image illustrating characterization of a reaction intermediate.

FIG. 31 shows an image illustrating characterization of a reaction intermediate.

FIG. 32 shows an image illustrating characterization of a reaction intermediate.

FIG. 33 shows an image illustrating ¹H NMR of 6a (dr=70:30).

FIG. 34 shows an image illustrating ¹³C NMR of 6a (dr=70:30).

FIG. 35 shows an image illustrating ¹H NMR of 6b (dr=90:10).

FIG. 36 shows an image illustrating ¹³C NMR of 6b (dr=90:10).

FIG. 37 shows an image illustrating ¹H NMR of 6b (major diastereomer).

FIG. 38 shows an image illustrating ¹³C NMR of 6b (major diastereomer).

FIG. 39 shows an image illustrating Crystal's Resolution: using a Cu monochromatized X-ray radiation: λ=1.54178 Å.

FIG. 40 shows an image illustrating asymmetrical unit's view.

FIG. 41 shows an image illustrating Crystal's Resolution: using a Mo monochromatized X-ray radiation: λ=0.71073 Å.

FIG. 42 shows an image illustrating asymmetrical unit's view.

FIGS. 43A-B show images illustrating NMR spectra data for compound 1aa. (A) ¹H NMR (500 MHz, CDCl₃) of compound 1aa. (B)¹³C NMR (125 MHz, CDCl₃) of compound 1aa.

FIGS. 44A-B show images illustrating NMR spectra data for compound 1ba. (A) ¹H NMR (500 MHz, CDCl₃) of compound 1ba. (B)¹³C NMR (125 MHz, CDCl₃) of compound 1ba.

FIGS. 45A-B show images illustrating NMR spectra data for compound 1ca. (A) ¹H NMR (500 MHz, CDCl₃) of compound 1ca. (B)¹³C NMR (125 MHz, CDCl₃) of compound 1ca.

FIGS. 46A-B show images illustrating NMR spectra data for compound 1da. (A) ¹H NMR (500 MHz, CDCl₃) of compound 1da. (B)¹³C NMR (125 MHz, CDCl₃) of compound 1da.

FIGS. 47A-B show images illustrating NMR spectra data for compound 1ea. (A) ¹H NMR (500 MHz, CDCl₃) of compound 1ea. (B)¹³C NMR (125 MHz, CDCl₃) of compound 1ea.

FIGS. 48A-B show images illustrating NMR spectra data for compound 1fa. (A)¹H NMR (500 MHz, CDCl₃) of compound 1fa. (B)¹³C NMR (125 MHz, CDCl₃) of compound 1fa.

FIGS. 49A-B show images illustrating NMR spectra data for compound 1ga. (A)¹H NMR (500 MHz, CDCl₃) of compound 1ga. (B)¹³C NMR (125 MHz, CDCl₃) of compound 1ga.

FIGS. 50A-B show images illustrating NMR spectra data for compound 1ha. (A)¹H NMR (500 MHz, CDCl₃) of compound 1ha. (B)¹³C NMR (125 MHz, CDCl₃) of compound 1ha.

FIGS. 51A-B show images illustrating NMR spectra data for compound 1ia. (A)¹H NMR (500 MHz, CDCl₃) of compound 11a. (B)¹³C NMR (125 MHz, CDCl₃) of compound 1ia.

FIGS. 52A-B show images illustrating NMR spectra data for compound 1ja. (A)¹H NMR (500 MHz, CDCl₃) of compound 1ja. (B)¹³C NMR (125 MHz, CDCl₃) of compound 1ja.

FIGS. 53A-B show images illustrating NMR spectra data for compound 1ka. (A)¹H NMR (500 MHz, CDCl₃) of compound 1ka. (B)¹³C NMR (125 MHz, CDCl₃) of compound 1ka.

FIGS. 54A-B show images illustrating NMR spectra data for compound 1ab. (A)¹H NMR (500 MHz, CDCl₃) of compound 1ab. (B)¹³C NMR (125 MHz, CDCl₃) of compound 1ab.

FIGS. 55A-B show images illustrating NMR spectra data for compound 1ac. (A)¹H NMR (500 MHz, CDCl₃) of compound 1ac. (B)¹³C NMR (125 MHz, CDCl₃) of compound 1ac.

FIGS. 56A-B show images illustrating NMR spectra data for compound 1ad. (A) ¹H NMR (500 MHz, CDCl₃) of compound 1ad. (B)¹³C NMR (125 MHz, CDCl₃) of compound 1ad.

FIGS. 57A-B show images illustrating NMR spectra data for compound 1ae. (A) ¹H NMR (500 MHz, CDCl₃) of compound 1ae. (B)¹³C NMR (125 MHz, CDCl₃) of compound 1ae.

FIGS. 58A-B show images illustrating NMR spectra data for compound 1af. (A) ¹H NMR (500 MHz, CDCl₃) of compound 1af. (B)¹³C NMR (125 MHz, CDCl₃) of compound 1af.

FIGS. 59A-B show images illustrating NMR spectra data for compound 1ag. (A) ¹H NMR (500 MHz, CDCl₃) of compound 1ag. (B)¹³C NMR (125 MHz, CDCl₃) of compound 1ag.

FIGS. 60A-B show images illustrating NMR spectra data for compound 1ah. (A) ¹H NMR (500 MHz, CDCl₃) of compound 1ah. (B)¹³C NMR (125 MHz, CDCl₃) of compound 1ah.

FIGS. 61A-B show images illustrating NMR spectra data for compound 1ai. (A) ¹H NMR (500 MHz, CDCl₃) of compound 1ai. (B)¹³C NMR (125 MHz, CDCl₃) of compound 1ai.

FIGS. 62A-B show images illustrating NMR spectra data for compound 1aj. (A)¹H NMR (500 MHz, CDCl₃) of compound 1aj. (B)¹³C NMR (125 MHz, CDCl₃) of compound 1aj.

FIGS. 63A-B show images illustrating NMR spectra data for compound 1ak. (A)¹H NMR (500 MHz, CDCl₃) of compound 1ak. (B)¹³C NMR (125 MHz, CDCl₃) of compound 1ak.

FIGS. 64A-B show images illustrating NMR spectra data for compound 1al. (A)¹H NMR (500 MHz, CDCl₃) of compound 1al. (B)¹³C NMR (125 MHz, CDCl₃) of compound 1al.

FIGS. 65A-B show images illustrating NMR spectra data for compound 1am. (A)¹H NMR (500 MHz, CDCl₃) of compound 1am. (B)¹³C NMR (125 MHz, CDCl₃) of compound 1am.

FIGS. 66A-B show images illustrating NMR spectra data for compound 1an. (A)¹H NMR (500 MHz, CDCl₃) of compound 1an. (B)¹³C NMR (125 MHz, CDCl₃) of compound 1an.

FIGS. 67A-B show images illustrating NMR spectra data for compound 1ao. (A)¹H NMR (500 MHz, CDCl₃) of compound 1ao. (B)¹³C NMR (125 MHz, CDCl₃) of compound 1ao.

FIGS. 68A-B show images illustrating NMR spectra data for compound 1ap. (A)¹H NMR (500 MHz, CDCl₃) of compound 1ap. (B)¹³C NMR (125 MHz, CDCl₃) of compound 1ap.

FIGS. 69A-B show images illustrating NMR spectra data for compound 1aq. (A) ¹H NMR (500 MHz, CDCl₃) of compound 1aq. (B)¹³C NMR (125 MHz, CDCl₃) of compound 1aq.

FIGS. 70A-B show images illustrating NMR spectra data for compound 1ar. (A) ¹H NMR (500 MHz, CDCl₃) of compound tar. (B)¹³C NMR (125 MHz, CDCl₃) of compound 1ar.

FIGS. 71A-B show images illustrating NMR spectra data for compound E-1as. (A) H NMR (500 MHz, CDCl₃) of compound E-1as. (B)¹³C NMR (125 MHz, CDCl₃) of compound E-1as.

FIGS. 72A-B show images illustrating NMR spectra data for compound Z-1as. (A) ¹H NMR (500 MHz, CDCl₃) of compound Z-1as. (B)¹³C NMR (125 MHz, CDCl₃) of compound Z-1as.

FIGS. 73A-D show images illustrating NMR spectra and chiral HPLC data for compound 2aa. (A) ¹H NMR (500 MHz, CDCl₃) of compound 2aa. (B)¹³C NMR (125 MHz, CDCl₃) of compound 2aa. (C-D) HPLC data for compound 2aa.

FIGS. 74A-D show images illustrating NMR spectra data for compound 2ba. (A) ¹H NMR (500 MHz, CDCl₃) of compound 2ba. (B)¹³C NMR (125 MHz, CDCl₃) of compound 2ba. (C-D) HPLC data for compound 2ba.

FIGS. 75A-E show images illustrating NMR spectra data for compound 2ca. (A) ¹H NMR (500 MHz, CDCl₃) of compound 2ca. (B)¹³C NMR (125 MHz, CDCl₃) of compound 2ca. (C)¹⁹F NMR (470 MHz, CDCl₃) of compound 2ca. (D-E) HPLC data for compound 2ca.

FIGS. 76A-E show images illustrating NMR spectra data for compound 2da. (A)¹H NMR (500 MHz, CDCl₃) of compound 2da. (B)¹³C NMR (125 MHz, CDCl₃) of compound 2da. (C)¹⁹F NMR (470 MHz, CDCl₃) of compound 2da. (D-E) HPLC data for compound 2da.

FIGS. 77A-D show images illustrating NMR spectra data for compound 2ea. (A)¹H NMR (500 MHz, CDCl₃) of compound 2ea. (B)¹³C NMR (125 MHz, CDCl₃) of compound 2ea. (C-D) HPLC data for compound 2ea.

FIGS. 78A-D show images illustrating NMR spectra data for compound 2fa. (A)¹H NMR (500 MHz, CDCl₃) of compound 2fa. (B)¹³C NMR (125 MHz, CDCl₃) of compound 2fa. (C-D) HPLC data for compound 2fa.

FIGS. 79A-E show images illustrating NMR spectra data for compound 2ga. (A) ¹H NMR (500 MHz, CDCl₃) of compound 2ga. (B)¹³C NMR (125 MHz, CDCl₃) of compound 2ga. (C)¹⁹F NMR (470 MHz, CDCl₃) of compound 2ga. (D-E) HPLC data for compound 2ga.

FIGS. 80A-E show images illustrating NMR spectra data for compound 2ha. (A)¹H NMR (500 MHz, CDCl₃) of compound 2ha. (B)¹³C NMR (125 MHz, CDCl₃) of compound 2ha. (C)¹⁹F NMR (470 MHz, CDCl₃) of compound 2ha. (D-E) HPLC data for compound 2ha.

FIGS. 81A-D show images illustrating NMR spectra data for compound 2ia. (A)¹H NMR (500 MHz, CDCl₃) of compound 2ia. (B)¹³C NMR (125 MHz, CDCl₃) of compound 2ia. (C-D) HPLC data for compound 2ia.

FIGS. 82A-E show images illustrating NMR spectra data for compound 2ja. (A)¹H NMR (500 MHz, CDCl₃) of compound 2ja. (B)¹³C NMR (125 MHz, CDCl₃) of compound 2ja. (C)¹⁹F NMR (470 MHz, CDCl₃) of compound 2ja. (D-E) HPLC data for compound 2ja.

FIGS. 83A-D show images illustrating NMR spectra data for compound 2ka. (A) ¹H NMR (500 MHz, CDCl₃) of compound 2ka. (B)¹³C NMR (125 MHz, CDCl₃) of compound 2ka. (C-D) HPLC data for compound 2ka.

FIGS. 84A-D show images illustrating NMR spectra data for compound 2ab. (A) ¹H NMR (500 MHz, CDCl₃) of compound 2ab. (B)¹³C NMR (125 MHz, CDCl₃) of compound 2ab. (C-D) HPLC data for compound 2ab.

FIGS. 85A-D show images illustrating NMR spectra data for compound 2ac. (A) ¹H NMR (500 MHz, CDCl₃) of compound 2ac. (B)¹³C NMR (125 MHz, CDCl₃) of compound 2ac. (C-D) HPLC data for compound 2ac.

FIGS. 86A-D show images illustrating NMR spectra data for compound 2ad. (A) ¹H NMR (500 MHz, CDCl₃) of compound 2ad. (B)¹³C NMR (125 MHz, CDCl₃) of com87 pound 2ad. (C-D) HPLC data for compound 2ad.

FIGS. 87A-D show images illustrating NMR spectra data for compound 2ae. (A) ¹H NMR (500 MHz, CDCl₃) of compound 2ae. (B)¹³C NMR (125 MHz, CDCl₃) of compound 2ae. (C-D) HPLC data for compound 2ae.

FIGS. 88A-D show images illustrating NMR spectra data for compound 2af. (A) H NMR (500 MHz, CDCl₃) of compound 2af. (B)¹³C NMR (125 MHz, CDCl₃) of compound 2af. (C-D) HPLC data for compound 2af.

FIGS. 89A-D show images illustrating NMR spectra data for compound 2ag. (A) H NMR (500 MHz, CDCl₃) of compound 2ag. (B)¹³C NMR (125 MHz, CDCl₃) of compound 2ag. (C-D) HPLC data for compound 2ag.

FIGS. 90A-E show images illustrating NMR spectra data for compound 2ah. (A) H NMR (500 MHz, CDCl₃) of compound 2ah. (B)¹³C NMR (125 MHz, CDCl₃) of compound 2ah. (C)¹⁹F NMR (470 MHz, CDCl₃) of compound 2ah. (D-E) HPLC data for compound 2ah.

FIGS. 91A-E show images illustrating NMR spectra data for compound 2ai. (A) H NMR (500 MHz, CDCl₃) of compound 2ai. (B)¹³C NMR (125 MHz, CDCl₃) of compound 2ai. (C)¹⁹F NMR (470 MHz, CDCl₃) of compound 2ai. (D-E) HPLC data for compound 2ai.

FIGS. 92A-E show images illustrating NMR spectra data for compound 2aj. (A) H NMR (500 MHz, CDCl₃) of compound 2aj. (B)¹³C NMR (125 MHz, CDCl₃) of compound 2aj. (C)¹⁹F NMR (470 MHz, CDCl₃) of compound 2aj. (D-E) HPLC data for compound 2aj.

FIGS. 93A-E show images illustrating NMR spectra data for compound 2ak. (A) ¹H NMR (500 MHz, CDCl₃) of compound 2ak. (B)¹³C NMR (125 MHz, CDCl₃) of compound 2ak. (C)¹⁹F NMR (470 MHz, CDCl₃) of compound 2ak. (D-E) HPLC data for compound 2ak.

FIGS. 94A-E show images illustrating NMR spectra data for compound 2al. (A) ¹H NMR (500 MHz, CDCl₃) of compound 2al. (B)¹³C NMR (125 MHz, CDCl₃) of compound 2al. (C)¹⁹F NMR (470 MHz, CDCl₃) of compound 2al. (D-E) HPLC data for compound 2al.

FIGS. 95A-E show images illustrating NMR spectra data for compound 2am. (A) ¹H NMR (500 MHz, CDCl₃) of compound 2am. (B)¹³C NMR (125 MHz, CDCl₃) of compound 2am. (C)¹⁹F NMR (470 MHz, CDCl₃) of compound 2am. (D-E) HPLC data for compound 2am.

FIGS. 96A-D show images illustrating NMR spectra data for compound 2an. (A) ¹H NMR (500 MHz, CDCl₃) of compound 2an. (B)¹³C NMR (125 MHz, CDCl₃) of compound 2an. (C-D) HPLC data for compound 2an.

FIGS. 97A-D show images illustrating NMR spectra data for compound 2ao. (A) ¹H NMR (500 MHz, CDCl₃) of compound 2ao. (B)¹³C NMR (125 MHz, CDCl₃) of compound 2ao. (C-D) HPLC data for compound 2ao.

FIGS. 98A-D show images illustrating NMR spectra data for compound 2ap. (A) ¹H NMR (500 MHz, CDCl₃) of compound 2ap. (B)¹³C NMR (125 MHz, CDCl₃) of compound 2ap. (C-D) HPLC data for compound 2ap.

FIGS. 99A-D show images illustrating NMR spectra data for compound 2aq. (A) ¹H NMR (500 MHz, CDCl₃) of compound 2aq. (B)¹³C NMR (125 MHz, CDCl₃) of compound 2aq. (C-D) HPLC data for compound 2aq.

FIGS. 100A-D show images illustrating NMR spectra data for compound 2ar. (A) ¹H NMR (500 MHz, CDCl₃) of compound 2ar. (B)¹³C NMR (125 MHz, CDCl₃) of compound 2ar. (C-D) HPLC data for compound 2ar.

FIGS. 101A-D show images illustrating NMR spectra data for compound 2as. (A) ¹H NMR (500 MHz, CDCl₃) of compound 2as. (B)¹³C NMR (125 MHz, CDCl₃) of compound 2as. (C-D) HPLC data for compound 2as.

FIGS. 102A-D show images illustrating NMR spectra data for compound 3a. (A) ¹H NMR (500 MHz, CDCl₃) of compound 3a. (B)¹³C NMR (125 MHz, CDCl₃) of compound 3a. (C-D) HPLC data for compound 3a.

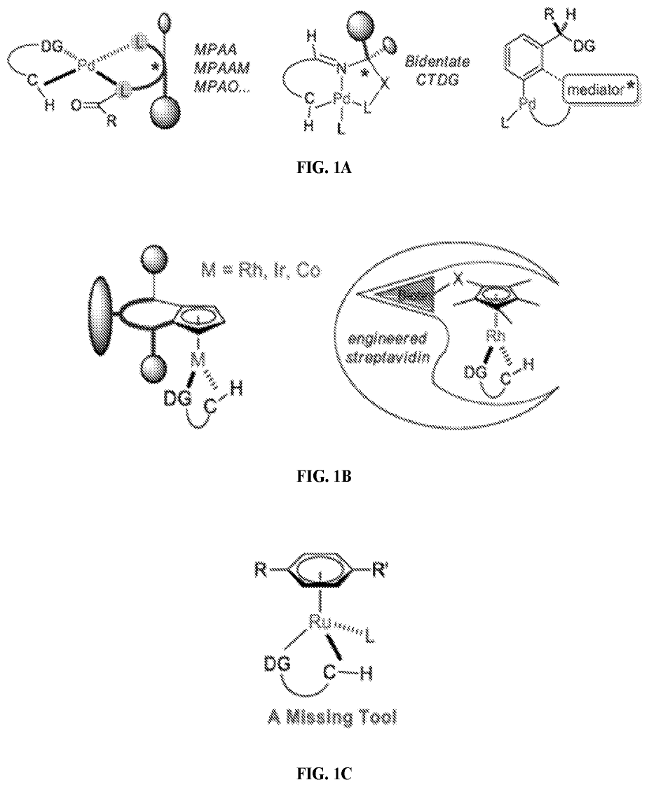

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims, unless the context clearly dictates otherwise. Thus, for example, reference to “a polypeptide” includes one or more of such polypeptides, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Provided herein, in some embodiments, is a method of Ru(II)-catalyzed enantioselective synthesis of a cyclic compound. In some embodiments, the method includes Ru(II)-catalyzed enantioselective C—H activation/hydroarylation of a precursor compound. In one embodiment, the precursor compound includes any suitable compound having an unfunctionalized C—H bond. In another embodiment, the method includes functionalizing the unfunctionalized C—H bond in the precursor compound. In a further embodiment, functionalizing the unfunctionalized C—H bond includes reacting the precursor compound in the presence of co-catalysts including a Ru(II) arene complex and a chiral transient directing group (CTDG).

In some embodiments, the Ru(II) arene complex includes a complex according to Formula I:

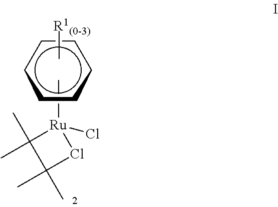

Where R¹ includes a branched or unbranched alkyl. In some embodiments, the alkyl of R¹ includes a C₁-C₃ alkyl. For example, suitable Ru(II) arene complexes according to Formula I include, but are not limited to, one or more of the following:

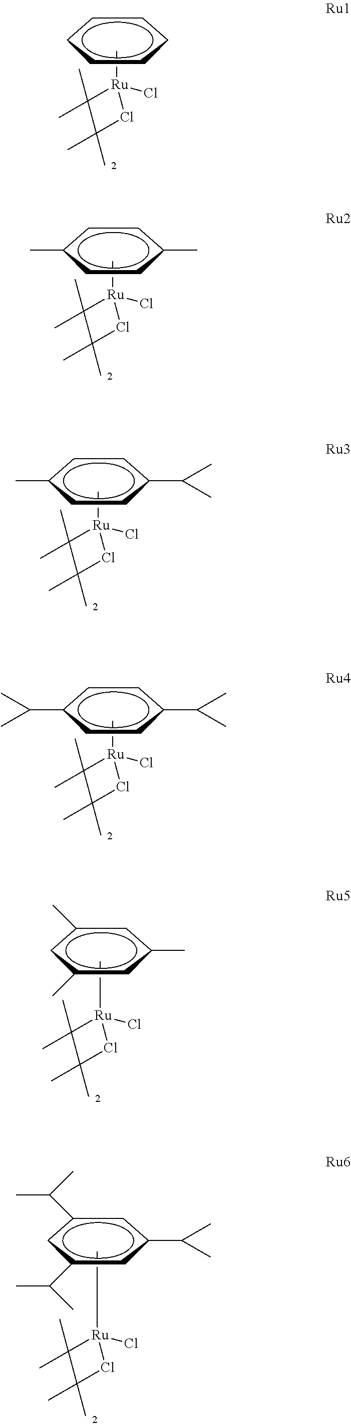

Suitable CTDGs include, but are not limited to, α-branched chiral amines. For example, in some embodiments, the α-branched chiral amines include chiral α-methylamines, such as, but not limited to, one or more of the following:

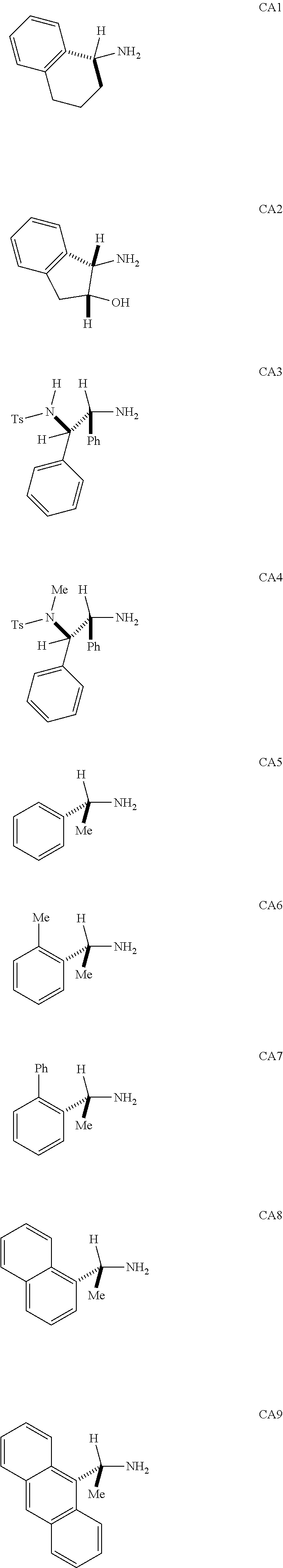

The co-catalysts may include any suitable combination of the Ru(II) arene complex and the CTDG. For example, in one embodiment, the co-catalysts include Ru4 and CA8.

As will be appreciated by those skilled in the art, the structure of the cyclic compound synthesized according to one or more of the embodiments disclosed herein will depend on the precursor compound being used. For example, in some embodiments, the method includes synthesizing an indoline derivative (e.g., functionalized chiral indoline) through reaction of the unfunctionalized C—H bond in the precursor compound according to Formula II:

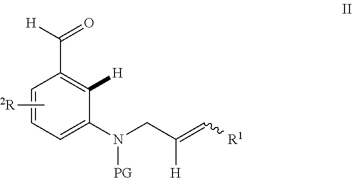

Where R¹ includes alkyl, substituted alkyl, aryl, substituted aryl, electron withdrawing group, or a combination thereof, R² includes H, alkyl, alkoxy, CF₃, halogen, or a combination thereof, and PG includes a protecting group. Suitable protecting groups include, but are not limited to, tosyl, nosyl, or any other suitable protecting group. In some embodiments, the precursor compound includes any one or more of the compounds shown in the Examples below. Additionally or alternatively, in some embodiments, the method includes synthesizing a chromane derivative (e.g., functionalized chiral chromane) through reaction of the unfunctionalized C—H bond in the precursor compound according to Formula III:

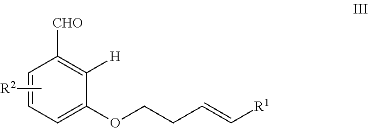

Where R¹ includes alkyl, substituted alkyl, aryl, substituted aryl, electron withdrawing group, or a combination thereof, and R² includes H, alkyl, alkoxy, CF₃, halogen, or a combination thereof. Other derivatives that may be formed according to one or more of the embodiments disclosed herein include, but are not limited to, isochromane derivatives, 9-fluorene derivatives, any other suitable derivative, or a combination thereof.

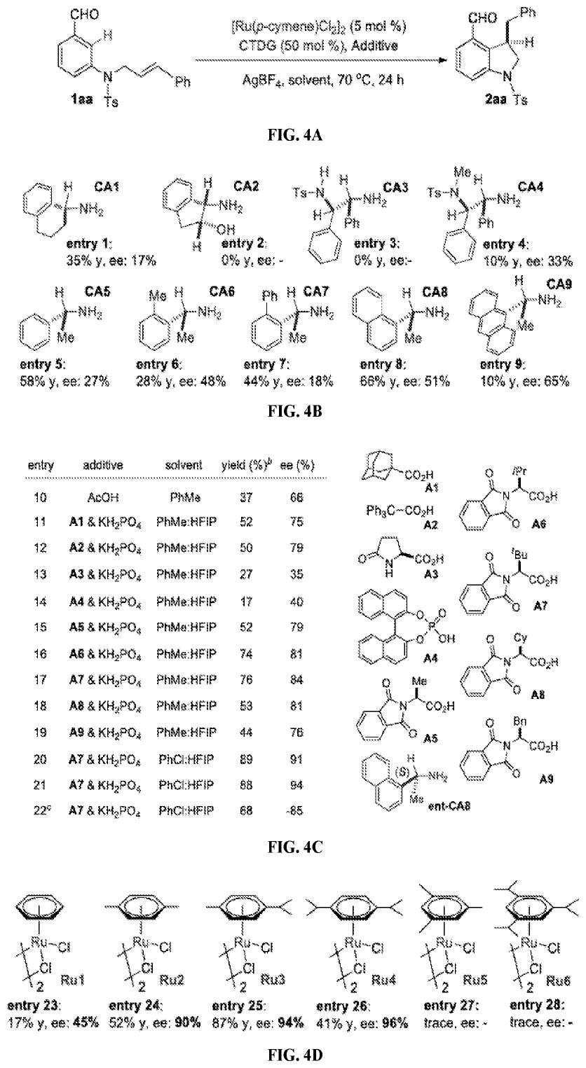

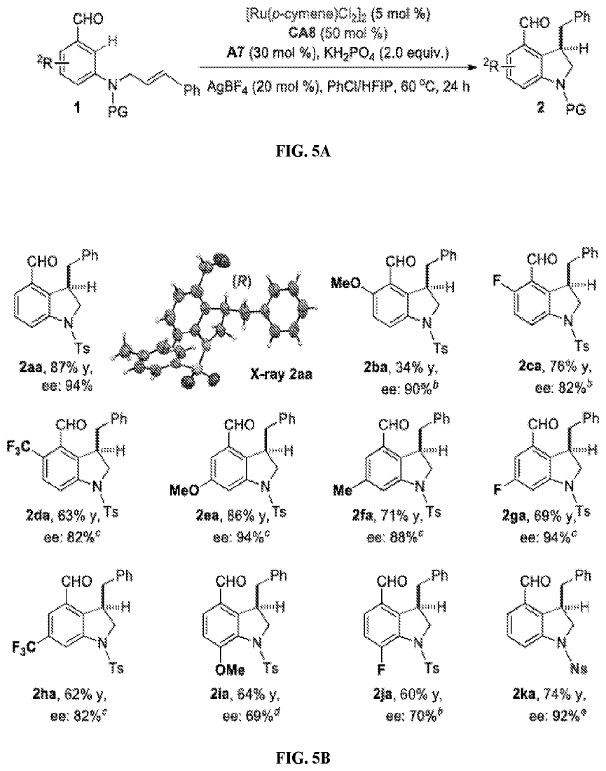

The method may also include any suitable solvent, additive, and/or reaction condition based upon the precursor compound, co-catalysts, and desired cyclic compound being synthesized. In some embodiments, the method includes reacting the unfunctionalized C—H bond(s) in the precursor compound in the presence of AgBF₄, a solvent, and/or one or more additives. Suitable solvents include, but are not limited to, PhMe, PhMe:HFIP, PhCl:HFIP, or any other suitable solvent. Suitable additives include, but are not limited to, AcOH, KH₂PO₄ and any of A1-A9 in FIG. 4C, any other suitable additive, or a combination thereof. Suitable reaction conditions include, but are not limited to, temperatures of between 60° C. and 90° C., a length of between 24 hours and 48 hours, or any combination thereof. For example, in one embodiment, the synthesis of an indoline derivative includes any suitable combination of the co-catalysts, additives, solvents, and reaction conditions shown in FIGS. 4A-D. In another embodiment, the synthesis of a chromane derivative includes any suitable combination of the co-catalysts, additives, solvents, and reaction conditions shown in FIGS. 4A-D and 12A-B.

Without wishing to be bound by theory, it is believed that the methods disclosed herein represent the first Ru(II)-catalyzed enantioselective C—H activation/hydroarylation. One or more of these methods provide a highly enantioselective synthesis of indoline, chromane, isochromane, and/or 9-fluorene derivatives via catalytic C—H activation. For example, in some embodiments, based on a sterically rigidified chiral transient directing group, the methods disclosed herein produce multi-substituted indolines in up to 92% yield with 96% ee. Not only are these methods efficient, the use of Ru(II) as a catalyst is substantially less expensive than existing methods that typically use palladium and rhodium catalysts. Furthermore, the use of chiral amines as the co-catalyst largely reduces the cost and environmental effect of the synthesis, as compared to the existing phosphine-based or the chiral cyclopentadienyl (Cp^{x}) ligands.

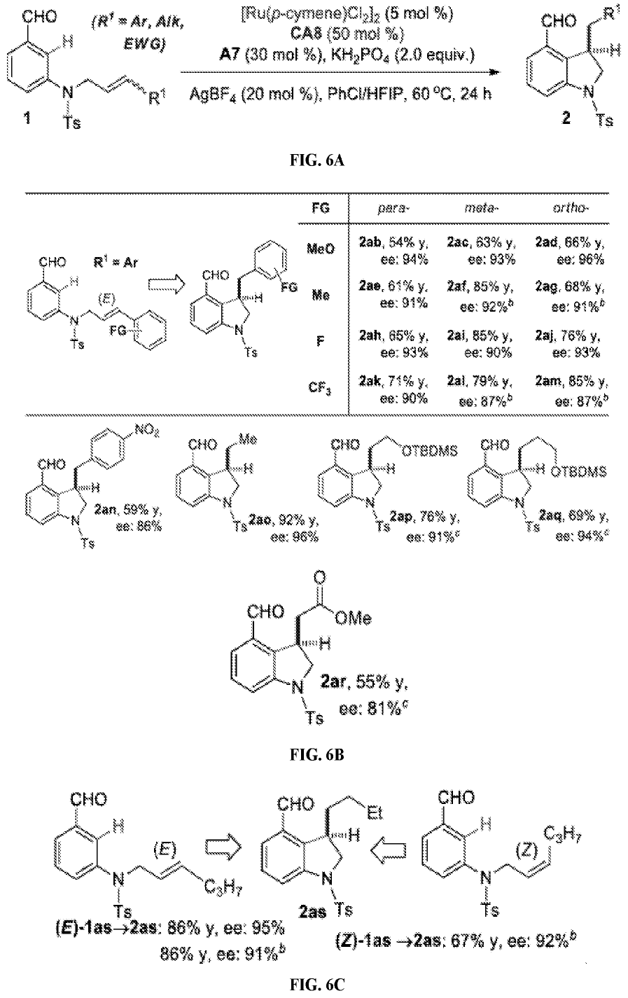

Also provided, in some embodiments, are indoline, chromane, isochromane, and/or 9-fluorene derivatives synthesized according to one or more of the methods disclosed herein. In some embodiments, the indoline derivatives include 4-formylindoline derivatives. In some embodiments, the indoline derivatives include one or more of the compounds shown in FIG. 5B, FIG. 6B, or a combination thereof. In some embodiments, the indoline derivatives include one or more of the following compounds:

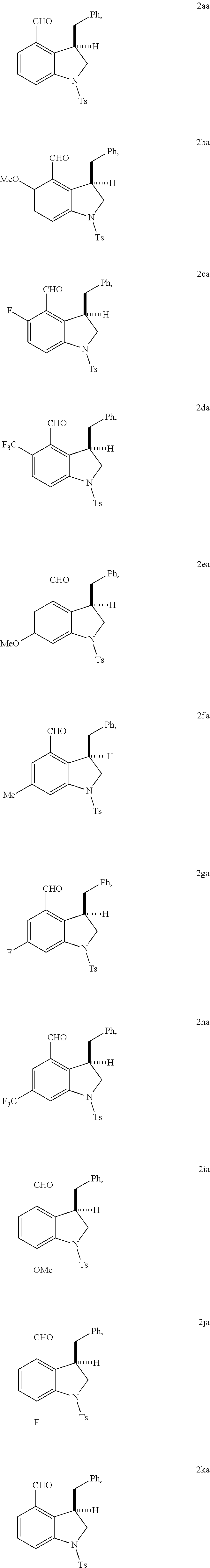

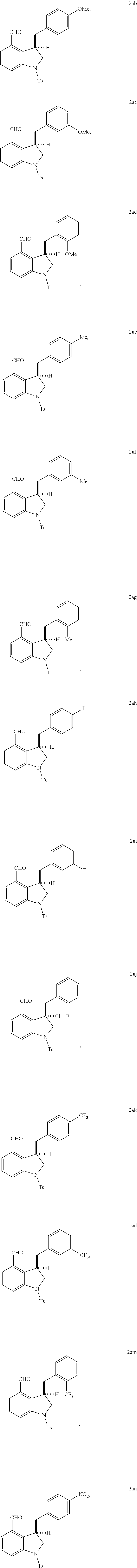

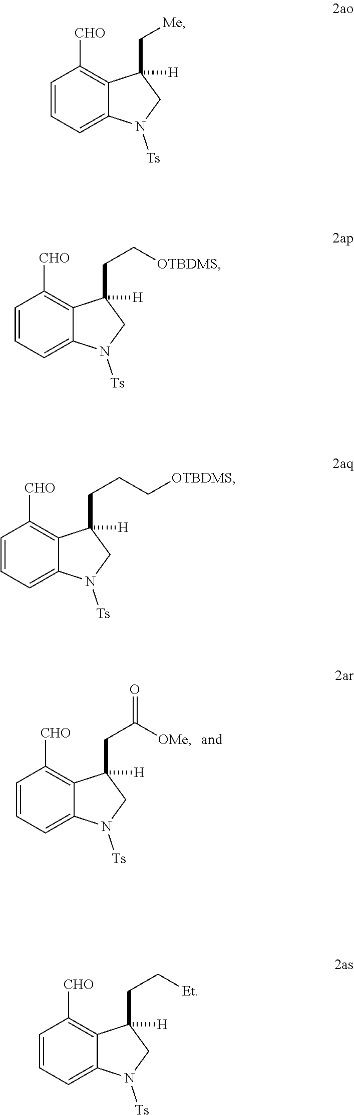 [SMILES: CC[C@H]1CN(S(=O)(=O)c2ccc(C)cc2)c2cccc(C=O)c21 ; Cc1ccc(S(=O)(=O)N2C[C@H](CCO[Si](C)(C)C(C)(C)C)c3c(C=O)cccc32)cc1 ; Cc1ccc(S(=O)(=O)N2C[C@H](CCCO[Si](C)(C)C(C)(C)C)c3c(C=O)cccc32)cc1 ; COC(=O)C[C@H]1CN(S(=O)(=O)c2ccc(C)cc2)c2cccc(C=O)c21 ; CCCC[C@H]1CN(S(=O)(=O)c2ccc(C)cc2)c2cccc(C=O)c21]

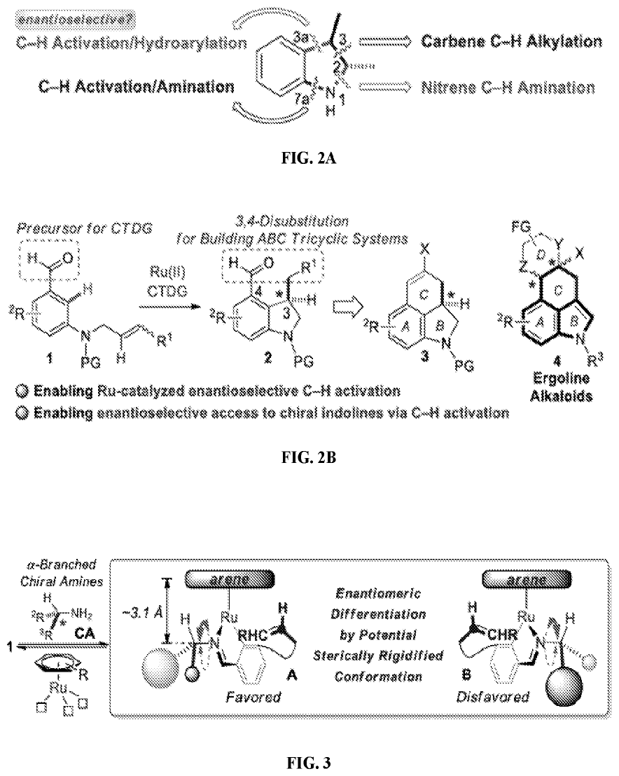

In some embodiments, the chromane derivatives include one or more of the compounds shown in FIG. 12B. In some embodiments, the indoline, chromane, isochromane, and/or 9-fluorene derivatives with chiral centers form intermediates for pharmaceuticals and biologically active reagents. Accordingly, further provided herein, in some embodiments, are pharmaceuticals and/or biologically active reagents formed from the derivatives synthesized herein. For example, in one embodiment, the pharmaceuticals and/or biologically active reagents include optically active tricyclic compounds formed from further transformation of the 4-formylindoline disclosed herein (FIGS. 2B, 10, 11). In another embodiment, the pharmaceuticals and/or biologically active reagents include ergot analogs synthesized through use of the optically active tricyclic compounds disclosed herein (FIG. 2B). In a further embodiment, the pharmaceuticals and/or biologically active reagents include tricyclic compounds and/or ergot analogs formed from the chromane, isochroman, and/or 9-fluorene derivatives disclosed herein.

The presently-disclosed subject matter is further illustrated by the following specific but non-limiting examples. The following examples may include compilations of data that are representative of data gathered at various times during the course of development and experimentation related to the presently-disclosed subject matter.

### EXAMPLES

### Example 1

This Example discusses development of Ru(II)-catalyzed enantioselective C—H activation/hydroarylation. This reaction demonstrated a highly enantioselective synthesis of indoline derivatives via catalytic C—H activation. Commercially available Ru(II) arene complexes and chiral α-methylamines were employed as highly enantioselective catalysts. Based on a sterically rigidified chiral transient directing group, multi-substituted indolines were produced in up to 92% yield with 96% ee. Further transformation of the resulting 4-formylindoline enabled access to an optically active tricyclic compound that is of potential biological and pharmaceutical interest.

Optically active indolines are important motifs in both synthetic and medicinal chemistry. C—H activation and functionalization have enabled approaches that are capable of constructing any of the four non-aromatic bonds (FIG. 2A). The enantioselective formation of C2-C3 and C2-N1 bonds can rely on carbene C—H alkylation and nitrene C—H amination, respectively. While the Pd(II)-catalyzed C—H activation was successful for making the C7a-N1 bond, which do not generate chirality, the hydroarylation via catalytic C—H activation provides a practical pathway to enantioselectively construct the C3-C3a bond. Nevertheless, this enantioselective synthesis of indolines has been underdeveloped, while enantioselective hydroarylation has been successfully developed for the syntheses of other related cyclic skeletons, especially with Rh(I)-catalyzed C—H activation systems by Bergman and Ellman. Recent disclosure of Ru(II) as effective catalysts for intramolecular hydroarylation encourages the development of the potential enantioselective processes. Considering the cost-effectiveness and the availability of the simple Ru(II) arene complexes, the present inventors sought to identify a suitable imine-based CTDGs strategy. Referring to FIG. 2B, starting with benzaldehyde 1, the resulting 3,4-disubstituted indolines 2 are particularly desirable as they allow for cyclization with the 4-formyl group, which produces tricyclic skeleton 3 that is relevant to the synthesis of ergot analogs (4).

Since only monodentate TDGs may work with Ru(II) arene catalysts, it was envisioned that chiral α-branched amines CA with an α-hydrogen would be adaptable (FIG. 3). Suggested by the measurement on a known structure, the distance between the nearly parallel C—N bond and arene unit in a proposed key intermediate A may be ˜3.1 Å. Without wishing to be bound by theory, it was believed that the limited distance would restrict the free rotation of the C—N bond in the postulated intermediates A and B, with the hydrogen briefly facing the top arene. This amine-arene rigidified conformation would make a stationary arrangement of the big and small substituents on two sides of the ruthenacycle. During the enantio-determining insertion step, the alkene would approach with the less substituted side toward the top arene, and the double bond would approach from the less hindered side of the stereogenic carbon center to form the favored enantiomer.

Further thereto, reported herein for what is believed to be the first time is an Ru-catalyzed enantioselective C—H activation/hydroarylation reaction and its synthetic application for an ergot alkaloid-relevant tricyclic structure. This reaction enables a highly enantioselective synthesis of indolines via catalytic C—H activation. Readily available Ru(II) complexes and chiral α-methylamines are employed as catalysts and afforded various chiral 4-formylindolines in up to 96% ee.

Initial efforts focused on the hydroarylation of m-amidobenzaldehyde 1aa with [Ru(p-cymene)Cl₂]₂ and AgBF₄ in 1,2-dichloroethane (DCE) (FIG. 4A). Acetic acid (5 equiv) was used for accelerating both the C—H activation and the reversible imine formation. At 70° C., relatively rigid chiral cyclic amine CA1 afforded indoline 2aa in 35% yield and 17% ee (FIG. 4B, entry 1). Chiral amines CA2 and CA3 were shown ineffective, presumably due to additional coordination by the OH and NH units (FIG. 4B, entries 2 and 3). With a protected NH (CA4), the reaction occurred with 33% ee, however, in low yield (FIG. 4B, entry 4). Productive reactions were observed with α-methylbenzylamine analogs CA5-CA9, and a general trend in enantiocontrol has emerged (FIG. 4B, entries 5-9). Increased steric hindrance at the ortho-position of the phenyl, presumably orienting toward the reaction center, led to increases in enantioselectivity. Notably, all (R)-amines gave the same sense of asymmetric induction, which is consistent with the proposed model (FIG. 3).

Toluene as the solvent was later found to offer higher enantiocontrol but decreased yield with chiral amine CA8 (FIG. 4C, entry 10). A mixed solvent system with toluene and hexafluoroisopropanol (HIP) turned out to be effective and allowed for KH₂PO₄ as the proton source. This condition requires only catalytic amount of the carboxylic acid, thus making the employment of some functionalized acids practical. A 30 mol % of bulky acids A1 and A2 effectively increased both yield and ee, respectively (FIG. 4C, entries 11 and 12). Examination of other N-protected amino acids (FIG. 4C, entries 13-19) afforded 2aa in 76% yield and 84% ee (FIG. 4C, entry 17). With protected L-tert-leucine (A7), further optimization resulted in 88% yield of 2aa in 94% ee in PhCl/HTIP solvent at 60° C. (FIG. 4C, entries 20 and 21). Notably, the sense of the chiral induction is dominantly determined by the chiral amine, as evidenced by the resulting −85% ee with ent-CA8 and A7 (FIG. 4C, entry 22).

Based on the postulated model of enantiocontrol, the sterics of the arene ligands would have impacts on enantiocontrol. A clear trend showed that increasing steric bulkiness on the arene led to improved enantioselectivity (FIG. 4D, entries 23-26), although trisubstituted arene ligands deactivated the catalysts (FIG. 4D, entries 27 and 28).

Under the optimized conditions, various substituents on the benzaldehyde unit of 1 were studied (FIGS. 5A-B). The R configuration of 2aa was confirmed by single-crystal X-ray diffraction. The ortho-substituents to the aldehyde would have significant influences both sterically and electronically. As demonstrated by 2ba-2da with MeO—, F—, CF₃— groups, respectively, electronically and sterically different ortho-functional groups were well tolerated at slightly elevated temperature. The meta-substituents, being para- to the reacting C—H bond, would electronically influence both C—H activation and insertion steps. Remarkably, 1 with electron-donating and withdrawing, alkyl, and halogen groups were all fruitfully transformed to indoline 2ea-2ha with up to 94% ee. Moreover, when a methoxy group and a fluorine atom were located on the para-position, catalytic reactions were also performed smoothly and enantioselectively (2ia and 2ja). Moreover, an N-nosyl group was successfully tolerated, as demonstrated by the production of 2ka in 74% yield and 92% ee.

Subsequent efforts went on with amidobenzaldehyde 1 bearing different types of internal alkene units (FIGS. 6A-B). Respectively, (E)-styrenyl groups containing MeO—, Me-, F—, CF₃—, NO₂— groups at all possible positions were systematically investigated and afforded the corresponding indoline 2 in up to 85% yields with ee values mostly above 90% (2ab-2an). Aliphatic alkenyl groups were also effective substituents for producing 2ao-2aq in good yields with up to 96% ee. Remarkably, the catalytic system was successful with electron-deficient alkene units, as exemplified by the formation of 2ar from the corresponding acrylate-containing benzaldehyde. Additionally, the performances of the E and Z isomers were compared (FIG. 6C). At 70° C., (E)-1as produced the same product 2as in higher yield than (Z)-1as, while their ee values were almost the same, indicating the configuration of the internal alkene was not decisive for the enantiocontrol. Finally, slightly lower temperature for the reaction of (E)-1as produced indoline 2as with 95% ee.

For probing the mechanism, H/D exchange reactions were carried out with the racemic form of amine CA5 in DCE. Reversible H/D exchange did not occur at 30° C. (FIG. 7, 1). In contrast, at 40° C., significant H/D exchange was observed in both the product and recovered 1aa (FIG. 7, 2). Moreover, a control reaction without amines gave neither the product 2aa nor detectable C—H activation (FIG. 7, 3).

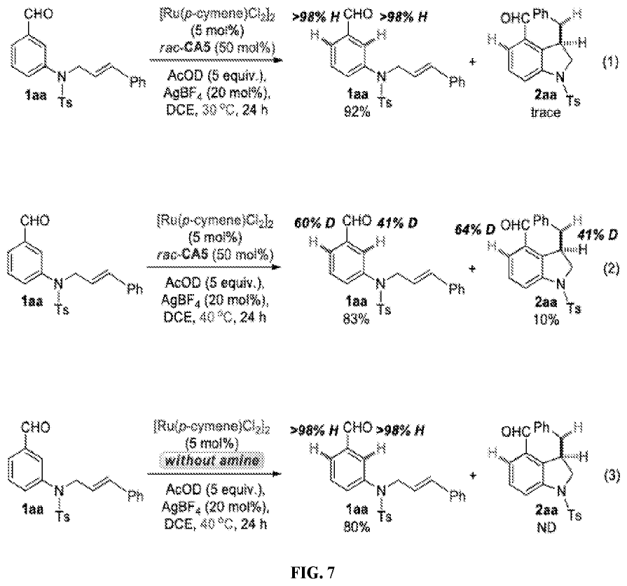

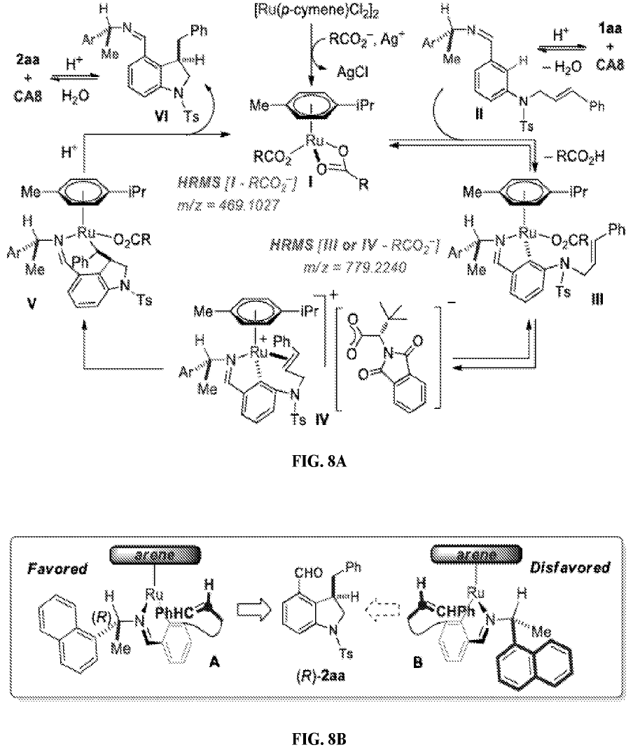

Based on data above and results from an existing study, a proposed mechanism is formulated to begin with a reversible Ru(II)-based C—H activation of the transient imine intermediate II in acidic media, forming ruthenacycle III (FIG. 8A). Besides assisting the metalation/deprotonation step, the bulky carboxylate presumably formed an ion pair with the cationic part of the intermediate IV, which would count for their observed impact on the enantiomeric control. HRMS study on the reaction system indicated a major species matching the intermediate I without a carboxylate anion, and another major species matching the cationic part of either intermediate III or IV (see Appendix A for details). Based on the postulated asymmetric induction model, the alkene unit should prefer to approach the Ru center from the same side of the conformationally rigidified methyl group on the chiral carbon (FIG. 8B). Notably, the proposed model leads to the R configuration of the chiral center in 2aa, which is in accord with the observation from its single crystal.

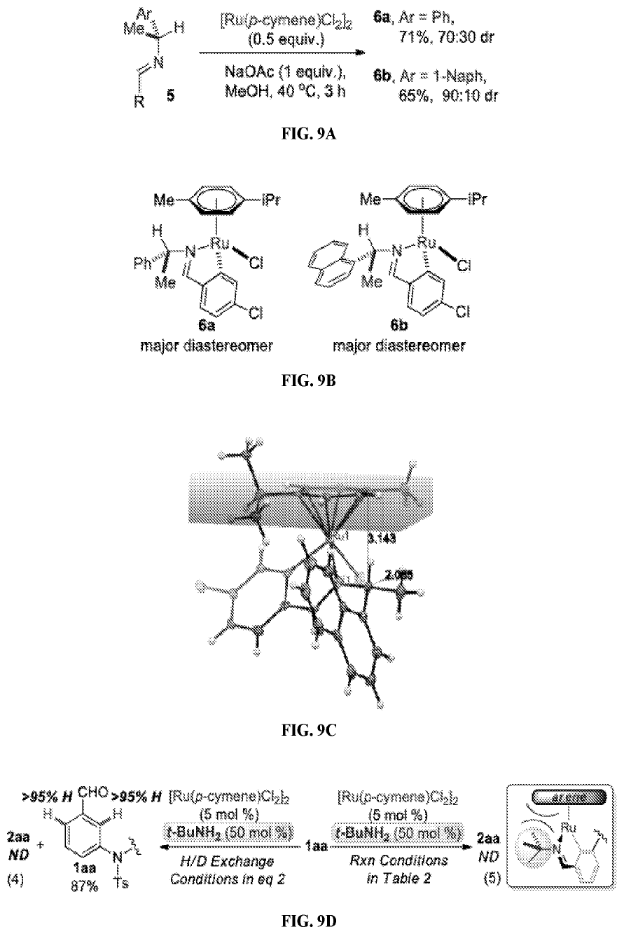

For better understand the asymmetric induction, chiral imine 5 was converted to ruthenacycles 6a and 6b as simplified models to the key intermediate III (FIGS. 9A-B). The single crystal structure of 6b confirmed the α-hydrogen of the chiral amine moiety indeed faced the top arene (FIG. 9C). The perpendicular distance from the chiral carbon to the arene plane appears to be 3.143 Å, while the distance from the chiral carbon to the hydrogen center of the α-methyl group is 2.065 Å. The comparison suggests even a CH₃— group may sterically restrict the rotational freedom of the C—N bond. Consistently, both H/D exchange (FIG. 9D, 4) and catalytic reactions (FIG. 9D, 5) employing tert-butylamine resulted in barely any H/D exchange and no indoline product.

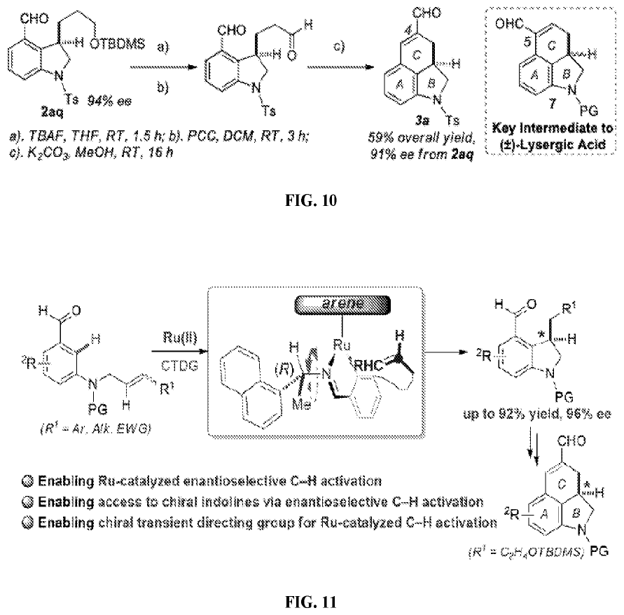

Chiral indolines serve as important precursors for constructing complex structures. ABC tricyclic aldehyde 7 has been a key intermediate for building the D ring in the total synthesis of (±)-lysergic acid (FIG. 10). Terminal group modification of indoline 2aq followed by an aldol condensation with the 4-formyl group afforded tricyclic 3a in 91% ee. In contrast to the 5-formyl group in 7, 3a would open potential asymmetric access to new non-naturally occurring ergot analogs.

In summary, Ru-catalyzed enantioselective C—H activation/hydroarylation reaction has been developed for the first time. The cooperation of the α-methyl chiral amine has enabled an effective application of enantioselective C—H activation for synthesis of indoline derivatives. The new system features practicality with the employment of the commercially available and cost-effective Ru(II) complex and chiral amine. This method provides opportunities for the enantioselective access to various indoline-based bicyclic and polycyclic structures (FIG. 11). More broadly, the process brought in a new tool that would stimulate further exploration of enantioselective C—H functionalization reactions.

### Example 2

This Example describes the synthesis of chromane derivatives utilizing the same general method discussed in Example 1 for the formation of indoline derivatives. More specifically, as shown in FIG. 12A, the method for forming indoline derivatives may be used to form chromane derivatives by selecting a different precursor compound. In particular, by replacing the N in the precursor compound of the indoline synthesis with O, and moving the carbon-carbon double bond preceding R¹, the same general method can be used to form a cyclic compound where the 5-membered N containing ring of indoline is replaced by the 6-membered O containing ring of chromane.

Using the reaction conditions shown in FIG. 12A, this method of synthesizing chromane derivatives produced multisubstituted chromanes in up to 82% yield with 93% ee. Additionally, by varying the substituents at R¹ and R² the method provided the various chromane derivatives shown in FIG. 12B.

### Example 3

### 1. General

Experimental: Unless otherwise noted, all solvents were dried with sodium benzophenone and distilled before use. All reactions were set up under N2 atmosphere utilizing glassware that was flame-dried and cooled under vacuum. All non-aqueous manipulations were using standard Schlenk techniques. Reactions were monitored using thin-layer chromatography (TLC) on Silica Gel plates. Visualization of the developed plates was performed under UV light (254 nm) or KMnO4 stain. Silica-gel flash column chromatography was performed on SYNTHWARE 40-63 m silica gel.

Materials: Unless otherwise indicated, starting catalysts and materials were obtained from Sigma Aldrich, Oakwood, Strem, or Acros Co. Ltd. Moreover, commercially available reagents were used without additional purification.

Instrumentation: NMR spectra were recorded at 500 MHz (¹H NMR) and 125 MHz (¹³C NMR) using TMS as an internal standard. Chemical shifts are given relative to TMS or CDCl3 (0 ppm for ¹H NMR, 77.16 ppm for ¹³C NMR). Data are represented as follows: chemical shift (multiplicity, coupling constant (s) in Hz, integration). Multiplicities are denoted as follows: br=broad, s=singlet, d=doublet, t=triplet, q=quartet, m=multiplet. Mass spectroscopy data of the products were collected on an HRMS-TOF instrument using APCI ionization.

### 2. Preparation of Substrate

### Procedure 1. The synthesis of N-(3-formylphenyl)-4-methylbenzenesulfonamide (S1)

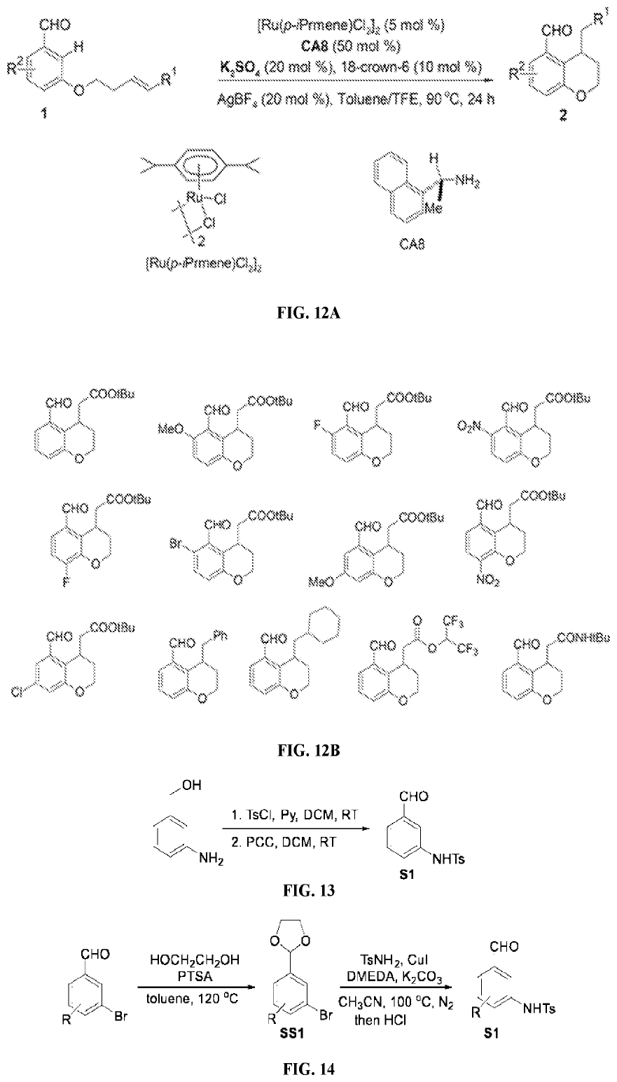

Method A¹ (FIG. 13):

A 50 mL one-neck round flask equipped with a magnetic stirring bar was charged with a solution of 3-aminobenzyl alcohol (4.06 mmol) in DCM (20 mL). Pyridine (0.4 mL) and TsCl (1.1 equiv.) were added, and the mixture was stirred at room temperature for 12 h. After that, the solvent was removed through evaporation in vacuo. The residue was dissolved by DCM (20 mL) and followed by adding PCC (1.1 equiv.) and stirred at room temperature for 5 h. The reaction mixture was filtered through silica and purified by flash chromatography (ethyl acetate hexanes=1:3).

Method B² (FIG. 14):

A 25 mL one-neck round flask equipped with a magnetic stirring bar was added 3-bromobenzaldehyde (3 mmol) and ethane-1,2-diol (3.0 g, 15 equiv.) in toluene (8 mL). TsOH (2 mol %) was added at room temperature, and then the reaction mixture was allowed to stirred at 120° C. for 10 h. The reaction was brought to room temperature, and quenched by adding H₂O (20 mL) and EtOAc (15×2 mL), dried over anhydrous Na₂SO₄, filtration and concentration of solvent afforded 2-(3-bromophenyl)-1,3-dioxolanes (SS1). To a 25 mL Schlenk tube were added SS1 (3 mmol), CuI (50 mol %), TsNH2 (1.2 equiv.), and K₂CO₃ (3.0 equiv.). The mixture was then evacuated and backfilled with nitrogen for three times. After that, DMEDA (1.0 equiv.) and MeCN (6 mL) were added subsequently. After stirring at 100° C. for 12 h, the reaction mixture was cooled to room temperature. The reaction was quenched by diluted HCl and the desired product (S1) was purified by flash chromatography (ethyl acetate:hexanes=1:3).

### Procedure 2. The Synthesis of Allyl Bromide (S2)

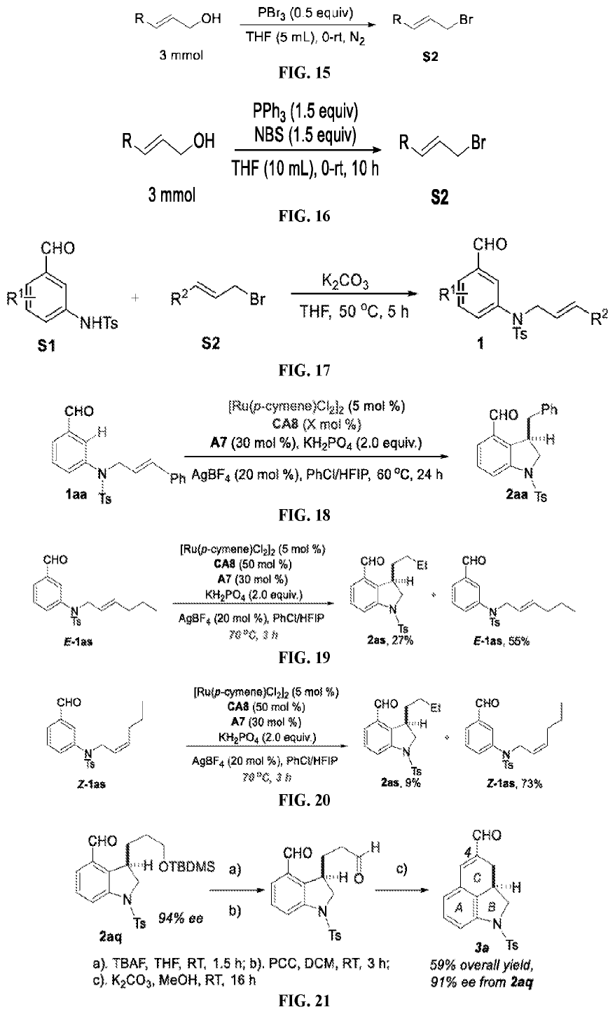

Method C³ (FIG. 15):

To a 25 mL Schlenk flask equipped with a stirring bar was added allyl alcohol (3 mmol). The mixture was then evacuated and backfilled with nitrogen for three times. After that, THE (5 mL) and PBr₃ (0.5 equiv.) were added by syringe at 0° C. The mixture was then stirred at room temperature for another 2 h. the reaction was quenched by adding H₂O and saturated NaHCO₃, extracted by ethyl acetate. After removing all of the solvent, the product was used for the next step directly without any purification.

Method D⁴ (FIG. 16):

To a 25 mL one-neck round flask equipped with stirring bar were added allyl alcohol (3 mmol), PPh₃ (1.5 equiv.) and THE (10 mL). The mixture was stirred at 0° C. for 20 min, then followed by adding NBS (1.5 equiv.) in three portion. After stirring at room temperature for 10 h, the reaction was quenched by adding hexanes. The residue was filtered through silica, and washed by hexanes. After removing all of the solvent, the product was used for the next step directly without any purification.

### Procedure 3. The Synthesis of Substrate 1 (FIG. 17)

To a 50 mL one-neck round flask equipped with a stirring bar was added S1 (2 mmol) and K2CO3 (5 mmol) in THE (10 mL), followed by allyl bromide (2.4 mmol). The mixture was stirred at 50° C. for 5 h. The reaction was quenched by adding H2O (20 mL) and EtOAc (20 mL). Dried over anhydrous Na2SO4, filtration and removed all of organic solvent. The residue was purified by flash chromatography (ethyl acetate:hexanes=1:5 to 1:3) to get substrates 1.

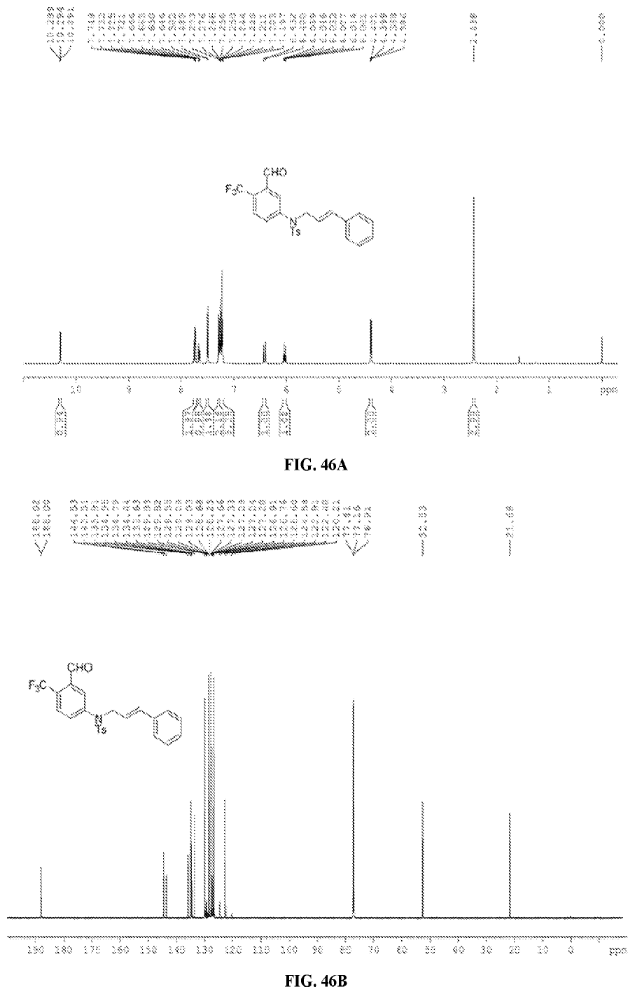

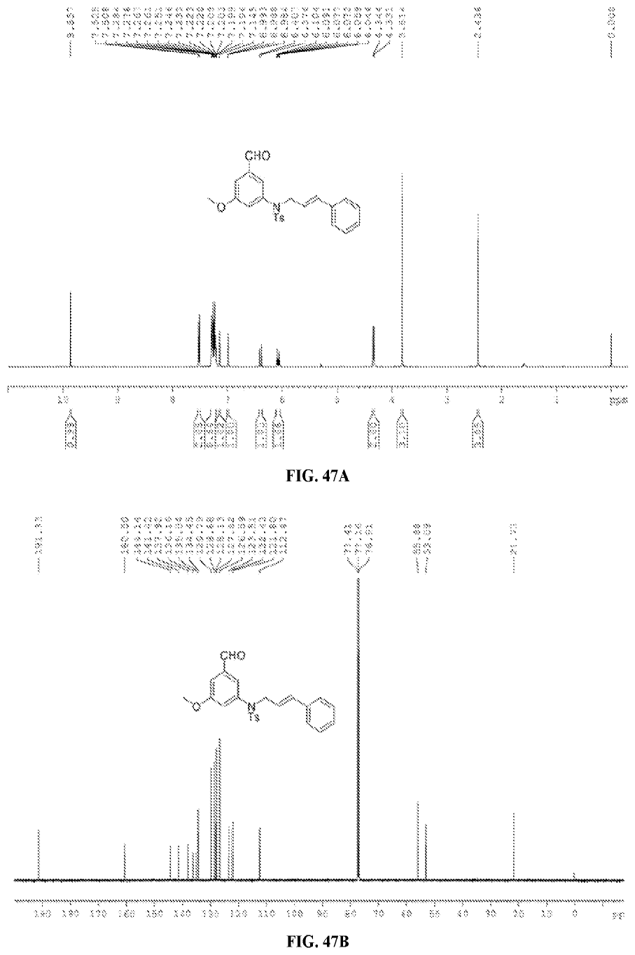

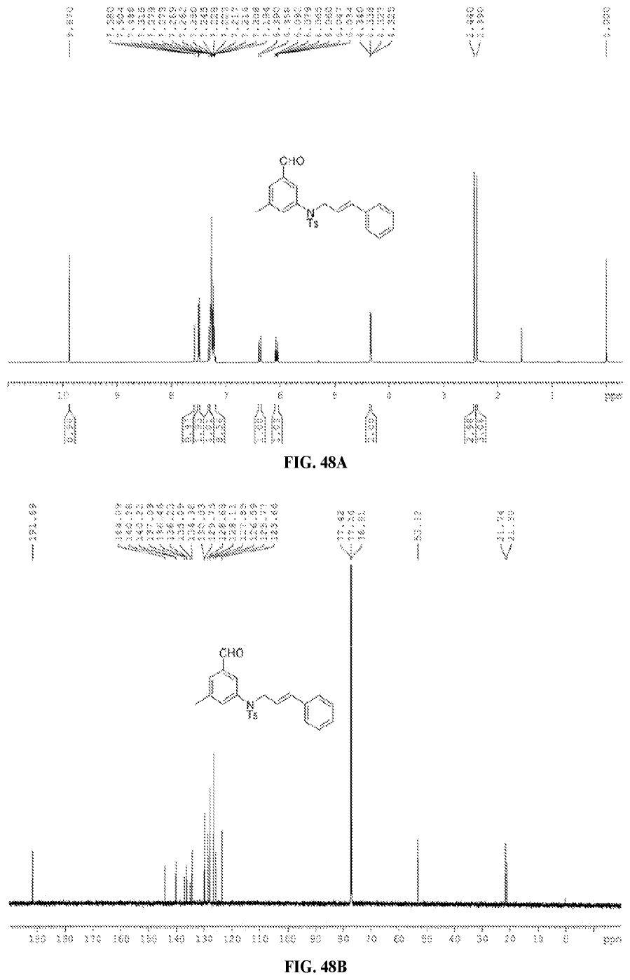

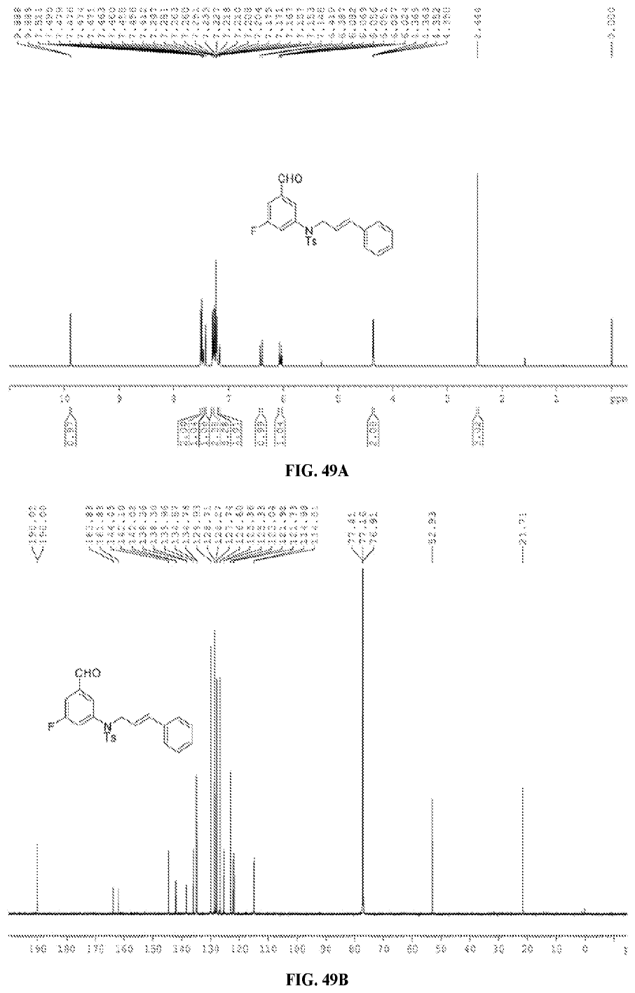

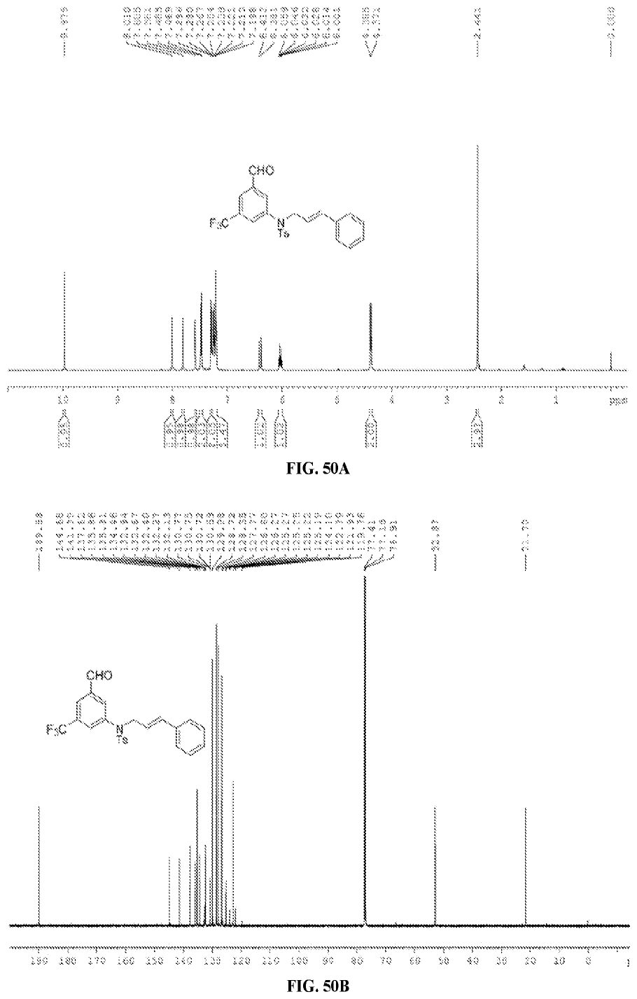

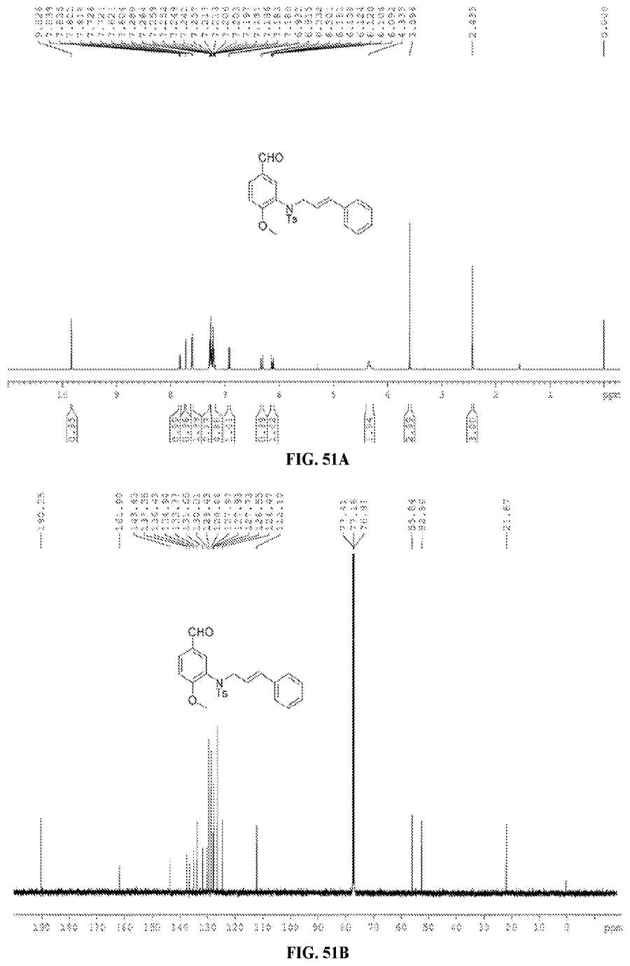

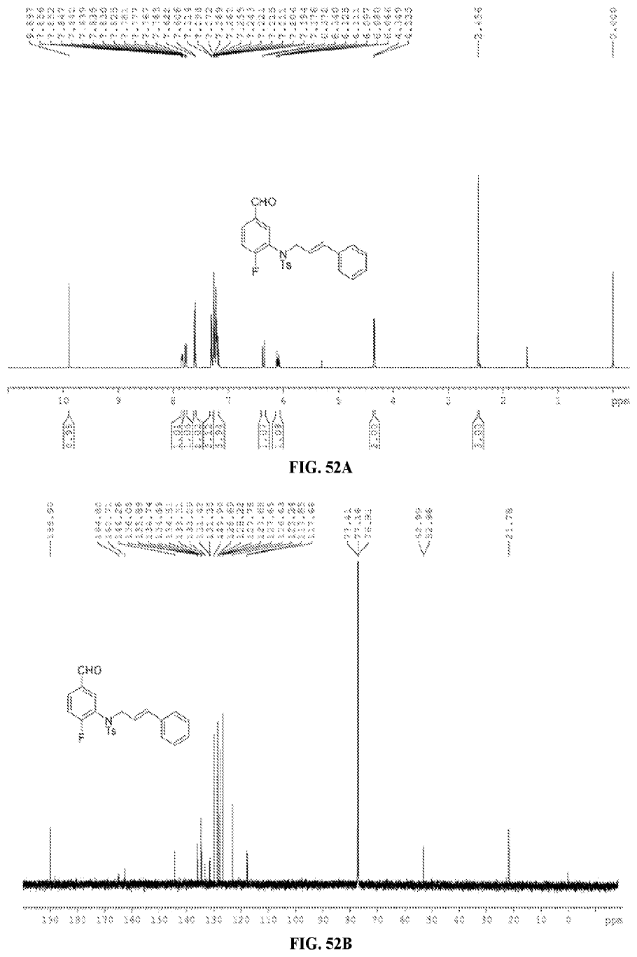

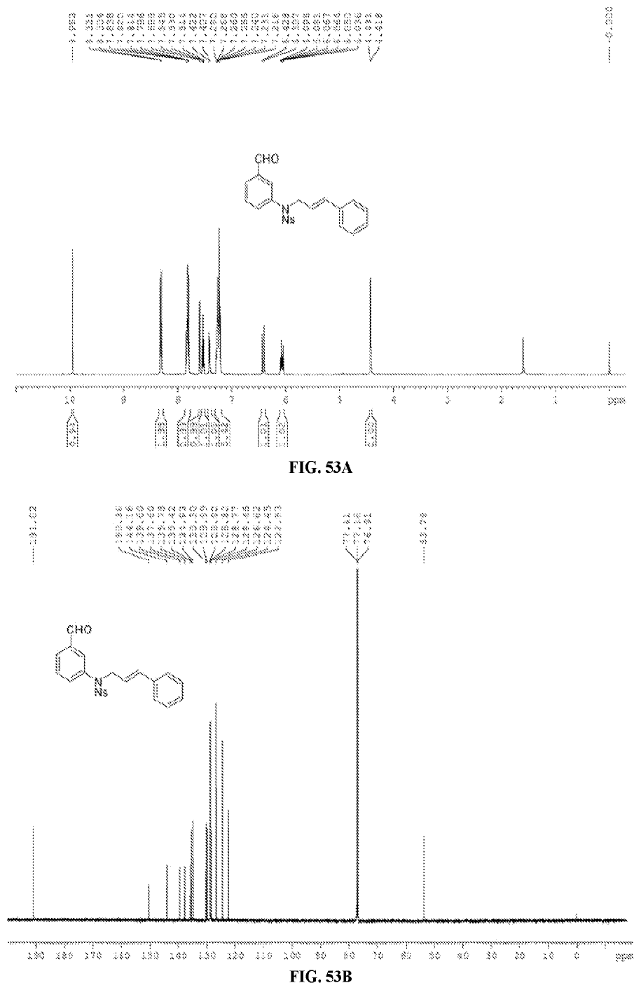

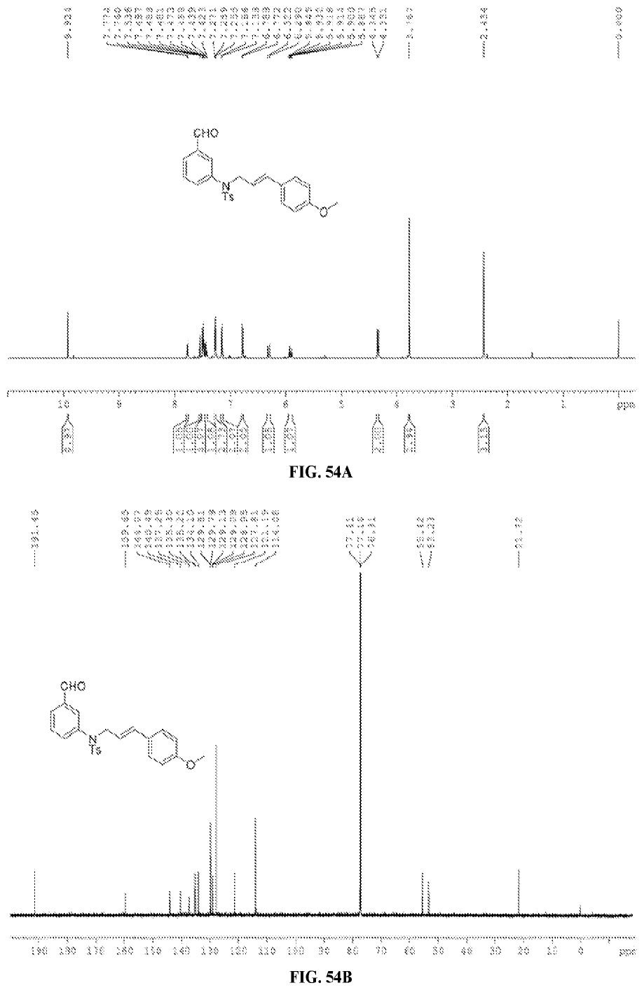

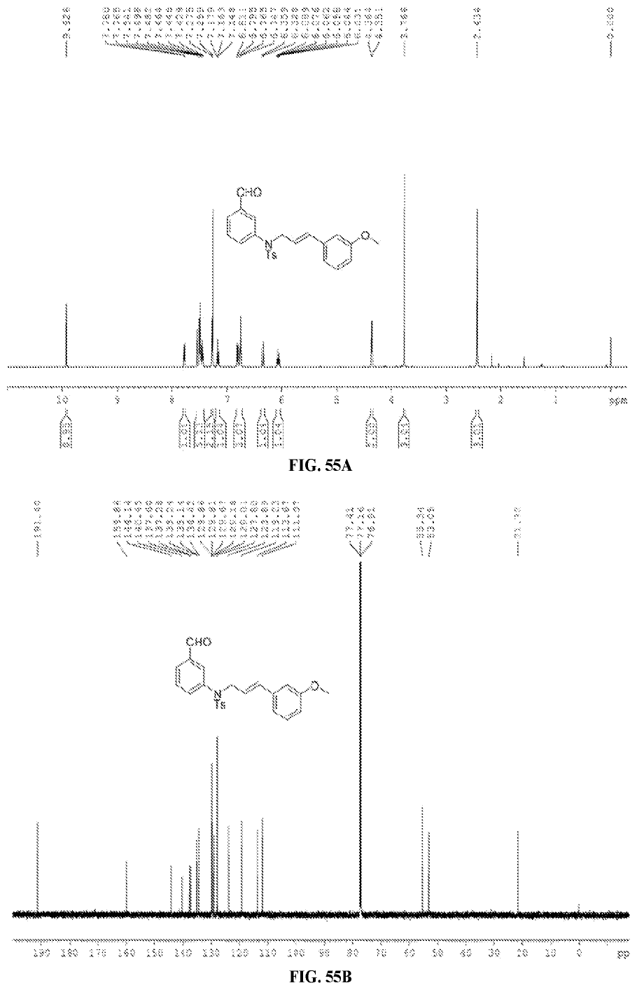

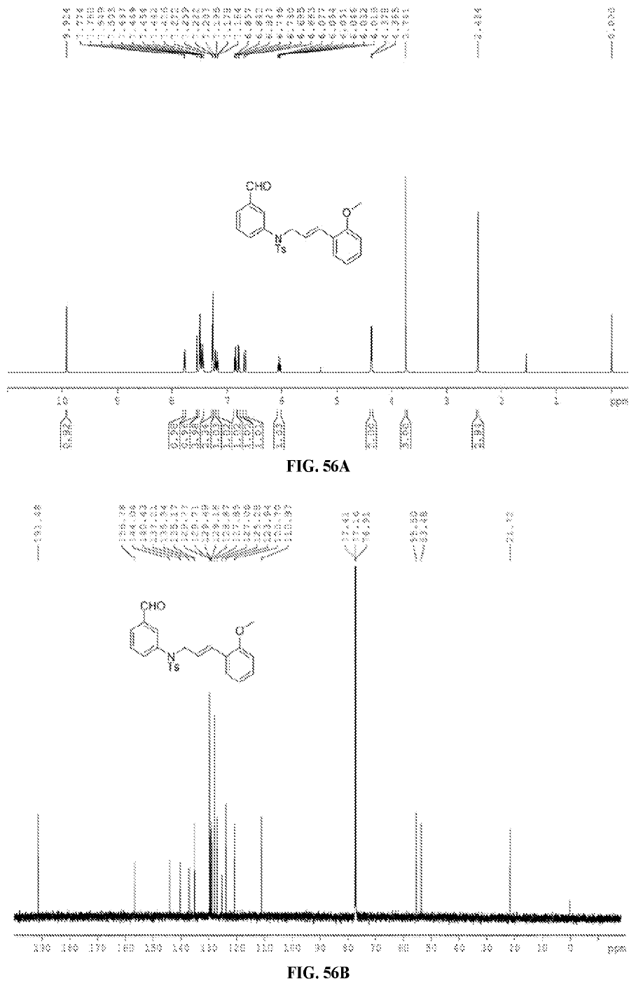

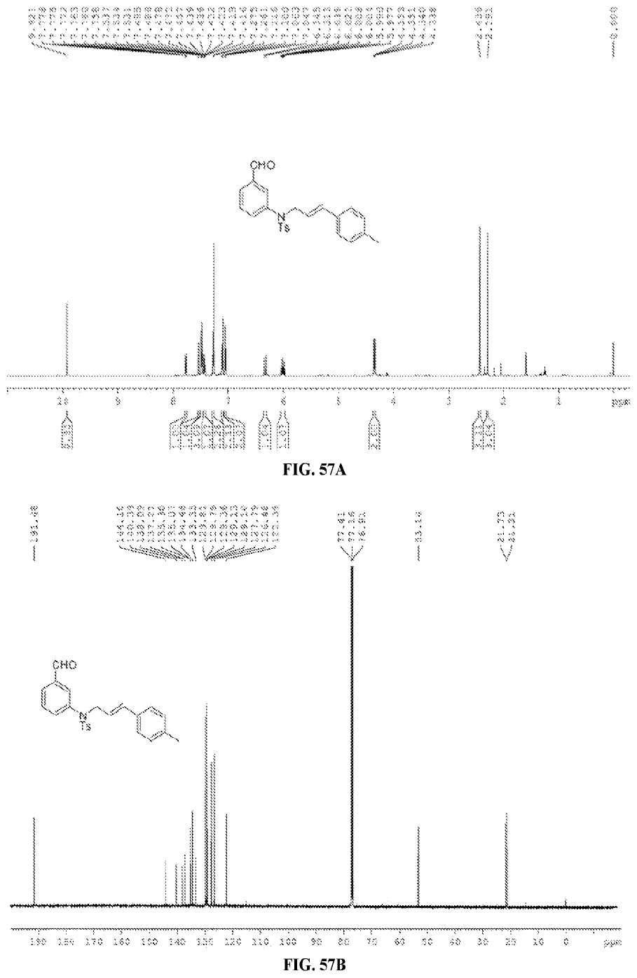

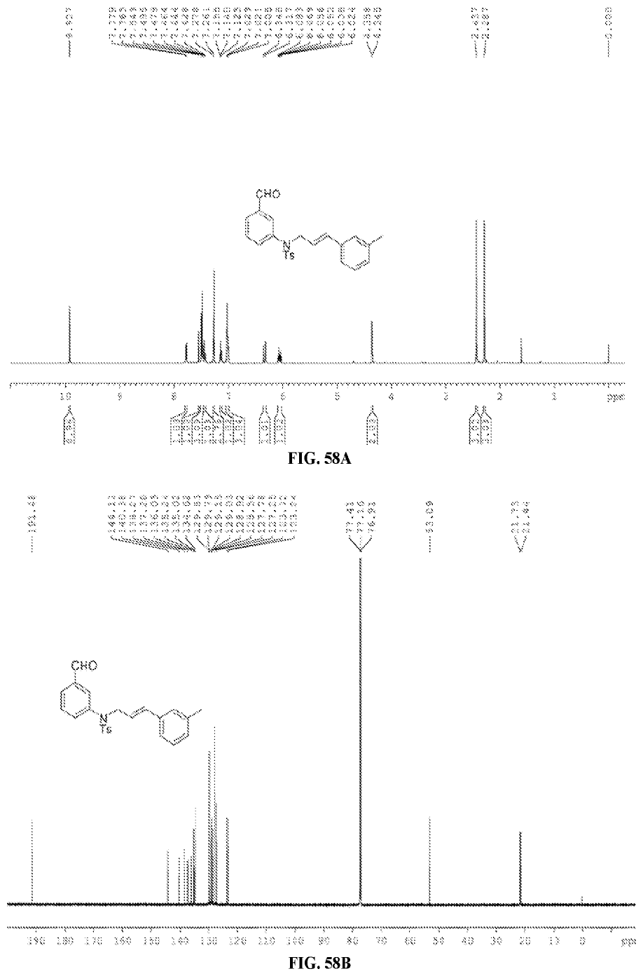

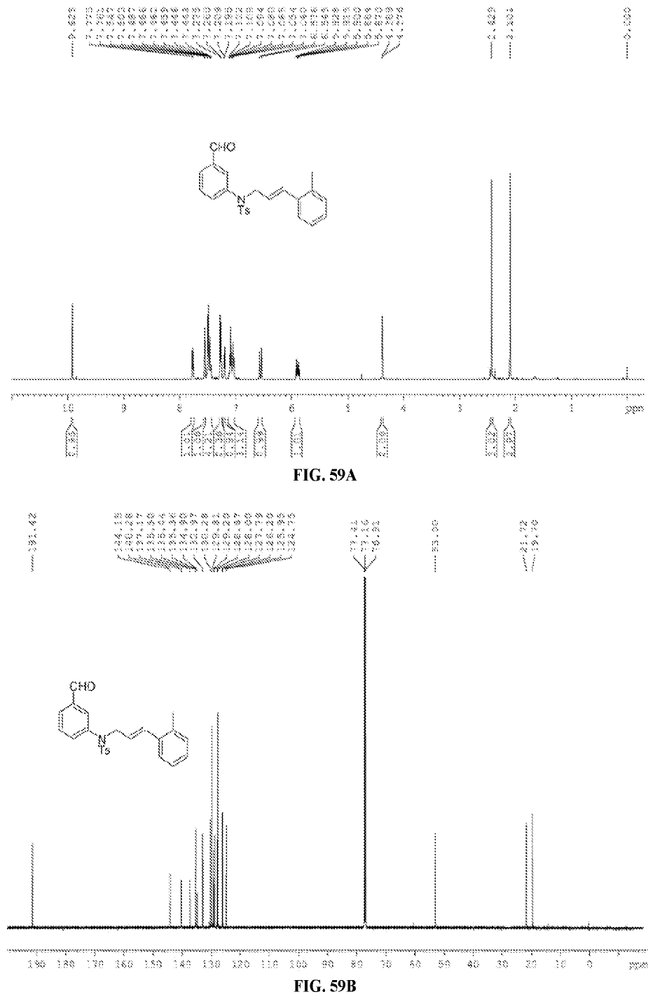

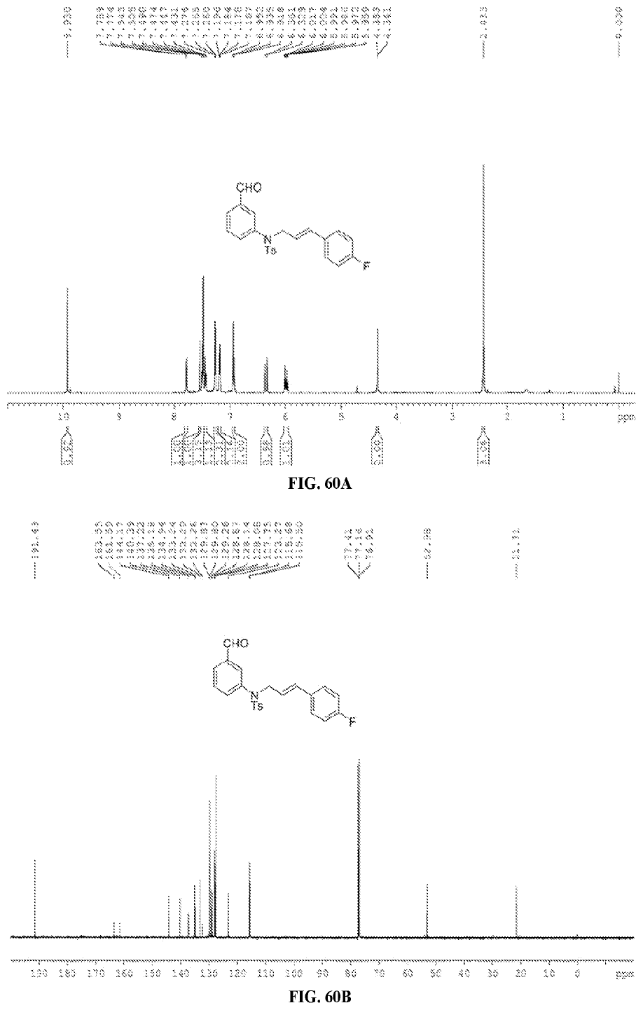

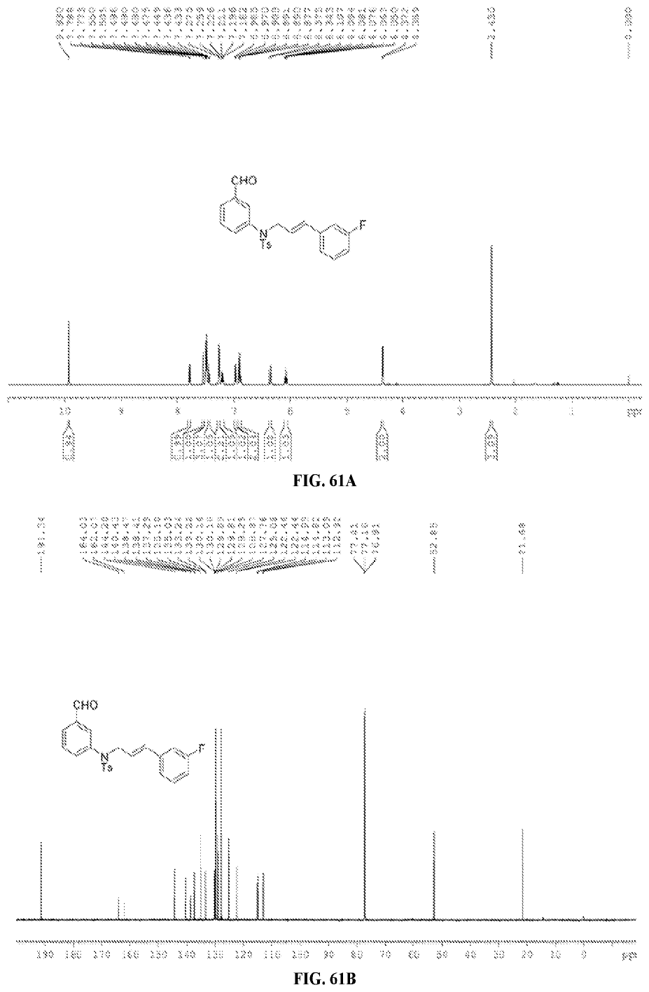

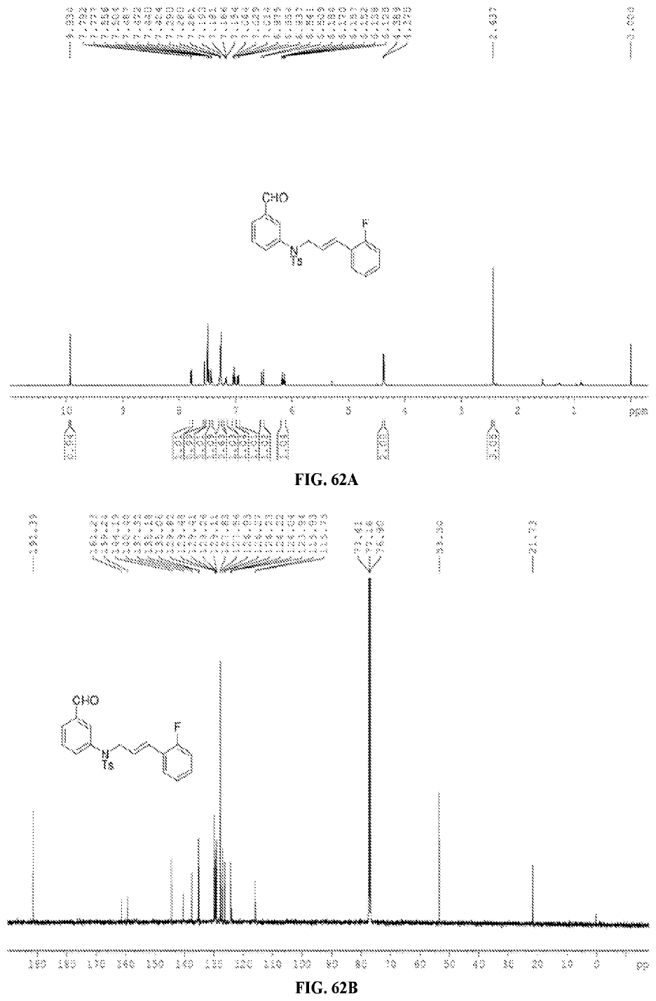

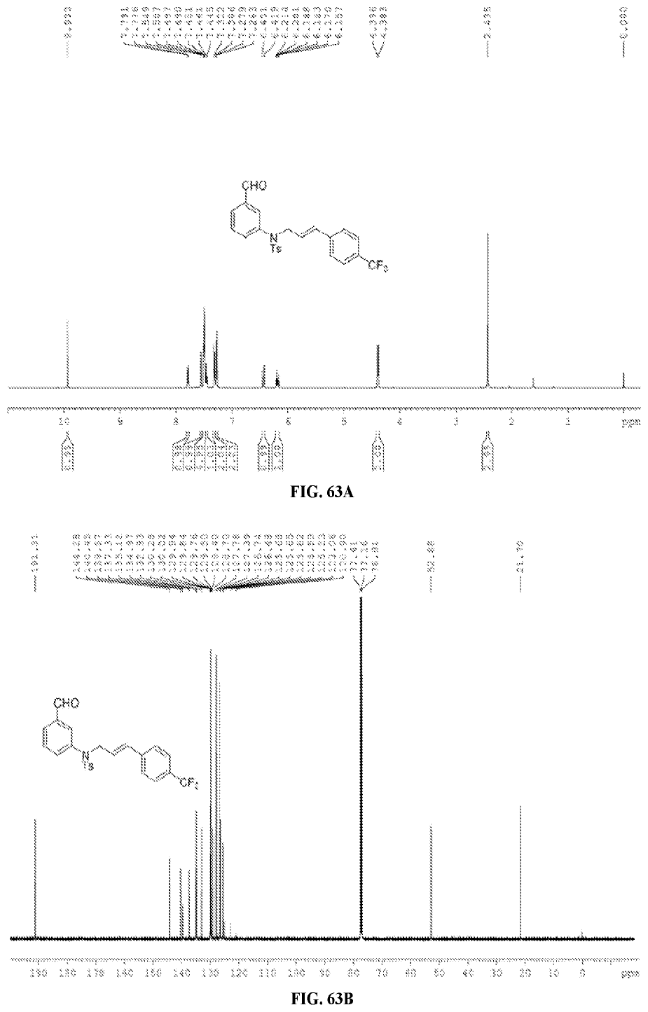

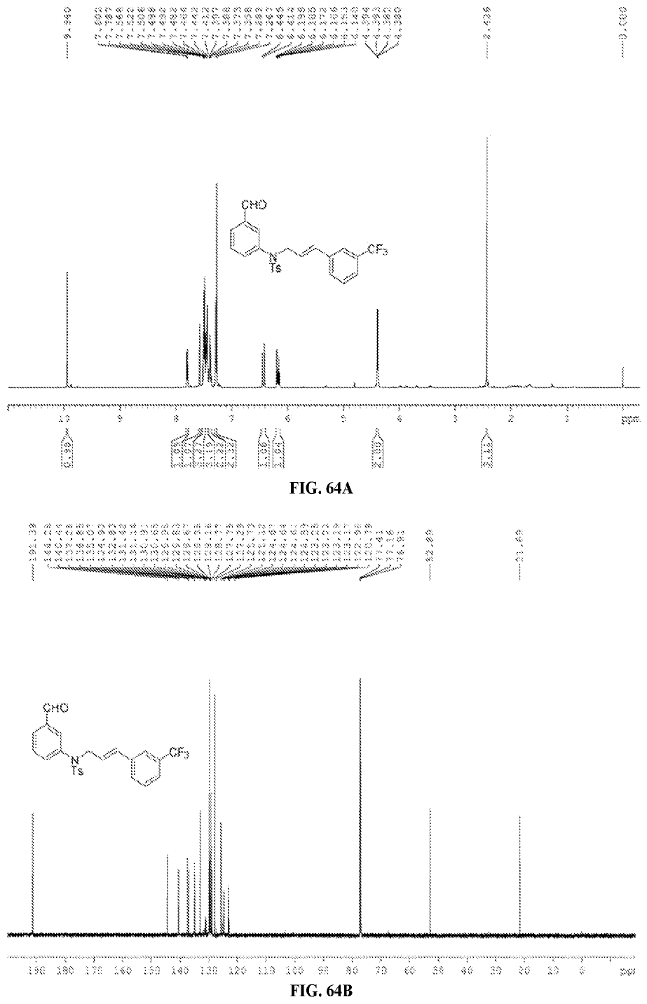

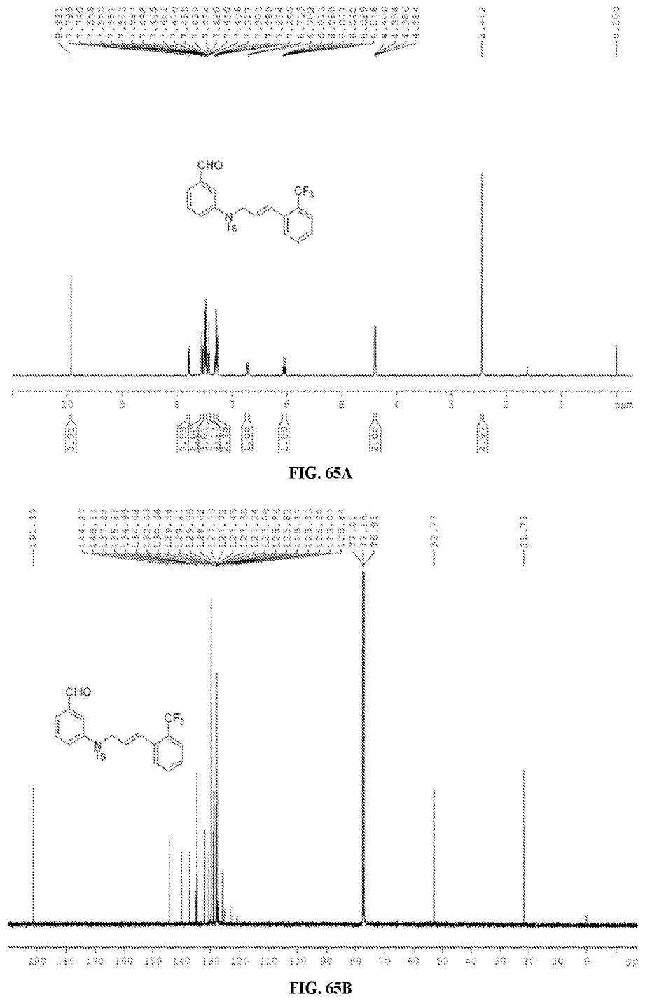

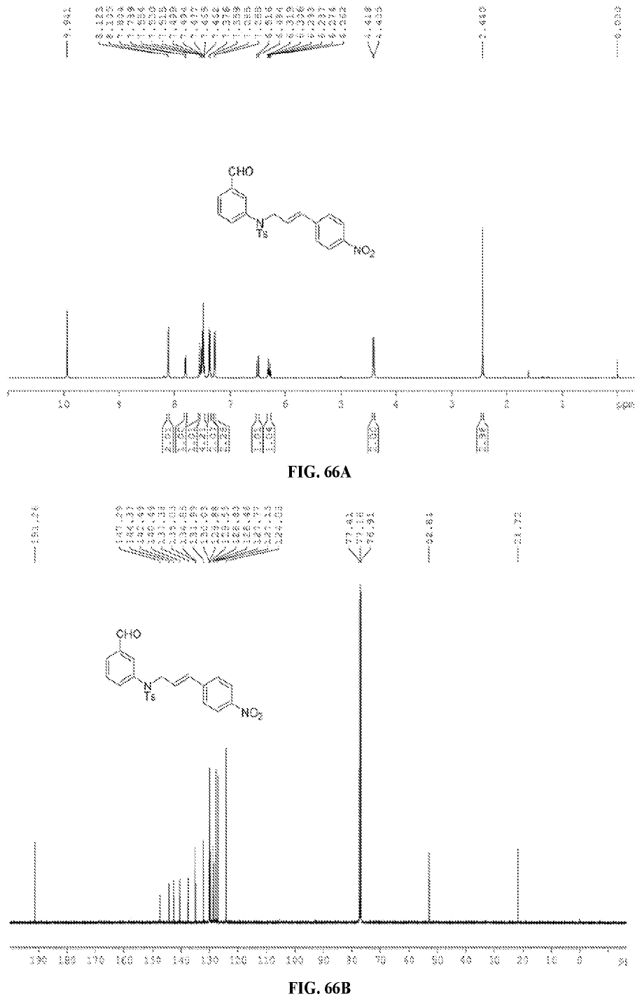

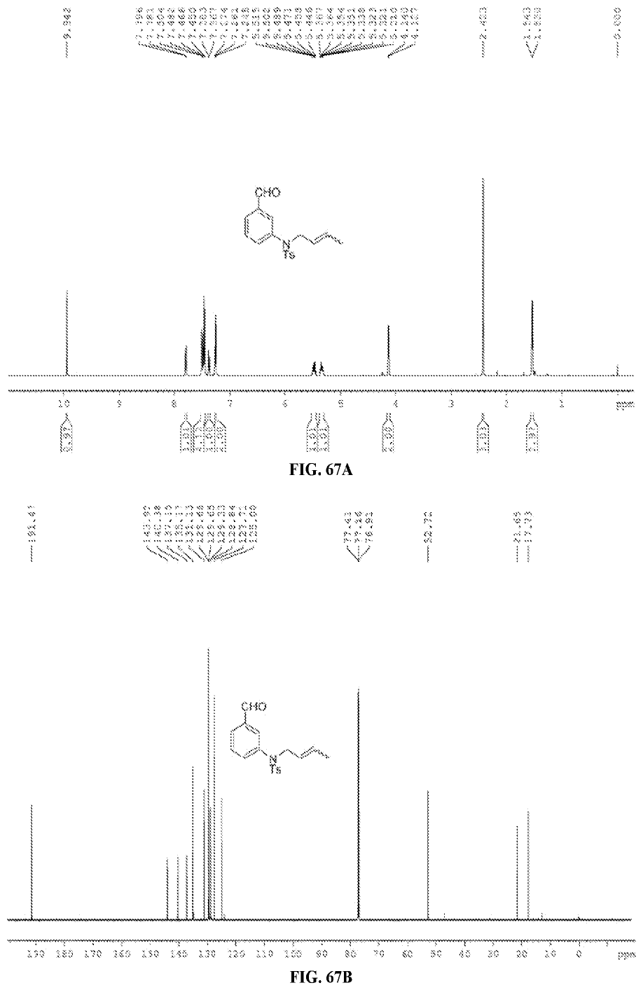

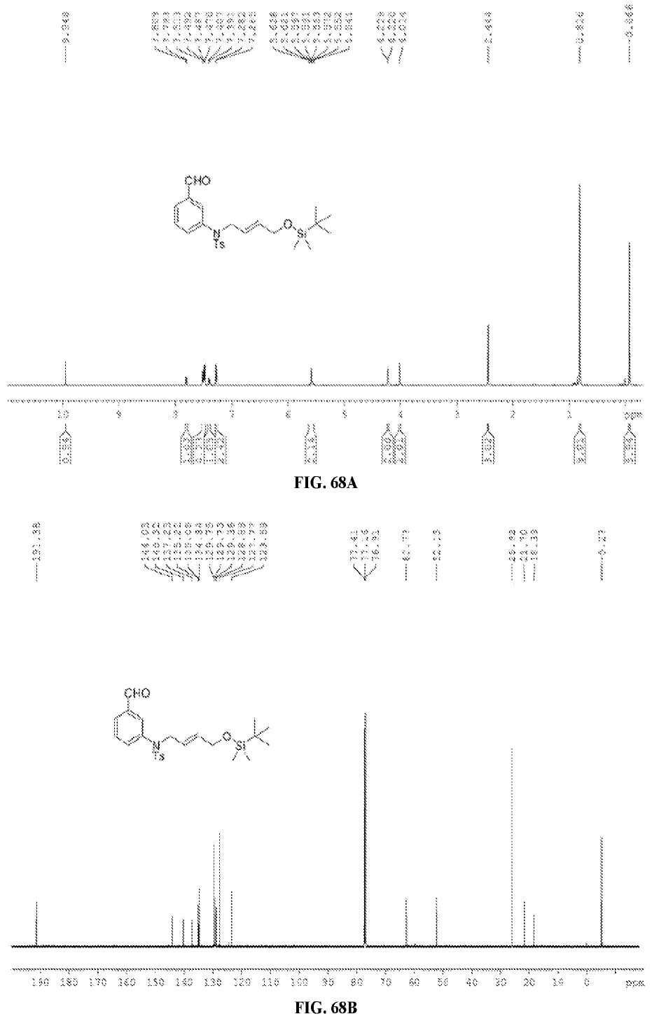

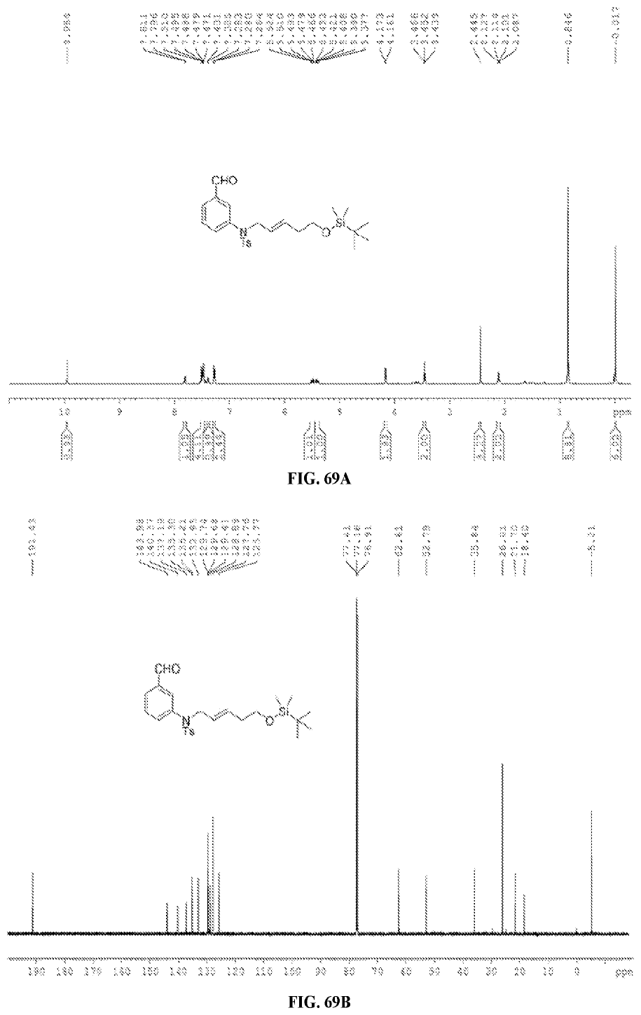

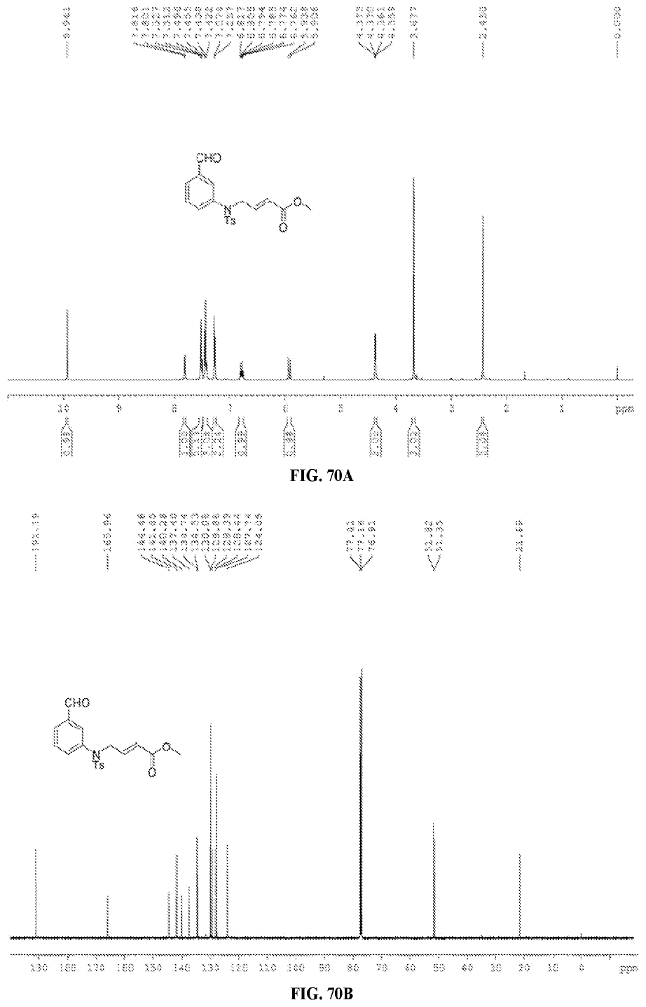

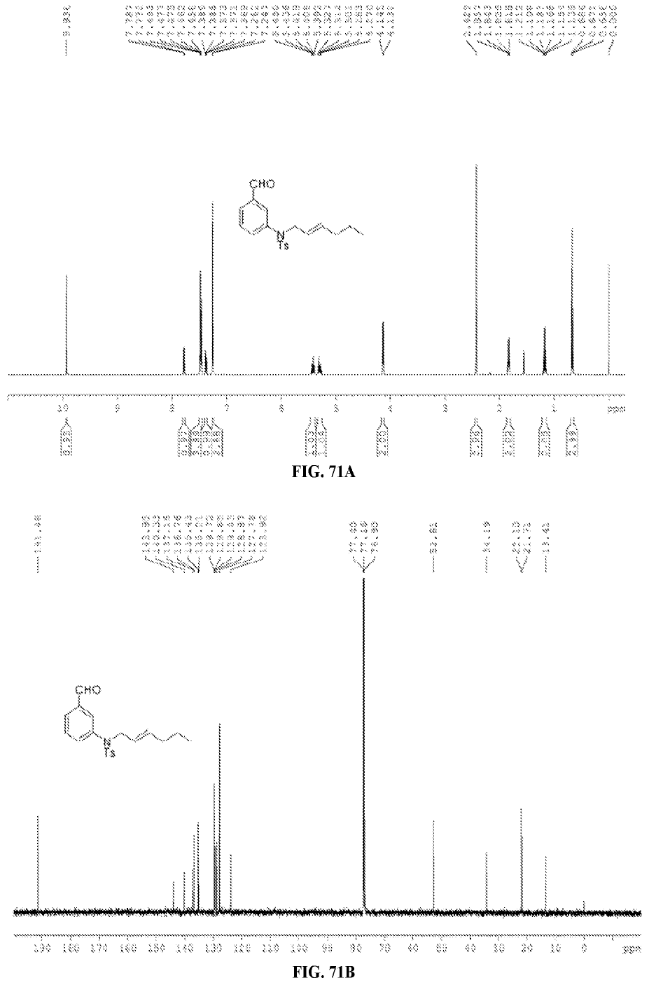

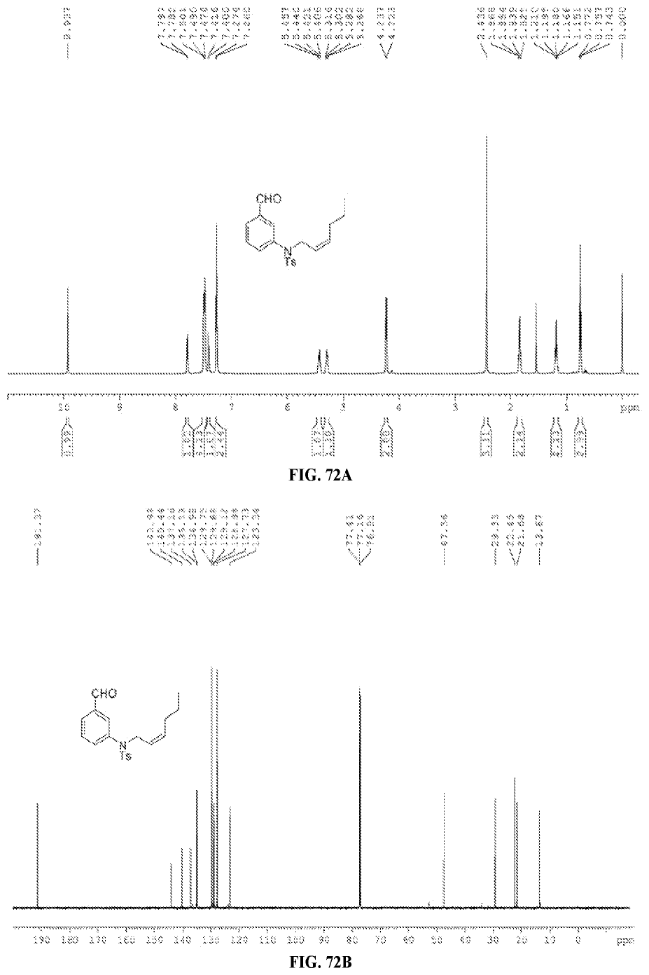

### 3. Characterization of Substrate 1 (FIGS. 43A-72B)

### N-Cinnamyl-N-(3-formylphenyl)-4-methylbenzenesulfonamide

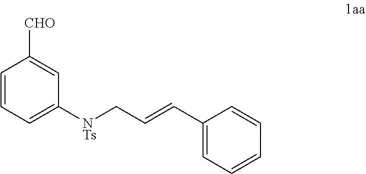 [SMILES: Cc1ccc(S(=O)(=O)N(C/C=C/c2ccccc2)c2cccc(C=O)c2)cc1]

White solid; ¹H NMR (500 MHz, CDCl3) 5 9.92 (s, 1H), 7.77 (d, J=7.4 Hz, 1H), 7.55 (s, 1H), 7.51-7.45 (m, 3H), 7.44 (d, J=8.2 Hz, 1H), 7.29-7.17 (m, 7H), 6.38 (d, J=15.8 Hz, 1H), 6.06 (dt, J=15.8, 6.7 Hz, 1H), 4.36 (d, J=6.7 Hz, 2H), 2.43 (s, 3H); ¹³C NMR (125 MHz, CDCl3) 5 191.4, 144.1, 140.4, 137.3, 136.2, 135.2, 135.1, 134.5, 129.83, 129.79 (2C), 129.1, 129.0, 128.7 (2C), 128.1, 127.8 (2C), 126.6 (2C), 123.5, 53.1, 21.7; HRMS (ESI) Calcd for C23H21NO3SK [M+K]⁺ 430.0874; found 430.0858.

### N-Cinnamyl-N-(3-formyl-4-methoxyphenyl)-4-methylbenzenesulfonamide

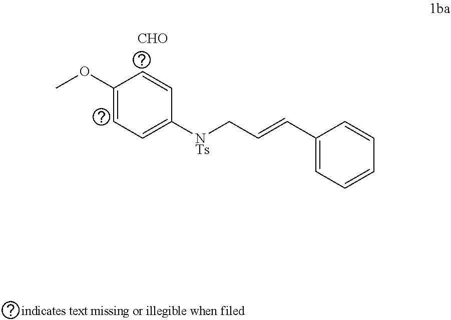

White solid; ¹H NMR (500 MHz, CDCl3) 5 10.34 (s, 1H), 7.53-7.47 (m, 3H), 7.29 (d, J=2.8 Hz, 1H), 7.28-7.19 (m, 7H), 6.95 (d, J=9.0 Hz, 1H), 6.35 (d, J=15.8 Hz, 1H), 6.06 (dt, J=15.8, 6.7 Hz, 1H), 4.28 (dd, J=6.7, 0.8 Hz, 2H), 3.92 (s, 3H), 2.44 (s, 3H); ¹¹C NMR (125 MHz, CDCl3) 5 188.9, 161.2, 144.0, 138.3, 136.3, 135.1, 134.4, 132.3, 129.8 (2C), 128.6 (2C), 128.0, 127.8 (2C), 127.0, 126.6 (2C), 124.8, 123.8, 112.6, 56.0, 53.2, 21.7; HRMS (ESI) Calcd for C24H23NO4SK [M+K]⁺ 460.0979; found 460.0977.

### N-Cinnamyl-N-(4-fluoro-3-formylphenyl)-4-methylbenzenesulfonamide

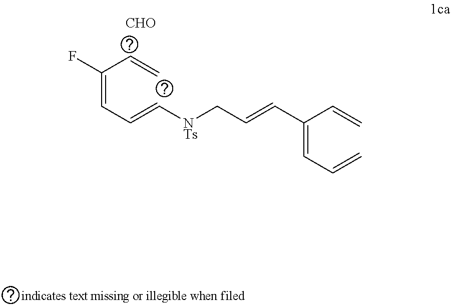

White solid; ¹H NMR (500 MHz, CDCl3) 5 10.25 (s, 1H), 7.52 (ddd, J=9.1, 4.8, 2.9 Hz, 1H), 7.49 (d, J=8.1 Hz, 2H), 7.39 (dd, J=6.0, 2.9 Hz, 1H), 7.28 (d, J=8.1 Hz, 2H), 7.27-7.19 (m, 5H), 7.14 (d, J=9.1 Hz, 1H), 6.35 (d, J=15.8 Hz, 1H), 6.03 (dt, J=15.8, 6.7 Hz, 1H), 4.30 (dd, J=6.7, 1.2 Hz, 2H), 2.45 (s, 3H); ¹³C NMR (125 MHz, CDCl3) 5 186.3 (d, J=6.0 Hz), 163.6 (d, J=259.2 Hz), 144.3, 138.2 (d, J=9.4 Hz), 136.1 (d, J=2.9 Hz), 136.0, 134.8, 134.7, 129.9 (2C), 128.7 (2C), 128.2, 127.8 (2C), 127.4 (d, J=2.3 Hz), 126.6 (2C), 124.3 (d, J=9.2 Hz), 123.3, 117.6 (d, J=21.7 Hz), 53.2, 21.8; HRMS (ESI) Calcd for C23H21FNO3S [M+H]⁺ 410.1221; found 410.1209.

### N-Cinnamyl-N-(3-formyl-4-(trifluoromethyl)phenyl)-4-methylbenzenesulfonamide

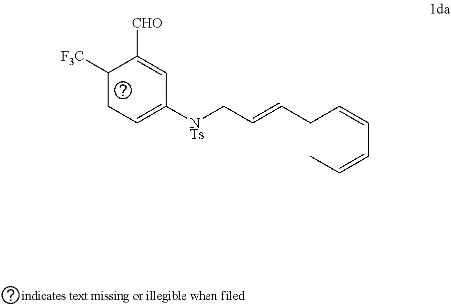

White solid; ¹H NMR (500 MHz, CDCl3) 5 10.30 (q, J=2.0 Hz, 1H), 7.76-7.71 (m, 2H), 7.66 (dd, J=8.4, 1.7 Hz, 1H), 7.49 (d, J=8.3 Hz, 2H), 7.29 (d, J=8.1 Hz, 2H), 7.27-7.19 (m, 5H), 6.42 (d, J=15.9 Hz, 1H), 6.03 (dt, J=15.9, 6.7 Hz, 1H), 4.39 (dd, J=6.7, 1.1 Hz, 2H), 2.44 (s, 3H); ¹³C NMR (125 MHz, CDCl3) 5 188.0 (q, J=2.3 Hz), 144.5, 143.5, 135.9, 135.0, 134.8, 134.4, 133.6, 130.0 (2C), 129.4 (q, J=32.8 Hz), 128.7 (2C), 128.3, 127.7 (2C), 127.3 (q, J=5.6 Hz), 126.9, 126.6 (2C), 123.5 (q, J=272.8 Hz), 122.9, 52.2, 21.7; HRMS (ESI) Calcd for C24H20F3NO3SK [M+K]⁺ 498.0748; found 498.0745.

### N-Cinnamyl-N-(3-formyl-5-methoxyphenyl)-4-methylbenzenesulfonamide

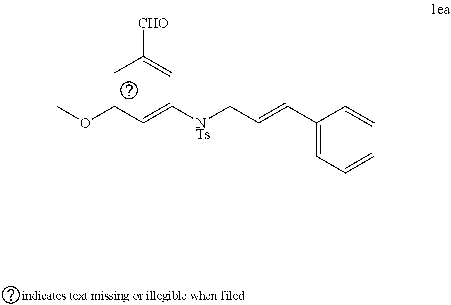

White solid; ¹H NMR (500 MHz, CDCl3) 5 9.86 (s, 1H), 7.52 (d, J=8.2 Hz, 2H), 7.30-7.20 (m, 8H), 7.15 (s, 1H), 6.99 (t, J=2.1 Hz, 1H), 6.39 (d, J=15.8 Hz, 1H), 6.08 (dt, J=15.8, 6.6 Hz, 1H), 4.34 (d, J=6.6 Hz, 2H), 3.81 (s, 3H), 2.44 (s, 3H); 13C NMR (125 MHz, CDCl3) 5 191.3, 160.5, 144.1, 141.4, 137.9, 136.2, 135.0, 134.5, 129.8 (2C), 128.7 (2C), 128.1, 127.8 (2C), 126.6 (2C), 123.5, 122.4, 121.8, 112.5, 55.9, 53.1, 21.7; HRMS (ESI) Calcd for C24H23NO4SK [M+K]⁺ 460.0979; found 460.0975.

### N-Cinnamyl-N-(3-formyl-5-methylphenyl)-4-methylbenzenesulfonamide

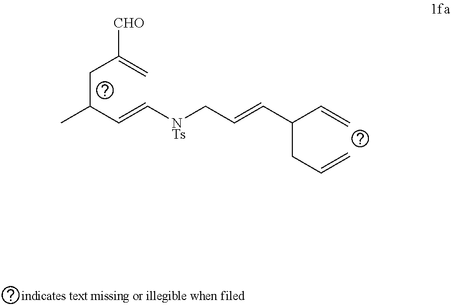

White solid; ¹H NMR (500 MHz, CDCl3) 5 9.87 (s, 1H), 7.58 (s, 1H), 7.50 (d, J=8.3 Hz, 2H), 7.32 (s, 1H), 7.29-7.19 (m, 8H), 6.37 (d, J=15.8 Hz, 1H), 6.06 (dt, J=15.8, 6.7 Hz, 1H), 4.33 (dd, J=6.7, 1.0 Hz, 2H), 2.44 (s, 3H), 2.39 (s, 3H); 13C NMR (125 MHz, CDCl3) 5 191.7, 144.1, 140.3, 140.2, 137.1, 136.5, 136.2, 135.1, 134.4, 130.0, 129.8 (2C), 128.7 (2C), 128.1, 127.9 (2C), 126.6 (2C), 125.8, 123.7, 53.1, 21.7, 21.3; HRMS (ESI) Calcd for C24H23NO3SK [M+K]⁺ 444.1030; found 444.1016.

### N-Cinnamyl-N-(3-fluoro-5-formylphenyl)-4-methylbenzenesulfonamide

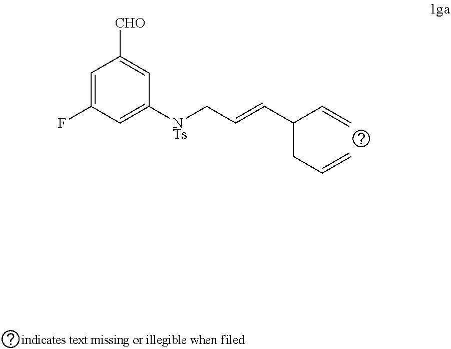

White solid; ¹H NMR (500 MHz, CDCl3) 5 9.89 (d, J=1.8 Hz, 1H), 7.50 (d, J=8.3 Hz, 2H), 7.47 (ddd, J=7.8, 2.3, 1.2 Hz, 1H), 7.41 (s, 1H), 7.29 (d, J=8.3 Hz, 2H), 7.27-7.20 (m, 5H), 7.16 (dt, J=9.1, 2.3 Hz, 1H), 6.40 (d, J=15.8 Hz, 1H), 6.05 (dt, J=15.8, 6.7 Hz, 1H), 4.36 (dd, J=6.7, 1.0 Hz, 2H), 2.44 (s, 3H); 13C NMR (125 MHz, CDCl3) 5 190.0 (d, J=2.4 Hz), 162.8 (d, J=250.0 Hz), 144.5, 142.1 (d, J=9.1 Hz), 138.3 (d, J=7.0 Hz), 136.0, 134.9, 134.8, 129.9 (2C), 128.7 (2C), 128.3, 127.7 (2C), 126.6 (2C), 125.3 (d, J=2.8 Hz), 123.0, 121.8 (d, J=23.2 Hz), 114.9 (d, J=21.8 Hz), 52.9, 21.7; HRMS (ESI) Calcd for C23H20FNO3SNa [M+Na]⁺ 432.1040; found 432.1031.

### N-Cinnamyl-N-(3-formyl-5-(trifluoromethyl)phenyl)-4-methylbenzenesulfonamide

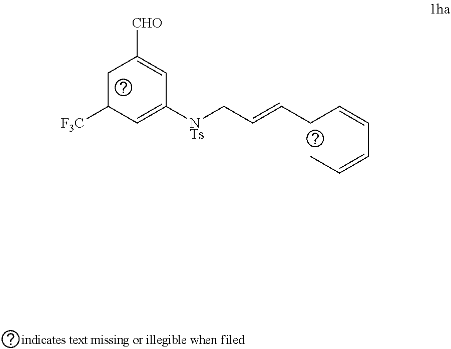

White solid; ¹H NMR (500 MHz, CDCl3) 5 9.98 (s, 1H), 8.01 (s, 1H), 7.81 (s, 1H), 7.58 (s, 1H), 7.48 (d, J=8.1 Hz, 2H), 7.29 (d, J=8.1 Hz, 2H), 7.27-7.19 (m, 5H), 6.40 (d, J=15.8 Hz, 1H), 6.03 (dt, J=15.8, 6.7 Hz, 1H), 4.38 (d, J=6.7 Hz, 2H), 2.44 (s, 3H); ¹³C NMR (125 MHz, CDCl3) 5 189.9, 144.7, 141.4, 137.6, 135.9, 135.3, 134.5, 132.5 (q, J=33.6 Hz), 132.3, 130.7 (q, J=3.3 Hz), 130.0 (2C), 128.7 (2C), 128.4, 127.8 (2C), 126.6 (2C), 125.2 (q, J=3.6 Hz), 123.0 (q, J=271.4 Hz), 122.8, 52.9, 21.7; HRMS (ESI) Calcd for C24H20F3NO3SK [M+K]⁺ 498.0748; found 498.0737.

### N-Cinnamyl-N-(5-formyl-2-methoxyphenyl)-4-methylbenzenesulfonamide

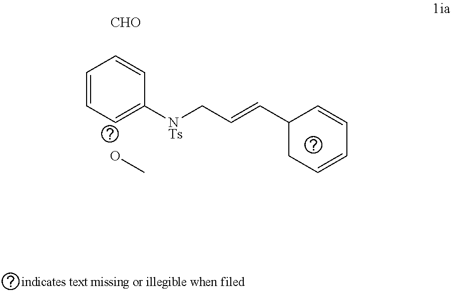

White solid; ¹H NMR (500 MHz, CDCl3) 5 9.83 (s, 1H), 7.83 (dd, J=8.6, 2.1 Hz, 1H), 7.72 (d, J=2.1 Hz, 1H), 7.61 (d, J=8.1 Hz, 2H), 7.27 (d, J=8.1 Hz, 2H), 7.26-7.17 (m, 5H), 6.92 (d, J=8.6 Hz, 1H), 6.32 (d, J=15.8 Hz, 1H), 6.12 (dt, J=15.8, 6.8 Hz, 1H), 4.34 (br, 2H), 3.60 (s, 3H), 2.44 (s, 3H); ¹³C NMR (125 MHz, CDCl3) 5 190.3, 161.9, 143.4, 137.4, 136.4, 134.9, 133.8, 131.7, 130.0, 129.4 (2C), 128.7 (2C), 128.0, 127.9, 127.7 (2C), 126.6 (2C), 124.5, 112.1, 55.8, 52.6, 21.7; HRMS (ESI) Calcd for C24H23NO4SK [M+K]⁺ 460.0979; found 460.0973.

### N-Cinnamyl-N-(2-fluoro-5-formylphenyl)-4-methylbenzenesulfonamide

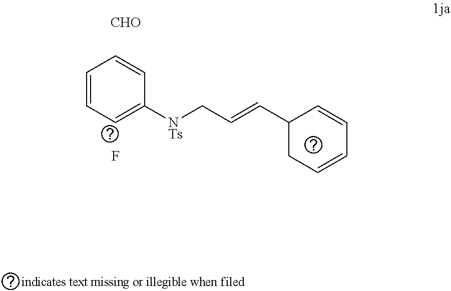

White solid; ¹H NMR (500 MHz, CDCl3) 5 9.90 (s, 1H), 7.84 (ddd, J=8.5, 4.7, 2.1 Hz, 1H), 7.77 (dd, J=7.2, 2.1 Hz, 1H), 7.61 (d, J=8.2 Hz, 2H), 7.31 (d, J=8.2 Hz, 2H), 7.29-7.17 (m, 6H), 6.36 (d, J=15.8 Hz, 1H), 6.10 (dt, J=15.8, 6.8 Hz, 1H), 4.34 (d, J=6.8 Hz, 2H), 2.46 (s, 3H); ¹³C NMR (125 MHz, CDCl3) 5 189.9, 163.8 (d, J=261.0 Hz), 144.3, 136.1, 135.9, 134.7, 134.5 (d, J=2.3 Hz), 133.3 (d, J=2.6 Hz), 131.4 (d, J=9.8 Hz), 129.9 (2C), 128.7 (2C), 128.2, 127.70 (d, J=12.6 Hz), 127.68 (2C), 126.6 (2C), 123.2, 117.8 (d, J=21.7 Hz), 53.0 (d, J=2.8 Hz), 21.8; HRMS (ESI) Calcd for C23H20FNO3SK [M+K]⁺ 448.0780; found 448.0774.

### N-Cinnamyl-N-(3-formylphenyl)-4-nitrobenzenesulfonamide

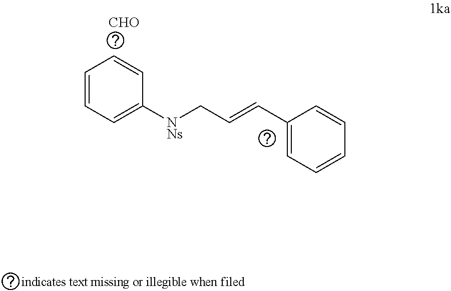

Yellow solid; ¹H NMR (500 MHz, CDCl3) 5 9.95 (s, 1H), 8.31 (d, J=8.7 Hz, 2H), 7.85-7.78 (m, 3H), 7.59 (s, 1H), 7.53 (t, J=7.8 Hz, 1H), 7.41 (d, J=8.0 Hz, 1H), 7.29-7.19 (m, 5H), 6.41 (d, J=15.8 Hz, 1H), 6.07 (dt, J=15.8, 6.7 Hz, 1H), 4.42 (d, J=6.7 Hz, 2H); ¹³C NMR (125 MHz, CDCl3) 5 191.0, 150.4, 144.2, 139.6, 137.6, 135.8, 135.4, 134.9, 130.3, 130.0, 128.9 (2C), 128.82, 128.77 (2C), 128.5, 126.6 (2C), 124.5 (2C), 122.5, 53.8; HRMS (ESI) Cald for C22H18N2O5SK [M+K]⁺ 461.0568; found 461.0562.

### (E)-N-(3-formylphenyl)-N-(3-(4-methoxyphenyl)allyl)-4-methylbenzenesulfonamide

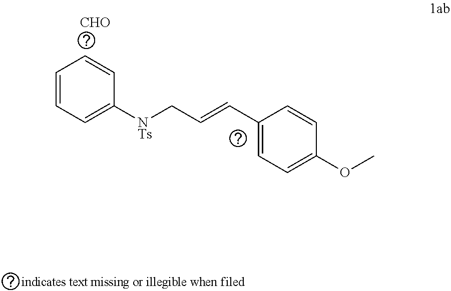

White solid; ¹H NMR (500 MHz, CDCl3) 5 9.92 (s, 1H), 7.77 (d, J=7.4 Hz, 1H), 7.54 (s, 1H), 7.51-7.45 (m, 3H), 7.43 (d, J=8.0 Hz, 1H), 7.26 (d, J=8.1 Hz, 2H), 7.15 (d, J=8.7 Hz, 2H), 6.78 (d, J=8.7 Hz, 2H), 6.31 (d, J=15.8 Hz, 1H), 5.92 (dt, J=15.8, 6.8 Hz, 1H), 4.34 (d, J=6.8 Hz, 2H), 3.77 (s, 3H), 2.43 (s, 3H); ¹³C NMR (125 MHz, CDCl3) 5 191.5, 159.7, 144.1, 140.5, 137.3, 135.3, 135.2, 134.1, 129.81, 129.79 (2C), 129.13, 129.09, 129.0, 127.8 (4C), 121.2, 114.1 (2C), 55.4, 53.2, 21.7; HRMS (ESI) Calcd for C24H23NO4SK [M+K]+460.0979; found 460.0966.

### (E)-N-(3-formylphenyl)-N-(3-(3-methoxyphenyl)allyl)-4-methylbenzenesulfonamide

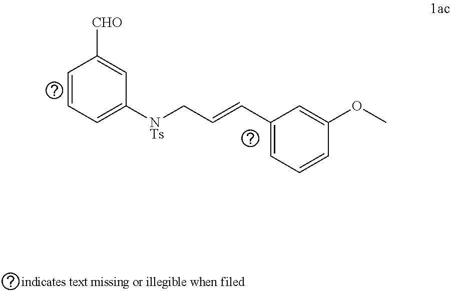

White solid; 1H NMR (500 MHz, CDCl3) 5 9.93 (s, 1H), 7.77 (d, J=7.4 Hz, 1H), 7.54 (s, 1H), 7.51-7.41 (m, 4H), 7.29-7.25 (m, 2H), 7.16 (t, J=7.8 Hz, 1H), 6.836.73 (m, 3H), 6.34 (d, J=15.8 Hz, 1H), 6.06 (dt, J=15.8, 6.6 Hz, 1H), 4.36 (d, J=6.6 Hz, 2H), 3.77 (s, 3H), 2.43 (s, 3H); ¹³C NMR (125 MHz, CDCl3) 5 191.4, 159.9, 144.1, 140.5, 137.6, 137.3, 135.2, 135.1, 134.4, 129.9, 129.8 (2C), 129.7, 129.2, 129.0, 127.8 (2C), 123.9, 119.2, 113.7, 112.0, 55.3, 53.1, 21.7; HRMS (ESI) Calcd for C24H23NO4SK [M+K]⁺460.0979; found 460.0978.

### (E)-N-(3-formylphenyl)-N-(3-(2-methoxyphenyl)allyl)-4-methylbenzenesulfonamide

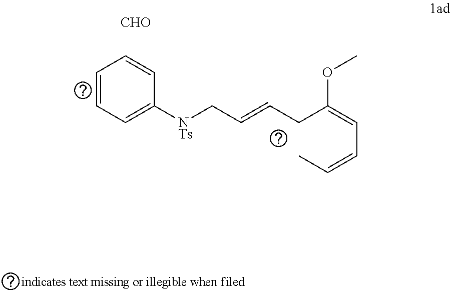

White solid; ¹H NMR (500 MHz, CDCl3) 5 9.92 (s, 1H), 7.77 (d, J=7.4 Hz, 1H), 7.55 (s, 1H), 7.51-7.42 (m, 4H), 7.28-7.25 (m, 2H), 7.21 (d, J=7.6 Hz, 1H), 7.18 (t, J=7.8 Hz, 1H), 6.84 (t, J=7.5 Hz, 1H), 6.79 (d, J=8.2 Hz, 1H), 6.68 (d, J=16.0 Hz, 1H), 6.05 (dt, J=16.0, 6.7 Hz, 1H), 4.37 (d, J=6.7 Hz, 2H), 3.74 (s, 3H), 2.43 (s, 3H); ¹³C NMR (125 MHz, CDCl3) 5 191.5, 156.8, 144.0, 140.4, 137.2, 135.3, 135.2, 129.8 (3C), 129.7, 129.5, 129.2, 128.9, 127.9 (2C), 127.1, 125.3, 123.9, 120.7, 111.0, 55.5, 53.5, 21.7; HRMS (ESI) Calcd for C24H23NO4SK [M+K]⁺ 460.0979; found 460.0965.

### (E)-N-(3-formylphenyl)-4-methyl-N-(3-(p-tolyl)allyl)benzenesulfonamide

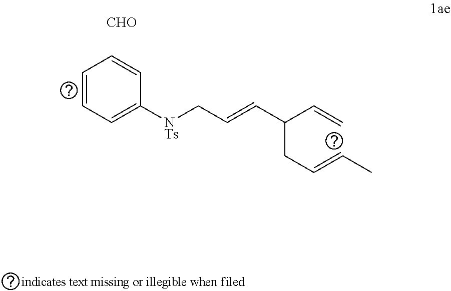

White solid; ¹H NMR (500 MHz, CDCl3) 5 9.92 (s, 1H), 7.77 (dt, J=7.5, 1.3 Hz, 1H), 7.53 (t, J=1.6 Hz, 1H), 7.51-7.45 (m, 3H), 7.43 (ddd, J=8.0, 2.0, 1.4 Hz, 1H), 7.29-7.25 (m, 2H), 7.11 (d, J=8.1 Hz, 2H), 7.06 (d, J=8.1 Hz, 2H), 6.33 (d, J=15.8 Hz, 1H), 6.01 (dt, J=15.8, 6.8 Hz, 1H), 4.35 (dd, J=6.8, 0.9 Hz, 2H), 2.44 (s, 3H), 2.29 (s, 3H); ¹³C NMR (125 MHz, CDCl3) 5 191.5, 144.1, 140.4, 138.1, 137.2, 135.3, 135.1, 134.5, 133.4, 129.81, 129.79 (2C), 129.4 (2C), 129.13, 129.10, 127.8 (2C), 126.5 (2C), 122.4, 53.1, 21.7, 21.3; HRMS (ESI) Calcd for C24H23NO3SK [M+K]⁺ 444.1030; found 444.1015.

### (E)-N-(3-formylphenyl)-4-methyl-N-(3-(m-tolyl)allyl)benzenesulfonamide

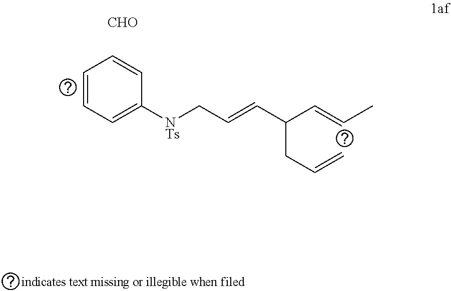

White solid; ¹H NMR (500 MHz, CDCl3) 5 9.93 (s, 1H), 7.77 (d, J=7.4 Hz, 1H), 7.54 (s, 1H), 7.51-7.46 (m, 3H), 7.44 (d, J=7.9 Hz, 1H), 7.29-7.25 (m, 2H), 7.14 (t, J=7.6 Hz, 1H), 7.04-7.00 (m, 3H), 6.33 (d, J=15.8 Hz, 1H), 6.05 (dt, J=15.8, 6.7 Hz, 1H), 4.35 (d, J=6.7 Hz, 2H), 2.44 (s, 3H), 2.29 (s, 3H); ¹³C NMR (125 MHz, CDCl3) 5 191.5, 144.1, 140.4, 138.3, 137.2, 136.1, 135.2, 135.0, 134.6, 129.83, 129.79 (2C), 129.2, 129.0, 128.9, 128.6, 127.8 (2C), 127.3, 123.7, 123.2, 53.1, 21.7, 21.4; HRMS (ESI) Calcd for C24H23NO3SK [M+K]* 444.1030; found 444.1016.

### (E)-N-(3-formylphenyl)-4-methyl-N-(3-(o-tolyl)allyl)benzenesulfonamide

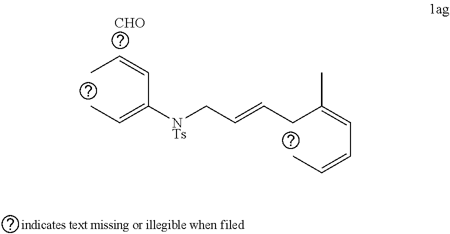

White solid; ¹H NMR (500 MHz, CDCl3) 5 9.93 (s, 1H), 7.77 (d, J=7.2 Hz, 1H), 7.55 (s, 1H), 7.53-7.43 (m, 4H), 7.29-7.24 (m, 2H), 7.20 (d, J=7.1 Hz, 1H), 7.137.03 (m, 3H), 6.56 (d, J=15.7 Hz, 1H), 5.90 (dt, J=15.7, 6.7 Hz, 1H), 4.38 (d, J=6.7 Hz, 2H), 2.43 (s, 3H), 2.10 (s, 3H); ¹³C NMR (125 MHz, CDCl3) 5 191.4, 144.2, 140.3, 137.2, 135.5, 135.44, 135.36, 134.9, 133.0, 130.3, 129.8 (3C), 129.2, 128.9, 128.0, 127.8 (2C), 126.2, 126.0, 124.8, 53.0, 21.7, 19.7; HRMS (ESI) Calcd for C24H23NO3SK [M+K]⁺ 444.1030; found 444.1023.

### (E)-N-(3-(4-fluorophenyl)allyl)-N-(3-formylphenyl)-4-methylbenzenesulfonamide

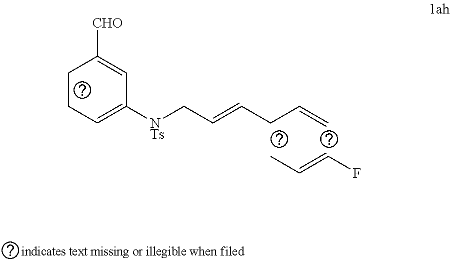

White solid; ¹H NMR (500 MHz, CDCl3) 5 9.93 (s, 1H), 7.78 (d, J=7.5 Hz, 1H), 7.54 (s, 1H), 7.52-7.46 (m, 3H), 7.44 (d, J=8.1 Hz, 1H), 7.27 (d, J=8.1 Hz, 2H), 7.18 (dd, J=8.7, 5.5 Hz, 2H), 6.94 (t, J=8.7 Hz, 2H), 6.35 (d, J=15.8 Hz, 1H), 5.99 (dt, J=15.8, 6.7 Hz, 1H), 4.35 (d, J=6.7 Hz, 2H), 2.43 (s, 3H); ¹³C NMR (125 MHz, CDCl3) 5 191.4, 162.6 (d, J=245.8 Hz), 144.2, 140.4, 137.2, 135.2, 134.9, 133.2, 132.3 (d, J=3.6 Hz), 129.9, 129.8 (2C), 129.3, 128.9, 128.1 (d, J=7.7 Hz, 2C), 127.8 (2C), 123.3, 115.6 (d, J=21.6 Hz, 2C), 53.0, 21.7; HRMS (ESI) Calcd for C23H20FNO3SK [M+K]⁺ 448.0780; found 448.0772.

### (E)-N-(3-(3-fluorophenyl)allyl)-N-(3-formylphenyl)-4-methylbenzenesulfonamide

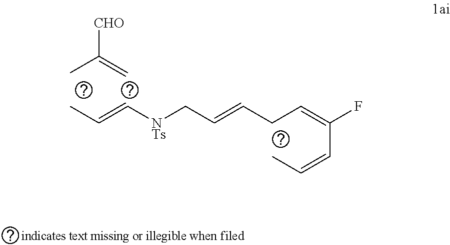

White solid; ¹H NMR (500 MHz, CDCl3) 5 9.93 (s, 1H), 7.78 (d, J=7.5 Hz, 1H), 7.55 (s, 1H), 7.51-7.46 (m, 3H), 7.44 (d, J=8.1 Hz, 1H), 7.27 (d, J=8.1 Hz, 2H), 7.23-7.18 (m, 1H), 6.98 (d, J=7.8 Hz, 1H), 6.92-6.86 (m, 2H), 6.36 (d, J=15.8 Hz, 1H), 6.08 (dt, J=15.8, 6.6 Hz, 1H), 4.37 (d, J=6.6 Hz, 2H), 2.43 (s, 3H); 13C NMR (125 MHz, CDCl3) 5 191.3, 163.1 (d, J=244.6 Hz), 144.2, 140.4, 138.4 (d, J=7.8 Hz), 137.3, 135.1, 135.0, 133.2 (d, J=2.4 Hz), 130.1 (d, J=8.3 Hz), 129.9, 129.8 (2C), 129.2, 128.9, 127.8 (2C), 125.1, 122.5 (d, J=2.6 Hz), 114.9 (d, J=21.3 Hz), 113.0 (d, J=21.7 Hz), 52.9, 21.7; HRMS (ESI) Calcd for C23H20FNO3SK [M+K]⁺ 448.0780; found 448.0769.

### (E)-N-(3-(2-fluorophenyl)allyl)-N-(3-formylphenyl)-4-methylbenzenesulfonamide

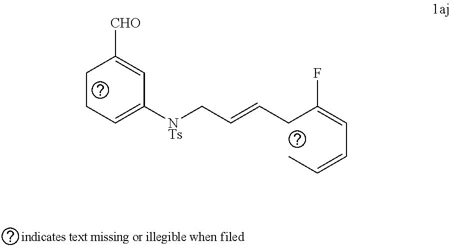

White solid; 1H NMR (500 MHz, CDCl3) 5 9.93 (s, 1H), 7.78 (dt, J=7.5, 1.3 Hz, 1H), 7.56 (t, J=1.7 Hz, 1H), 7.51-7.46 (m, 3H), 7.43 (ddd, J=8.0, 2.1, 1.3 Hz, 1H), 7.30-7.25 (m, 3H), 7.20-7.14 (m, 1H), 7.03 (t, J=7.5 Hz, 1H), 6.96 (ddd, J=10.7, 8.3, 1.0 Hz, 1H), 6.53 (d, J=16.0 Hz, 1H), 6.15 (dt, J=16.0, 6.6 Hz, 1H), 4.38 (dd, J=6.6, 1.1 Hz, 2H), 2.44 (s, 3H); ¹³C NMR (125 MHz, CDCl3) 5 191.4, 160.2 (d, J=248.8 Hz), 144.2, 140.4, 137.3, 135.2, 135.1, 129.8 (2C), 129.5, 129.4, 129.3, 129.1, 127.8 (2C), 127.6, 127.0, 126.3 (d, J=5.9 Hz), 124.2, 124.0 (d, J=12.1 Hz), 115.8 (d, J=24.6 Hz), 53.3, 21.7; HRMS (ESI) Calcd for C23H20FNO3SK [M+K]⁺ 448.0780; found 448.0763.

### (E)-N-(3-formylphenyl)-4-methyl-N-(3-(4-(trifluoromethyl)phenyl)allyl)benzenesulfonamide

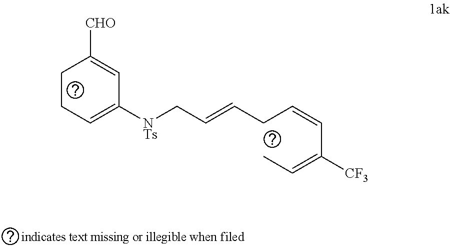

White solid; ¹H NMR (500 MHz, CDCl3) 5 9.93 (s, 1H), 7.79 (d, J=7.5 Hz, 1H), 7.55 (s, 1H), 7.52-7.47 (m, 5H), 7.45 (d, J=8.1 Hz, 1H), 7.31 (d, J=8.2 Hz, 2H), 7.29-7.25 (m, 2H), 6.43 (d, J=15.9 Hz, 1H), 6.19 (dt, J=15.9, 6.5 Hz, 1H), 4.39 (d, J=6.5 Hz, 2H), 2.44 (s, 3H); ¹³C NMR (125 MHz, CDCl3) 5 191.3, 144.3, 140.5, 139.6, 137.3, 135.1, 135.0, 132.9, 129.94, 129.89 (q, J=32.3 Hz), 129.8 (2C), 129.4, 128.7, 127.8 (2C), 126.7 (2C), 126.5, 125.6 (q, J=3.8 Hz, 2C), 124.2 (q, J=270.3 Hz), 52.9, 21.7; HRMS (ESI) Calcd for C24H20F3NO3SNa [M+Na]⁺ 482.1008; found 482.0994.

### (E)-N-(3-formylphenyl)-4-methyl-N-(3-(3-(trifluoromethyl)phenyl)allyl)benzenesulfonamide

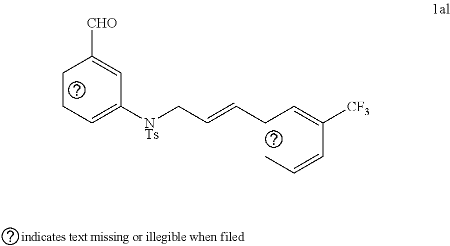

White solid; 1H NMR (500 MHz, CDCl3) 5 9.94 (s, 1H), 7.80 (d, J=7.5 Hz, 1H), 7.57 (s, 1H), 7.53-7.47 (m, 3H), 7.47-7.43 (m, 3H), 7.42-7.35 (m, 2H), 7.29-7.26 (m, 2H), 6.43 (d, J=15.9 Hz, 1H), 6.17 (dt, J=15.9, 6.5 Hz, 1H), 4.39 (dd, J=6.5, 1.0 Hz, 2H), 2.44 (s, 3H); ¹³C NMR (125 MHz, CDCl3) 5 191.4, 144.3, 140.4, 137.3, 136.9, 135.1, 134.9, 132.8, 131.0 (q, J=32.0 Hz), 130.0, 129.8 (2C), 129.7, 129.4, 129.2, 128.8, 127.8 (2C), 125.7, 124.6 (q, J=3.2 Hz), 124.0 (q, J=270.7 Hz), 123.2 (q, J=3.3 Hz), 52.9, 21.7; HRMS (ESI) Calcd for C24H21F3NO3S [M+H]⁺ 460.1189; found 460.1184.

### (E)-N-(3-formylphenyl)-4-methyl-N-(3-(2-(trifluoromethyl)phenyl)allyl)benzenesulfonamide

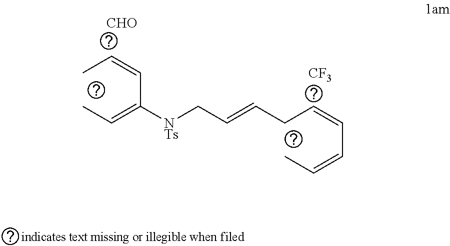

White solid; 1H NMR (500 MHz, CDCl3) 5 9.93 (s, 1H), 7.79 (d, J=7.6 Hz, 1H), 7.57-7.52 (m, 2H), 7.51-7.46 (m, 3H), 7.46-7.39 (m, 3H), 7.33-7.27 (m, 3H), 6.72 (d, J=15.7 Hz, 1H), 6.04 (dt, J=15.7, 6.6 Hz, 1H), 4.39 (dd, J=6.6, 1.1 Hz, 2H), 2.44 (s, 3H), ¹³C NMR (125 MHz, CDCl3) 5 191.4, 144.3, 140.1, 137.3, 135.2, 135.0, 134.7, 132.0, 130.7, 129.9 (3C), 129.2, 129.0, 128.0, 127.8 (3C), 127.7, 127.4 (q, J=30.3 Hz), 125.8 (q, J=5.6 Hz), 124.1 (q, J=272.0 Hz), 52.8, 21.7; HRMS (ESI) Calcd for C24H20F3NO3SNa [M+Na]⁺ 482.1008; found 482.0996.

### (E)-N-(3-formylphenyl)-4-methyl-N-(3-(4-nitrophenyl)allyl)benzenesulfonamide

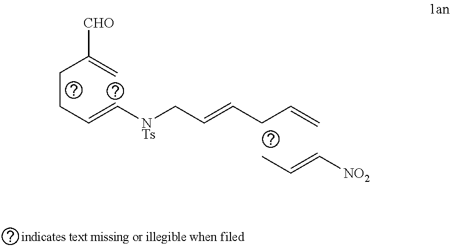

Light yellow solid; ¹H NMR (500 MHz, CDCl3) 5 9.94 (s, 1H), 8.11 (d, J=8.8 Hz, 2H), 7.80 (d, J=7.4 Hz, 1H), 7.55 (s, 1H), 7.54-7.45 (m, 4H), 7.37 (d, J=8.8 Hz, 2H), 7.30-7.26 (m, 2H), 6.50 (d, J=15.9 Hz, 1H), 6.29 (dt, J=15.9, 6.3 Hz, 1H), 4.41 (d, J=6.3 Hz, 2H), 2.44 (s, 3H); ¹³C NMR (125 MHz, CDCl3) 5 191.3, 147.3, 144.4, 142.5, 140.5, 137.4, 135.0, 134.9, 132.0, 130.0, 129.9 (2C), 129.6, 128.8, 128.5, 127.8 (2C), 127.2 (2C), 124.1 (2C), 52.8, 21.7; HRMS (ESI) Calcd for C23H21N2O5S [M+H]⁺ 437.1166; found 437.1168.

### N-(But-2-en-1-yl)-N-(3-formylphenyl)-4-methylbenzenesulfonamide

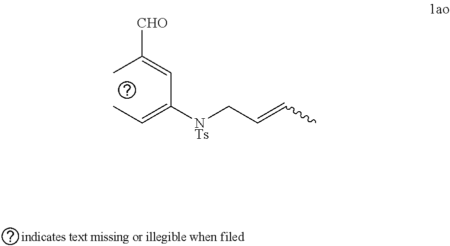

White solid; ¹H NMR (500 MHz, CDCl3) 5 9.94 (s, 1H), 7.79 (d, J=7.6 Hz, 1H), 7.52-7.44 (m, 4H), 7.38 (d, J=8.0 Hz, 1H), 7.25 (d, J=8.1 Hz, 2H), 5.52-5.44 (m, 1H), 5.38-5.30 (m, 1H), 4.14 (d, J=6.5 Hz, 2H), 2.42 (s, 3H), 1.54 (d, J=6.4 Hz, 3H); ¹³C NMR (125 MHz, CDCl3) 5 191.5, 143.9, 140.4, 137.2, 135.2 (2C), 131.1, 129.68 (2C), 129.65, 129.3, 128.8, 127.7 (2C), 125.0, 52.7, 21.7, 17.7; HRMS (ESI) Calcd for C18H20NO3S [M+H]⁺ 330.1158; found 330.1156.

### (E)-N-(4-((tert-Butyldimethylsilyl)oxy)but-2-en-1-yl)-N-(3-formylphenyl)-4-methylbenzenesulfonamide

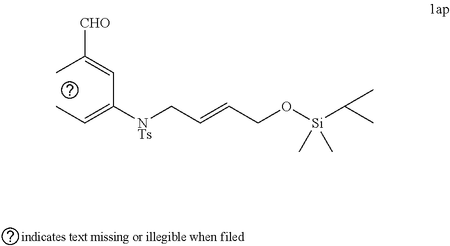

White solid; 1H NMR (500 MHz, CDCl3) 5 9.95 (s, 1H), 7.80 (d, J=7.6 Hz, 1H), 7.52-7.46 (m, 4H), 7.40 (d, J=8.0 Hz, 1H), 7.29-7.25 (m, 2H), 5.63-5.53 (m, 2H), 4.23 (d, J=4.6 Hz, 2H), 4.03-3.99 (m, 2H), 2.44 (s, 3H), 0.82 (s, 9H), −0.07 (s, 6H); 13C NMR (125 MHz, CDCl3) 5 191.4, 144.0, 140.0, 137.2, 135.2, 135.1, 134.8, 129.8 (2C), 129.7, 129.4, 128.9, 127.8 (2C), 123.6, 62.8, 52.2, 25.9 (3C), 21.7, 18.4, −5.3 (2C); HRMS (ESI) Calcd for C24H33NO4SSiK [M+K]⁺ 498.1531; found 498.1529.

### (E)-N-(5-((tert-butyldimethylsilyl)oxy)pent-2-en-1-yl)-N-(3-formylphenyl)-4-methylbenzenesulfonamide

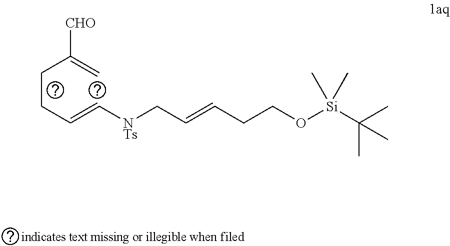

White solid; ¹H NMR (500 MHz, CDCl3) 5 9.96 (s, 1H), 7.80 (d, J=7.6 Hz, 1H), 7.52-7.45 (m, 4H), 7.39 (d, J=8.0 Hz, 1H), 7.29-7.25 (m, 2H), 5.53-5.46 (m, 1H), 5.44-5.37 (m, 1H), 4.17 (d, J=6.4 Hz, 2H), 3.45 (t, J=6.7 Hz, 2H), 2.45 (s, 3H), 2.14-2.08 (m, 2H), 0.85 (s, 9H), −0.02 (s, 6H); ¹³C NMR (125 MHz, CDCl3) 5 191.4, 144.0, 140.4, 137.2, 135.3, 135.2, 133.0, 129.74 (2C), 129.68, 129.4, 128.9, 127.8 (2C), 125.8, 62.6, 52.8, 35.8, 26.0 (3C), 21.7, 18.4, −5.21 (2C); HRMS (ESI) Calcd for C25H35NO4SSiK [M+K]⁺ 512.1688; found 512.1682.

### Methyl (E)-4-((N-(3-formylphenyl)-4-methylphenyl)sulfonamido)but-2-enoate

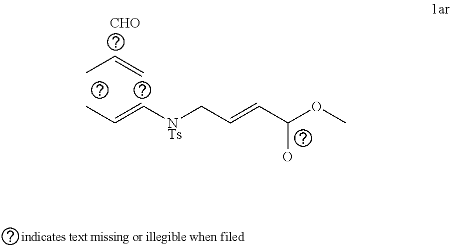

White solid; 1H NMR (500 MHz, CDCl3) 5 9.94 (s, 1H), 7.81 (d, J=7.6 Hz, 1H), 7.54-7.49 (m, 2H), 7.47-741 (m, 3H), 7.29-7.24 (m, 2H), 6.79 (dt, J=15.7, 5.7 Hz, 1H), 5.92 (d, J=15.7 Hz, 1H), 4.37 (dd, J=5.7, 1.2 Hz, 2H), 3.68 (s, 3H), 2.43 (s, 3H); ¹³C NMR (125 MHz, CDCl3) 5 191.2, 166.0, 144.5, 141.9, 140.3, 137.4, 134.7, 134.5, 130.1, 129.9 (2C), 129.4, 128.4, 127.7 (2C), 124.1, 51.8, 51.4, 21.7; HRMS (ESI) Calcd for C19H19NO5SK [M+K]⁺ 412.0616; found 412.0604.

### (E)-N-(3-Formylphenyl)-N-(hex-2-en-1-yl)-4-methylbenzenesulfonamide

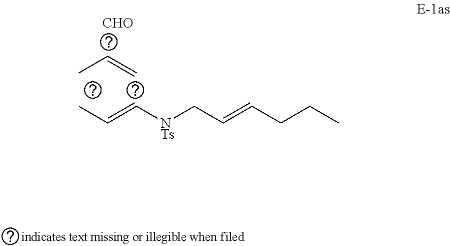

White solid; ¹H NMR (500 MHz, CDCl3) 5 9.94 (s, 1H), 7.78 (d, J=7.8 Hz, 1H), 7.50-7.45 (m, 4H), 7.40-7.36 (m, 1H), 7.27-7.24 (m, 2H), 5.42 (dt, J=15.3, 6.8 Hz, 1H), 5.30 (dt, J=15.3, 6.5 Hz, 1H), 4.14 (d, J=6.5 Hz, 2H), 2.43 (s, 3H), 1.87-1.81 (m, 2H), 1.22-1.15 (m, 2H), 0.67 (t, J=7.4 Hz, 3H); ¹³C NMR (125 MHz, CDCl3) 5 191.5, 144.0, 140.3, 137.2, 136.8, 135.4, 135.2, 129.72 (2C), 129.65, 129.4, 128.9, 127.8 (2C), 123.9, 52.8, 34.2, 22.1, 21.7, 13.4; HRMS (ESI) Calcd for C20H23NO3SK [M+K]⁺ 396.1030; found 396.1019.

### (Z)—N-(3-Formylphenyl)-N-(hex-2-en-1-yl)-4-methylbenzenesulfonamide

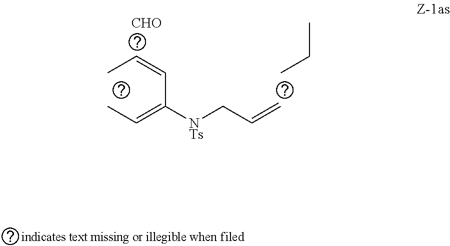

White solid; ¹H NMR (500 MHz, CDCl3) 5 9.94 (s, 1H), 7.79 (d, J=7.4 Hz, 1H), 7.52-7.45 (m, 4H), 7.41 (d, J=7.9 Hz, 1H), 7.28-7.25 (m, 2H), 5.47-5.40 (m, 1H), 5.32-5.25 (m, 1H), 4.23 (d, J=6.9 Hz, 2H), 2.44 (s, 3H), 1.87-1.81 (m, 2H), 1.231.14 (m, 2H), 0.76 (t, J=7.3 Hz, 3H); ¹³C NMR (125 MHz, CDCl3) 5 191.4, 144.0, 140.4, 137.2, 135.1 (2C), 135.0, 129.8 (2C), 129.7, 129.1, 129.0, 127.7 (2C), 123.3, 47.4, 29.3, 22.5, 21.7, 13.7; HRMS (ESI) Calcd for C20H24NO3S [M+H]⁺ 358.1471; found 358.1462.

### 4. General Procedure for Ruthenium Catalyzed Enantioselective C—H Functionalization to Synthesize Indoline Derivatives

To a 10 mL of Schlenk tube equipped with a magnetic stirring bar were added substrate 1 (0.1 mmol), [Ru(p-cymene)Cl₂]₂ (5 mol %), AgBF₄ (20 mol %), KH₂PO₄ (0.2 mmol) and acid (0.3 equiv.) under air. The mixture was then evacuated and backfilled with nitrogen for three times. After that, chiral amine (50 mol %), HFIP (0.4 mL) and PhCl (0.4 mL) were added subsequently. After stirring at suitable temperature (60-90° C.) for 24 h, the reaction mixture was cooled to room temperature. The solvent was removed under reduced pressure, and the residue was purified by flash chromatography on silica gel with hexanes/ethyl acetate/dichloromethane as the eluent to give the corresponding product 2.

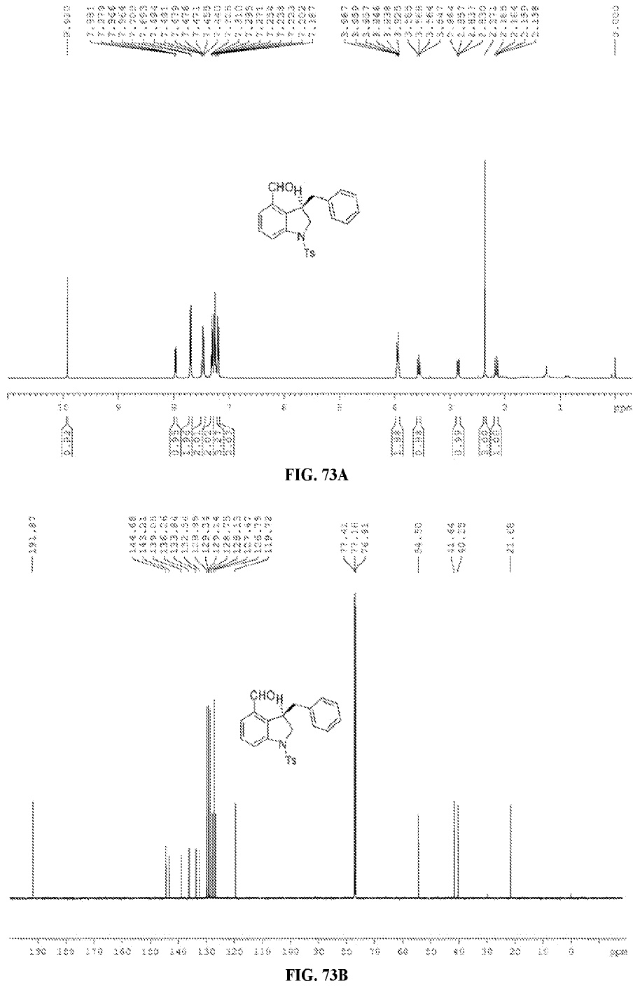

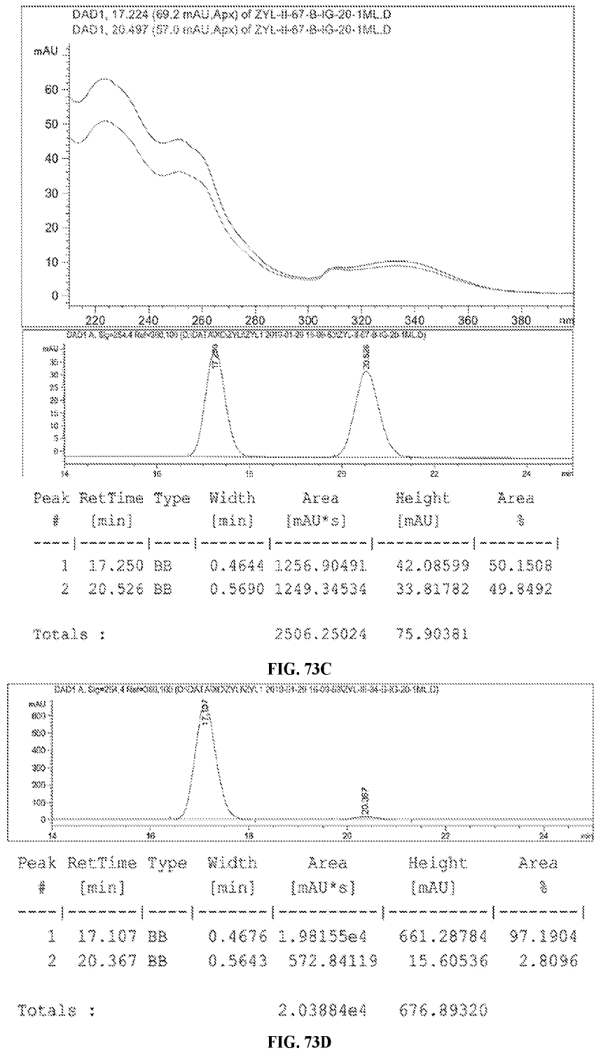

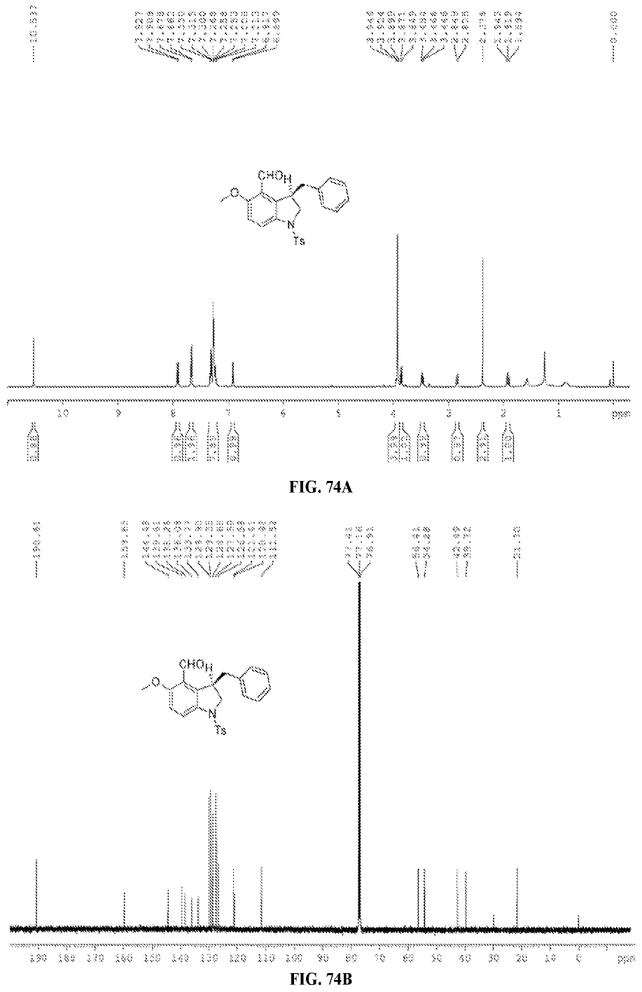

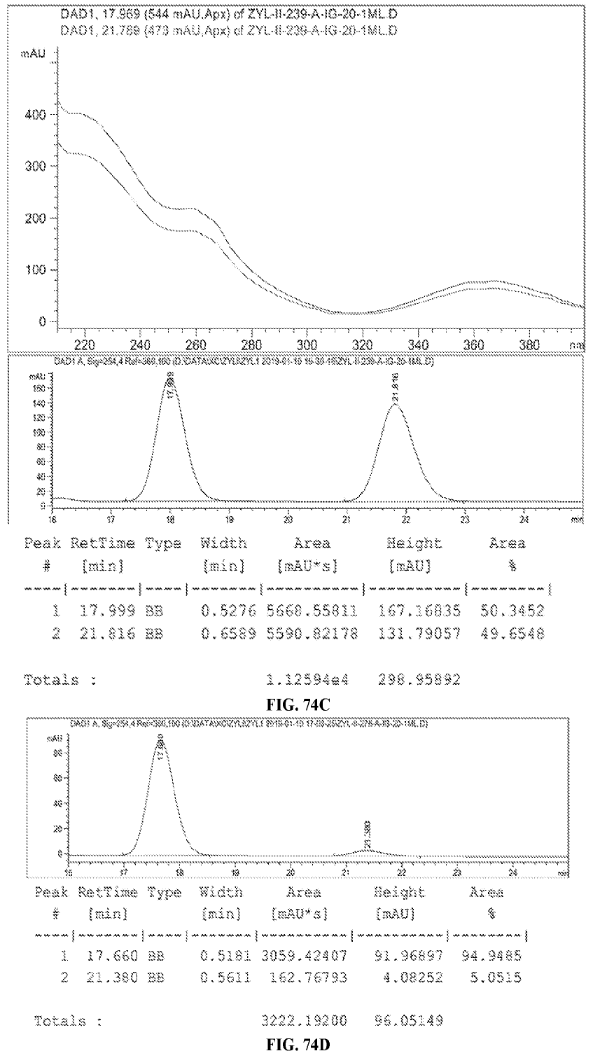

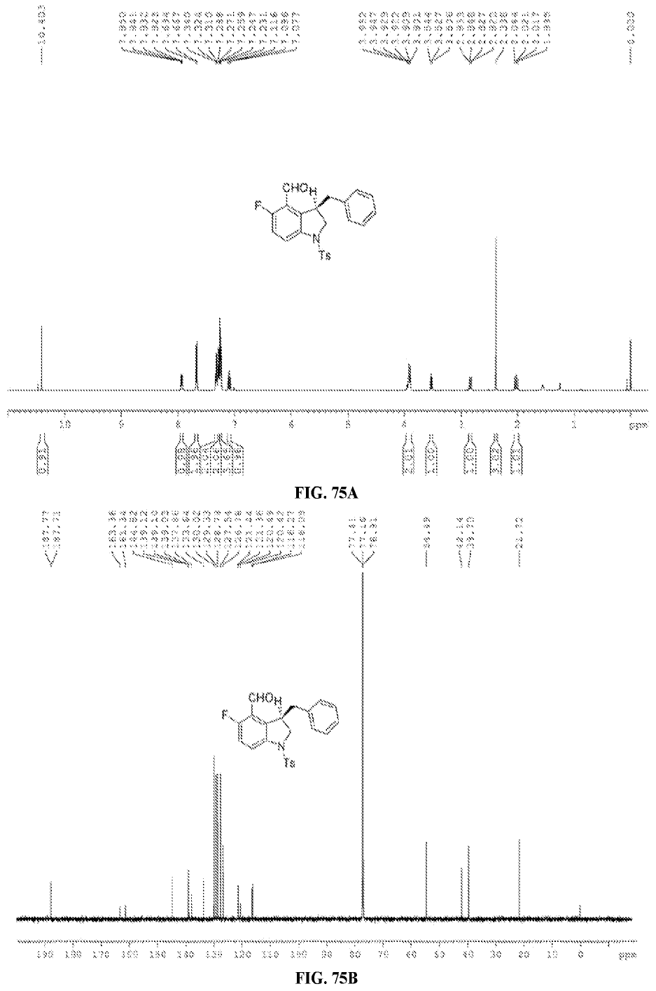

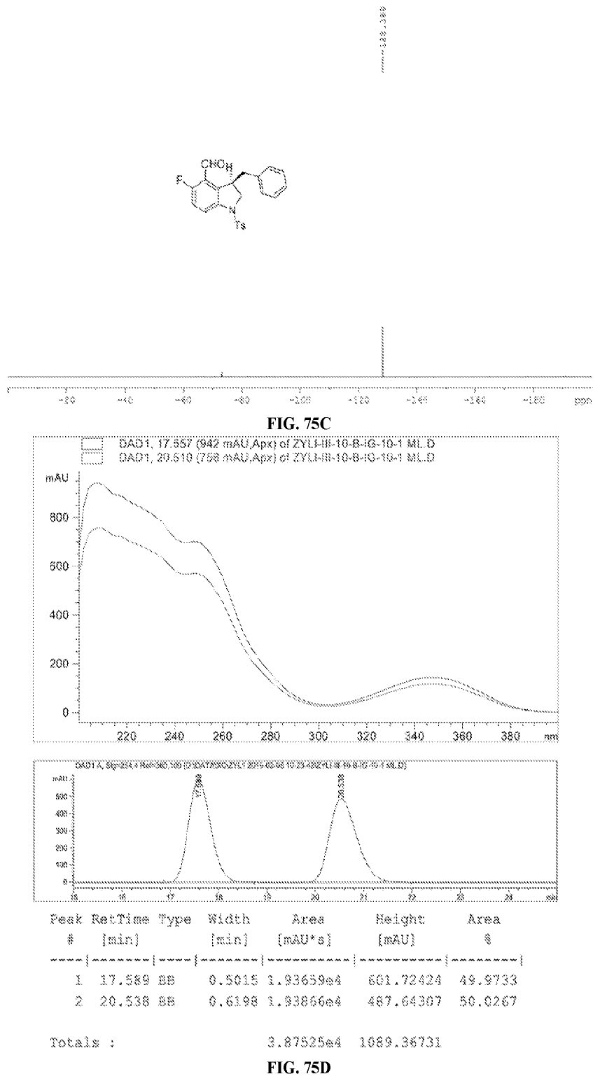

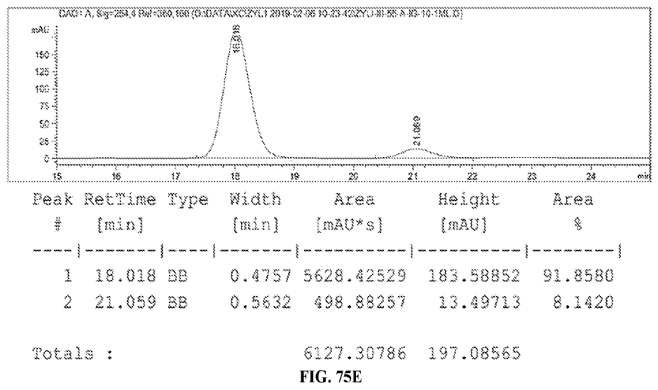

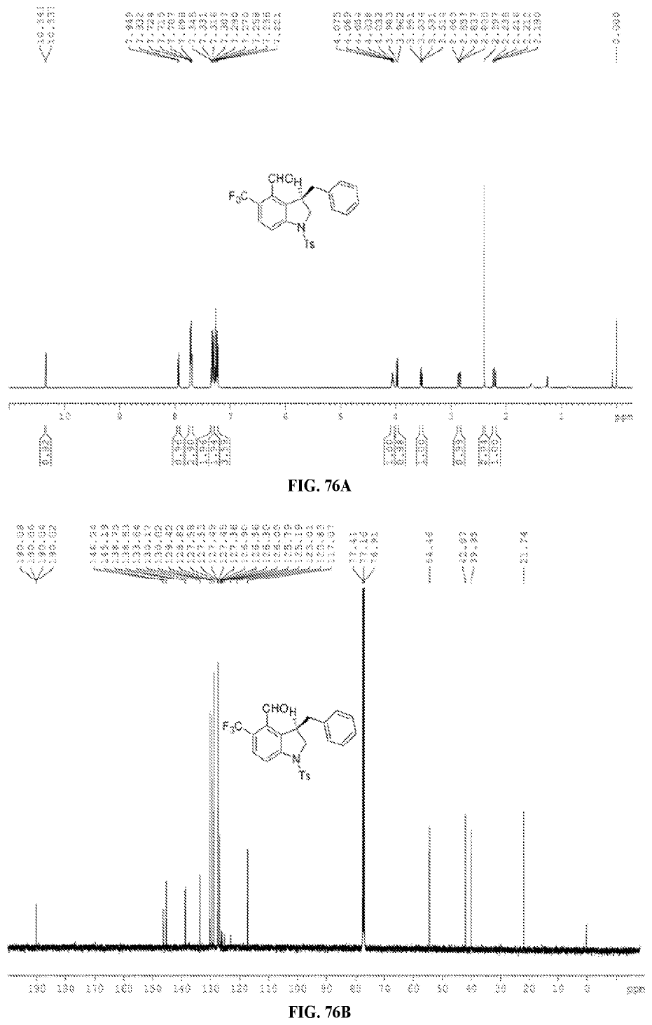

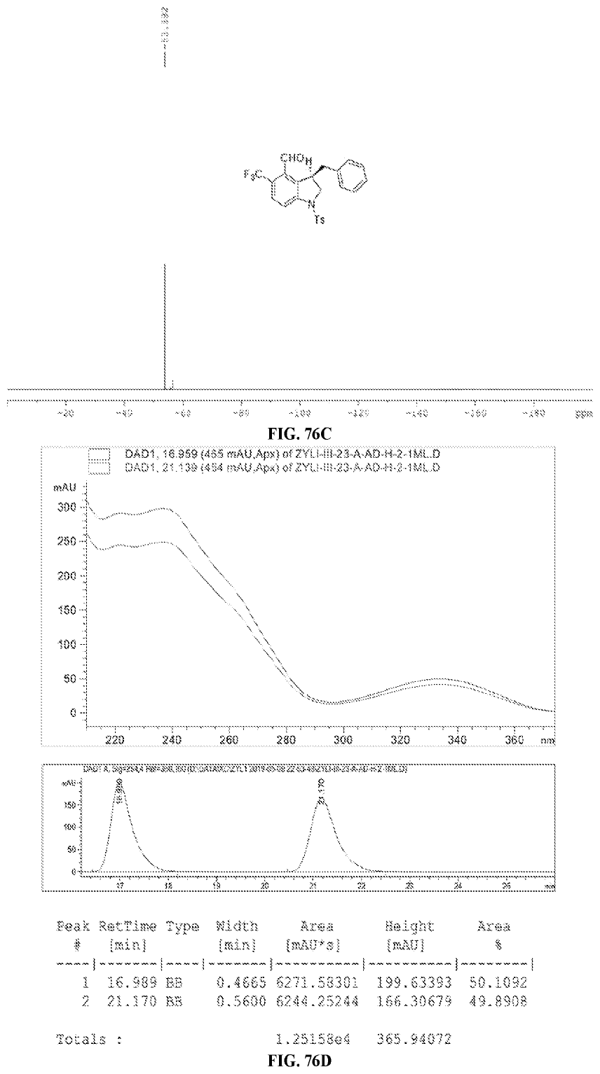

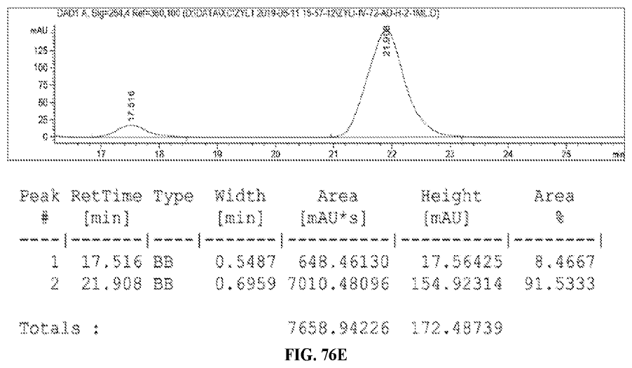

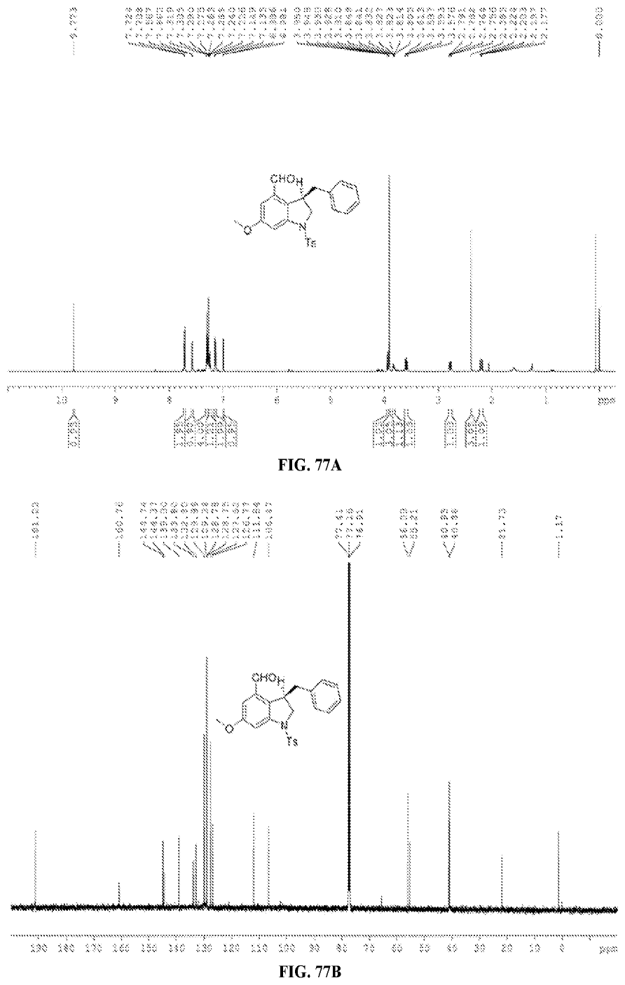

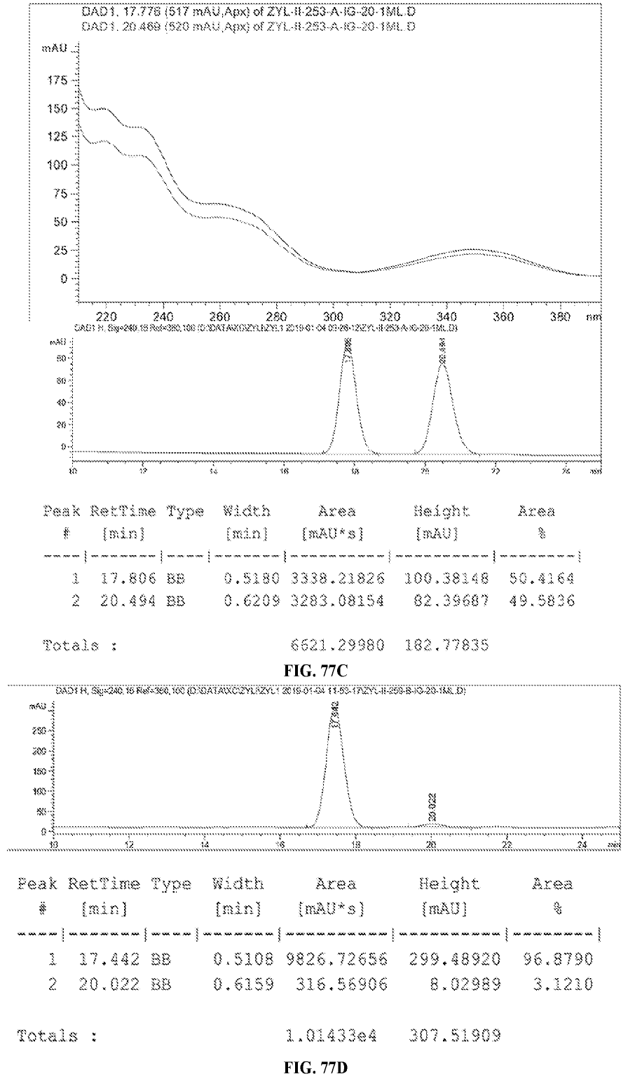

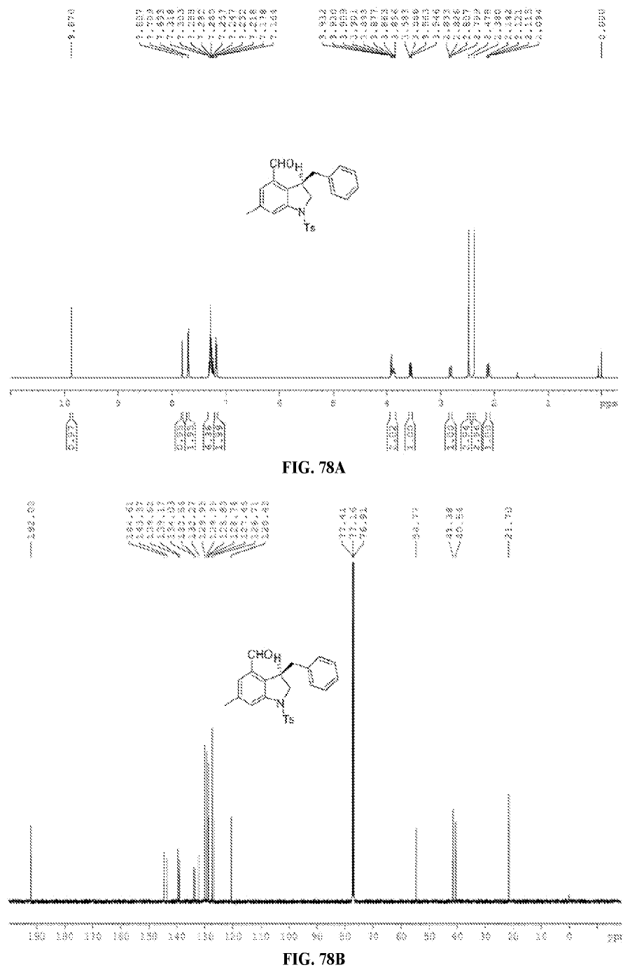

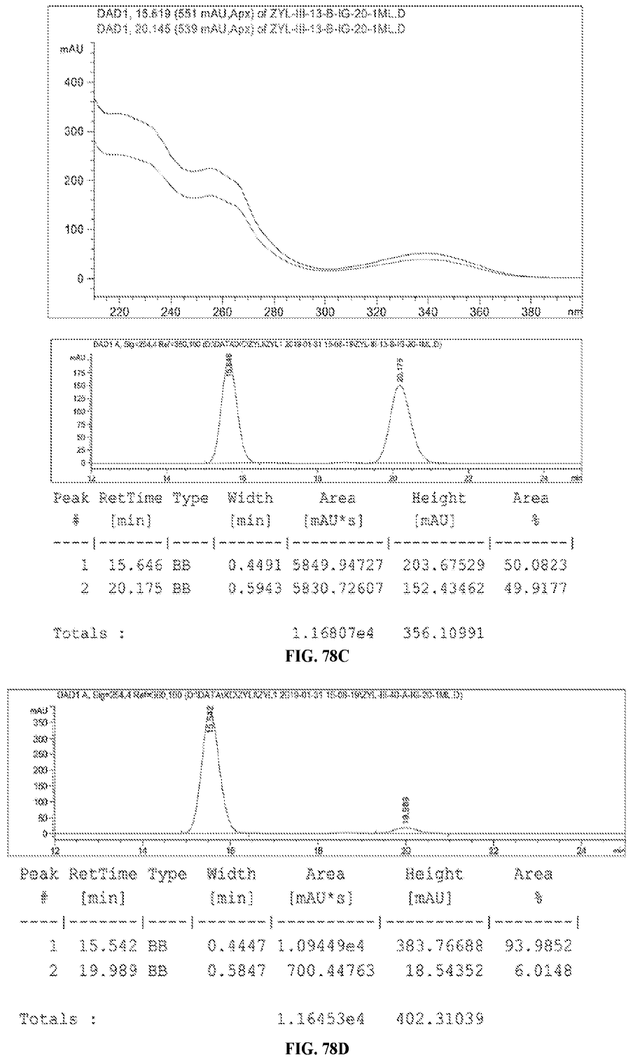

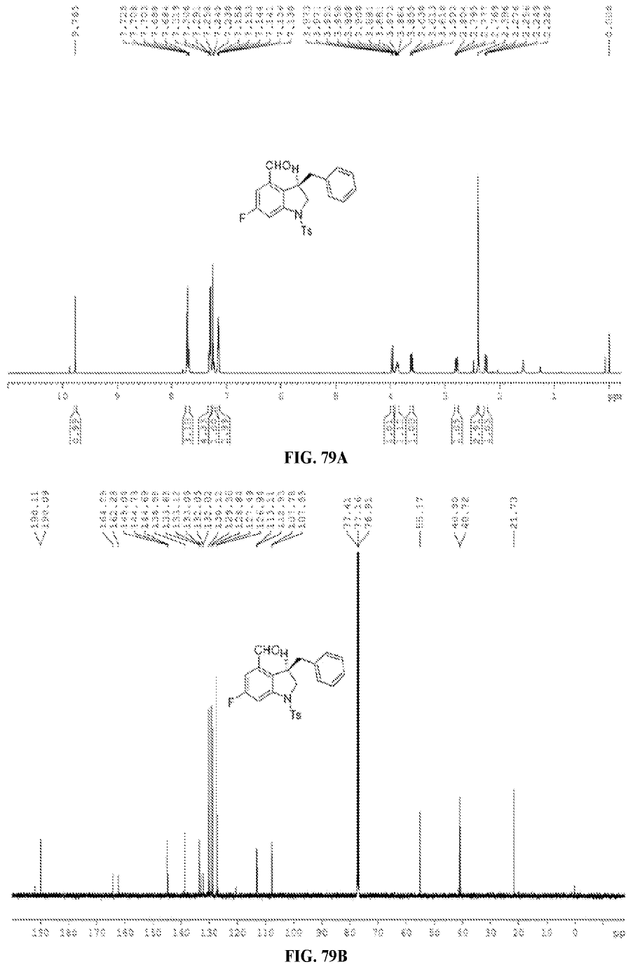

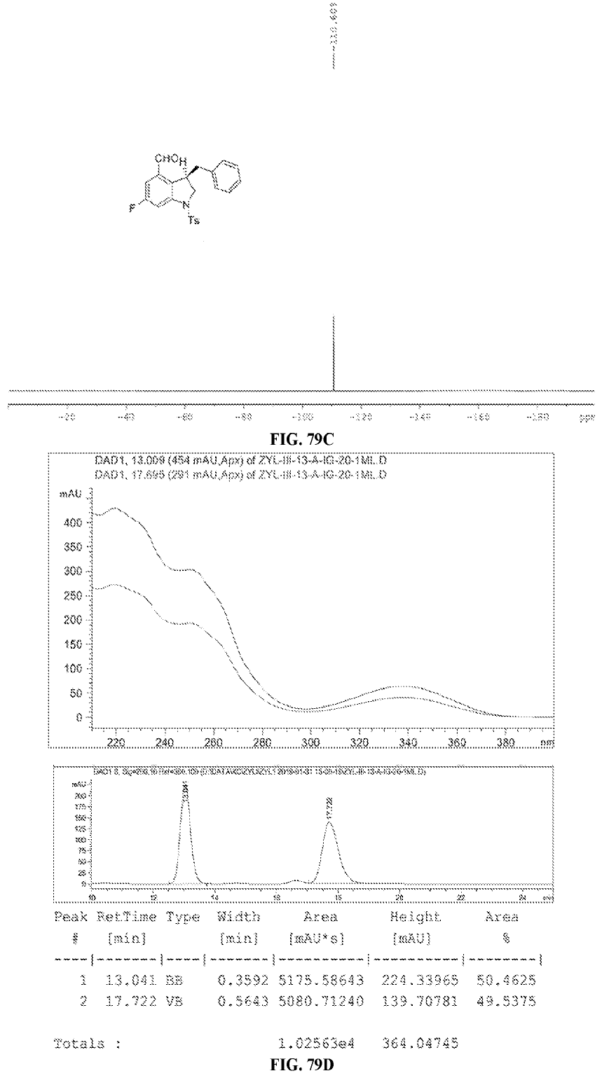

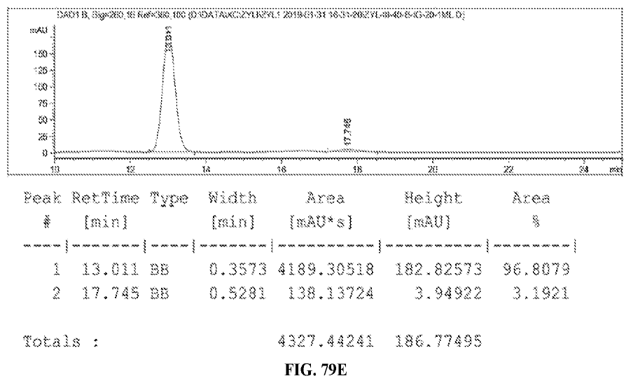

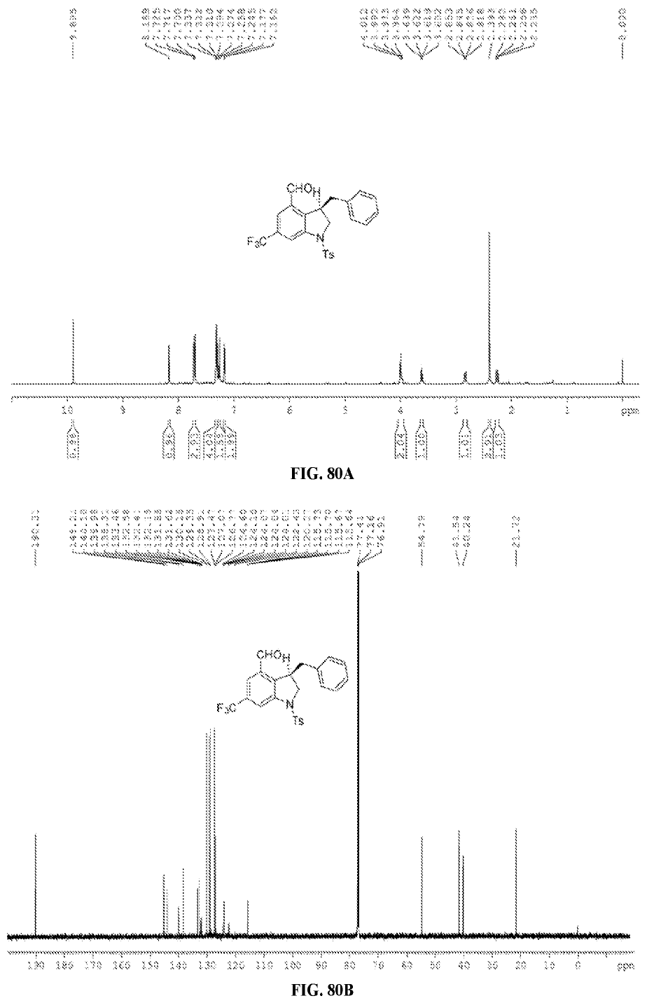

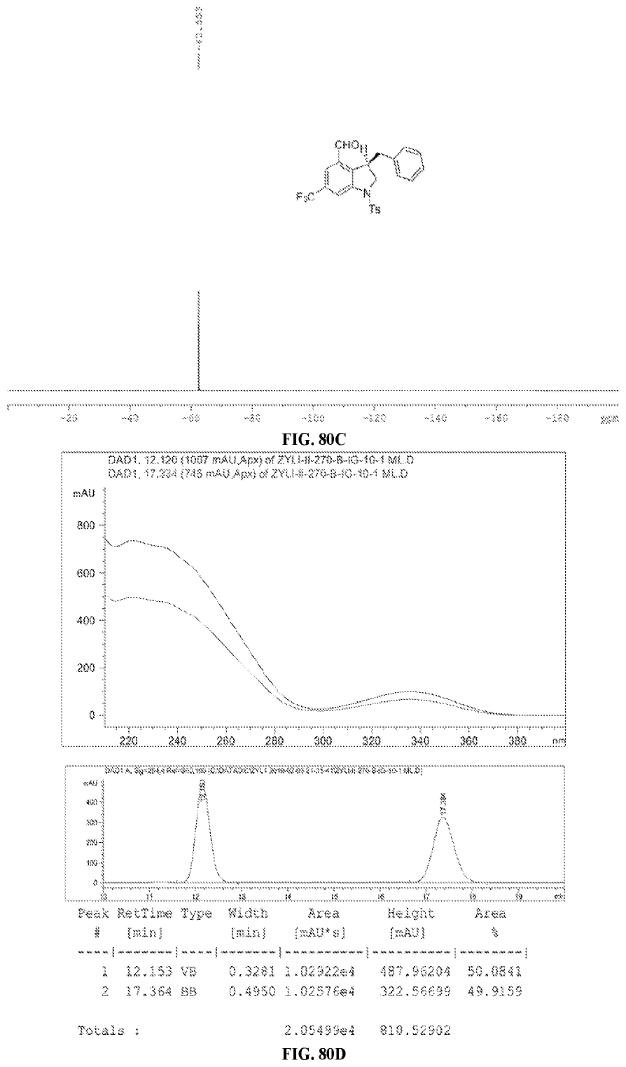

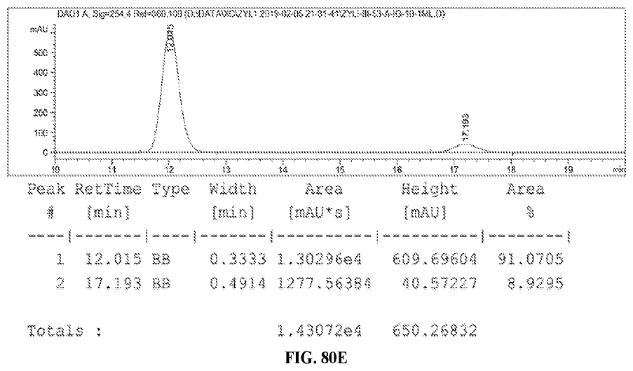

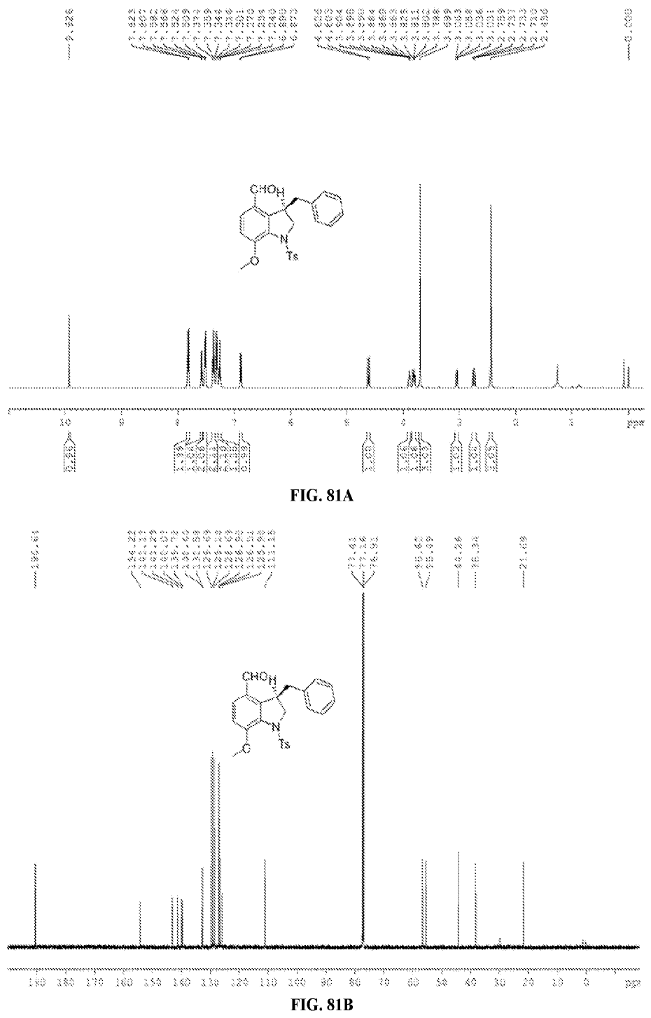

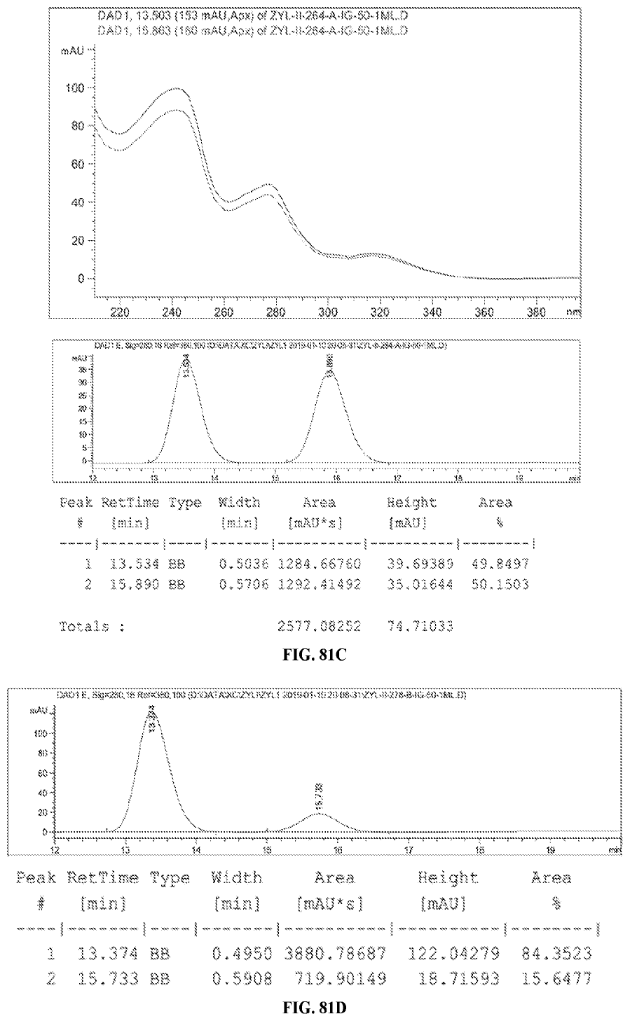

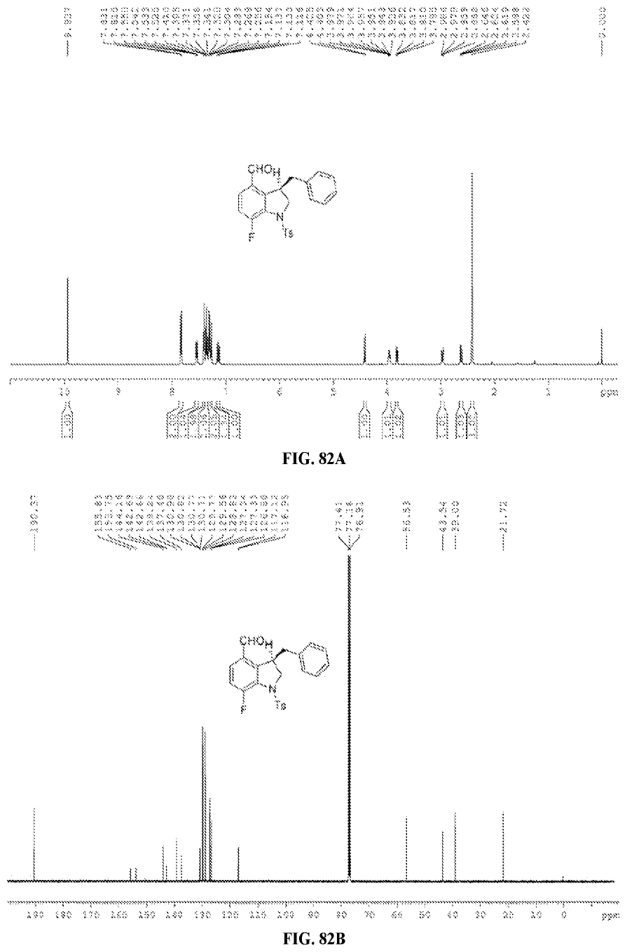

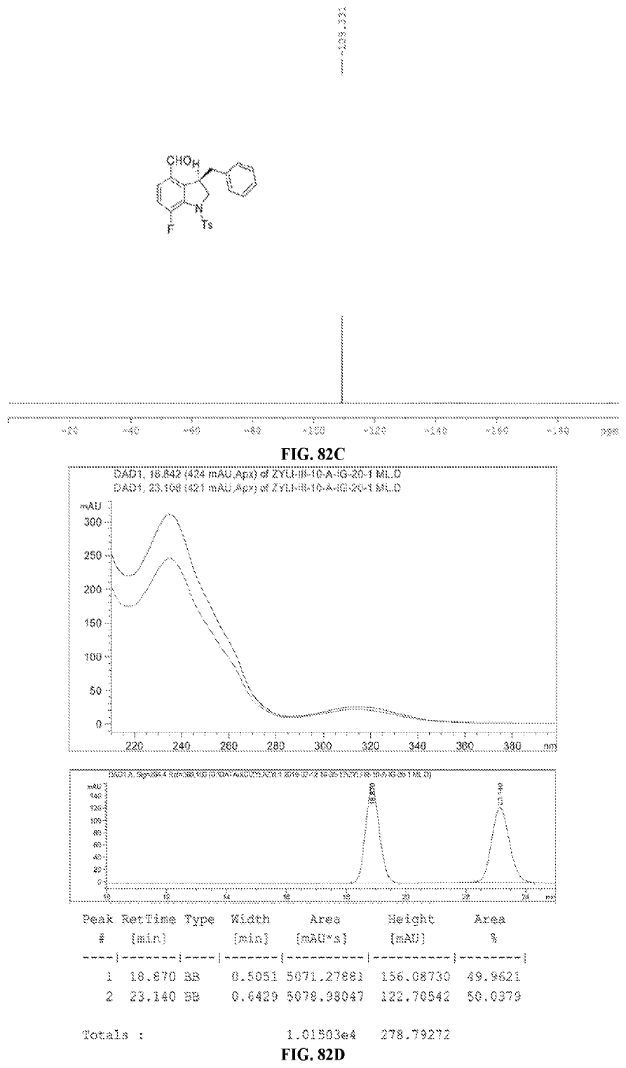

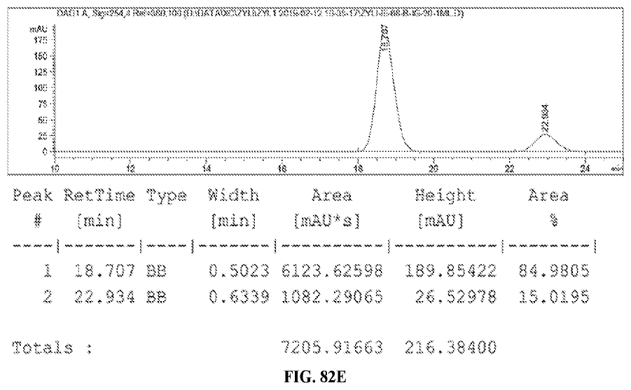

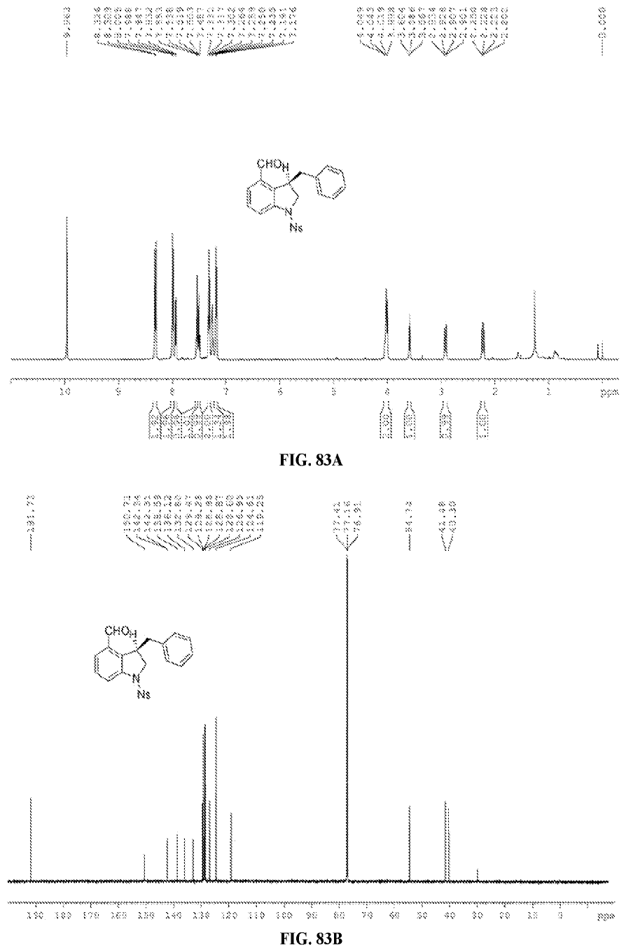

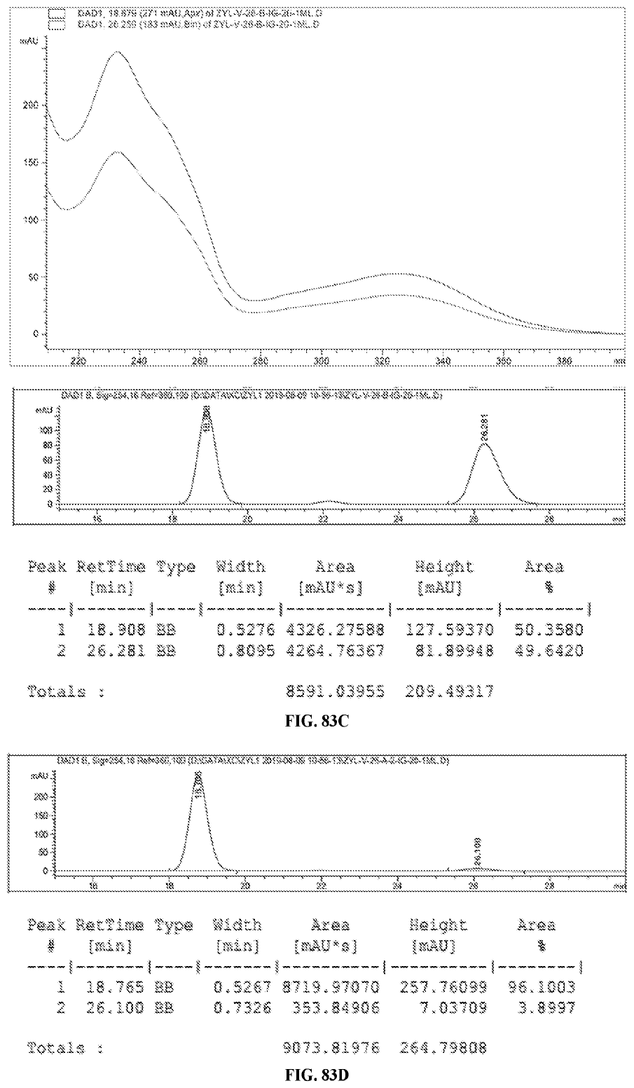

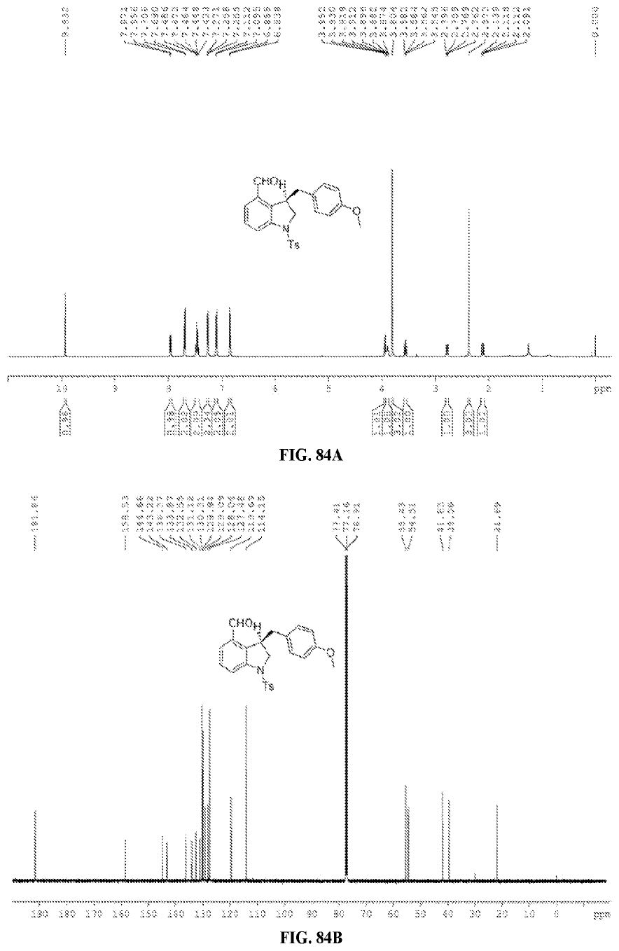

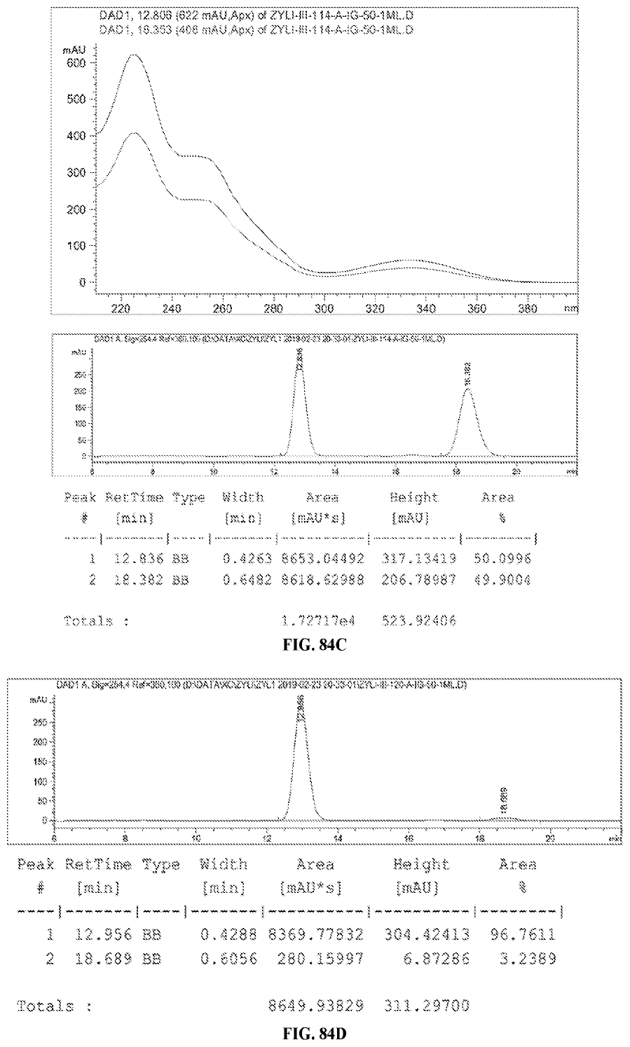

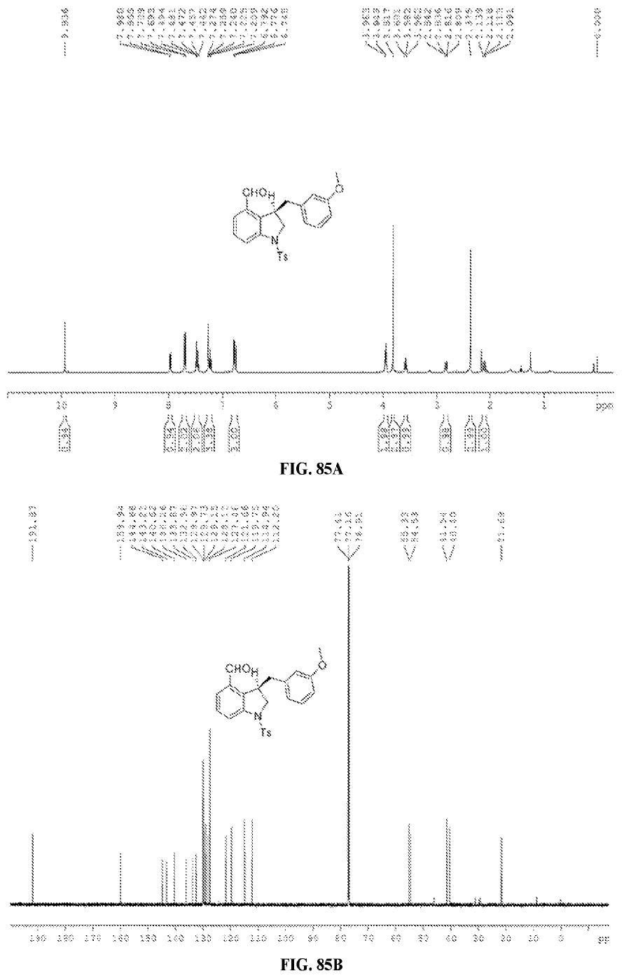

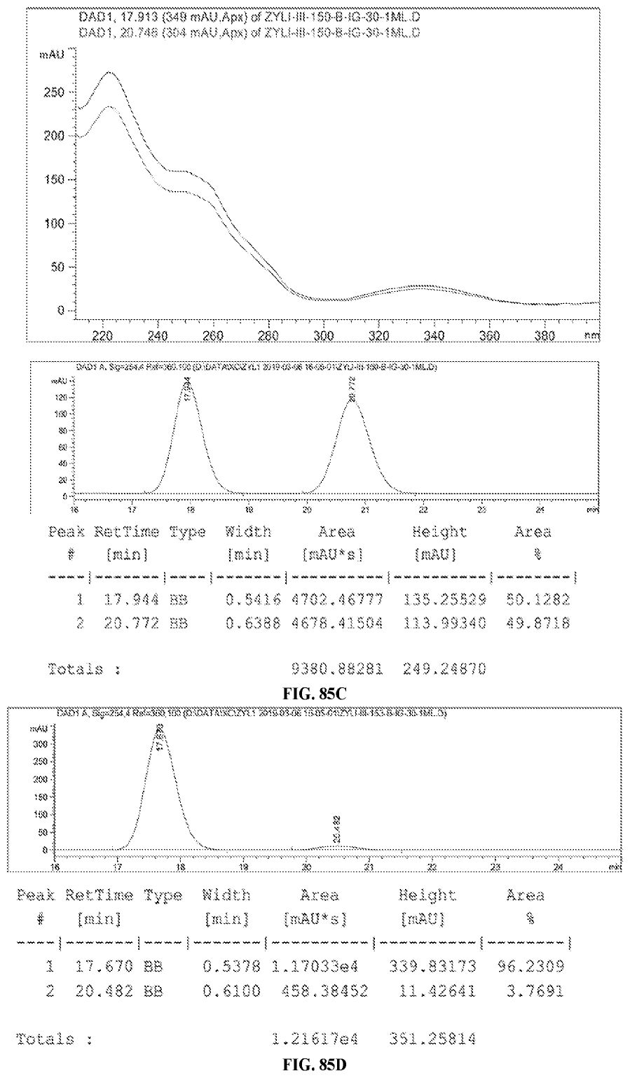

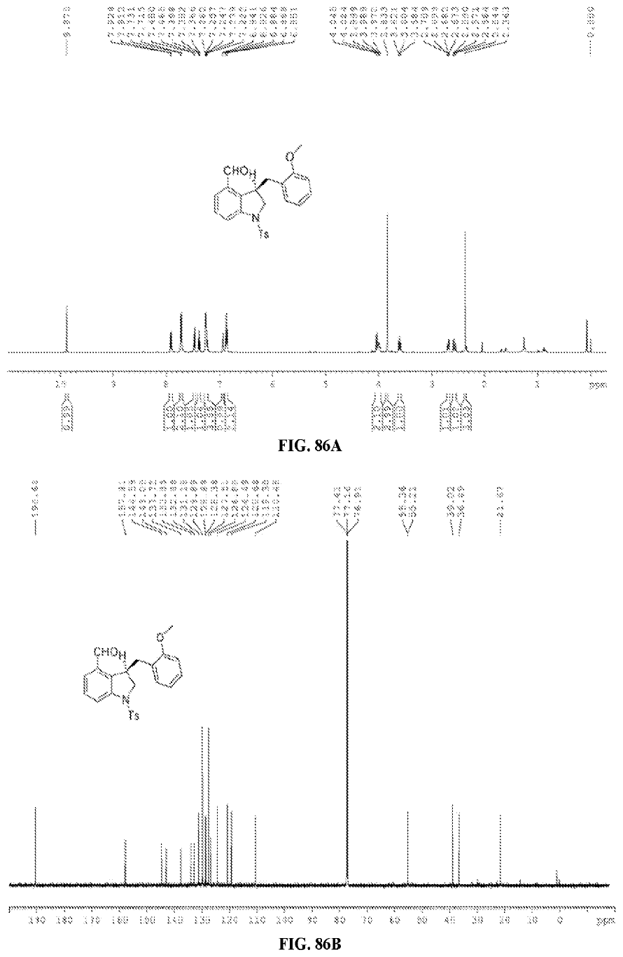

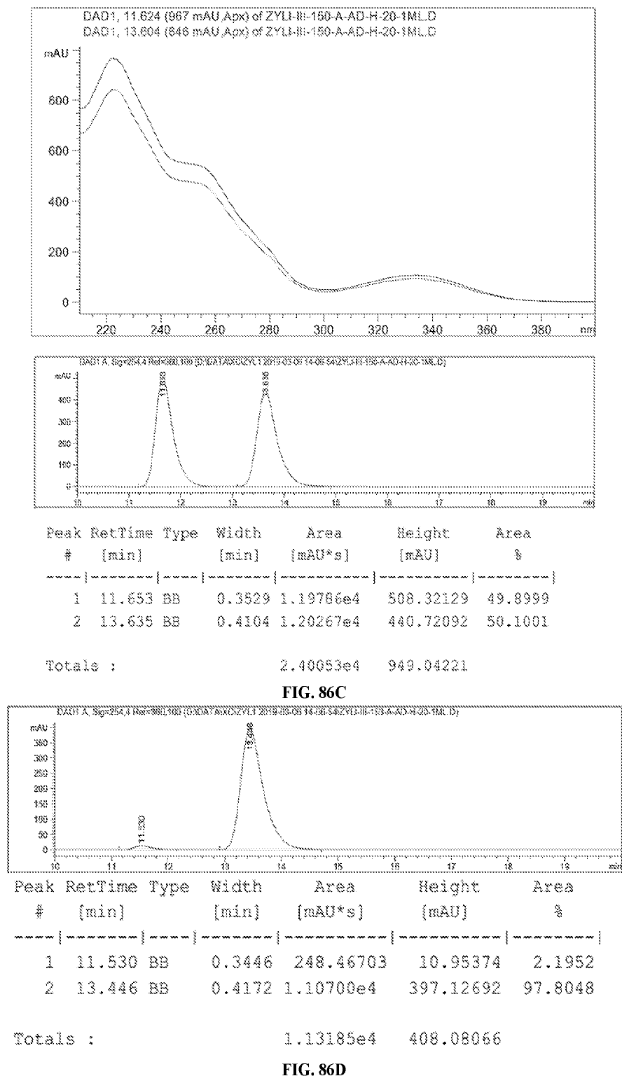

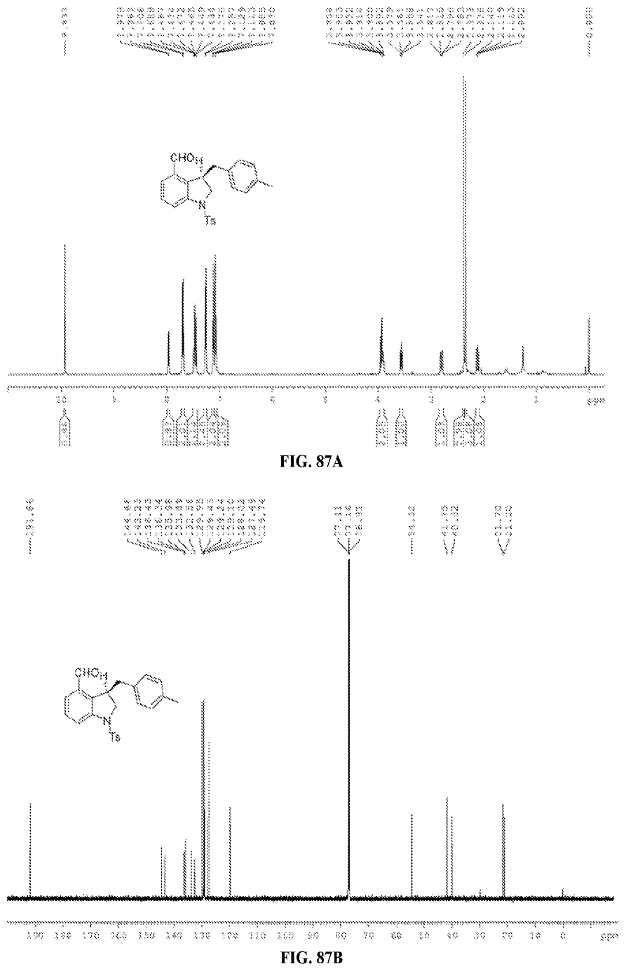

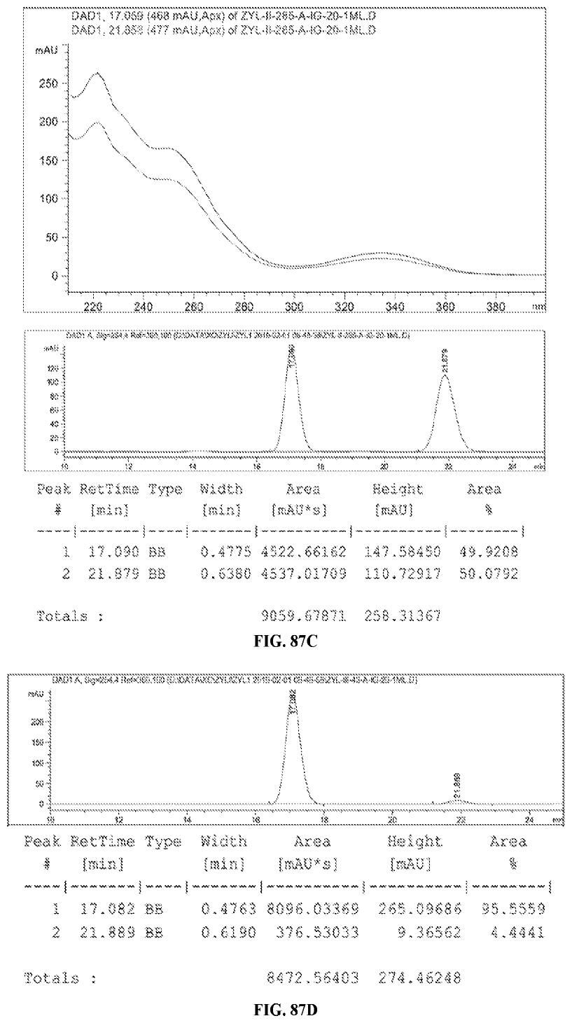

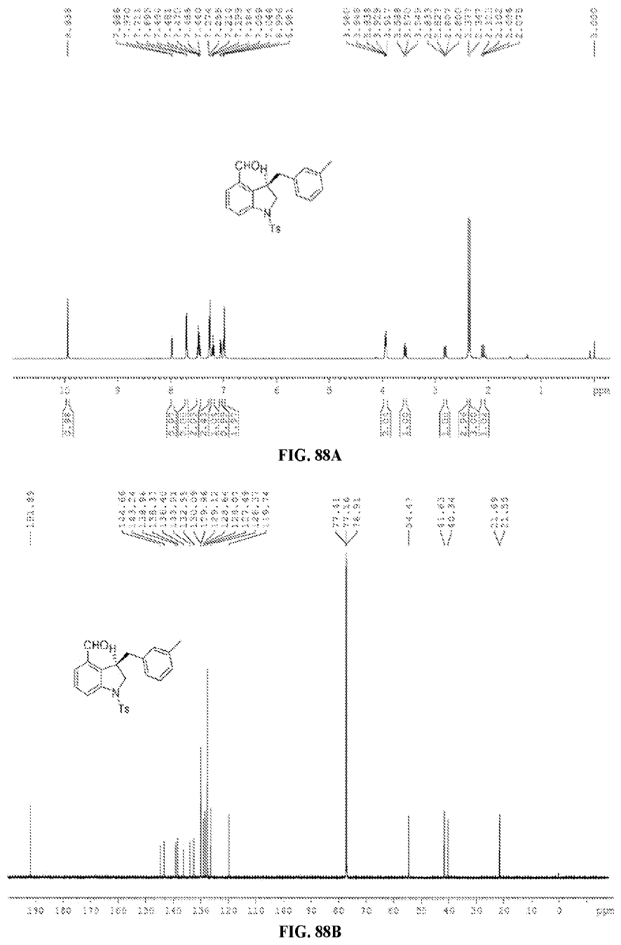

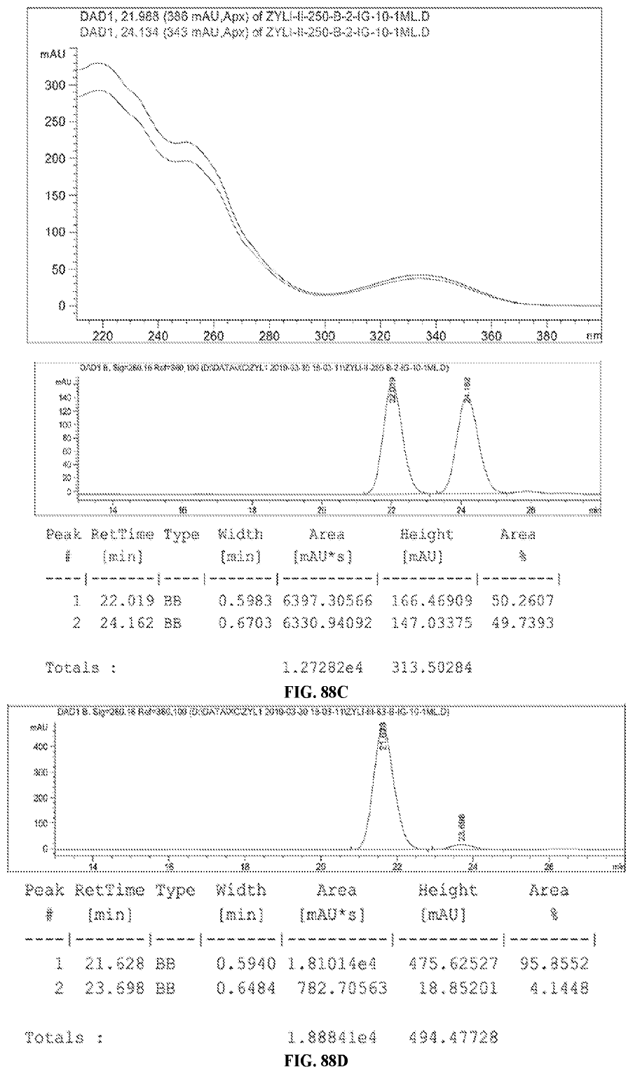

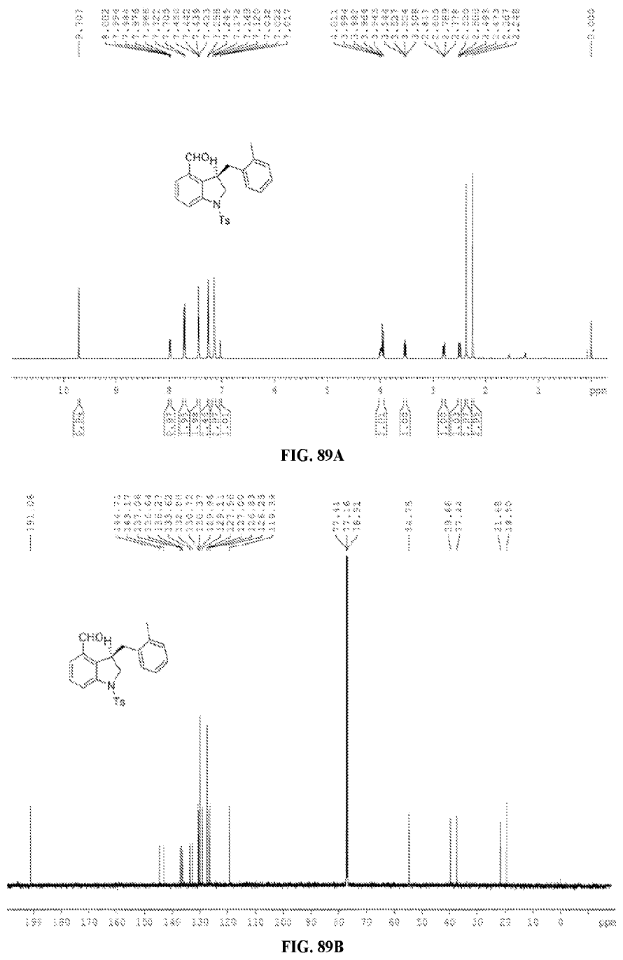

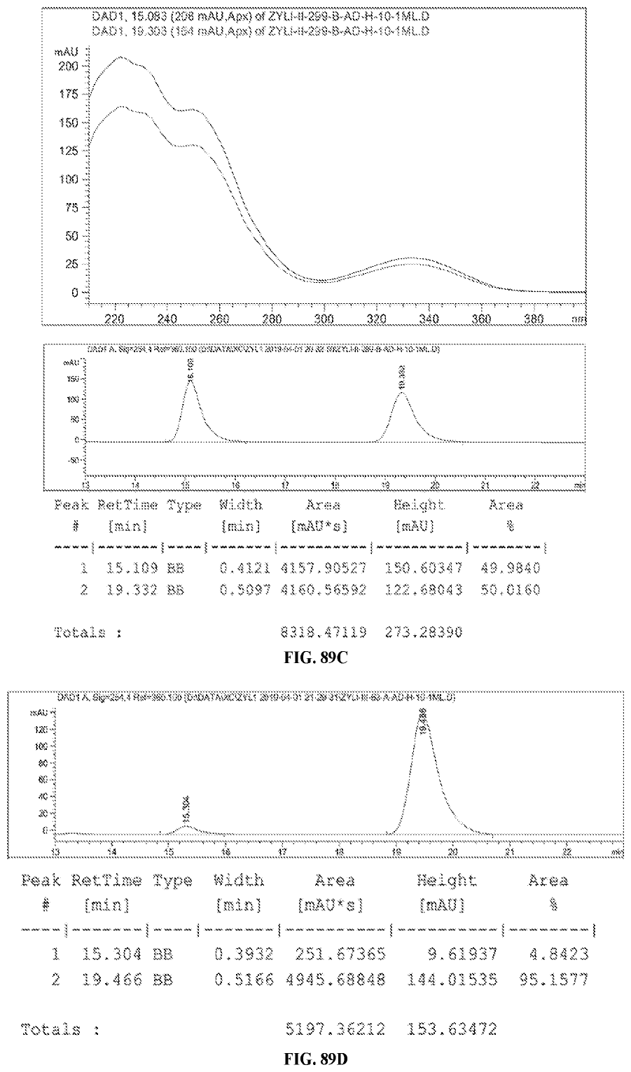

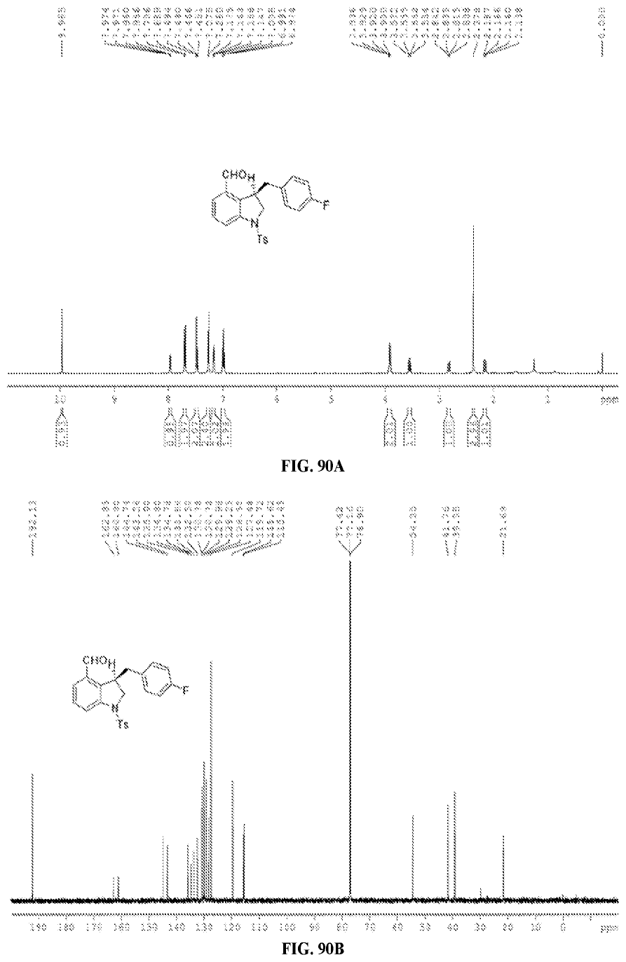

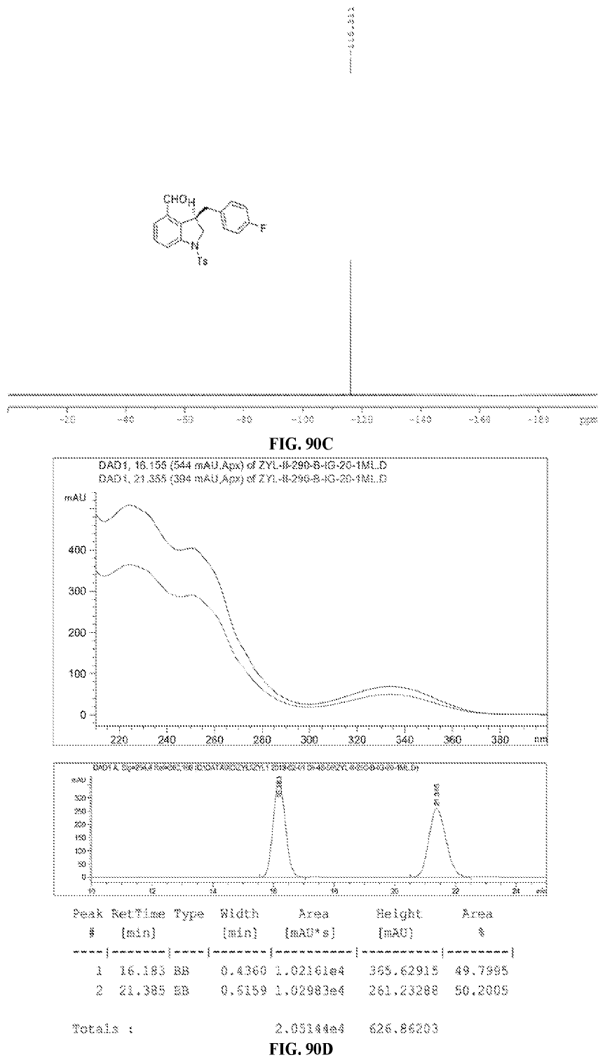

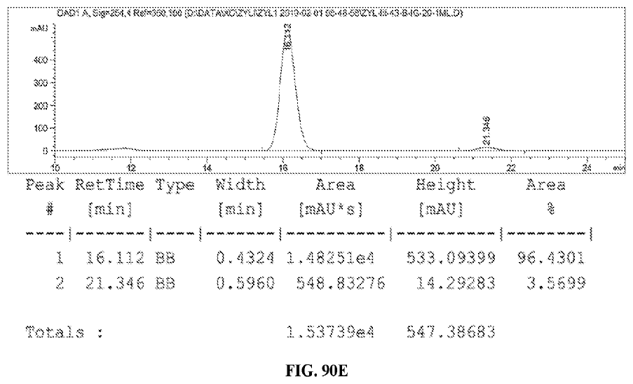

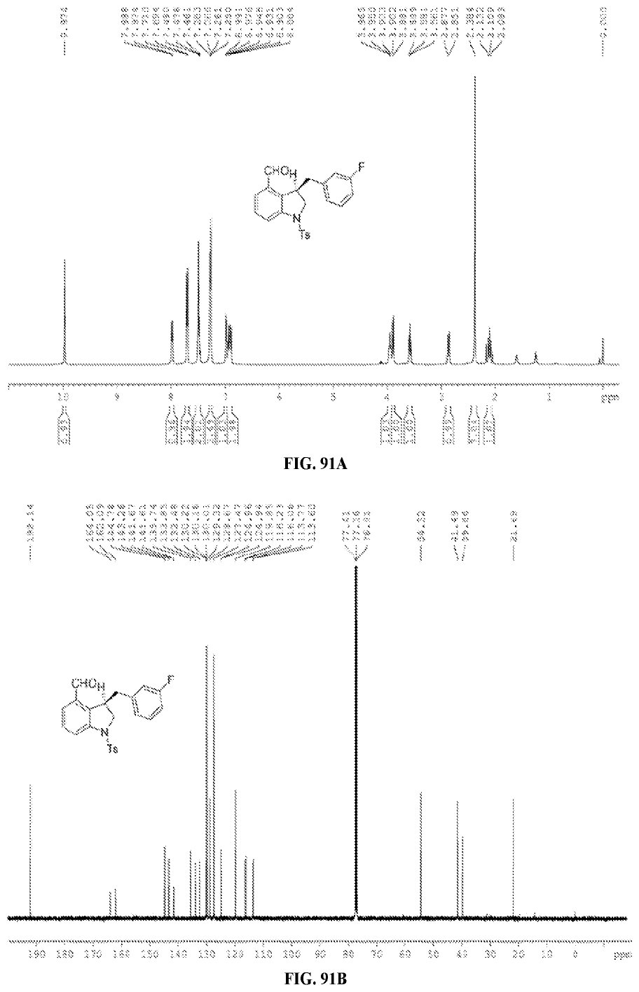

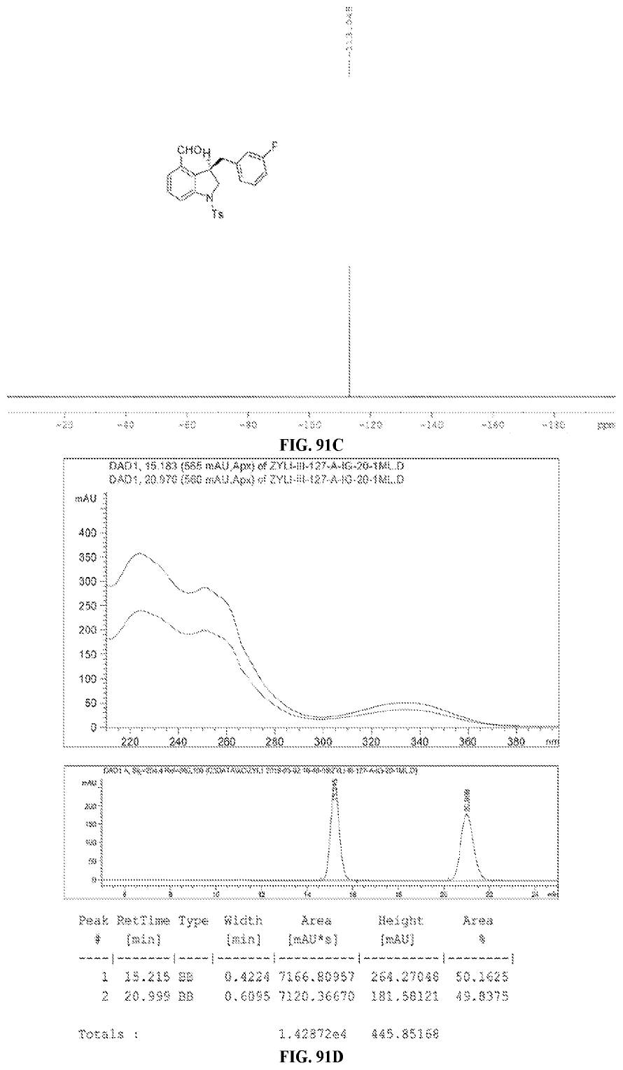

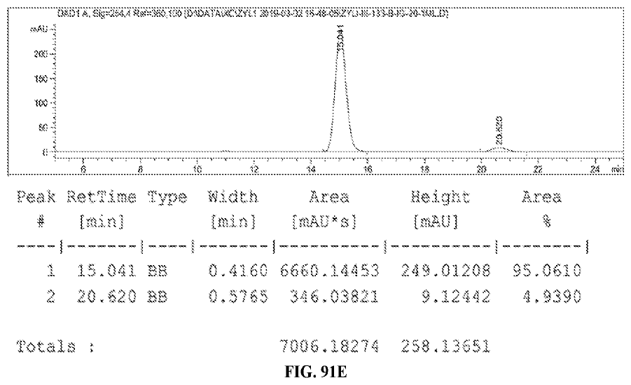

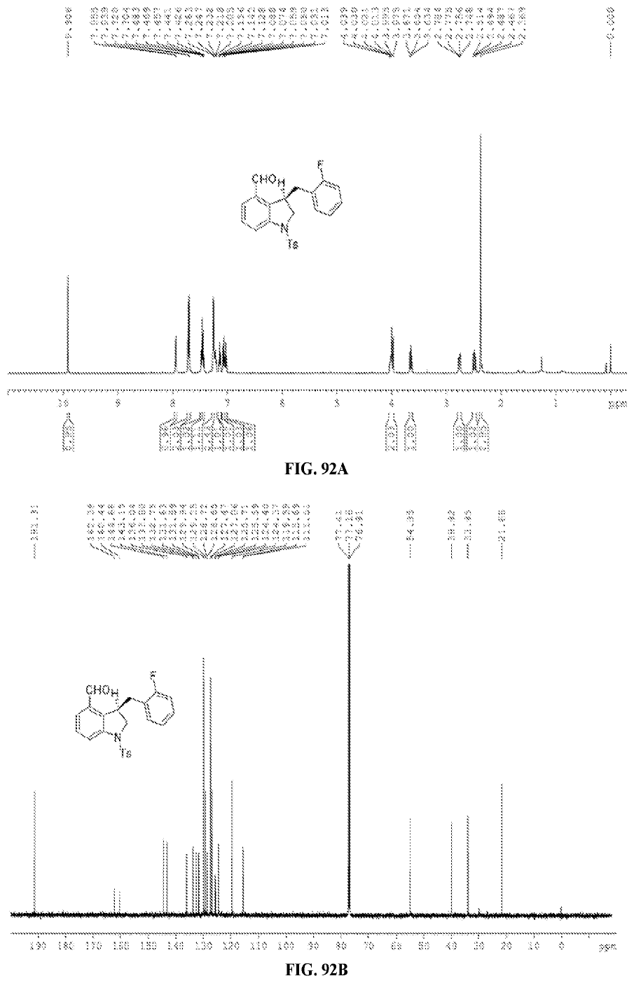

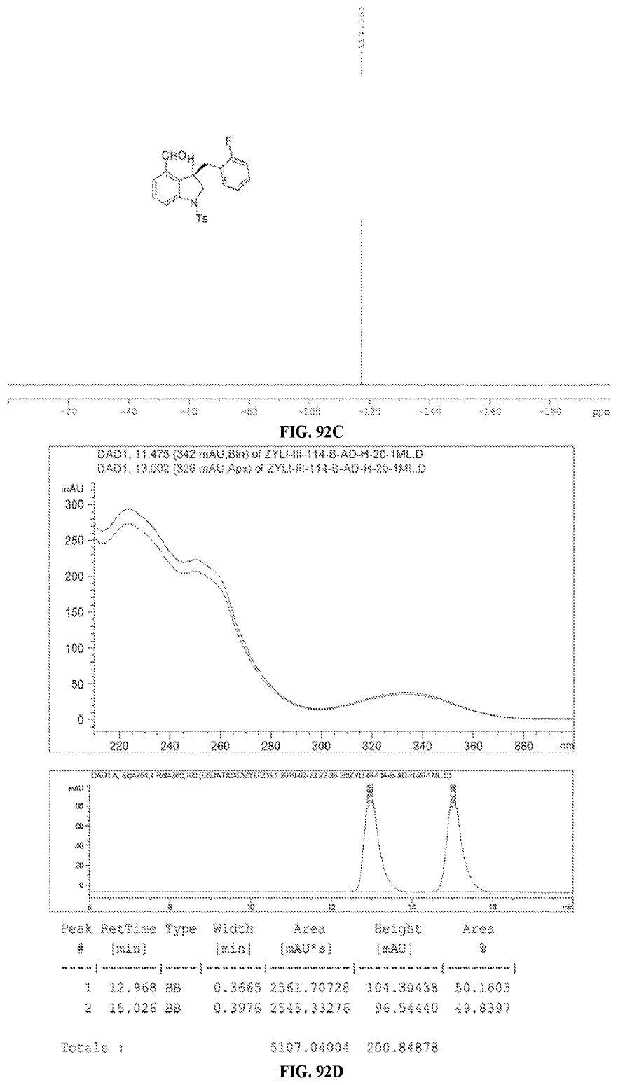

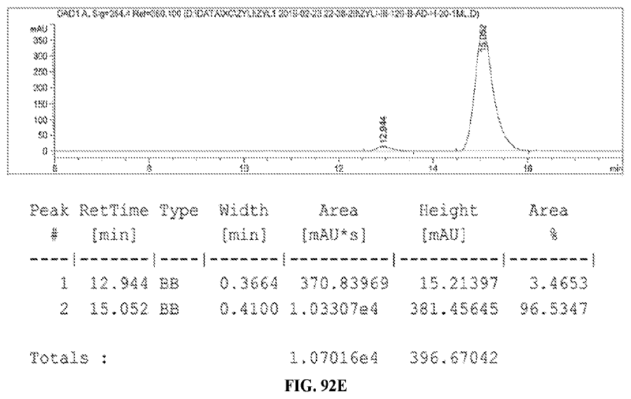

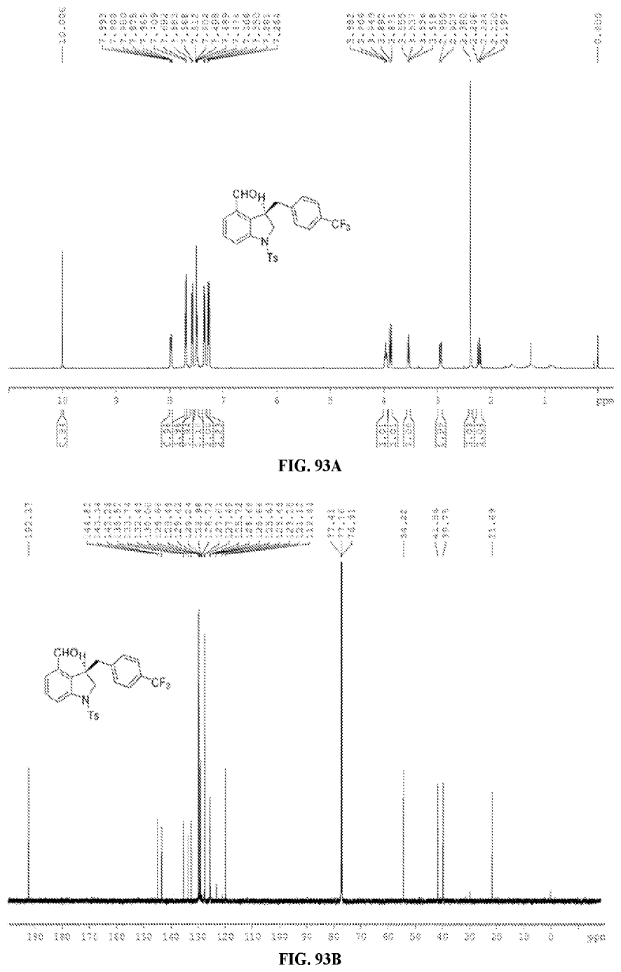

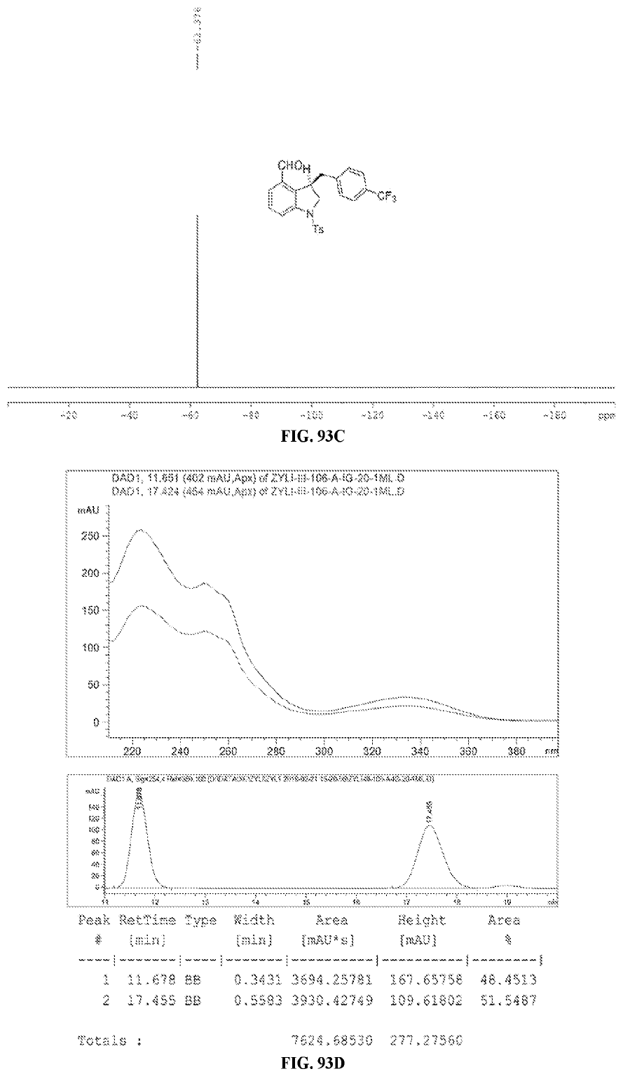

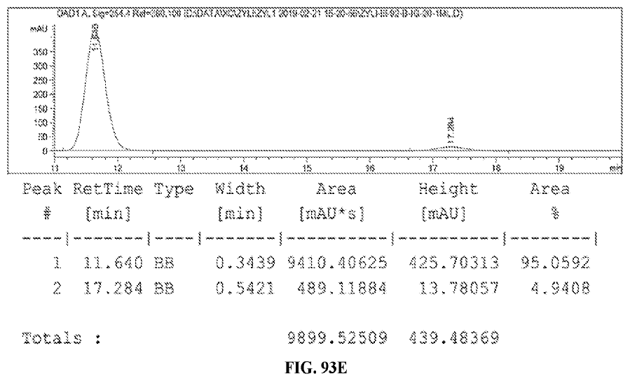

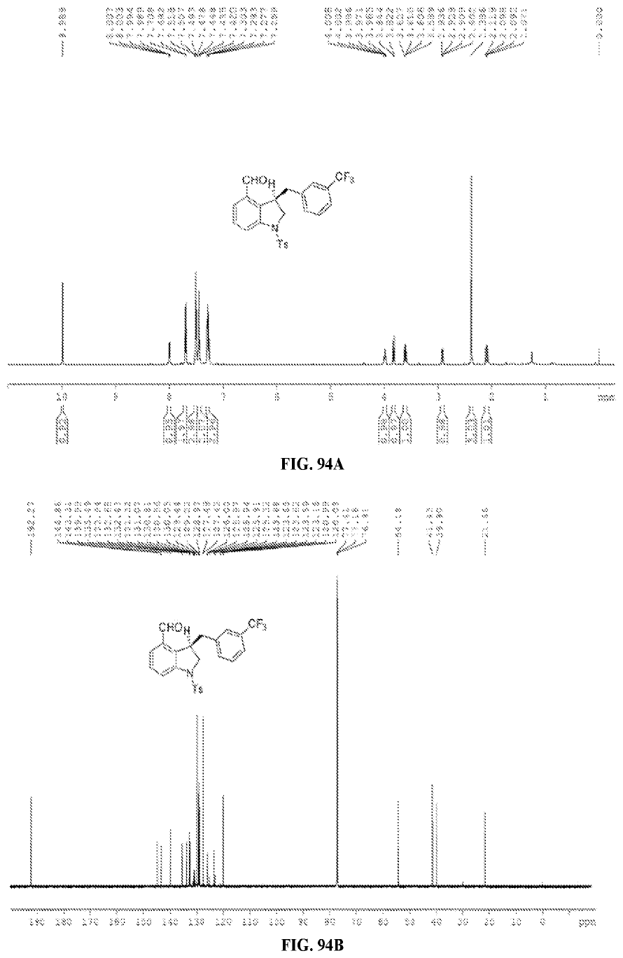

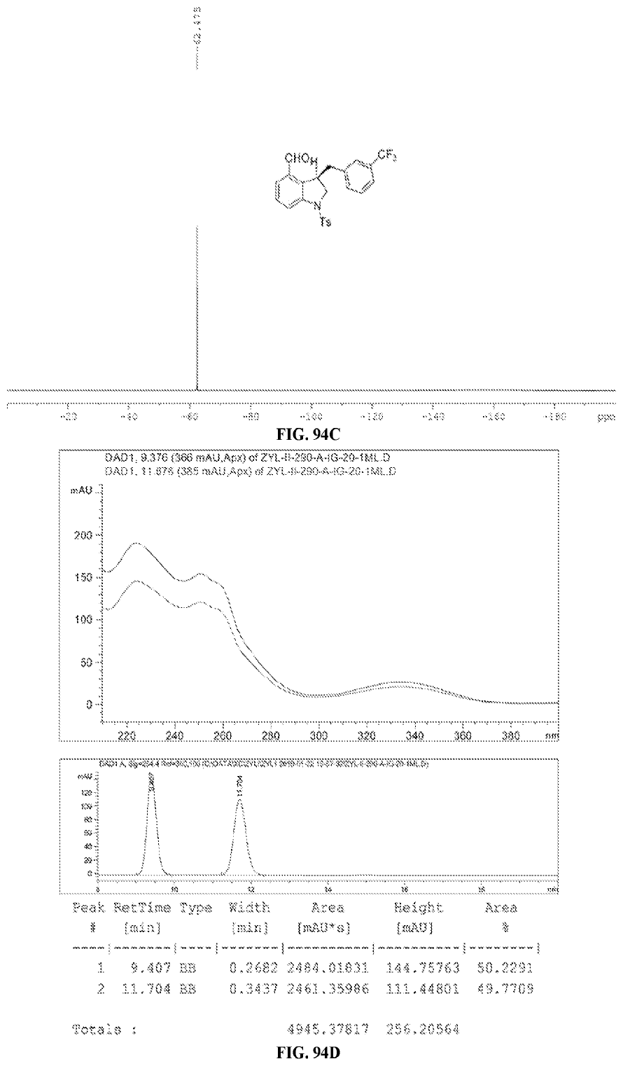

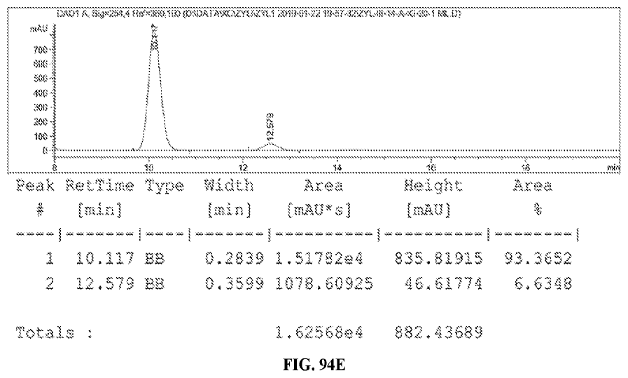

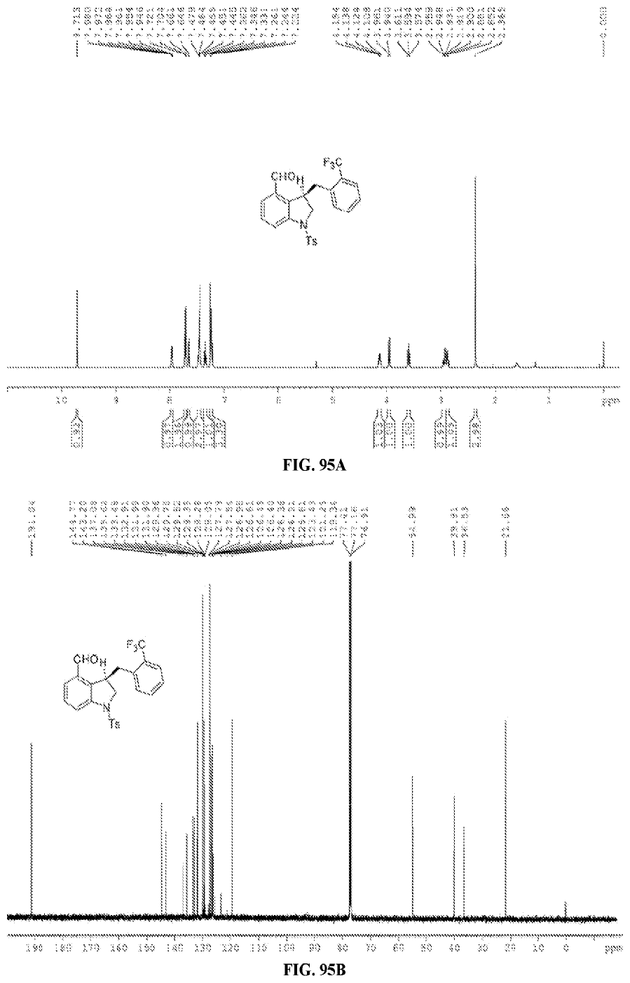

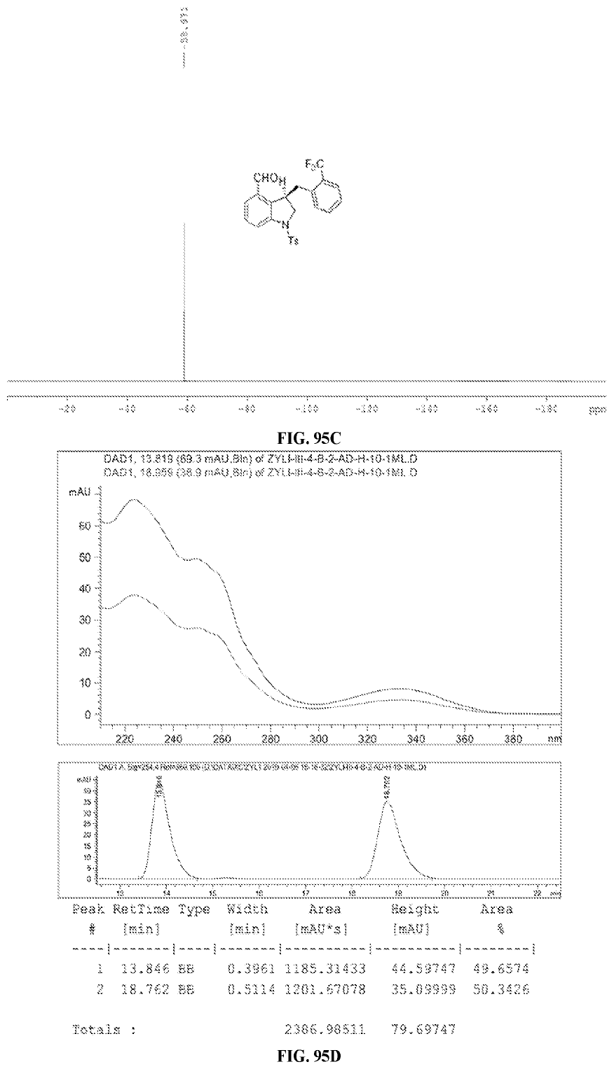

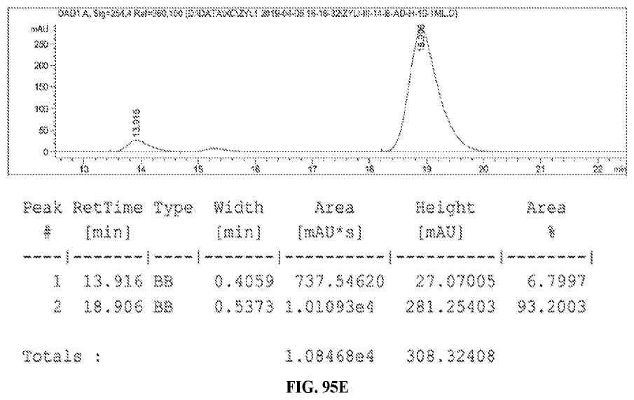

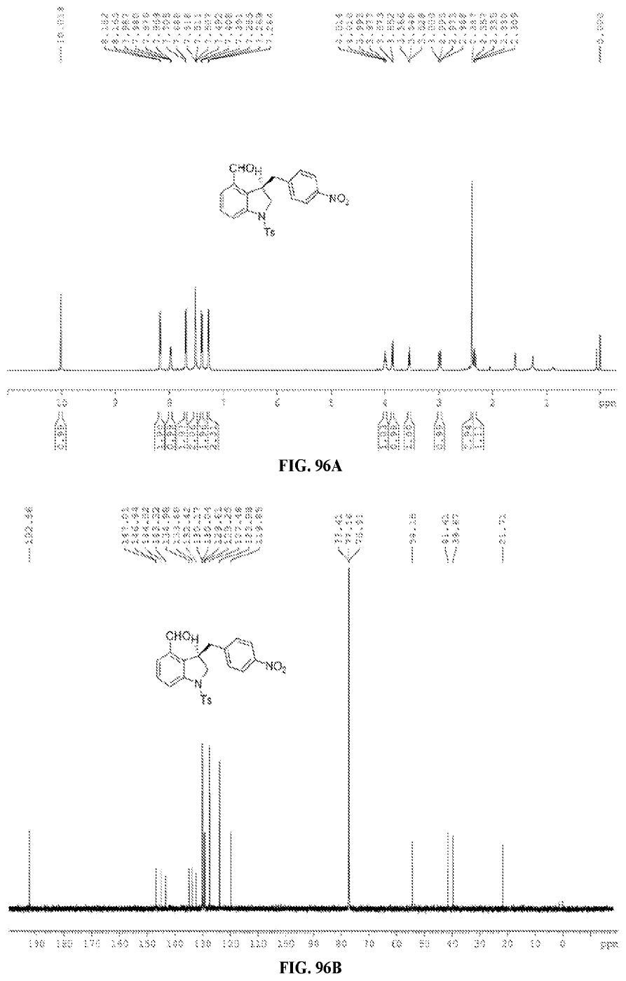

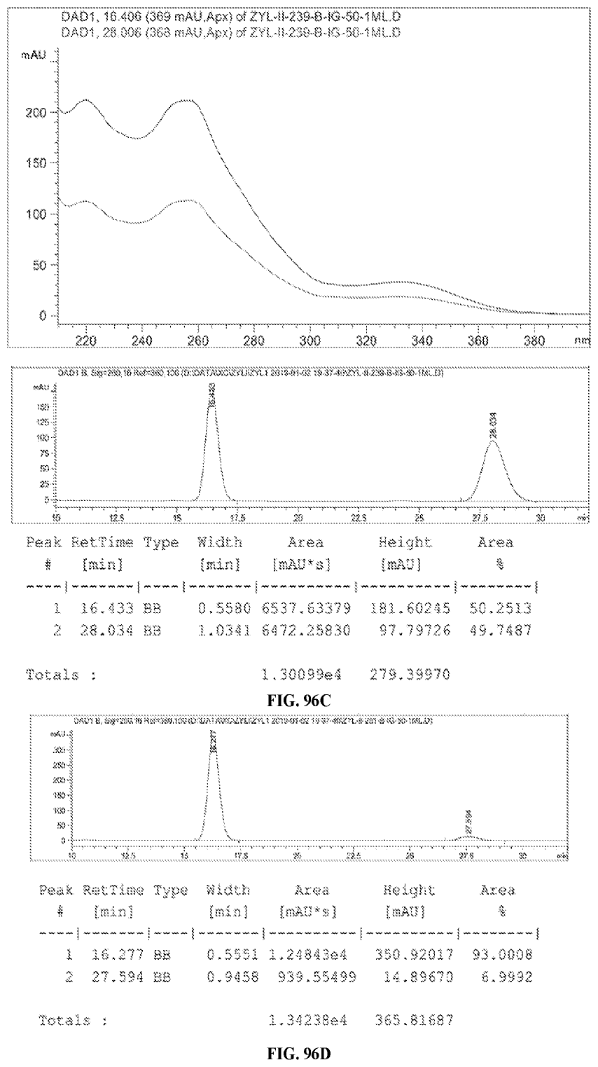

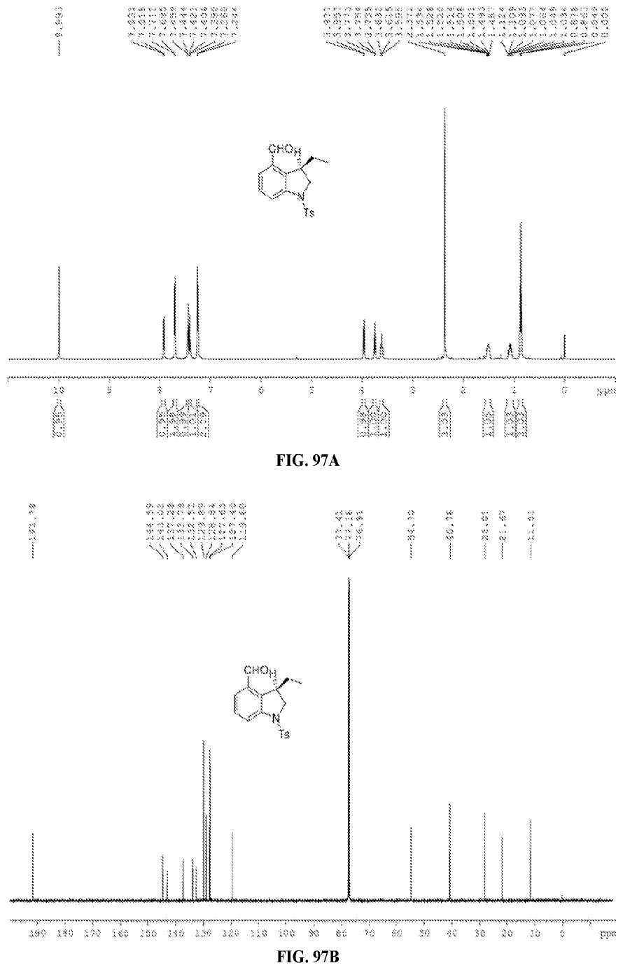

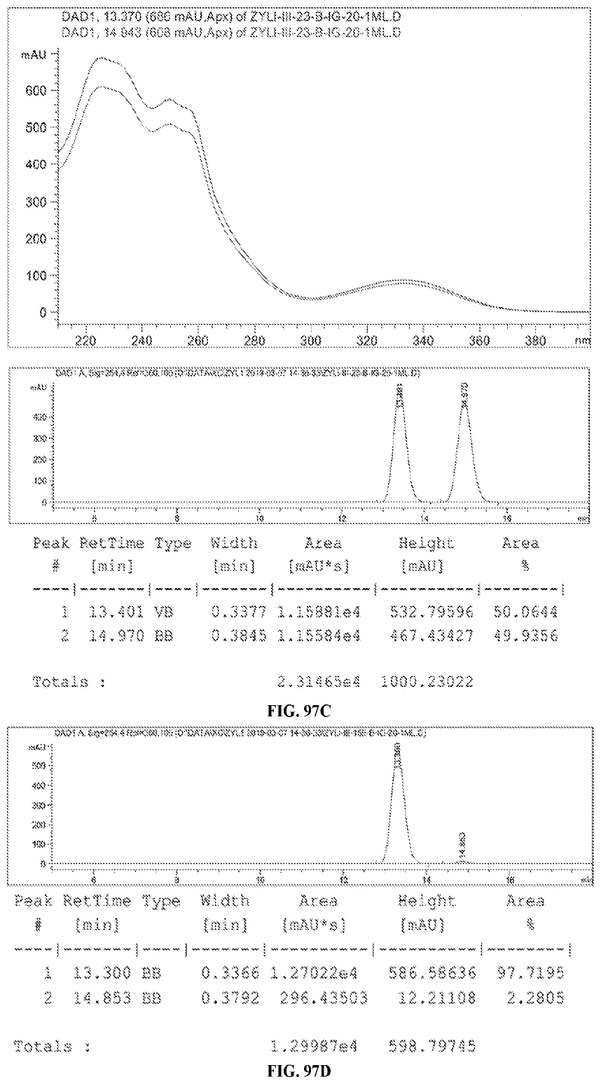

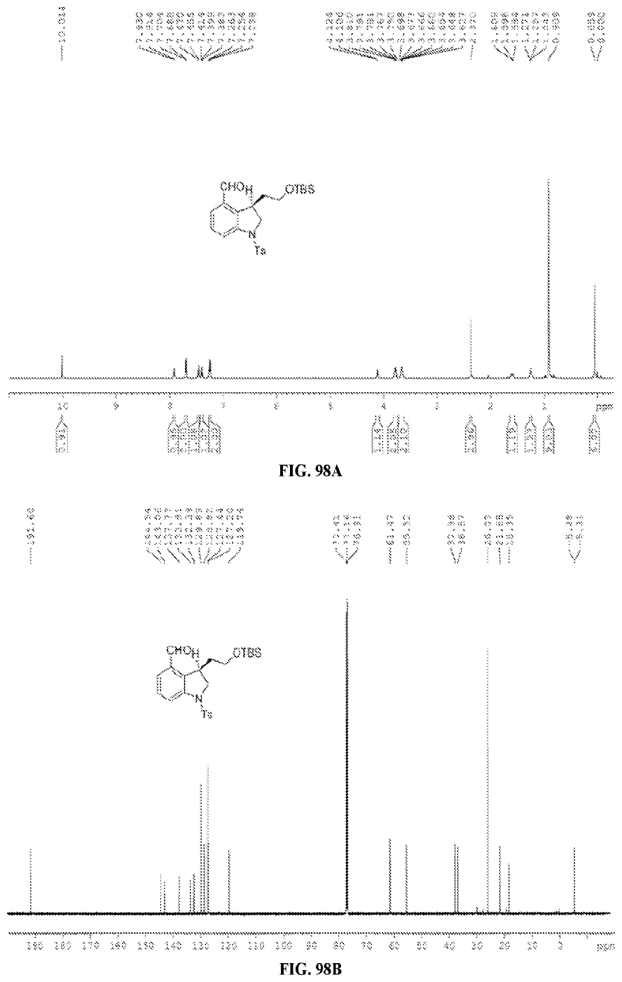

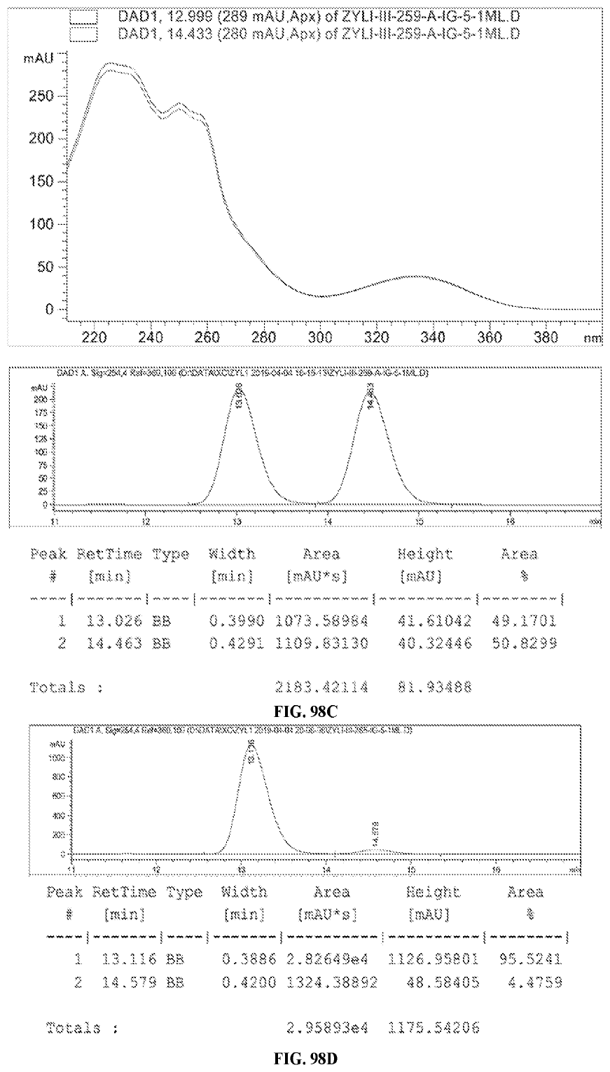

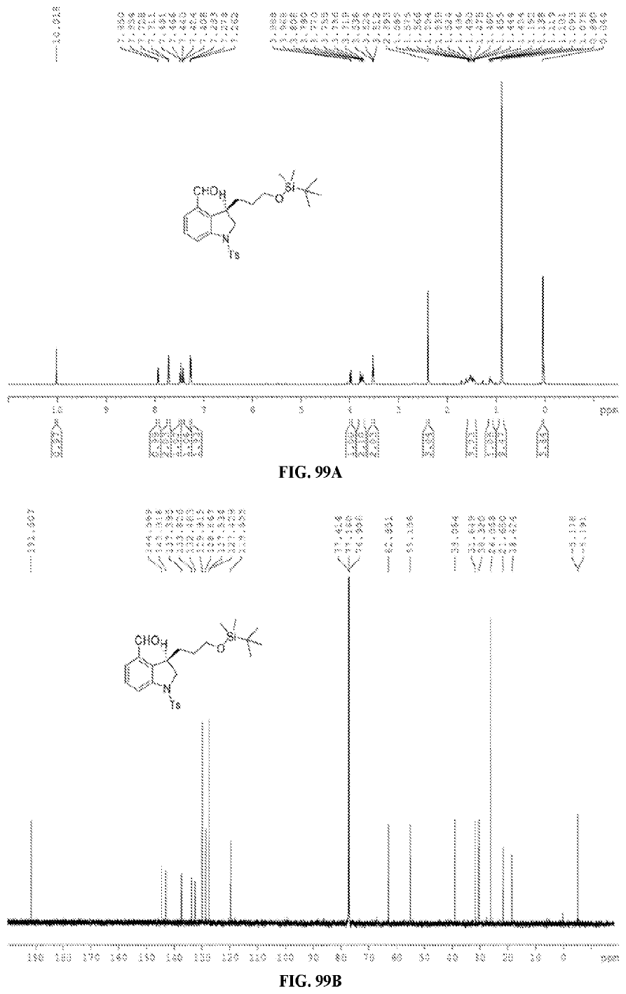

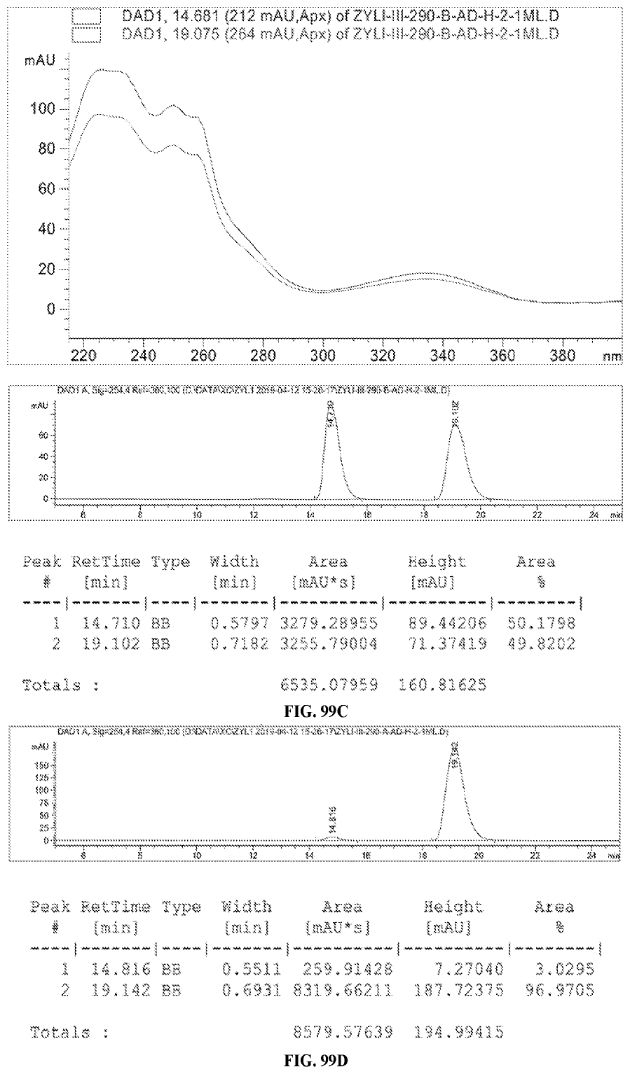

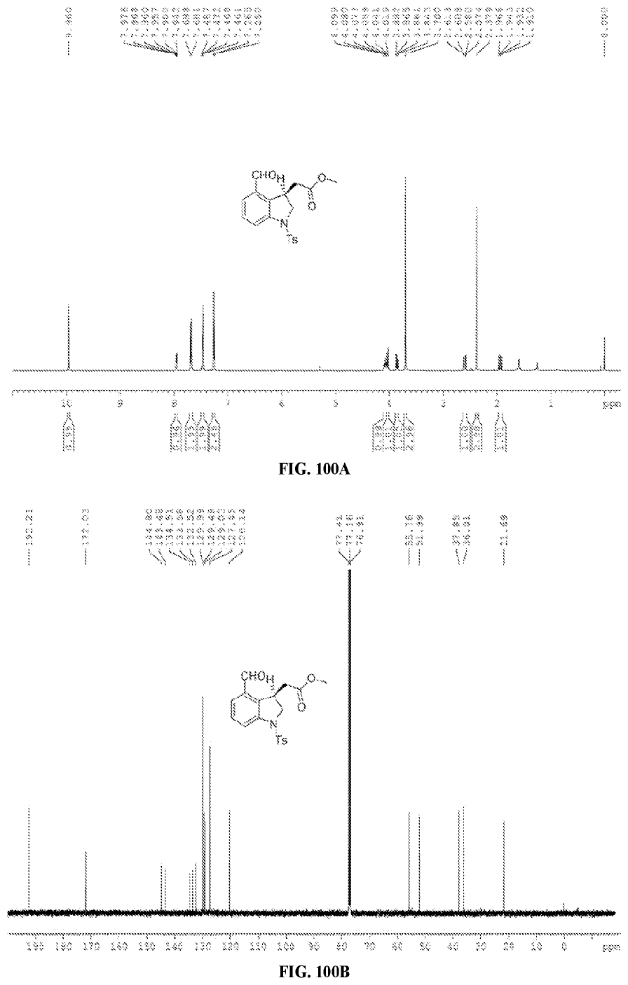

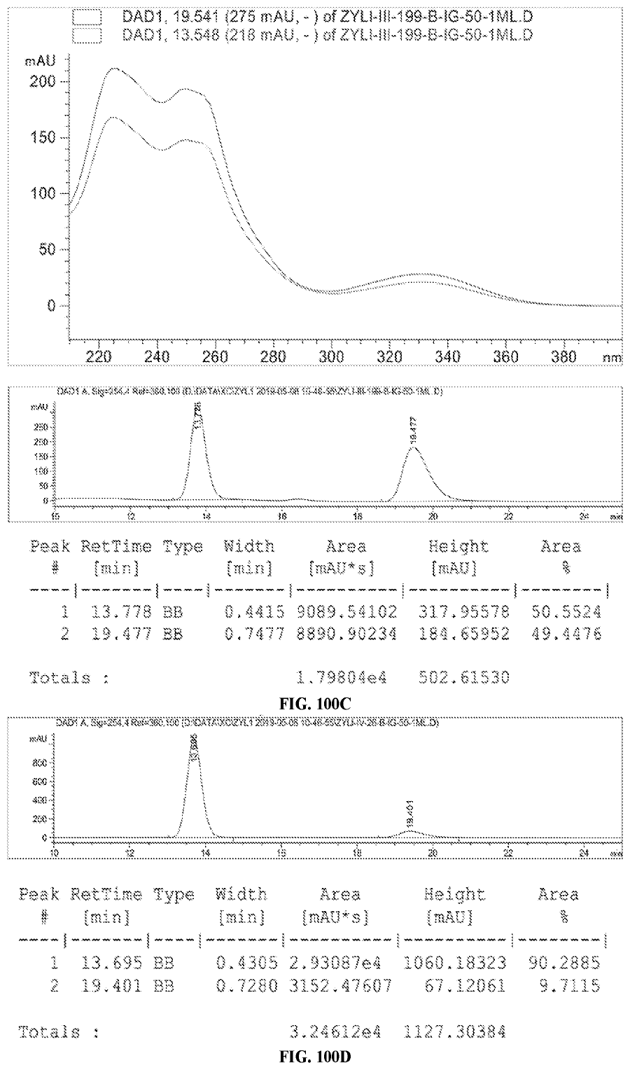

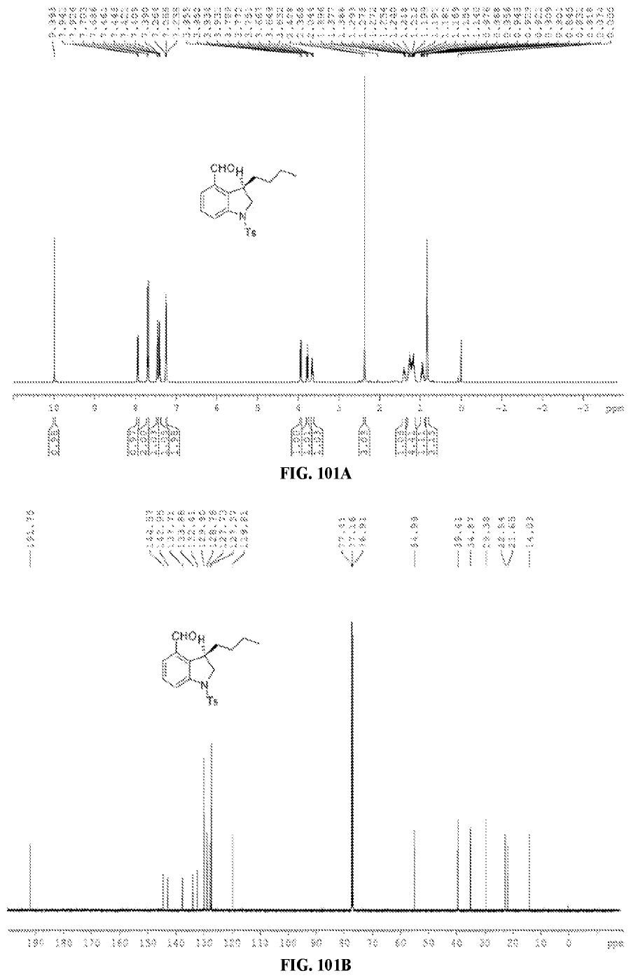

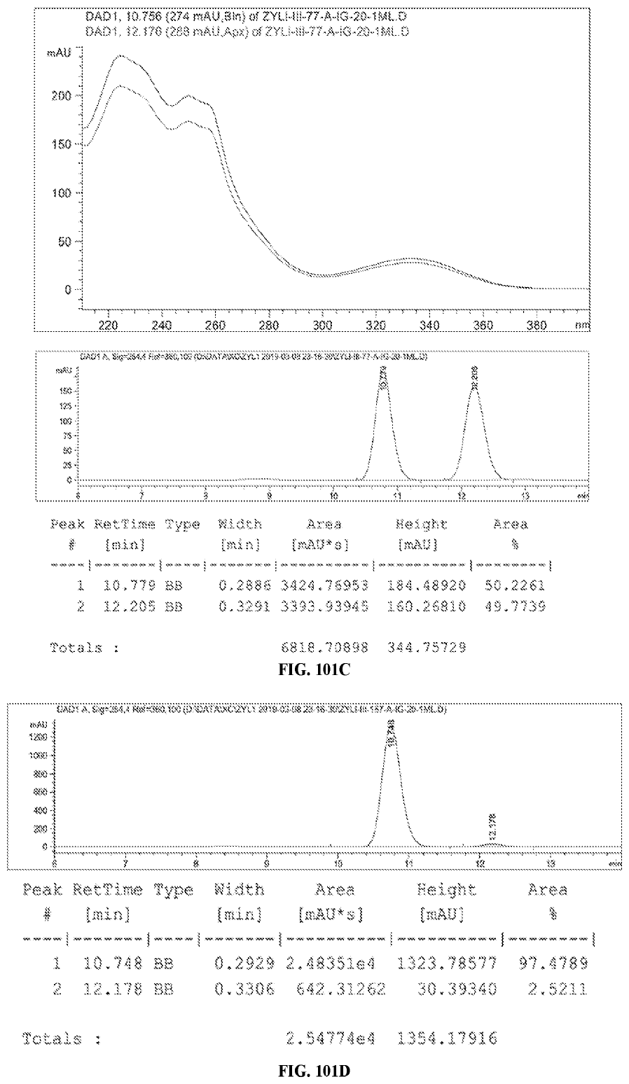

### 5. Characterization of Products (FIGS. 73A-101D)

### (R)-3-Benzyl-1-tosylindoline-4-carbaldehyde

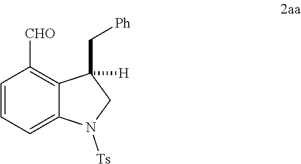 [SMILES: Cc1ccc(S(=O)(=O)N2C[C@H](Cc3ccccc3)c3c(C=O)cccc32)cc1]

By following the general procedure, the reaction of 1aa (39.2 mg, 0.1 mmol) with [Ru(p-cymene)C12]2 (3.2 mg, 0.005 mmol), KH2PO4 (27.1 mg, 0.2 mmol), AgBF4 (3.9 mg, 0.02 mmol), (R)-(+)-1-(1-naphthyl)ethylamine (8.0 μL, 0.05 mmol), and N-Phthaloyl-L-tert-leucine (7.9 mg, 0.03 mmol) afforded 2aa (34.0 mg, 87% yield).

White solid; ¹H NMR (500 MHz, CDCl3) 5 9.93 (s, 1H), 7.97 (dd, J=7.6, 1.0 Hz, 1H), 7.70 (d, J=8.3 Hz, 2H), 7.50-7.43 (m, 2H), 7.31 (t, J=7.4 Hz, 2H), 7.28-7.21 (m, 3H), 7.19 (d, J=7.2 Hz, 2H), 3.98-3.91 (m, 2H), 3.57 (dd, J=10.5, 8.8 Hz, 1H), 2.85 (dd, J=13.4, 3.5 Hz, 1H), 2.37 (s, 3H), 2.16 (dd, J=13.4, 10.5 Hz, 1H); 13C NMR (125 MHz, CDCl3) 5 191.9, 144.7, 143.2, 139.1, 136.3, 133.8, 132.6, 130.0 (2C), 129.3 (2C), 129.1, 128.8 (2C), 128.1, 127.5 (2C), 126.8, 119.7, 54.5, 41.6, 40.4, 21.7; HRMS (ESI) Calcd for C23H21NO3SK [M+K]⁺ 430.0874; found 430.0871; [α]²³D=−22.0 (c=0.5, CHCl3); HPLC analysis: ee=94%; CHIRALPAK®IG (80% hexanes: 20% isopropanol, 1 mL/min) tmajor=17.1 min, tminor=20.4 min.

TABLE S1
Catalytic Reactions with Varied Loadings of
the Chiral Amine CA8 (FIG. 18)

| entry | X mol % | yield (%)ª | ee (%) |
|---|---|---|---|
| 1 | 15 | 13 | 94 |
| 2 | 30 | 61 | 93 |
| 3 | 50 | 88 | 94 |

^{a}Determined by ¹H NMR with PhNO2 as internal standard.

### (R)-3-Benzyl-5-methoxy-1-tosylindoline-4-carbaldehyde

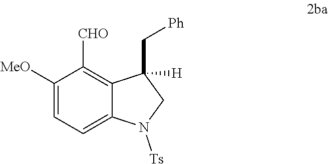 [SMILES: COc1ccc2c(c1C=O)[C@@H](Cc1ccccc1)CN2S(=O)(=O)c1ccc(C)cc1]

By following the general procedure, the reaction of 1ba (41.8 mg, 0.1 mmol) with [Ru(p-cymene)C12]2 (3.4 mg, 0.005 mmol), KH2PO4 (27.2 mg, 0.2 mmol), AgBF4 (4.3 mg, 0.02 mmol), (R)-(+)-1-(1-naphthyl)ethylamine (8.0 μL, 0.05 mmol), and N-Phthaloyl-L-tert-leucine (7.8 mg, 0.03 mmol) afforded 2ba (14.8 mg, 34% yield).

Yellow solid; ¹H NMR (500 MHz, CDCl3) 5 10.54 (s, 1H), 7.92 (d, J=9.0 Hz, 1H), 7.67 (d, J=8.1 Hz, 2H), 7.35-7.20 (m, 7H), 6.91 (d, J=9.0 Hz, 1H), 3.96-3.89 (m, 4H), 3.86 (d, J=10.7 Hz, 1H), 3.50-3.43 (m, 1H), 2.84 (d, J=11.8 Hz, 1H), 2.38 (s, 3H), 1.96-1.88 (m, 1H); ¹³C NMR (125 MHz, CDCl3) 5 190.6, 159.7, 144.5, 139.6, 138.3, 136.1, 133.8, 129.9 (2C), 129.4 (2C), 128.7 (2C), 127.6 (2C), 126.6, 121.4, 120.9, 111.5, 56.4, 54.3, 42.5, 39.7, 21.7; HRMS (ESI) Calcd for C24H24NO4S [M+H]⁺ 422.1421; found 422.1419; [α]²³D=+34.8 (c=0.5, CHCl3); HPLC analysis: ee=90%; CHIRALPAK®IG (80% hexanes: 20% isopropanol, 1 mL/min) tmajor=17.7 min, tminor=20.4 min.

### (R)-3-Benzyl-5-fluoro-1-tosylindoline-4-carbaldehyde

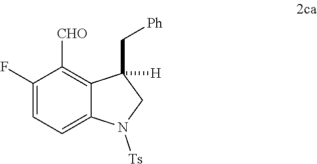 [SMILES: Cc1ccc(S(=O)(=O)N2C[C@H](Cc3ccccc3)c3c2ccc(F)c3C=O)cc1]

By following the general procedure, the reaction of 1ca (40.5 mg, 0.1 mmol) with [Ru(p-cymene)C12]2 (3.5 mg, 0.005 mmol), KH2PO4 (27.9 mg, 0.2 mmol), AgBF4 (4.1 mg, 0.02 mmol), (R)-(+)-1-(1-naphthyl)ethylamine (8.0 μL, 0.05 mmol), and N-Phthaloyl-L-tert-leucine (7.9 mg, 0.03 mmol) afforded 2ca (30.9 mg, 76% yield).

Yellow solid; ¹H NMR (500 MHz, CDCl3) 5 10.40 (s, 1H), 7.94 (dd, J=9.0, 4.4 Hz, 1H), 7.68 (d, J=8.2 Hz, 2H), 7.35-7.30 (m, 2H), 7.28 (d, J=8.2 Hz, 2H), 7.26-7.22 (m, 3H), 7.10 (t, J=9.7 Hz, 1H), 3.97-3.88 (m, 2H), 3.53 (dd, J=10.3, 8.8 Hz, 1H), 2.84 (dd, J=13.3, 2.6 Hz, 1H), 2.39 (s, 3H), 2.02 (dd, J=13.3, 11.3 Hz, 1H); 13C NMR (125 MHz, CDCl3) 5 187.7 (d, J=8.3 Hz), 161.4 (d, J=253.0 Hz), 144.8, 139.1 (d, J=2.3 Hz), 139.0, 137.9, 133.6, 130.0 (2C), 129.3 (2C), 128.8 (2C), 127.5 (2C), 126.8, 121.4 (d, J=9.6 Hz), 120.5 (d, J=9.1 Hz), 116.2 (d, J=22.9 Hz), 54.5, 42.1, 39.7, 21.7; ¹⁹F NMR (470 MHz, CDCl3) 5-128.36; HRMS (ESI) Calcd for C23H21FNO3S [M+H]⁺ 410.1221; found 410.1209; [α]²³D=−2.0 (c=0.5, CHCl3); HPLC analysis: ee=84%; CHIRALPAK@IG (90% hexanes: 10% isopropanol, 1 mL/min) tmajor=18.0 min, tminor=21.1 min.

### (R)-3-Benzyl-1-tosyl-5-(trifluoromethyl)indoline-4-carbaldehyde

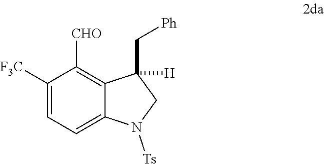 [SMILES: Cc1ccc(S(=O)(=O)N2C[C@H](Cc3ccccc3)c3c2ccc(C(F)(F)F)c3C=O)cc1]

By following the general procedure, the reaction of 1da (45.7 mg, 0.1 mmol) with [Ru(p-cymene)C12]2 (3.3 mg, 0.005 mmol), KH2PO4 (27.4 mg, 0.2 mmol), AgBF4 (4.4 mg, 0.02 mmol), (R)-(+)-1-(1-naphthyl)ethylamine (8.0 μL, 0.05 mmol), and N-Phthaloyl-L-tert-leucine (7.9 mg, 0.03 mmol) afforded 2da (28.3 mg, 62% yield).

White solid; 1H NMR (500 MHz, CDCl3) 5 10.35-10.33 (m, 1H), 7.94 (d, J=8.5 Hz, 1H), 7.74-7.69 (m, 3H), 7.33 (t, J=7.5 Hz, 2H), 7.30 (d, J=8.2 Hz, 2H), 7.287.21 (m, 3H), 4.09-4.02 (m, 1H), 3.97 (d, J=10.6 Hz, 1H), 3.53 (dd, J=10.0, 8.4 Hz, 1H), 2.85 (dd, J=13.3, 3.3 Hz, 1H), 2.40 (s, 3H), 2.21 (dd, J=13.3, 10.6 Hz, 1H); ¹³C NMR (125 MHz, CDCl3) 5 190.1 (q, J=2.7 Hz), 146.2, 145.2, 138.8, 138.5, 133.6, 130.2 (2C), 130.0, 129.4 (2C), 128.8 (2C), 127.5 (q, J=6.0 Hz), 127.45 (2C), 126.9, 126.2 (q, J=32.0 Hz), 124.1 (q, J=272.1 Hz), 117.1, 54.5, 42.1, 40.0, 21.7; ¹⁹F NMR (470 MHz, CDCl3) 5-53.89; HRMS (ESI) Calcd for C24H21F3NO3S [M+H]⁺ 460.1189; found 460.1173; [α]²³D=−39.8 (c=0.5, CHCl3); HPLC analysis: ee=83%; CHIRALPAK®AD-H (98% hexanes: 2% isopropanol, 1 mL/min) tminor=17.5 min, tmajor=21.9 min.

### (R)-3-Benzyl-6-methoxy-1-tosylindoline-4-carbaldehyde

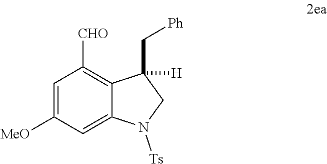 [SMILES: COc1cc(C=O)c2c(c1)N(S(=O)(=O)c1ccc(C)cc1)C[C@@H]2Cc1ccccc1]

By following the general procedure, the reaction of 1ea (44.0 mg, 0.1 mmol) with [Ru(p-cymene)C12]2 (3.2 mg, 0.005 mmol), KH2PO4 (27.8 mg, 0.2 mmol), AgBF4 (4.1 mg, 0.02 mmol), (R)-(+)-1-(1-naphthyl)ethylamine (8.0 μL, 0.05 mmol), and N-Phthaloyl-L-tert-leucine (7.9 mg, 0.03 mmol) afforded 2ea (37.8 mg, 86% yield).

Yellow oil; ¹H NMR (500 MHz, CDCl3) 5 9.77 (s, 1H), 7.72 (d, J=8.3 Hz, 2H), 7.56 (d, J=2.3 Hz, 1H), 7.33-7.27 (m, 4H), 7.26-7.22 (m, 1H), 7.14 (d, J=7.1 Hz, 2H), 6.98 (d, J=2.3 Hz, 1H), 3.94 (dd, J=10.4, 0.9 Hz, 1H), 3.91 (s, 3H), 3.863.80 (m, 1H), 3.59 (dd, J=10.3, 8.3 Hz, 1H), 2.77 (dd, J=13.4, 4.3 Hz, 1H), 2.39 (s, 3H), 2.20 (dd, J=13.4, 10.2 Hz, 1H); ¹³C NMR (125 MHz, CDCl3) 5 191.2, 160.8, 144.7, 144.4, 139.0, 133.8, 132.8, 130.0 (2C), 129.4 (2C), 128.78 (2C), 128.75, 127.5 (2C), 126.8, 111.8, 106.5, 56.1, 55.2, 41.0, 40.9, 21.7; HRMS (ESI) Calcd for C24H24NO4S [M+H]⁺ 422.1421; found 422.1410; [α]²³D=−1.6 (c=0.5, CHCl3); HPLC analysis: ee=94%; CHIRALPAK@IG (80% hexanes: 20% isopropanol, 1 mL/min) tmajor=17.4 min, tminor=20.0 min.

### (R)-3-Benzyl-6-methyl-1-tosylindoline-4-carbaldehyde

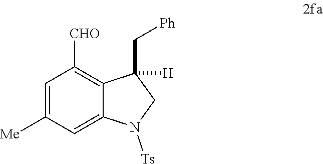 [SMILES: Cc1ccc(S(=O)(=O)N2C[C@H](Cc3ccccc3)c3c(C=O)cc(C)cc32)cc1]

By following the general procedure, the reaction of 1fa (40.9 mg, 0.1 mmol) with [Ru(p-cymene)Cl2]2 (3.2 mg, 0.005 mmol), KH2PO4 (27.9 mg, 0.2 mmol), AgBF4 (4.6 mg, 0.02 mmol), (R)-(+)-1-(1-naphthyl)ethylamine (8.0 μL, 0.05 mmol), and N-Phthaloyl-L-tert-leucine (8.3 mg, 0.03 mmol) afforded 2fa (29.0 mg, 71% yield).

Yellow oil; ¹H NMR (500 MHz, CDCl3) 5 9.87 (s, 1H), 7.81 (s, 1H), 7.70 (d, J=8.3 Hz, 2H), 7.33-7.21 (m, 6H), 7.17 (d, J=7.2 Hz, 2H), 3.94-3.84 (m, 2H), 3.56 (dd, J=10.4, 8.4 Hz, 1H), 2.82 (dd, J=13.4, 3.7 Hz, 1H), 2.48 (s, 3H), 2.38 (s, 3H), 2.12 (dd, J=13.4, 10.4 Hz, 1H); ¹³C NMR (125 MHz, CDCl3) 5 192.0, 144.6, 143.4, 139.5, 139.2, 134.0, 133.6, 132.3, 130.0 (2C), 129.4 (2C), 128.8, 128.7 (2C), 127.5 (2C), 126.7, 120.4, 54.8, 41.4, 40.5, 21.7 (2C); HRMS (ESI) Calcd for C24H24NO3S [M+H]⁺ 406.1471; found 406.1465; [α]²³D=2.8 (c=0.5, CHCl3); HPLC analysis: ee=88%; CHIRALPAK@IG (80% hexanes: 20% isopropanol, 1 mL/min) tmajor=15.5 min, tminor=20.0 min.

### (R)-3-Benzyl-6-fluoro-1-tosylindoline-4-carbaldehyde

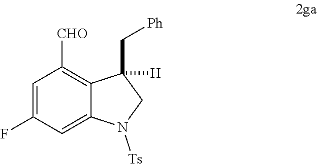 [SMILES: Cc1ccc(S(=O)(=O)N2C[C@H](Cc3ccccc3)c3c(C=O)cc(F)cc32)cc1]

By following the general procedure, the reaction of 1ga (40.8 mg, 0.1 mmol) with [Ru(p-cymene)C12]2 (3.3 mg, 0.005 mmol), KH2PO4 (27.6 mg, 0.2 mmol), AgBF4 (3.9 mg, 0.02 mmol), (R)-(+)-1-(1-naphthyl)ethylamine (8.0 μL, 0.05 mmol), and N-Phthaloyl-L-tert-leucine (8.1 mg, 0.03 mmol) afforded 2ga (28.2 mg, 69% yield).

Yellow oil; ¹H NMR (500 MHz, CDCl3) 5 9.77 (s, 1H), 7.74-7.67 (m, 3H), 7.33-7.28 (m, 4H), 7.26-7.22 (m, 1H), 7.16-7.12 (m, 3H), 3.96 (dd, J=10.3, 1.1 Hz, 1H), 3.91-3.85 (m, 1H), 3.61 (dd, J=10.3, 8.4 Hz, 1H), 2.79 (dd, J=13.4, 4.3 Hz, 1H), 2.40 (s, 3H), 2.25 (dd, J=13.4, 10.3 Hz, 1H); ¹³C NMR (125 MHz, CDCl3) 5 190.1 (d, J=1.7 Hz), 163.3 (d, J=245.8 Hz), 145.0, 144.7 (d, J=11.1 Hz), 138.6, 133.6, 133.1 (d, J=7.4 Hz), 132.0 (d, J=2.8 Hz), 130.1 (2C), 129.4 (2C), 128.8 (2C), 127.5 (2C), 126.9, 113.0 (d, J=23.2 Hz), 107.7 (d, J=28.3 Hz), 55.2, 41.0, 40.7, 21.7; ¹⁹F NMR (470 MHz, CDCl3) 5-110.61; HRMS (ESI) Calcd for C23H21FNO3S [M+H]⁺ 410.1221; found 410.1214; [α]²³D=−21.6 (c=0.5, CHCl3); HPLC analysis: ee=94%; CHIRALPAK@IG (80% hexanes: 20% isopropanol, 1 mL/min) tmajor=13.0 min, tminor=17.7 min.

### (R)-3-Benzyl-1-tosyl-6-(trifluoromethyl)indoline-4-carbaldehyde

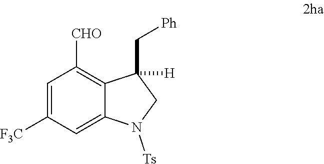 [SMILES: Cc1ccc(S(=O)(=O)N2C[C@H](Cc3ccccc3)c3c(C=O)cc(C(F)(F)F)cc32)cc1]

By following the general procedure, the reaction of 1ha (45.8 mg, 0.1 mmol) with [Ru(p-cymene)C12]2 (3.2 mg, 0.005 mmol), KH2PO4 (27.8 mg, 0.2 mmol), AgBF4 (4.2 mg, 0.02 mmol), (R)-(+)-1-(1-naphthyl)ethylamine (8.0 μL, 0.05 mmol), and N-Phthaloyl-L-tert-leucine (7.9 mg, 0.03 mmol) afforded 2ha (29.0 mg, 63% yield).

White solid; ¹H NMR (500 MHz, CDCl3) 5 9.90 (s, 1H), 8.17 (s, 1H), 7.74-7.69 (m, 3H), 7.35-7.28 (m, 4H), 7.28-7.24 (m, 1H), 7.17 (d, J=7.3 Hz, 2H), 4.04-3.95 (m, 2H), 3.62 (dd, J=10.2, 8.5 Hz, 1H), 2.84 (dd, J=13.4, 3.9 Hz, 1H), 2.39 (s, 3H), 2.26 (dd, J=13.4, 10.5 Hz, 1H); ¹³C NMR (125 MHz, CDCl3) 5 190.3, 145.2, 144.1, 140.0, 138.3, 133.5, 132.6, 132.0 (q, J=33.1 Hz), 130.2 (2C), 129.3 (2C), 128.9 (2C), 127.5 (2C), 127.1, 124.1 (q, J=3.7 Hz), 123.5 (q, J=271.5 Hz), 115.7 (q, J=3.6 Hz), 54.8, 41.5, 40.2,21.7; ¹⁹F NMR (470 MHz, CDCl3) 5-62.55; HRMS (ESI) Calcd for C24H20F3NO3SK [M+K]⁺ 498.0748; found 498.0748; [α]²³D=−20.0 (c=0.5, CHCl3); HPLC analysis: ee=82%; CHIRALPAK@IG (90% hexanes: 10% isopropanol, 1 mL/min) tmajor=12.0 min, tminor=17.2 min.

### (R)-3-Benzyl-7-methoxy-1-tosylindoline-4-carbaldehyde

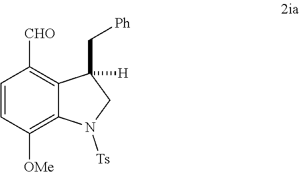 [SMILES: COc1ccc(C=O)c2c1N(S(=O)(=O)c1ccc(C)cc1)C[C@@H]2Cc1ccccc1]

By following the general procedure, the reaction of 11a (42.1 mg, 0.1 mmol) with [Ru(p-cymene)C12]2 (3.3 mg, 0.005 mmol), KH2PO4 (27.9 mg, 0.2 mmol), AgBF4 (3.8 mg, 0.02 mmol), (R)-(+)-1-(1-naphthyl)ethylamine (8.0 μL, 0.05 mmol), and N-Phthaloyl-L-tert-leucine (7.9 mg, 0.03 mmol) afforded 2ia (27.0 mg, 64% yield).

Yellow solid; ¹H NMR (500 MHz, CDCl3) 5 9.93 (s, 1H), 7.82 (s, J=8.1 Hz, 2H), 7.42 (d, J=8.5 Hz, 1H), 7.52 (d, J=7.4 Hz, 2H), 7.36 (t, J=7.5 Hz, 2H), 7.31 (d, J=8.1 Hz, 2H), 7.28-7.23 (m, 1H), 6.88 (d, J=8.5 Hz, 1H), 4.61 (d, J=11.4 Hz, 1H), 3.92-3.85 (m, 1H), 3.81 (dd, J=11.2, 7.1 Hz, 1H), 3.70 (s, 3H), 3.05 (dd, J=13.2, 2.3 Hz, 1H), 2.73 (dd, J=13.2, 11.2 Hz, 1H), 2.44 (s, 3H); ¹³C NMR (125 MHz, CDCl3) 5 190.6, 154.2, 143.2, 141.3, 140.1, 139.7, 132.7, 132.6, 129.7 (2C), 129.2 (2C), 128.7, (2C), 126.9 (2C), 126.5, 125.9, 111.2, 56.6, 55.5, 44.3, 38.3, 21.7; HRMS (ESI) Calcd for C24H23NO4SK [M+K]⁺ 460.0979; found 460.0971; [α]²³D=−3.8 (c=0.5, CHCl3); HPLC analysis: ee=69%; CHIRALPAK@IG (50% hexanes: 50% isopropanol, 1 mL/min) tmajor=13.4 min, tminor=15.7 min.

### (R)-3-Benzyl-7-fluoro-1-tosylindoline-4-carbaldehyde

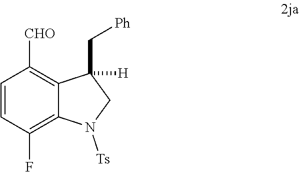 [SMILES: Cc1ccc(S(=O)(=O)N2C[C@H](Cc3ccccc3)c3c(C=O)ccc(F)c32)cc1]

By following the general procedure, the reaction of 1ja (41.0 mg, 0.1 mmol) with [Ru(p-cymene)C12]2 (3.2 mg, 0.005 mmol), KH2PO4 (27.2 mg, 0.2 mmol), AgBF4 (4.0 mg, 0.02 mmol), (R)-(+)-1-(1-naphthyl)ethylamine (8.0 μL, 0.05 mmol), and N-Phthaloyl-L-tert-leucine (8.2 mg, 0.03 mmol) afforded 2ja (24.8 mg, 60% yield).

White solid; 1H NMR (500 MHz, CDCl3) 5 9.94 (s, 1H), 7.82 (d, J=8.2 Hz, 2H), 7.54 (dd, J=8.5, 4.2 Hz, 1H), 7.40 (d, J=7.2 Hz, 2H), 7.37 (t, J=7.6 Hz, 2H), 7.31 (d, J=8.1 Hz, 2H), 7.29-7.25 (m, 1H), 7.14 (dd, J=10.4, 8.6 Hz, 1H), 4.41 (d, J=11.2 Hz, 1H), 3.96 (ddd, J=10.5, 7.5, 3.6 Hz, 1H), 3.81 (dd, J=11.1, 7.5 Hz, 1H), 2.97 (dd, J=13.4, 3.5 Hz, 1H), 2.62 (dd, J=13.4, 10.5 Hz, 1H), 2.42 (s, 3H); 13C NMR (125 MHz, CDCl3) 5 190.4, 154.8 (d, J=259.7 Hz), 144.2, 142.7 (d, J=4.0 Hz), 139.2, 137.4, 130.9 (d, J=10.9 Hz), 130.7 (d, J=8.2 Hz), 129.8 (2C), 129.6 (2C), 128.8 (3C), 127.3 (d, J=1.8 Hz, 2C), 126.8, 117.0 (d, J=21.5 Hz), 56.5, 43.5, 39.0, 21.7; ¹⁹F NMR (470 MHz, CDCl3) 5-109.33; HRMS (ESI) Calcd for C23H21FNO3S [M+H]⁺ 410.1221; found 410.1207; [α]²³D=+26.6 (c=0.5, CHCl3); HPLC analysis: ee=70%; CHIRALPAK@IG (80% hexanes: 20% isopropanol, 1 mL/min) tmajor=18.7 min, tminor=22.9 min.

### (R)-3-Benzyl-1-((4-nitrophenyl)sulfonyl)indoline-4-carbaldehyde

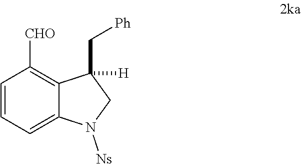

By following the general procedure, the reaction of 1ka (44.6 mg, 0.1 mmol) with [Ru(p-cymene)C12]2 (3.5 mg, 0.005 mmol), KH2PO4 (27.5 mg, 0.2 mmol), AgBF4 (4.2 mg, 0.02 mmol), (R)-(+)-1-(1-naphthyl)ethylamine (8.0 μL, 0.05 mmol), and N-Phthaloyl-L-tert-leucine (7.9 mg, 0.03 mmol) afforded 2ka (33.1 mg, 74% yield). Yellow solid; ¹H NMR (500 MHz, CDCl3) 5 9.96 (s, 1H), 8.32 (d, J=8.7 Hz, 2H), 8.00 (d, J=8.7 Hz, 2H), 7.94 (d, J=7.9 Hz, 1H), 7.55 (d, J=7.4 Hz, 1H), 7.50 (t, J=7.8 Hz, 1H), 7.32 (t, J=7.4 Hz, 2H), 7.28-7.23 (m, 1H), 7.18 (d, J=7.4 Hz, 2H), 4.06-3.98 (m, 2H), 3.62-3.55 (m, 1H), 2.92 (dd, J=13.5, 3.3 Hz, 1H), 2.23 (dd, J=13.3, 10.8 Hz, 1H); ¹³C NMR (125 MHz, CDCl3) 5 191.7, 150.7, 142.34, 142.31, 138.6, 136.1, 132.8, 129.5, 129.3 (2C), 129.0, 128.9 (2C), 128.6 (2C), 127.0, 124.6 (2C), 119.2, 54.7, 41.5, 40.3; HRMS (ESI) Calcd for C22H18N205SK [M+K]⁺ 461.0568; found 461.0563; [α]²³D=−22.6 (c=0.5, CHCl3); HPLC analysis: ee=92%; CHIRALPAK@IG (80% hexanes: 20% isopropanol, 1 mL/min) tmajor=18.8 min, tminor=26.1 min.

### (R)-3-(4-Methoxybenzyl)-1-tosylindoline-4-carbaldehyde

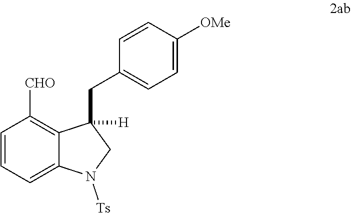 [SMILES: COc1ccc(C[C@H]2CN(S(=O)(=O)c3ccc(C)cc3)c3cccc(C=O)c32)cc1]

By following the general procedure, the reaction of 1ab (42.4 mg, 0.1 mmol) with [Ru(p-cymene)C12]2 (3.3 mg, 0.005 mmol), KH2PO4 (27.4 mg, 0.2 mmol), AgBF4 (4.0 mg, 0.02 mmol), (R)-(+)-1-(1-naphthyl)ethylamine (8.0 μL, 0.05 mmol), and N-Phthaloyl-L-tert-leucine (8.2 mg, 0.03 mmol) afforded 2ab (22.7 mg, 54% yield).

Yellow solid; ¹H NMR (500 MHz, CDCl3) 5 9.93 (s, 1H), 7.96 (d, J=7.6 Hz, 1H), 7.70 (d, J=8.3 Hz, 2H), 7.50-7.42 (m, 2H), 7.28-7.24 (m, 2H), 7.10 (d, J=8.4 Hz, 2H), 6.85 (d, J=8.4 Hz, 2H), 3.94 (d, J=10.7 Hz, 1H), 3.92-3.86 (m, 1H), 3.80 (s, 3H), 3.56 (dd, J=10.0, 8.7 Hz, 1H), 2.78 (dd, J=13.4, 3.6 Hz, 1H), 2.37 (s, 3H), 2.11 (dd, J=13.4, 10.6 Hz, 1H); ¹³C NMR (125 MHz, CDCl3) 5 191.9, 158.5, 144.7, 143.2, 136.4, 133.9, 132.6, 131.1, 130.3 (2C), 129.9 (2C), 129.1, 128.0, 127.5 (2C), 119.7, 114.2 (2C), 55.4, 54.5, 41.8, 39.6, 21.7; HRMS (ESI) Calcd for C24H23NO4SK [M+K]⁺ 460.0979; found 460.0967; [α]²³D=−15.6 (c=0.5, CHCl3); HPLC analysis: ee=94%; CHIRALPAK@IG (50% hexanes: 50% isopropanol, 1 mL/min) tmajor=13.0 min, tminor=18.7 min.

### (R)-3-(3-Methoxybenzyl)-1-tosylindoline-4-carbaldehyde

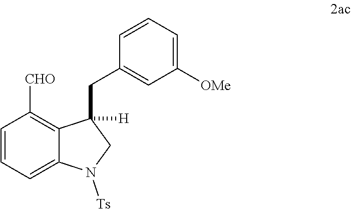 [SMILES: COc1cccc(C[C@H]2CN(S(=O)(=O)c3ccc(C)cc3)c3cccc(C=O)c32)c1]

By following the general procedure, the reaction of 1ac (43.0 mg, 0.1 mmol) with [Ru(p-cymene)C12]2 (3.3 mg, 0.005 mmol), KH2PO4 (27.9 mg, 0.2 mmol), AgBF4 (4.3 mg, 0.02 mmol), (R)-(+)-1-(1-naphthyl)ethylamine (8.0 μL, 0.05 mmol), and N-Phthaloyl-L-tert-leucine (7.7 mg, 0.03 mmol) afforded 2ac (27.1 mg, 63% yield).

Yellow oil; ¹H NMR (500 MHz, CDCl3) 5 9.94 (s, 1H), 7.95 (d, J=7.7 Hz, 1H), 7.70 (d, J=8.2 Hz, 2H), 7.51-7.43 (m, 2H), 7.29-7.20 (m, 3H), 6.81-6.73 (m, 3H), 3.97-3.91 (m, 2H), 3.82 (s, 3H), 3.58 (dd, J=10.0, 9.2 Hz, 1H), 2.83 (dd, J=13.3, 3.3 Hz, 1H), 2.38 (s, 3H), 2.12 (dd, J=13.3, 10.7 Hz, 1H); ¹³C NMR (125 MHz, CDCl3) 5 191.9, 159.9, 144.7, 143.2, 140.6, 136.3, 133.9, 132.6, 130.0 (2C), 129.7, 129.2, 128.1, 127.5 (2C), 121.7, 119.8, 114.9, 112.2, 55.3, 54.5, 41.5, 40.4, 21.7; HRMS (ESI) Calcd for C24H23NO4SK [M+K]⁺ 460.0979; found 460.0979; [α]²³D=−26.6 (c=0.5, CHCl3); HPLC analysis: ee=93%; CHIRALPAK@IG (70% hexanes: 30% isopropanol, 1 mL/min) tmajor=17.7 min, tminor=20.5 min.

### (R)-3-(2-Methoxybenzyl)-1-tosylindoline-4-carbaldehyde

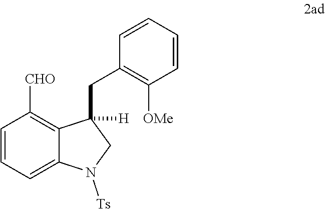 [SMILES: COc1ccccc1C[C@H]1CN(S(=O)(=O)c2ccc(C)cc2)c2cccc(C=O)c21]

By following the general procedure, the reaction of 1ad (42.9 mg, 0.1 mmol) with [Ru(p-cymene)C12]2 (3.4 mg, 0.005 mmol), KH2PO4 (28.1 mg, 0.2 mmol), AgBF4 (3.7 mg, 0.02 mmol), (R)-(+)-1-(1-naphthyl)ethylamine (8.0 μL, 0.05 mmol), and N-Phthaloyl-L-tert-leucine (7.8 mg, 0.03 mmol) afforded 2ad (28.2 mg, 66% yield).

Colorless oil; 1H NMR (500 MHz, CDCl3) 5 9.88 (s, 1H), 7.92 (d, J=8.0 Hz, 1H), 7.72 (d, J=8.1 Hz, 2H), 7.47 (d, J=7.7 Hz, 1H), 7.38 (t, J=7.9 Hz, 1H), 7.28-7.21 (m, 3H), 6.93 (d, J=7.0 Hz, 1H), 6.89-6.84 (m, 2H), 4.06-3.96 (m, 2H), 3.83 (s, 3H), 3.61 (dd, J=9.6, 8.7 Hz, 1H), 2.69 (dd, J=13.2, 4.9 Hz, 1H), 2.57 (dd, J=13.2, 9.7 Hz, 1H), 2.36 (s, 3H); ¹³C NMR (125 MHz, CDCl3) 5 190.6, 157.8, 144.6, 143.0, 137.7, 133.9, 132.9, 131.8, 129.9 (2C), 128.9, 128.4, 127.5 (2C), 126.8, 124.5, 120.7, 119.3, 110.5, 55.4, 55.2, 39.0, 36.5, 21.7; HRMS (ESI) Calcd for C24H24NO4S [M+H]⁺ 422.1421; found 422.1419; [α]²³D=−47.2 (c=0.5, CHCl3); HPLC analysis: ee=96%; CHIRALPAK®AD-H (80% hexanes: 20% isopropanol, 1 mL/min) tminor=11.5 min, tmajor=13.4 min.

### (R)-3-(4-Methylbenzyl)-1-tosylindoline-4-carbaldehyde

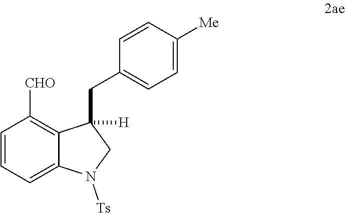 [SMILES: Cc1ccc(C[C@H]2CN(S(=O)(=O)c3ccc(C)cc3)c3cccc(C=O)c32)cc1]

By following the general procedure, the reaction of 1ae (40.5 mg, 0.1 mmol) with [Ru(p-cymene)C12]2 (3.3 mg, 0.005 mmol), KH2PO4 (27.8 mg, 0.2 mmol), AgBF4 (3.9 mg, 0.02 mmol), (R)-(+)-1-(1-naphthyl)ethylamine (8.0 μL, 0.05 mmol), and N-Phthaloyl-L-tert-leucine (7.7 mg, 0.03 mmol) afforded 2ae (24.9 mg, 61% yield).

Yellow solid; ¹H NMR (500 MHz, CDCl3) 5 9.93 (s, 1H), 7.97 (d, J=7.7 Hz, 1H), 7.70 (d, J=8.3 Hz, 2H), 7.50-7.43 (m, 2H), 7.28-7.24 (m, 2H), 7.12 (d, J=7.9 Hz, 2H), 7.08 (d, J=7.9 Hz, 2H), 3.96-3.88 (m, 2H), 3.56 (dd, J=10.3, 8.7 Hz, 1H), 2.80 (dd, J=13.6, 3.3 Hz, 1H), 2.37 (s, 3H), 2.34 (s, 3H), 2.12 (dd, J=13.3, 10.7 Hz, 1H); ¹³C NMR (125 MHz, CDCl3) 5 191.9, 144.7, 143.2, 136.4, 136.3, 136.0, 133.9, 132.6, 130.0 (2C), 129.4 (2C), 129.2 (2C), 129.1, 128.0, 127.5 (2C), 119.7, 54.5, 41.8, 40.0, 21.7, 21.2; HRMS (ESI) Calcd for C24H23NO3SK [M+K]⁺ 444.1030; found 444.1017; [α]²³D=−3.2 (c=0.5, CHCl3); HPLC analysis: ee=91%; CHIRALPAK@IG (80% hexanes: 20% isopropanol, 1 mL/min) tmajor=17.0 min, tminor=21.9 min.

### (R)-3-(3-Methylbenzyl)-1-tosylindoline-4-carbaldehyde

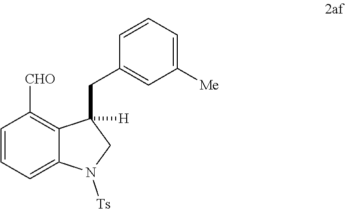 [SMILES: Cc1ccc(S(=O)(=O)N2C[C@H](Cc3cccc(C)c3)c3c(C=O)cccc32)cc1]

By following the general procedure, the reaction of 1af (40.8 mg, 0.1 mmol) with [Ru(p-cymene)C12]2 (3.4 mg, 0.005 mmol), KH2PO4 (28.1 mg, 0.2 mmol), AgBF4 (4.3 mg, 0.02 mmol), (R)-(+)-1-(1-naphthyl)ethylamine (8.0 μL, 0.05 mmol), and N-Phthaloyl-L-tert-leucine (8.1 mg, 0.03 mmol) afforded 2af (34.6 mg, 85% yield).

Colorless oil; 1H NMR (500 MHz, CDCl3) 5 9.94 (s, 1H), 7.98 (d, J=7.7 Hz, 1H), 7.70 (d, J=8.2 Hz, 2H), 7.50-7.43 (m, 2H), 7.28-7.25 (m, 2H), 7.20 (t, J=7.5 Hz, 1H), 7.05 (d, J=7.6 Hz, 1H), 7.01-697 (m, 2H), 3.97-3.90 (m, 2H), 3.57 (dd, J=10.3, 9.0 Hz, 1H), 2.82 (dd, J=13.3, 3.5 Hz, 1H), 2.38 (s, 3H), 2.35 (s, 3H), 2.10 (dd, J=13.3, 10.7 Hz, 1H); ¹³C NMR (125 MHz, CDCl3) 5 191.9, 144.7, 143.2, 139.0, 138.4, 136.4, 133.9, 132.6, 130.1, 130.0 (2C), 129.1, 128.6, 128.1, 127.5 (3C), 126.4, 119.7, 54.5, 41.6, 40.3, 21.7, 21.6; HRMS (ESI) Calcd for C24H24NO3S [M+H]⁺ 406.1471; found 406.1460; [α]²³D=−23.6 (c=0.5, CHCl3); HPLC analysis: ee=92%; CHIRALPAK@IG (90% hexanes: 10% isopropanol, 1 mL/min) tmajor=21.6 min, tminor=23.7 min.

### (R)-3-(2-Methylbenzyl)-1-tosylindoline-4-carbaldehyde

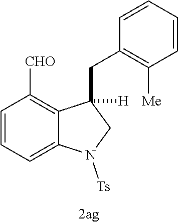 [SMILES: Cc1ccc(S(=O)(=O)N2C[C@H](Cc3ccccc3C)c3c(C=O)cccc32)cc1]

By following the general procedure, the reaction of 1ag (40.9 mg, 0.1 mmol) with [Ru(p-cymene)C12]2 (3.2 mg, 0.005 mmol), KH2PO4 (27.1 mg, 0.2 mmol), AgBF4 (5.3 mg, 0.02 mmol), (R)-(+)-1-(1-naphthyl)ethylamine (8.0 μL, 0.05 mmol), and N-Phthaloyl-L-tert-leucine (7.9 mg, 0.03 mmol) afforded 2ag (27.1 mg, 66% yield).

White solid; 1H NMR (500 MHz, CDCl3) 5 9.71 (s, 1H), 8.01-7.96 (m, 1H), 7.71 (d, J=8.2 Hz, 2H), 7.47-7.42 (m, 2H), 7.27-7.23 (m, 2H), 7.18-7.11 (m, 3H), 7.05-7.01 (m, 1H), 4.02-3.93 (m, 2H), 3.53 (dd, J=9.8, 8.4 Hz, 1H), 2.80 (dd, J=13.7, 5.6 Hz, 1H), 2.50 (dd, J=13.7, 10.2 Hz, 1H), 2.37 (s, 3H), 2.25 (s, 3H); ¹³C NMR (125 MHz, CDCl3) 5 191.1, 144.7, 143.2, 137.1, 136.6, 136.3, 133.6, 132.9, 130.7, 130.4, 130.0 (2C), 129.1, 127.6 (2C), 127.0, 126.8, 126.3, 119.4, 54.8, 39.7, 37.4, 21.7, 19.5; HRMS (ESI) Calcd for C24H23NO3SNa [M+Na]⁺ 428.1291; found 428.1275; [α]²³D=−73.6 (c=0.5, CHCl3); HPLC analysis: ee=90%; CHIRALPAK®AD-H (90% hexanes: 10% isopropanol, 1 mL/min) tminor=15.3 min, tmajor=19.5 min.

### (R)-3-(4-Fluorobenzyl)-1-tosylindoline-4-carbaldehyde

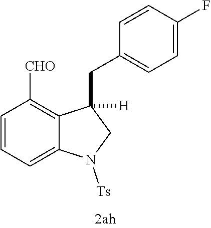 [SMILES: Cc1ccc(S(=O)(=O)N2C[C@H](Cc3ccc(F)cc3)c3c(C=O)cccc32)cc1]

By following the general procedure, the reaction of 1ah (40.4 mg, 0.1 mmol) with [Ru(p-cymene)C12]2 (3.2 mg, 0.005 mmol), KH2PO4 (27.7 mg, 0.2 mmol), AgBF4 (4.0 mg, 0.02 mmol), (R)-(+)-1-(1-naphthyl)ethylamine (8.0 μL, 0.05 mmol), and N-Phthaloyl-L-tert-leucine (7.9 mg, 0.03 mmol) afforded 2ah (26.1 mg, 65% yield).

Yellow solid; ¹H NMR (500 MHz, CDCl3) 5 9.97 (s, 1H), 7.97 (dd, J=7.2, 1.7 Hz, 1H), 7.70 (d, J=8.3 Hz, 2H), 7.50-7.44 (m, 2H), 7.29-7.25 (m, 2H), 7.16 (dd, J=8.3, 5.5 Hz, 2H), 6.99 (t, J=8.7 Hz, 2H), 3.95-3.88 (m, 2H), 3.55 (dd, J=10.2, 8.8 Hz, 1H), 2.83 (dd, J=13.5, 3.2 Hz, 1H), 2.38 (s, 3H), 2.16 (dd, J=13.5, 10.5 Hz, 1H); ¹³C NMR (125 MHz, CDCl3) 5 192.1, 161.9 (d, J=242.8 Hz), 144.7, 143.3, 135.9, 134.8 (d, J=2.9 Hz), 133.8, 132.5, 130.8 (d, J=7.7 Hz, 2C), 130.0 (2C), 129.3, 128.5, 127.5 (2C), 119.7, 115.5 (d, J=21.7 Hz, 2C), 54.4, 41.8, 39.4, 21.7; 19F NMR (470 MHz, CDCl3) 5-116.31; HRMS (ESI) Calcd for C23H21FNO3S [M+H]⁺ 410.1221; found 410.1221; [α]²³D=−15.8 (c=0.5, CHCl3); HPLC analysis: ee=93%; CHIRALPAK@IG (80% hexanes: 20% isopropanol, 1 mL/min) tmajor=16.1 min, tminor=21.3 min.

### (R)-3-(3-Fluorobenzyl)-1-tosylindoline-4-carbaldehyde

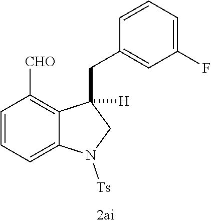 [SMILES: Cc1ccc(S(=O)(=O)N2C[C@H](Cc3cccc(F)c3)c3c(C=O)cccc32)cc1]

By following the general procedure, the reaction of 1ai (41.0 mg, 0.1 mmol) with [Ru(p-cymene)C12]2 (3.2 mg, 0.005 mmol), KH2PO4 (27.4 mg, 0.2 mmol), AgBF4 (4.3 mg, 0.02 mmol), (R)-(+)-1-(1-naphthyl)ethylamine (8.0 μL, 0.05 mmol), and N-Phthaloyl-L-tert-leucine (7.9 mg, 0.03 mmol) afforded 2ai (35.0 mg, 85% yield).

Yellow solid; ¹H NMR (500 MHz, CDCl3) 5 9.97 (s, 1H), 7.98 (d, J=7.1 Hz, 1H), 7.70 (d, J=7.9 Hz, 2H), 7.52-7.45 (m, 2H), 7.30-7.24 (m, 3H), 6.99 (d, J=7.5 Hz, 1H), 6.96-6.87 (m, 2H), 3.95 (t, J=7.9 Hz, 1H), 3.89 (d, J=10.6 Hz, 1H), 3.58 (dd, J=9.7, 9.2 Hz, 1H), 2.86 (d, J=13.1 Hz, 1H), 2.38 (s, 3H), 2.11 (dd, J=13.1, 11.5 Hz, 1H); ¹³C NMR (125 MHz, CDCl3) 5 192.1, 163.1 (d, J=244.8 Hz), 144.8, 143.3, 141.6 (d, J=7.1 Hz), 135.7, 133.9, 132.5, 130.2 (d, J=8.3 Hz), 130.0 (2C), 129.3, 128.7, 127.5 (2C), 125.0 (d, J=2.6 Hz), 119.9, 116.1 (d, J=20.9 Hz), 113.7 (d, J=20.9 Hz), 54.3, 41.5, 39.9,21.7; ¹⁹F NMR (470 MHz, CDCl3) δ −113.05; HRMS (ESI) Calcd for C23H20FNO3SNa [M+Na]⁺ 432.1040; found 432.1042; [α]²³D=−10.6 (c=0.5, CHCl3); HPLC analysis: ee=90%; CHIRALPAK@IG (80% hexanes: 20% isopropanol, 1 mL/min) tmajor=15.0 min, tminor=20.6 min.

### (R)-3-(2-Fluorobenzyl)-1-tosylindoline-4-carbaldehyde

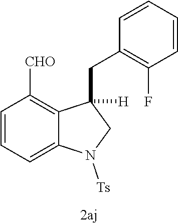 [SMILES: Cc1ccc(S(=O)(=O)N2C[C@H](Cc3ccccc3F)c3c(C=O)cccc32)cc1]

By following the general procedure, the reaction of 1aj (35.5 mg, 0.1 mmol) with [Ru(p-cymene)C12]2 (3.2 mg, 0.005 mmol), KH2PO4 (26.7 mg, 0.2 mmol), AgBF4 (4.1 mg, 0.02 mmol), (R)-(+)-1-(1-naphthyl)ethylamine (8.0 μL, 0.05 mmol), and N-Phthaloyl-L-tert-leucine (8.1 mg, 0.03 mmol) afforded 2aj (27.1 mg, 76% yield).

Yellow solid; ¹H NMR (500 MHz, CDCl3) 5 9.91 (s, 1H), 7.95 (d, J=7.7 Hz, 1H), 7.71 (d, J=8.2 Hz, 2H), 7.48 (d, J=7.0 Hz, 1H), 7.44 (t, J=7.7 Hz, 1H), 7.28-7.24 (m, 2H), 7.22 (t, J=6.9 Hz, 1H), 7.14 (t, J=7.1 Hz, 1H), 7.07 (t, J=7.5 Hz, 1H), 7.03 (t, J=9.2 Hz, 1H), 4.05-3.96 (m, 2H), 3.65 (dd, J=9.9, 8.9 Hz, 1H), 2.77 (dd, J=13.7, 4.1 Hz, 1H), 2.49 (dd, J=13.7, 10.1 Hz, 1H), 2.37 (s, 3H); ¹³C NMR (125 MHz, CDCl3) 5 191.3, 161.4 (d, J=244.3 Hz), 144.7, 143.2, 136.0, 133.8, 132.8, 131.6 (d, J=4.8 Hz), 129.9 (2C), 129.3, 128.7 (d, J=8.1 Hz), 127.5 (2C), 127.1, 125.7 (d, J=15.6 Hz), 124.4 (d, J=3.5 Hz), 119.6, 115.6 (d, J=21.9 Hz), 55.0, 39.9, 34.0, 21.7; ¹⁹F NMR (470 MHz, CDCl3) 5-117.35; HRMS (ESI) Calcd for C23H20FNO3SNa [M+Na]⁺ 432.1040; found 432.1032; [α]²³D=−7.2 (c=0.5, CHCl3); HPLC analysis: ee=93%; CHIRALPAK®AD-H (80% hexanes: 20% isopropanol, 1 mL/min) tminor=12.9 min, tmajor=15.0 min.

### (R)-3-(4-(Trifluoromethyl)benzyl)1-tosylindoline-4-carbaldehyde

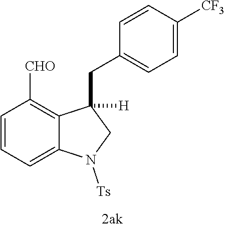 [SMILES: Cc1ccc(S(=O)(=O)N2C[C@H](Cc3ccc(C(F)(F)F)cc3)c3c(C=O)cccc32)cc1]

By following the general procedure, the reaction of 1ak (45.4 mg, 0.1 mmol) with [Ru(p-cymene)C12]2 (3.4 mg, 0.005 mmol), KH2PO4 (28.0 mg, 0.2 mmol), AgBF4 (4.2 mg, 0.02 mmol), (R)-(+)-1-(1-naphthyl)ethylamine (8.0 μL, 0.05 mmol), and N-Phthaloyl-L-tert-leucine (7.8 mg, 0.03 mmol) afforded 2ak (32.4 mg, 71% yield).

Yellow solid; ¹H NMR (500 MHz, CDCl3) 5 10.01 (s, 1H), 7.98 (dd, J=6.5, 2.5 Hz, 1H), 7.70 (d, J=8.2 Hz, 2H), 7.58 (d, J=7.9 Hz, 2H), 7.52-7.47 (m, 2H), 7.36 (d, J=7.9 Hz, 2H), 7.27 (d, J=8.2 Hz, 2H), 3.97 (t, J=8.3 Hz, 1H), 3.88 (d, J=10.6 Hz, 1H), 3.54 (dd, J=9.9, 9.0 Hz, 1H), 2.94 (d, J=11.7 Hz, 1H), 2.38 (s, 3H), 2.22 (dd, J=13.2, 11.1 Hz, 1H); ¹³C NMR (125 MHz, CDCl3) 5 192.4, 144.8, 143.34, 143.28, 135.5, 133.7, 132.5, 130.0 (2C), 129.7 (2C), 129.4, 129.1 (q, J=32.1 Hz), 129.0, 127.5 (2C), 125.7 (q, J=3.6 Hz, 2C), 124.4 (q, J=270.4 Hz), 119.8, 54.2, 41.6, 39.8, 21.7; ¹⁹F NMR (470 MHz, CDCl3) 5-62.38; HRMS (ESI) Calcd for C24H20F3NO3SNa [M+Na]⁺ 482.1008; found 482.1008; [α]²³D=−6.8 (c=0.5, CHCl3); HPLC analysis: ee=90%; CHIRALPAK@IG (80% hexanes: 20% isopropanol, 1 mL/min) tmajor=11.6 min, tminor=17.3 min.

### (R)-3-(3-(Trifluoromethyl)benzyl)1-tosylindoline-4-carbaldehyde

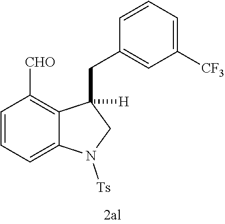 [SMILES: Cc1ccc(S(=O)(=O)N2C[C@H](Cc3cccc(C(F)(F)F)c3)c3c(C=O)cccc32)cc1]

By following the general procedure, the reaction of 1al (45.9 mg, 0.1 mmol) with [Ru(p-cymene)C12]2 (3.4 mg, 0.005 mmol), KH2PO4 (27.3 mg, 0.2 mmol), AgBF4 (4.3 mg, 0.02 mmol), (R)-(+)-1-(1-naphthyl)ethylamine (8.0 μL, 0.05 mmol), and N-Phthaloyl-L-tert-leucine (8.1 mg, 0.03 mmol) afforded 2al (36.4 mg, 79% yield).

Colorless oil; 1H NMR (500 MHz, CDCl3) 5 9.99 (s, 1H), 8.00 (dd, J=6.8, 2.2 Hz, 1H), 7.70 (d, J=8.2 Hz, 2H), 7.53-7.47 (m, 3H), 7.47-7.41 (m, 2H), 7.32-7.27 (m, 3H), 3.99 (td, J=9.2, 2.9 Hz, 1H), 3.83 (d, J=10.8 Hz, 1H), 3.61 (dd, J=10.5, 8.5 Hz, 1H), 2.92 (dd, J=13.5, 3.2 Hz, 1H), 2.39 (s, 3H), 2.10 (dd, J=13.5, 10.8 Hz, 1H); ¹³C NMR (125 MHz, CDCl3) 5 192.3, 144.9, 143.3, 140.0, 135.5, 133.9, 132.7, 132.5, 130.9 (q, J=31.9 Hz), 130.1 (2C), 129.4, 129.2, 129.0, 127.4 (2C), 126.0 (q, J=3.7 Hz), 124.2 (q, J=270.8 Hz), 123.6 (q, J=3.7 Hz), 120.0, 54.2, 41.4, 39.9, 21.7; ¹⁹F NMR (470 MHz, CDCl3) 5-62.48; HRMS (ESI) Calcd for C24H20F3NO3SK [M+K]⁺ 498.0748; found 498.0743; [α]²³D=−18.8 (c=0.5, CHCl3); HPLC analysis: ee=87%; CHIRALPAK@IG (80% hexanes: 20% isopropanol, 1 mL/min) tmajor=10.1 min, tminor=12.6 min.

### (R)-3-(2-(Trifluoromethyl)benzyl)1-tosylindoline-4-carbaldehyde

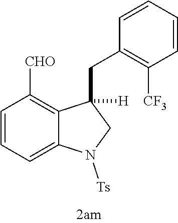 [SMILES: Cc1ccc(S(=O)(=O)N2C[C@H](Cc3ccccc3C(F)(F)F)c3c(C=O)cccc32)cc1]

By following the general procedure, the reaction of 1am (45.2 mg, 0.1 mmol) with [Ru(p-cymene)C12]2 (3.2 mg, 0.005 mmol), KH2PO4 (27.1 mg, 0.2 mmol), AgBF4 (4.0 mg, 0.02 mmol), (R)-(+)-1-(1-naphthyl)ethylamine (8.0 μL, 0.05 mmol), and N-Phthaloyl-L-tert-leucine (8.0 mg, 0.03 mmol) afforded 2am (38.3 mg, 85% yield).

Yellow solid; 1H NMR (500 MHz, CDCl3) 5 9.71 (s, 1H), 7.99-7.94 (m, 1H), 7.71 (d, J=8.2 Hz, 2H), 7.65 (d, J=7.9 Hz, 1H), 7.49-7.43 (m, 3H), 7.35 (t, J=7.6 Hz, 1H), 7.27-7.21 (m, 3H), 4.17-4.10 (m, 1H), 3.95 (d, J=10.2 Hz, 1H), 3.59 (dd, J=9.5, 8.8 Hz, 1H), 2.94 (dd, J=14.4, 5.8 Hz, 1H), 2.88 (dd, J=14.4, 9.5 Hz, 1H), 2.37 (s, 3H); ¹³C NMR (125 MHz, CDCl3) 5 191.0, 144.8, 143.2, 137.1, 135.6, 133.5, 132.9, 132.0, 131.9, 130.0 (2C), 129.40 (q, J=29.4 Hz), 129.35, 127.5 (2C), 127.0, 126.6, 126.4 (q, J=5.7 Hz), 124.5 (q, J=272.5 Hz), 119.3, 55.0, 39.9, 36.5, 21.7; ¹⁹F NMR (470 MHz, CDCl3) 5-58.97; HRMS (ESI) Calcd for C24H20F3NO3SK [M+K]⁺ 498.0748; found 498.0736; [α]²³D=−42.8 (c=0.5, CHCl3); HPLC analysis: ee=87%; CHIRALPAK®AD-H (90% hexanes: 10% isopropanol, 1 mL/min) tminor=13.9 min, tmajor=18.9 min.

### (R)-3-(4-Nitrobenzyl)-1-tosylindoline-4-carbaldehyde

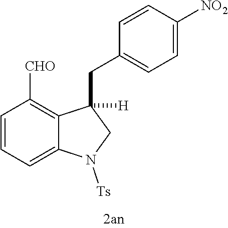 [SMILES: Cc1ccc(S(=O)(=O)N2C[C@H](Cc3ccc([N+](=O)[O-])cc3)c3c(C=O)cccc32)cc1]

By following the general procedure, the reaction of 1an (44.1 mg, 0.1 mmol) with [Ru(p-cymene)C12]2 (3.1 mg, 0.005 mmol), KH2PO4 (27.3 mg, 0.2 mmol), AgBF4 (4.2 mg, 0.02 mmol), (R)-(+)-1-(1-naphthyl)ethylamine (8.0 μL, 0.05 mmol), and N-Phthaloyl-L-tert-leucine (8.1 mg, 0.03 mmol) afforded 2an (26.2 mg, 59% yield).

White solid; 1H NMR (500 MHz, CDCl3) 5 10.02 (s, 1H), 8.17 (d, J=8.5 Hz, 2H), 7.98 (dd, J=5.4, 3.6 Hz, 1H), 7.70 (d, J=8.2 Hz, 2H), 7.54-7.48 (m, 2H), 7.40 (d, J=8.5 Hz, 2H), 7.28 (d, J=8.2 Hz, 2H), 3.99 (t, J=8.3 Hz, 1H), 3.86 (d, J=10.6 Hz, 1H), 3.55 (dd, J=10.1, 8.8 Hz, 1H), 2.98 (dd, J=13.3, 2.5 Hz, 1H), 2.39 (s, 3H), 2.33 (dd, J=13.3, 10.8 Hz, 1H); ¹³C NMR (125 MHz, CDCl3) 5 192.6, 147.0, 146.9, 144.9, 143.3, 135.0, 133.7, 132.4, 130.2 (2C), 130.0 (2C), 129.6, 129.3, 127.5 (2C), 124.0 (2C), 119.9, 54.2, 41.4, 39.7, 21.7; HRMS (ESI) Calcd for C23H21N2O5S [M+H]⁺ 437.1166; found 437.1154; [α]²³D=−40.6 (c=0.5, CHCl3); HPLC analysis: ee=86%; CHIRALPAK@IG (50% hexanes: 50% isopropanol, 1 mL/min) tmajor=16.3 min, tminor=27.6 min.

### (R)-3-Ethyl-1-tosylindoline-4-carbaldehyde

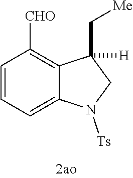 [SMILES: CC[C@H]1CN(S(=O)(=O)c2ccc(C)cc2)c2cccc(C=O)c21]

By following the general procedure, the reaction of 1ao (32.9 mg, 0.1 mmol) with [Ru(p-cymene)C12]2 (3.3 mg, 0.005 mmol), KH2PO4 (27.9 mg, 0.2 mmol), AgBF4 (4.0 mg, 0.02 mmol), (R)-(+)-1-(1-naphthyl)ethylamine (8.0 μL, 0.05 mmol), and N-Phthaloyl-L-tert-leucine (8.3 mg, 0.03 mmol) afforded 2ao (30.4 mg, 92% yield).

Colorless oil; ¹H NMR (500 MHz, CDCl3) 5 9.99 (s, 1H), 7.92 (d, J=7.9 Hz, 1H), 7.70 (d, J=8.1 Hz, 2H), 7.45 (d, J=7.5 Hz, 1H), 7.41 (t, J=7.8 Hz, 1H), 7.27-7.23 (m, 2H), 3.97 (d, J=10.4 Hz, 1H), 3.75 (t, J=9.5 Hz, 1H), 3.62 (t, J=8.5 Hz, 1H), 2.37 (s, 3H), 1.56-1.46 (m, 1H), 1.13-1.02 (m, 1H), 0.86 (t, J=7.3 Hz, 3H); 13C NMR (125 MHz, CDCl3) 5 191.8, 144.6, 143.0, 137.3, 133.8, 132.5, 129.9 (2C), 128.8, 127.7, 127.4 (2C), 119.6, 54.7, 40.8, 28.0, 21.7, 11.5; HRMS (ESI) Calcd for C18H19NO3SK [M+K]⁺ 368.0717; found 368.0713; [α]²³D=63.8 (c=0.5, CHCl3); HPLC analysis: ee=95%; CHIRALPAK@IG (80% hexanes: 20% isopropanol, 1 mL/min) tmajor=13.3 min, tminor=14.9 min.

### (R)-3-(2-((tert-Butyldimethylsilyl)oxy)ethyl)-1-tosylindoline-4-carbaldehyde

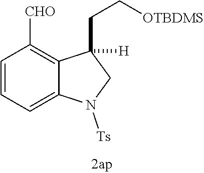 [SMILES: Cc1ccc(S(=O)(=O)N2C[C@H](CCO[Si](C)(C)C(C)(C)C)c3c(C=O)cccc32)cc1]

By following the general procedure, the reaction of 1ap (45.7 mg, 0.1 mmol) with [Ru(p-cymene)C12]2 (3.3 mg, 0.005 mmol), KH2PO4 (27.4 mg, 0.2 mmol), AgBF4 (3.9 mg, 0.02 mmol), (R)-(+)-1-(1-naphthyl)ethylamine (8.0 μL, 0.05 mmol), and N-Phthaloyl-L-tert-leucine (8.1 mg, 0.03 mmol) afforded 2ap (34.6 mg, 76% yield).

Colorless oil; ¹H NMR (500 MHz, CDCl3) 5 10.01 (s, 1H), 7.92 (d, J=8.0 Hz, 1H), 7.70 (d, J=8.1 Hz, 2H), 7.46 (d, J=7.5 Hz, 1H), 7.40 (t, J=7.8 Hz, 1H), 7.25 (d, J=8.1 Hz, 2H), 4.14-4.07 (m, 1H), 3.83-3.74 (m, 2H), 3.71-3.62 (m, 2H), 2.37 (s, 3H), 1.64-1.56 (m, 1H), 1.28-1.23 (m, 1H), 0.91 (s, 9H), 0.06 (s, 6H); ¹³C NMR (125 MHz, CDCl3) 5 191.6, 144.5, 143.1, 137.8, 133.9, 132.4, 129.9 (2C), 128.8, 127.4 (2C), 127.2, 119.7, 61.5, 55.5, 38.0, 36.9, 26.0 (3C), 21.7, 18.4, −5.27, −5.31; HRMS (ESI) Calcd for C24H33NO4SSiK [M+K]⁺ 498.1531; found 498.1524; [α]²³D=59.0 (c=0.5, CHCl3); HPLC analysis: ee=91%; CHIRALPAK@IG (95% hexanes: 5% isopropanol, 1 mL/min) tmajor=13.1 min, tminor=14.6 min.

### (R)-3-(3-((tert-Butyldimethylsilyl)oxy)propyl)-1-tosylindoline-4-carbaldehyde

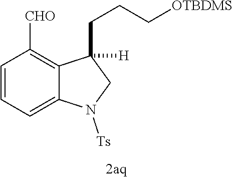 [SMILES: Cc1ccc(S(=O)(=O)N2C[C@H](CCCO[Si](C)(C)C(C)(C)C)c3c(C=O)cccc32)cc1]

By following the general procedure, the reaction of 1aq (47.3 mg, 0.1 mmol) with [Ru(p-cymene)C12]2 (3.2 mg, 0.005 mmol), KH2PO4 (27.1 mg, 0.2 mmol), AgBF4 (3.8 mg, 0.02 mmol), (R)-(+)-1-(1-naphthyl)ethylamine (8.0 μL, 0.05 mmol), and N-Phthaloyl-L-tert-leucine (8.2 mg, 0.03 mmol) afforded 2aq (32.5 mg, 69% yield).

Colorless oil; 1H NMR (500 MHz, CDCl3) 5 10.02 (s, 1H), 7.94 (d, J=8.0 Hz, 1H), 7.72 (d, J=8.1 Hz, 2H), 7.47 (d, J=7.6 Hz, 1H), 7.42 (t, J=7.8 Hz, 1H), 7.27 (d, J=8.1 Hz, 2H), 3.98 (d, J=10.1 Hz, 1H), 3.82-3.71 (m, 2H), 3.52 (t, J=6.1 Hz, 2H), 2.39 (s, 3H), 1.59-1.41 (m, 3H), 1.16-1.06 (m, 1H), 0.89 (s, 9H), 0.044 (s, 3H), 0.035 (s, 3H); ¹³C NMR (125 MHz, CDCl3) 5 191.6, 144.6, 143.0, 137.4, 133.8, 132.5, 129.9 (2C), 128.9, 127.5, 127.4 (2C), 119.7, 62.9, 55.1, 39.1, 31.7, 30.3, 26.1 (3C), 21.7, 18.4, −5.18, −5.19; HRMS (ESI) Calcd for C19H19N2O4S [M+H]⁺ 339.1161; found 339.1153. [α]²³D=42.0 (c=0.5, CHCl3); HPLC analysis: ee=94%. CHIRALPAK®AD-H (98% hexanes: 2% isopropanol, 1 mL/min) tminor=14.8 min, tmajor=19.1 min.

### Methyl (R)-2-(4-formyl-1-tosylindolin-3-yl)acetate

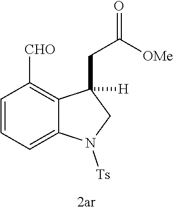 [SMILES: COC(=O)C[C@H]1CN(S(=O)(=O)c2ccc(C)cc2)c2cccc(C=O)c21]

By following the general procedure, the reaction of 1ar (37.3 mg, 0.1 mmol) with [Ru(p-cymene)C12]2 (3.2 mg, 0.005 mmol), KH2PO4 (27.2 mg, 0.2 mmol), AgBF4 (4.1 mg, 0.02 mmol), (R)-(+)-1-(1-naphthyl)ethylamine (8.0 μL, 0.05 mmol), and N-Phthaloyl-L-tert-leucine (7.9 mg, 0.03 mmol) afforded 2ar (20.5 mg, 55% yield).

Colorless oil; 1H NMR (500 MHz, CDCl3) 5 9.96 (s, 1H), 7.98-7.93 (m, 1H), 7.69 (d, J=8.2 Hz, 2H), 7.49-7.44 (m, 2H), 7.28-7.24 (m, 2H), 4.11-4.05 (m, 1H), 4.03 (d, J=11.2 Hz, 1H), 3.86 (dd, J=10.8, 8.8 Hz, 1H), 3.70 (s, 3H), 2.59 (dd, J=16.8, 2.8 Hz, 1H), 2.38 (s, 3H), 1.94 (dd, J=16.8, 11.2 Hz, 1H); ¹³C NMR (125 MHz, CDCl3) 5 192.2, 172.0, 144.8, 143.5, 134.5, 133.6, 132.5, 130.0 (2C), 129.5, 129.0, 127.5 (2C), 120.1, 55.8, 52.0, 37.9, 36.0, 21.7; HRMS (ESI) Calcd for C19H20NO5S [M+H]⁺ 374.1057; found 374.1048; [α]²³D=77.8 (c=0.5, CHCl3); HPLC analysis: ee=81%; CHIRALPAK@IG (50% hexanes: 50% isopropanol, 1 mL/min) tmajor=13.7 min, tminor=19.4 min.

### (R)-3-Butyl-1-tosylindoline-4-carbaldehyde

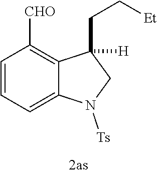 [SMILES: CCCC[C@H]1CN(S(=O)(=O)c2ccc(C)cc2)c2cccc(C=O)c21]

By following the general procedure, the reaction of E-1as (35.8 mg, 0.1 mmol) with [Ru(p-cymene)C12]2 (3.4 mg, 0.005 mmol), KH2PO4 (28.2 mg, 0.2 mmol), AgBF4 (4.1 mg, 0.02 mmol), (R)-(+)-1-(1-naphthyl)ethylamine (8.0 μL, 0.05 mmol), and N-Phthaloyl-L-tert-leucine (7.8 mg, 0.03 mmol) afforded 2as (30.8 mg, 86% yield).

Colorless oil; 1H NMR (500 MHz, CDCl3) 5 9.99 (s, 1H), 7.93 (d, J=7.3 Hz, 1H), 7.69 (d, J=8.2 Hz, 2H), 7.45 (d, J=7.5 Hz, 1H), 7.41 (t, J=7.8 Hz, 1H), 7.25 (d, J=8.2 Hz, 2H), 3.94 (dd, J=10.5, 1.6 Hz, 1H), 3.77 (dd, J=10.3, 8.7 Hz, 1H), 3.65 (t, J=9.0 Hz, 1H), 2.37 (s, 3H), 1.44-1.35 (m, 1H), 1.33-1.11 (m, 4H), 0.99-0.89 (m, 1H), 0.83 (t, J=7.0 Hz, 3H); ¹³C NMR (125 MHz, CDCl3) 5 191.8, 144.6, 143.0, 137.7, 133.9, 132.4, 129.9 (2C), 128.8, 127.7, 127.4 (2C), 119.8, 55.0, 39.4, 34.9, 29.4, 22.5, 21.7, 14.0; HRMS (ESI) Calcd for C20H24NO3S [M+H]⁺ 358.1471; found 358.1470; [α]²³D=69.8 (c=0.5, CHCl3); HPLC analysis: ee=95%; CHIRALPAK@IG (80% hexanes: 20% isopropanol, 1 mL/min) tmajor=10.7 min, tminor=12.2 min.

A catalytic reaction with (E)-1as was performed in 3 hours at 70° C. (FIG. 19). ¹H NMR of the recovered 1as was identical as the starting material (E)-1as, indicating no detectable E/Z isomerization occurred under the reaction conditions. Additionally, a catalytic reaction with (Z)-1as was performed in 3 hours at 70° C. (FIG. 20). ¹H NMR of the recovered 1as was identical as the starting material (Z)-1as, indicating no detectable E/Z isomerization occurred under the reaction conditions.

### 6. Synthesis of Compounds 3a

### (R)-1-Tosyl-1,2,2a,3-tetrahydrobenzo[cd]indole-4-carbaldehyde (FIG. 21)

To a 7 mL vial equipped with a stirring bar were added 2aq (0.1 mmol) and TBAF (1M in THF) (0.2 mmol, 200 μL) in THE (1.5 mL). The mixture was stirred at room temperature for 1.5 h. The reaction was quenched by adding H₂O (2 mL) and EtOAc (2 mL). The organic phase was dried over anhydrous Na₂SO₄, filtration and removed all of organic solvent.

The residue was dissolved in DCM (1.5 mL) and added PCC (0.15 mmol, 32.2 mg) in a 7 mL vial equipped with a stirring bar, and then stirred at room temperature for 3 h. after that, the mixture was filtered through short flash chromatography with silica (ethyl acetate hexanes=1:1).

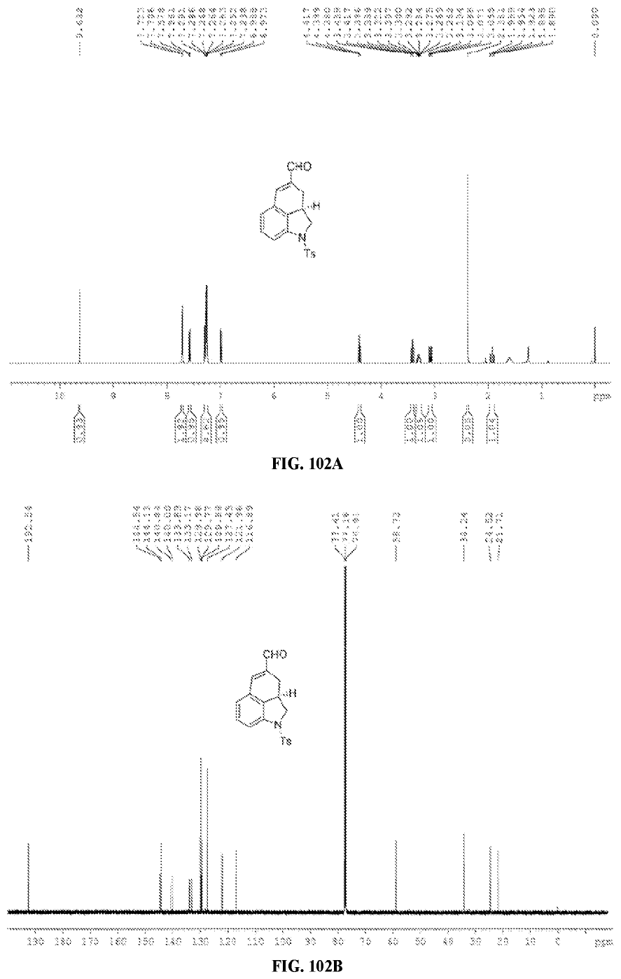

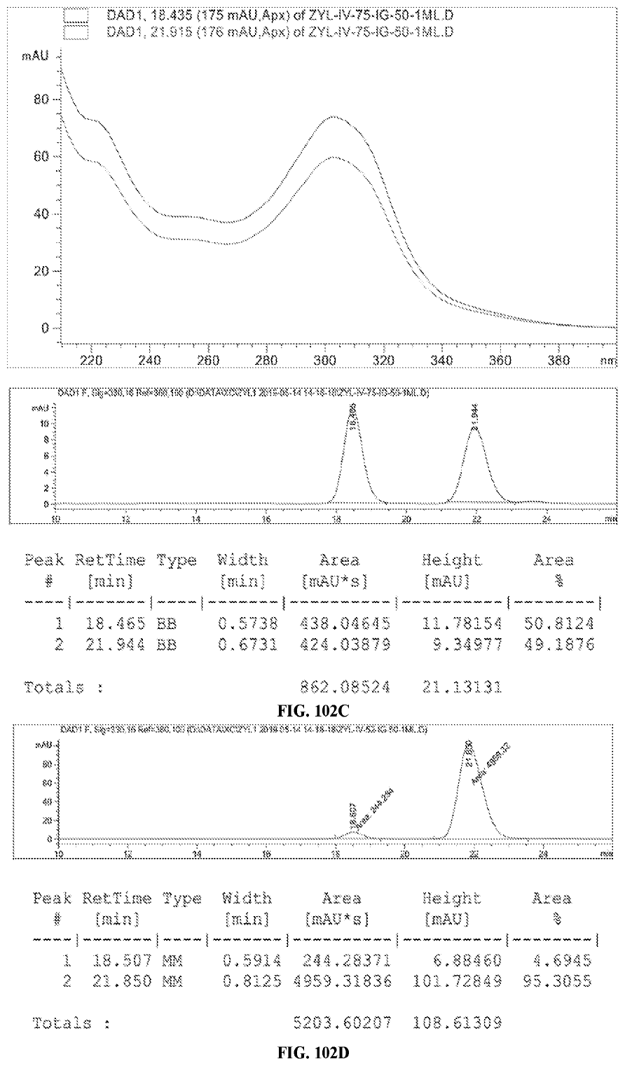

The crude product was transferred to a 7 mL vial in anhydrous MeOH (1 mL), and K2CO3 (0.5 mmol, 69.1 mg) was added. Then the mixture was stirred at room temperature for 16 h. the solvent was removed and the residue was purified by column (ethyl acetate:hexanes=1:5) to get the product 3a (19.6 mg, 59%). Brown oil; ¹H NMR (500 MHz, CDCl3) 5 9.63 (s, 1H), 7.71 (d, J=8.2 Hz, 2H), 7.57 (d, J=8.1 Hz, 1H), 7.30-7.23 (m, 4H), 6.98 (d, J=7.4 Hz, 1H), 4.42-4.37 (m, 1H), 3.45-3.39 (m, 1H), 3.35-3.24 (m, 1H), 3.08 (dd, J=16.6, 7.7 Hz, 1H), 2.38 (s, 3H), 1.92 (td, J=16.0, 2.5 Hz, 1H); ¹³C NMR (125 MHz, CDCl3) 5 192.5, 144.5, 144.1, 140.8, 140.0, 133.9, 133.2, 130.0 (2C), 129.8, 129.6, 127.5 (2C), 122.0, 116.9, 58.7, 34.2, 24.5, 21.7; HRMS (ESI) Calcd for C19H18NO3S [M+H]⁺ 340.1002; found 340.0994. [α]²³D=89.8 (c=0.5, CHCl3); HPLC analysis: ee=91%. CHIRALPAK@IG (50% hexanes: 50% isopropanol, 1 mL/min) tmajor=18.5 min, tminor=21.9 min (FIGS. 102A-D).

### 7. H/D Exchange Experiments

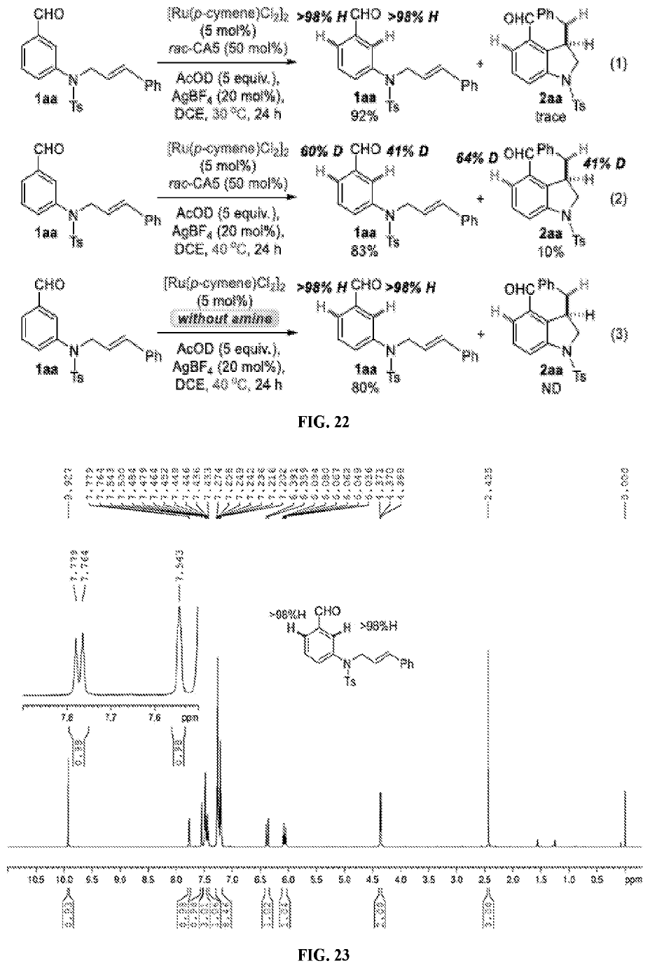

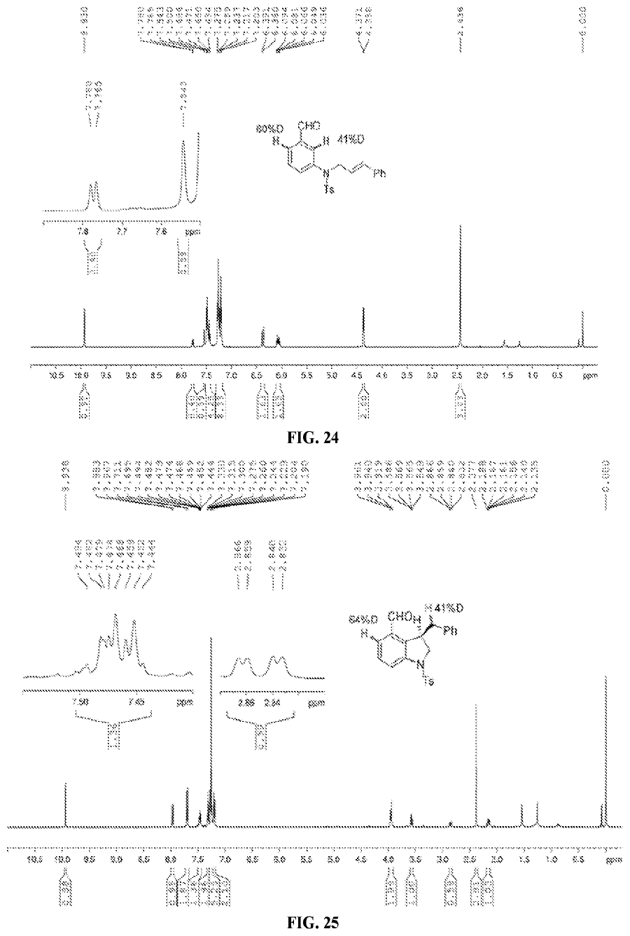

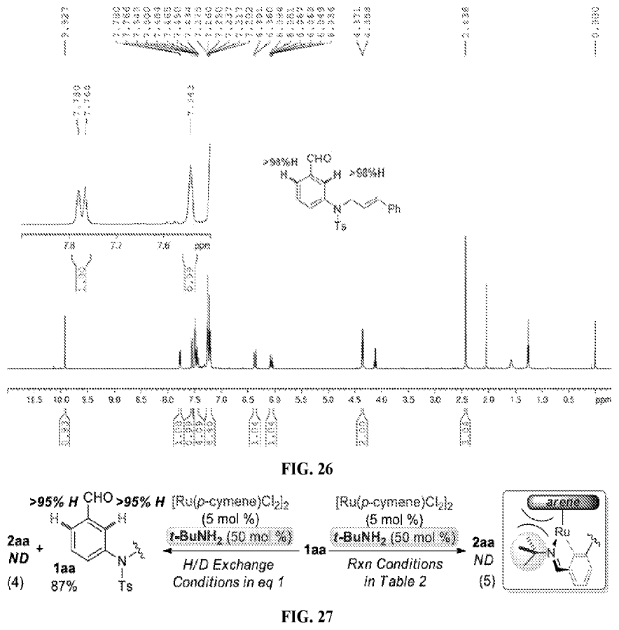

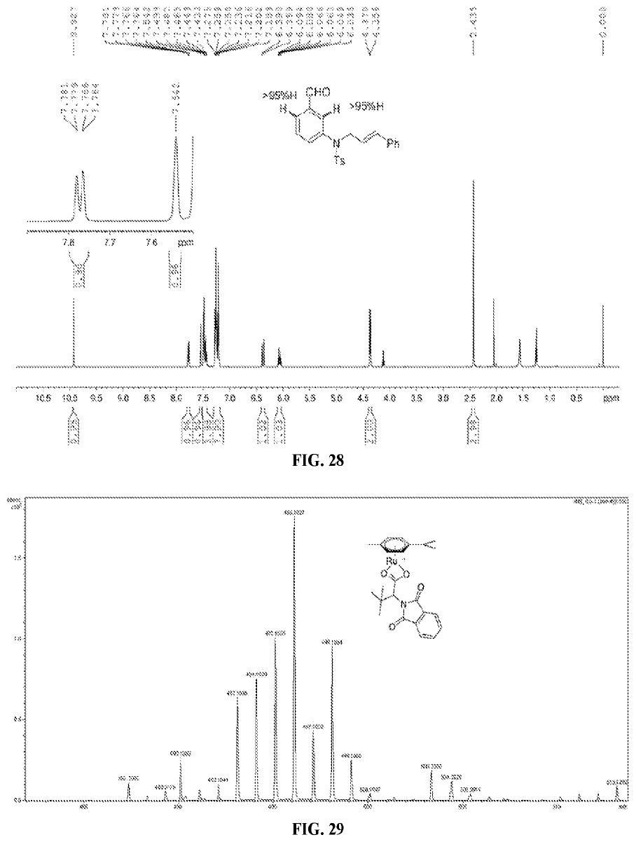

To a 10 mL of Schlenck tube equipped with a magnetic stirring bar were added substrate 1aa (0.05 mmol), [Ru(p-cymene)C12]₂ (5 mol %) and AgBF4 (20 mol %) under air. The mixture was then evacuated and backfilled with nitrogen for three times. After that, 1-phenylethylamine (50 mol %) or without amine, CH₃COOD (0.03 mL) and DCE (0.3 mL) were added subsequently. After stirring at 30° C. or 40° C. for 24 h, the reaction mixture was cooled to room temperature. The solvent was removed under reduced pressure, and the residue was purified by flash chromatography on silica gel with hexanes/ethyl acetate as the eluent to give the remained starting materials 1aa and corresponding product 2aa (FIG. 22). NMR shows the H/D exchange ratios (FIGS. 23-26). A comparison of different reaction conditions is shown in FIGS. 27-28.

### 8. Mechanistic Study on Reaction Intermediates by HRMS Experiments

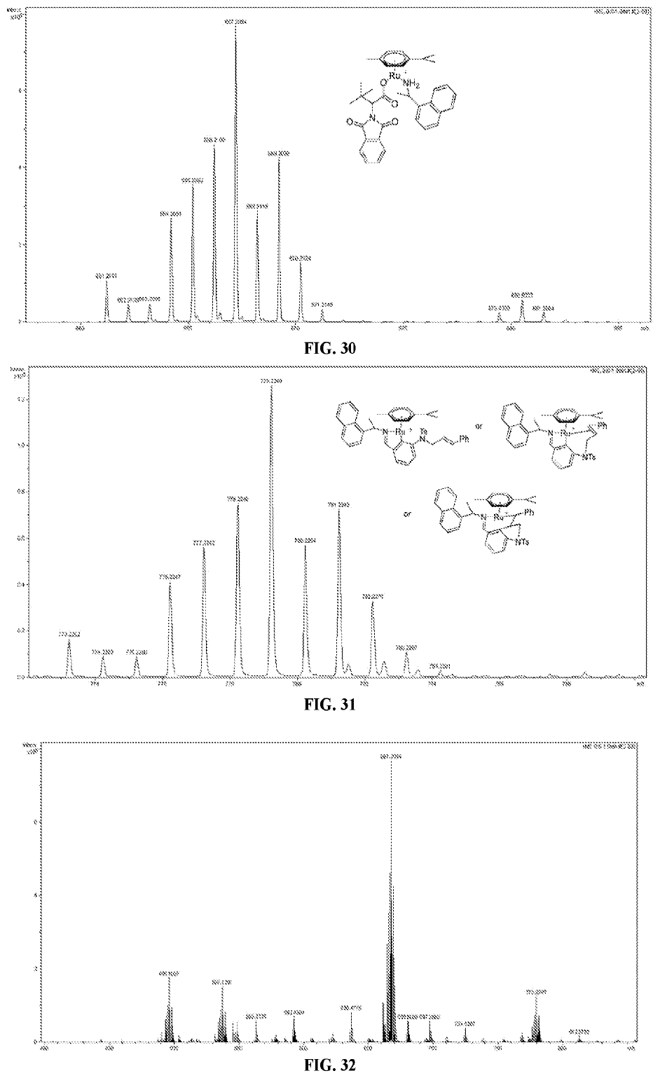

To a 10 mL of Schlenk tube equipped with a magnetic stirring bar were added substrate 1aa (0.05 mmol), [Ru(p-cymene)C12]2 (5 mol %), AgBF₄ (20 mol %), KH₂PO₄ (0.1 mmol) and acid (0.3 equiv.) under air. The mixture was then evacuated and backfilled with nitrogen for three times. After that, chiral amine (50 mol %), HFIP (0.2 mL) and PhCl (0.2 mL) were added subsequently. After stirring at 60° C. for 5 h, the reaction mixture was cooled to room temperature and filtered through celite. Then the mixture was diluted, followed by direct injection into HRMS. The appearance of major signals at 496.1027 (Calcd for 496.1056), 667.2094 (Calcd for 667.2104) and 779.2240 (Calcd for 779.2240) well matched the compounds I-RCO2⁻, III and IV (V) in both mass and isotope pattern (FIGS. 29-32).

### 9. Synthesis of Intermediate 6a and 6b

To a 7 mL vial were added amine (0.8 mmol), benzaldehyde (1.0 mmol) and MgSO4 (2.0 mmol). The mixture was added DCM (2 mL) and stirred at room temperature for 12 h. After that, the residue was filtered through celite and evaporated the solvent to get the corresponding imine product. The crude product was used for the next step without any purification.

To a 7 mL vial were added imine (0.05 mmol), [Ru(p-cymene)C12]2 (0.025 mmol), NaOAc (0.05 mmol), benzaldehyde (0.025 mmol) (to suppress the potential hydrolysis of the imine that releases the amine) and MeOH (1 mL). The mixture was stirred at 40° C. for 3 h. After that, the residue was filtered through celite and washed with DCM to get a red solution. After removing the solvent, the crude mixture was purified by column chromatography on silica gel (ethyl acetate:hexanes=1:3) to get the pure complex.

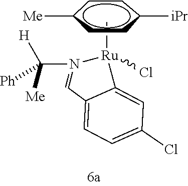

Orange solid (71%); dr=70:30; 1H NMR (500 MHz, CDCl3) 5 8.23 (s, 1×0.3H), 8.14 (s, 1×0.7H), 8.11 (d, J=1.7 Hz, 1×0.7H), 8.07 (d, J=1.7 Hz, 1×0.3H), 7.507.36 (m, 5×0.7H+4×0.3H), 7.33-7.30 (m, 1×0.7H+2×0.3H), 6.98-6.95 (m, 1×0.7H+1×0.3H), 5.66 (q, J=6.8 Hz, 1×0.7H), 5.53-5.49 (m, 2×0.3H), 5.46 (d, J=6.0 Hz, 1×0.7H), 5.41 (d, J=5.8 Hz, 1×0.7H), 5.21 (q, J=6.8 Hz, 1×0.3H), 4.534.50 (m, 1×0.7H+1×0.3H), 4.30 (d, J=5.8 Hz, 1×0.7H), 3.81 (d, J=5.8 Hz, 1×0.3H), 2.42-2.32 (m, 1×0.7H+1×0.3H), 2.10 (s, 3×0.3H), 2.05 (s, 3×0.7H), 2.02 (d, J=6.8 Hz, 3×0.3H), 1.80 (d, J=7.0 Hz, 3×0.7H), 1.02 (d, J=6.9 Hz, 3×0.7H), 0.99 (d, J=6.9 Hz, 3×0.3H), 0.65 (d, J=6.9 Hz, 3×0.7H), 0.55 (d, J=6.9 Hz, 3×0.3H); ¹³C NMR (125 MHz, CDCl3) 5 190.4 (0.7C), 189.9 (0.3C), 171.9 (0.3C), 169.2 (0.7C), 144.1 (0.7C), 142.9 (0.3C), 142.4 (0.7C), 140.8 (0.3C), 138.4 (0.7C), 138.3 (0.3C), 135.6 (0.3C), 135.3 (0.7C), 129.9 (0.7C), 129.8 (0.3C), 129.1 (2×0.7C), 128.6 (2×0.3C), 128.34 (0.3C), 128.29 (0.7C), 127.8 (2×0.3C), 127.2 (2×0.7C), 122.7 (0.7C+0.3C), 104.5 (0.7C+0.3C), 102.5 (0.7C+0.3C), 92.7 (0.3C), 92.0 (0.7C), 90.3 (0.7C), 89.8 (0.3C), 81.8 (0.3C), 80.3 (0.7C), 78.3 (0.7C), 76.2 (0.3C), 72.4 (0.7C), 71.4 (0.3C), 30.9 (0.7C), 30.8 (0.3C), 24.7 (0.7C), 24.1 (0.3C), 23.6 (0.7C), 21.1 (0.7C), 20.7 (0.3C), 19.5 (0.3C), 19.1 (0.7C), 18.8 (0.3C); HRMS (ESI) Calcd for C25H27ClNRu [M-C1]* 478.0870; found 478.0873.

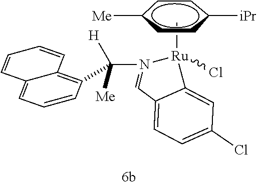

Orange solid (65%); dr=90:10; 1H NMR (500 MHz, CDCl3) 5 8.50 (s, 1×0.1H), 8.23 (s, 1×0.9H), 8.21 (d, J=8.6 Hz, 1×0.9H+1×0.1H), 8.13 (s, 1×0.9H), 8.03 (s, 1×0.1H), 8.00 (d, J=8.1 Hz, 1×0.9H), 7.96 (d, J=8.2 Hz, 1×0.1H), 7.92 (d, J=8.2 Hz, 1×0.9H+1×0.1H), 7.67 (t, J=7.5 Hz, 1×0.9H+1×0.1H), 7.61 (t, J=7.5 Hz, 1×0.9H+1×0.1H), 7.51-7.47 (m, 1×0.9H+1×0.1H), 7.41 (d, J=8.1 Hz, 1×0.9H+1×0.1H), 7.29-7.25 (m, 1×0.9H+1×0.1H), 6.98 (dd, J=8.0, 1.5 Hz, 1×0.9H+1×0.1H), 6.49 (q, J=6.8 Hz, 1×0.9H), 5.96 (q, J=6.8 Hz, 1×0.1H), 5.36 (d, J=6.0 Hz, 1×0.1H), 5.30 (d, J=6.0 Hz, 1×0.9H), 4.95 (d, J=5.8 Hz, 1×0.1H), 4.91 (d, J=5.8 Hz, 1×0.9H), 4.47 (d, J=6.0 Hz, 1×0.9H), 4.28 (d, J=6.0 Hz, 1×0.1H), 4.07 (d, J=5.8 Hz, 1×0.9H), 3.59 (d, J=5.8 Hz, 1×0.1H), 2.45-2.36 (m, 1×0.9H), 2.112.05 (m, 1×0.1H), 2.00 (s, 3×0.9H), 1.93 (d, J=6.9 Hz, 3×0.9H), 1.84 (s, 3×0.1H), 1.74 (d, J=6.9 Hz, 3×0.1H), 1.00 (d, J=7.0 Hz, 3×0.9H), 0.79 (d, J=7.0 Hz, 3×0.1H), 0.61 (d, J=6.9 Hz, 3×0.9H), 0.33 (d, J=6.9 Hz, 3×0.1H); ¹³C NMR (125 MHz, CDCl3) 5 190.6, 169.5, 144.2, 138.42, 138.41, 135.4, 134.2, 131.1, 130.0, 129.5, 128.9, 127.2, 126.4, 125.3, 123.8, 122.7, 122.6, 104.5, 102.6, 92.9, 89.4, 81.5, 76.7, 67.8, 30.8, 24.0, 23.6, 20.6, 18.9; HRMS (ESI) Calcd for C29H29Cl2NRu [M]⁺ 563.0715; found 563.0704.

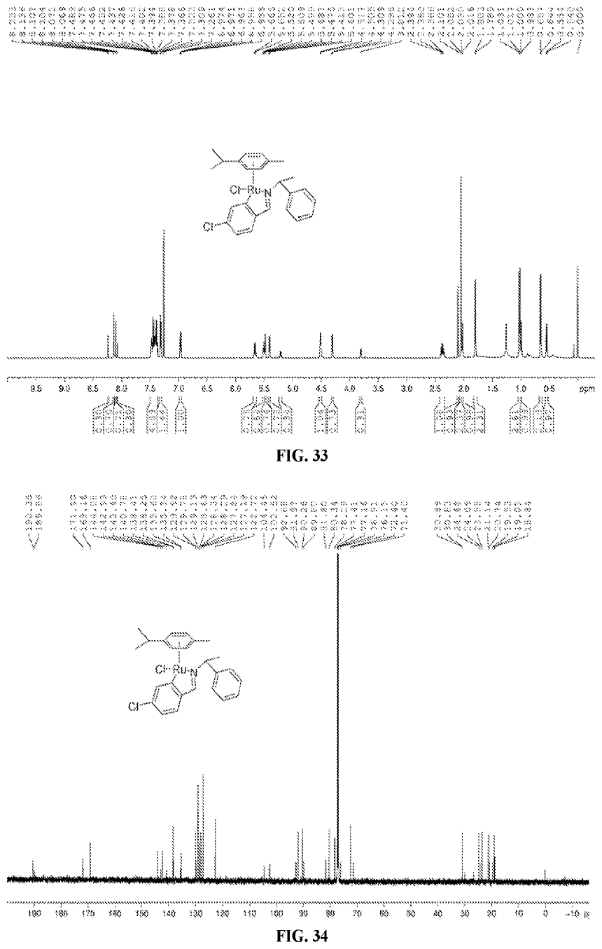

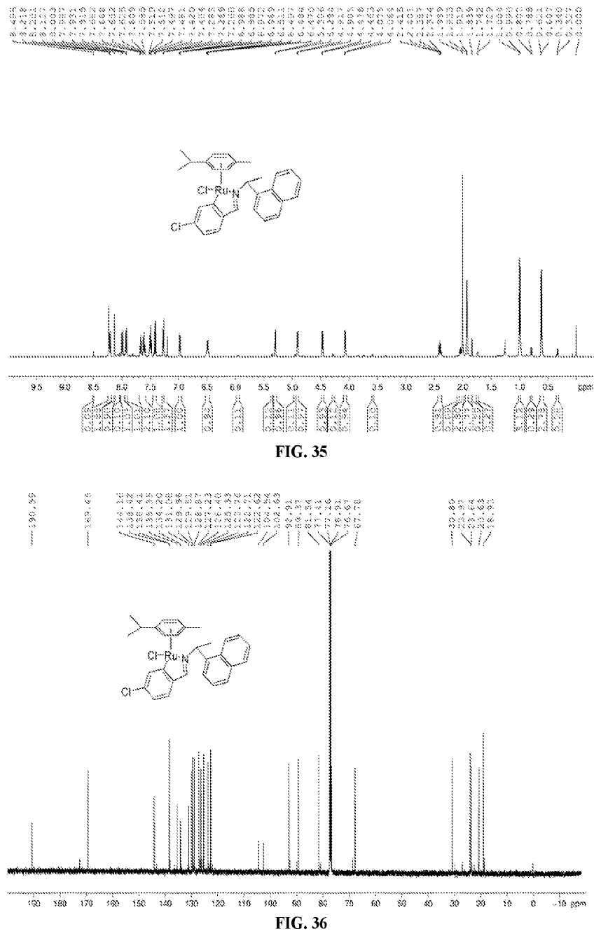

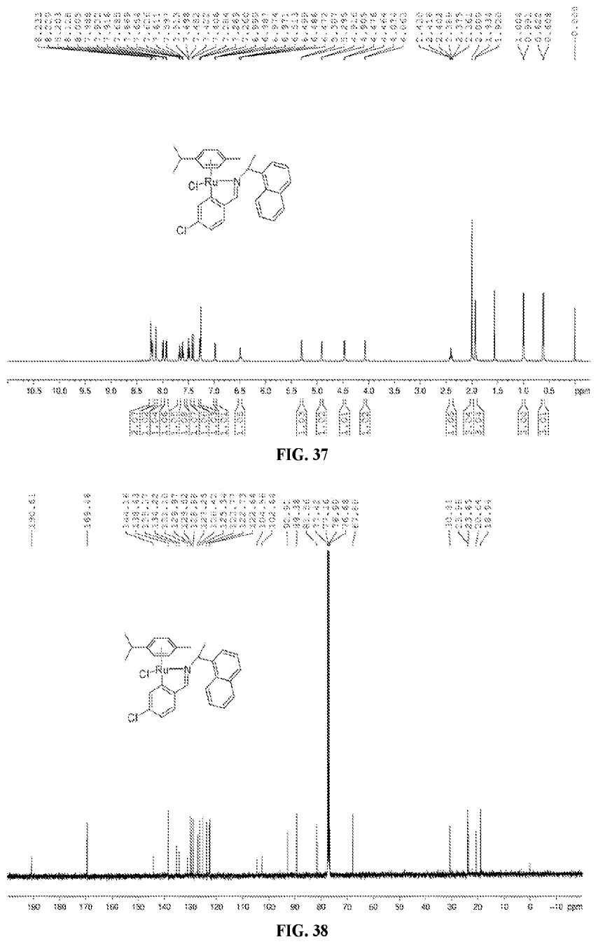

After recrystallization in DCM and hexanes, the major diastereomer of 6b was isolated. The ¹H NMR and ¹³C NMR spectra of the major diastereomer are shown in FIGS. 33-38. Single-crystal X-ray diffraction of the major diastereomer of 6b was obtained as described in the following section.

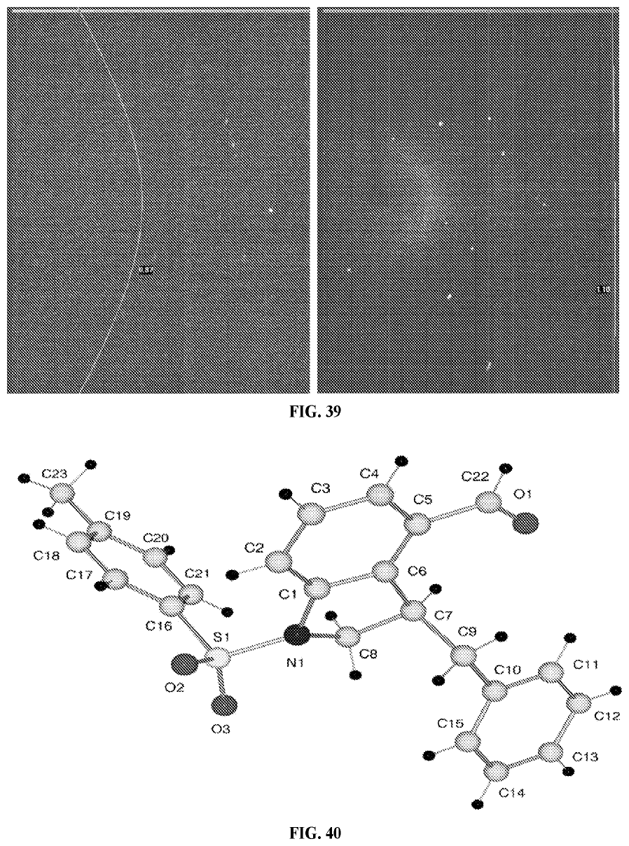

### 10. X-Ray Crystallography for 2Aa (FIGS. 39-40)

A colorless crystal platelet like of C23H21NO3S, approximate dimensions (0.078×0.145×0.352) mm³, was selected for the X-ray crystallographic analysis and mounted on a cryoloop. The X-ray intensity data was measured at room temperature (T=293K), using a three circles goniometer Kappa geometry with a fixed Kappa angle at =54.74 deg Bruker AXS D8 Venture, equipped with a Photon 100 CMOS active pixel sensor detector. A Copper monochromatized X-ray radiation (k=1.54178 Å) was chosen for the measurement. The frames were integrated with the Bruker SAINT software using a narrow-frame algorithm. The integration of the data using a monoclinic unit cell yielded a total of 16356 reflections to a maximum 0 angle of 68.33° (0.83 Å resolution), of which 3552 were independent (average redundancy 4.605, completeness=99.9%, Rint=9.40%, Rsig=6.44%) and 2647 (74.52%) were greater than 2a (F²). The final cell constants of a=5.5476(3) Å, b=8.4528(5) Å, c=21.0022(13) Å, β=97.001(4) °, volume=977.51(10) Å³, are based upon the refinement of the XYZ-centroids of 404 reflections above 20 σ (I) with 8.483°<2θ<134.0°. Data were corrected for absorption effects using the Multi-Scan method (SADABS). The ratio of minimum to maximum apparent transmission was 0.851. The calculated minimum and maximum transmission coefficients (based on crystal size) are 0.5920 and 0.8810. The structure was solved and refined using the Bruker SHELXT-Software Package, using the chiral space group P 1 2(1) 1, with Z=2 for the formula unit, C23H21N03S. Refinement of the structure was carried out by least squares procedures on weighted F² values using the SHELXTL 2016/6 (Sheldrick, 2016) included in the APEX3 v2018, 1.0, AXS Bruker program and the Integrated System of Windows Programs for the Solution, Refinement and Analysis of Single Crystal X-Ray Diffraction Data: WinGX—Version 2018.3. Hydrogen atoms were localized on difference Fourier maps but then introduced in the refinement as fixed contributors in idealized geometry with an isotropic thermal parameters fixed at 20% higher than those carbons atoms they were connected. The final anisotropic full-matrix least-squares refinement on F2 with 255 variables converged at R1=4.28%, for the observed data and wR2=9.02% for all data. The goodness-of-fit: GOF was 1.025. The largest peak in the final difference electron density synthesis was 0.153 e⁻/Å³ and the largest hole was-0.190 e⁻/Å³ with an RMS deviation of 0.035 e⁻/Å³. Based on the final model, the calculated density was 1.330 g/cm³ and F (000), 412 e. Graphical was performed using ORTEP3 for Windows. Cif file was formatted using: CIF format: Hall, S. R.; McMahon, B. International Tables for Crystallography Volume G: Definition and exchange of crystallographic data. Dordrecht: Springer 2005.

TABLE S1
Sample and crystal data for Z_YL_II_15A.

| Identification code | Z_YL_II_15A | |
|---|---|---|
| Chemical formula | C₂₃H₂₁NO₃S | |
| Formula weight | 391.47 g/mol | |
| Temperature | 294(2) K | |
| Wavelength | 1.54178 Å | |
| Crystal size | (0.078 × 0.145 × 0.352) mm³ | |
| Crystal system | monoclinic | |
| Space group | P 12(1) 1 | |
| Unit cell dimensions | a = 5.5476(3) Å | α = 90º |
| | b = 8.4528(5) Å | β = 97.001(4)º |
| | c = 21.0022(13) Å | γ = 90º |
| Volume | 977.51(10) Å3 | |
| Z | 2 | |
| Density (calculated) | 1.330 g/cm³ | |
| Absorption coefficient | 1.665 mm⁻¹ | |
| F(000) | 412 | |

TABLE S2
Data collection and structure refinement for Z_YL_II_15A.

| | | |
|---|---|---|
| Theta range for data collection | 2.12 to 68.33º | |
| Index ranges | −6 <= h <= 5, −10 <= k <= 10, | |
| | −25 <= 1 <= 25 | |
| Reflections collected | 16356 | |
| Independent reflections | 3552 [R(int) = 0.0940] | |
| Coverage of independent | 99.9% | |
| reflections | | |
| Absorption correction | Multi-Scan | |
| Max. and min. transmission | 0.8810 and 0.5920 | |
| Structure solution technique | direct methods | |
| Structure solution program | XT, VERSION 2014/5 | |
| Refinement method | Full-matrix least-squares on F² | |
| Refinement program | SHELXL-2017/1 (Sheldrick, 2017) | |
| Function minimized | Σ w(Fₒ² − F_{c}²)² | |
| Data / restraints / parameters | 3552/1/255 | |
| Goodness-of-fit on F² | 1.025 | |
| Final R indices | 2647 data; I > 2σ(I) | R1 = 0.0428, wR2 = 0.0802 |
| | all data | R1 = 0.0689, wR2 = 0.0902 |
| Weighting scheme | w = 1/[σ²(Fₒ²) + (0.0214P)² + 0.1424P] | |
| | where P=(Fₒ² + 2F_{c}²)/3 | |
| Absolute structure parameter | 0.071(17)* | |
| Extinction coefficient | 0.0073(8) | |
| Largest diff. peak and hole | 0.153 and −0.190 eÅ⁻³ | |
| R.M.S. deviation from mean | 0.035 eÅ⁻³ | |

*Flack x determined using 953 quotients [(I+) − (I−)]/[(I+) + (I−)]
(Parsons, Flack and Wagner, Acta Crust. B69 (2013) 249-259)

TABLE S3
Atomic coordinates and equivalent isotropic atomic displacement
parameters (Å²) for Z_YL_II_15A.
U(eq) is defined as one third of the trace of the orthogonalized Uᵢⱼ tensor.

| | x/a | y/b | z/c | U(eq) |
|---|---|---|---|---|
| S1 | 0.31963(18) | 0.55344(13) | 0.33495(5) | 0.0519(3) |
| N1 | 0.4628(6) | 0.4905(3) | 0.27665(17) | 0.0482(9) |
| O1 | 0.0593(8) | 0.2748(5) | 0.1332(2) | 0.0938(13) |
| O2 | 0.1301(5) | 0.4439(4) | 0.34202(16) | 0.0630(9) |
| O3 | 0.2617(6) | 0.7150(3) | 0.32027(16) | 0.0681(9) |
| C1 | 0.5388(8) | 0.3316(5) | 0.2689(2) | 0.0464(10) |
| C2 | 0.4635(9) | 0.1953(5) | 0.2974(2) | 0.0602(12) |
| C3 | 0.5646(9) | 0.0528(7) | 0.2820(2) | 0.0676(12) |
| C4 | 0.7356(9) | 0.0472(7) | 0.2395(2) | 0.0672(12) |
| C5 | 0.8056(8) | 0.1837(5) | 0.2097(2) | 0.0573(11) |
| C6 | 0.7032(8) | 0.3289(5) | 0.2244(2) | 0.0472(10) |
| C7 | 0.7294(8) | 0.4912(5) | 0.19644(19) | 0.0473(11) |
| C8 | 0.6327(7) | 0.5956(4) | 0.2466(2) | 0.0499(11) |
| C9 | 0.5778(9) | 0.5060(5) | 0.1303(2) | 0.0607(13) |
| C10 | 0.6124(8) | 0.6625(6) | 0.09870(19) | 0.0528(11) |
| C11 | 0.8043(9) | 0.6843(6) | 0.0637(2) | 0.0682(13) |
| C12 | 0.8383(11) | 0.8263(8) | 0.0333(3) | 0.0805(16) |
| C13 | 0.6807(12) | 0.9502(8) | 0.0389(3) | 0.0855(18) |
| C14 | 0.4907(11) | 0.9304(7) | 0.0736(3) | 0.0837(17) |
| C15 | 0.4575(9) | 0.7879(7) | 0.1031(2) | 0.0729(15) |
| C16 | 0.5219(6) | 0.5499(5) | 0.40629(18) | 0.0462(9) |
| C17 | 0.4800(8) | 0.4529(6) | 0.4561(2) | 0.0596(12) |
| C18 | 0.6334(8) | 0.4585(6) | 0.5135(2) | 0.0646(13) |
| C19 | 0.8310(7) | 0.5569(6) | 0.52141(19) | 0.0537(10) |
| C20 | 0.8733(8) | 0.6513(6) | 0.4704(2) | 0.0592(12) |
| C21 | 0.7196(8) | 0.6506(5) | 0.4134(2) | 0.0574(11) |
| C22 | 0.9883(10) | 0.1704(7) | 0.1644(3) | 0.0768(15) |
| C23 | 0.9987(9) | 0.5625(7) | 0.5831(2) | 0.0741(13) |

TABLE S4
Bond lengths (A) for Z_YL_II_15A.

| | | | |
|---|---|---|---|
| S1-O2 | 1.422(3) | S1-O3 | 1.428(3) |
| S1-N1 | 1.628(4) | S1-C16 | 1.759(4) |
| N1-C1 | 1.423(5) | N1-C8 | 1.490(5) |
| O1-C22 | 1.194(6) | C1-C6 | 1.383(6) |
| C1-C2 | 1.387(6) | C2-C3 | 1.384(7) |
| C2-H2 | 0.93 | C3-C4 | 1.380(7) |
| C3-H3 | 0.93 | C4-C5 | 1.389(7) |
| C4-H4 | 0.93 | C5-C6 | 1.403(6) |
| C5-C22 | 1.477(7) | C6-C7 | 1.507(6) |
| C7-C8 | 1.521(6) | C7-C9 | 1.538(6) |
| C7-H7 | 0.98 | C8-H8A | 0.97 |
| C8-H8B | 0.97 | C9-C10 | 1.503(6) |
| C9-H9A | 0.97 | C9-H9B | 0.97 |
| C10-C15 | 1.375(7) | C10-C11 | 1.378(6) |
| C11-C12 | 1.383(7) | C11-H11 | 0.93 |
| C12-C13 | 1.378(8) | C12-H12 | 0.93 |
| C13-C14 | 1.363(8) | C13-H13 | 0.93 |
| C14-C15 | 1.377(7) | C14-H14 | 0.93 |
| C15-H15 | 0.93 | C16-C17 | 1.371(6) |
| C16-C21 | 1.382(6) | C17-C18 | 1.390(6) |
| C17-H17 | 0.93 | C18-C19 | 1.370(7) |
| C18-H18 | 0.93 | C19-C20 | 1.379(6) |
| C19-C23 | 1.501(6) | C20-C21 | 1.382(6) |
| C20-H20 | 0.93 | C21-H21 | 0.93 |
| C22-H22 | 0.93 | C23-H23A | 0.96 |
| C23-H23B | 0.96 | C23-H23C | 0.96 |

TABLE S5
Bond angles (º) for Z_YL_II_15A.

| | | | |
|---|---|---|---|
| O2-S1-O3 | 119.7(2) | O2-S1-N1 | 107.50(18) |
| O3-S1-N1 | 105.51(19) | O2-S1-C16 | 107.6(2) |
| O3-S1-C16 | 107.7(2) | N1-S1-C16 | 108.43(18) |
| C1-N1-C8 | 107.6(3) | C1-N1-S1 | 124.9(3) |
| C8-N1-S1 | 121.1(2) | C6-C1-C2 | 122.4(4) |
| C6-C1-N1 | 108.8(4) | C2-C1-N1 | 128.8(4) |
| C3-C2-C1 | 118.0(4) | C3-C2-H2 | 121.0 |
| C1-C2-H2 | 121.0 | C4-C3-C2 | 120.8(5) |
| C4-C3-H3 | 119.6 | C2-C3-H3 | 119.6 |
| C3-C4-C5 | 120.9(5) | C3-C4-H4 | 119.5 |
| C5-C4-H4 | 119.5 | C4-C5-C6 | 119.0(4) |
| C4-C5-C22 | 118.6(5) | C6-C5-C22 | 122.4(4) |
| C1-C6-C5 | 118.8(4) | C1-C6-C7 | 110.6(4) |
| C5-C6-C7 | 130.6(4) | C6-C7-C8 | 101.5(3) |
| C6-C7-C9 | 111.0(3) | C8-C7-C9 | 112.2(4) |
| C6-C7-H7 | 110.6 | C8-C7-H7 | 110.6 |
| C9-C7-H7 | 110.6 | N1-C8-C7 | 104.0(3) |
| N1-C8-H8A | 111.0 | C7-C8-H8A | 111.0 |
| N1-C8-H8B | 111.0 | C7-C8-H8B | 111.0 |
| H8A-C8-H8B | 109.0 | C10-C9-C7 | 112.7(3) |
| C10-C9-H9A | 109.1 | C7-C9-H9A | 109.1 |
| C10-C9-H9B | 109.1 | C7-C9-H9B | 109.1 |
| H9A-C9-H9B | 107.8 | C15-C10-C11 | 117.5(5) |
| C15-C10-C9 | 122.3(4) | C11-C10-C9 | 120.2(5) |
| C10-C11-C12 | 121.3(5) | C10-C11-H11 | 119.3 |
| C12-C11-H11 | 119.3 | C13-C12-C11 | 119.8(5) |
| C13-C12-H12 | 120.1 | C11-C12-H12 | 120.1 |
| C14-C13-C12 | 119.5(5) | C14-C13-H13 | 120.3 |
| C12-C13-H13 | 120.3 | C13-C14-C15 | 120.1(6) |
| C13-C14-H14 | 119.9 | C15-C14-H14 | 119.9 |
| C10-C15-C14 | 121.7(5) | C10-C15-H15 | 119.1 |
| C14-C15-H15 | 119.1 | C17-C16-C21 | 119.5(4) |
| C17-C16-S1 | 120.6(3) | C21-C16-S1 | 119.8(3) |
| C16-C17-C18 | 119.9(4) | C16-C17-H17 | 120.1 |
| C18-C17-H17 | 120.1 | C19-C18-C17 | 121.5(4) |
| C19-C18-H18 | 119.2 | C17-C18-H18 | 119.2 |
| C18-C19-C20 | 117.8(4) | C18-C19-C23 | 121.8(5) |
| C20-C19-C23 | 120.5(5) | C19-C20-C21 | 121.7(4) |
| C19-C20-H20 | 119.2 | C21-C20-H20 | 119.2 |
| C20-C21-C16 | 119.6(4) | C20-C21-H21 | 120.2 |
| C16-C21-H21 | 120.2 | O1-C22-C5 | 126.5(5) |
| O1-C22-H22 | 116.7 | C5-C22-H22 | 116.7 |
| C19-C23-H23A | 109.5 | C19-C23-H23B | 109.5 |
| H23A-C23-H23B | 109.5 | C19-C23-H23C | 109.5 |
| H23A-C23-H23C | 109.5 | H23B-C23-H23C | 109.5 |

TABLE S6
Torsion angles (°) for Z_YL_II_15A.

| | | | |
|---|---|---|---|
| O2-S1-N1-C1 | −46.2(4) | O3-S1-N1-C1 | −174.9(3) |
| C16-S1-N1-C1 | 69.9(4) | O2-S1-N1-C8 | 165.5(3) |
| O3-S1-N1-C8 | 36.7(3) | C16-S1-N1-C8 | −78.4(3) |
| C8-N1-C1-C6 | −13.7(4) | S1-N1-C1-C6 | −165.5(3) |
| C8-N1-C1-C2 | 168.5(4) | S1-N1-C1-C2 | 16.6(6) |
| C6-C1-C2-C3 | 2.3(7) | N1-C1-C2-C3 | 179.8(4) |
| C1-C2-C3-C4 | 0.1(7) | C2-C3-C4-C5 | −1.9(7) |
| C3-C4-C5-C6 | 1.3(7) | C3-C4-C5-C22 | −179.6(4) |
| C2-C1-C6-C5 | −2.9(6) | N1-C1-C6-C5 | 179.2(4) |
| C2-C1-C6-C7 | 173.7(4) | N1-C1-C6-C7 | −4.2(5) |
| C4-C5-C6-C1 | 1.1(6) | C22-C5-C6-C1 | −178.0(4) |
| C4-C5-C6-C7 | −174.8(4) | C22-C5-C6-C7 | 6.2(7) |
| C1-C6-C7-C8 | 19.6(4) | C5-C6-C7-C8 | −164.4(4) |
| C1-C6-C7-C9 | −99.9(4) | C5-C6-C7-C9 | 76.2(6) |
| C1-N1-C8-C7 | 25.5(4) | S1-N1-C8-C7 | 178.7(3) |
| C6-C7-C8-N1 | −26.3(4) | C9-C7-C8-N1 | 92.2(4) |
| C6-C7-C9-C10 | −176.0(4) | C8-C7-C9-C10 | 71.2(5) |
| C7-C9-C10-C15 | −95.4(6) | C7-C9-C10-C11 | 84.7(5) |
| C15-C10-C11-C12 | −0.8(7) | C9-C10-C11-C12 | 179.1(5) |
| C10-C11-C12-C13 | 1.2(8) | C11-C12-C13-C14 | −1.0(9) |
| C12-C13-C14-C15 | 0.4(9) | C11-C10-C15-C14 | 0.2(7) |
| C9-C10-C15-C14 | −179.7(5) | C13-C14-C15-C10 | 0.0(8) |
| O2-S1-C16-C17 | −0.2(4) | O3-S1-C16-C17 | 130.1(4) |
| N1-S1-C16-C17 | −116.2(4) | O2-S1-C16-C21 | −177.5(3) |
| O3-S1-C16-C21 | −47.2(4) | N1-S1-C16-C21 | 66.5(4) |
| C21-C16-C17-C18 | 1.2(7) | S1-C16-C17-C18 | −176.1(4) |
| C16-C17-C18-C19 | −1.6(7) | C17-C18-C19-C20 | 0.2(7) |
| C17-C18-C19-C23 | −179.4(4) | C18-C19-C20-C21 | 1.6(7) |
| C23-C19-C20-C21 | −178.8(4) | C19-C20-C21-C16 | −1.9(7) |
| C17-C16-C21-C20 | 0.5(6) | S1-C16-C21-C20 | 177.8(3) |
| C4-C5-C22-O1 | 176.7(5) | C6-C5-C22-O1 | −4.2(8) |

TABLE S7
Anisotropic atomic displacement parameters (Å²) for Z_YL_II_15A.
The anisotropic atomic displacement factor exponent
takes the form: −2π²[h²a*²U₁₁+ . . . +2 h k a*b*U₁₂]

| | U11 | U22 | U33 | U23 | U13 | U12 |
|---|---|---|---|---|---|---|
| S1 | 0.0465(5) | 0.0462(5) | 0.0632(7) | 0.0038(6) | 0.0070(4) | −0.0002(6) |
| N1 | 0.057(2) | 0.0394(19) | 0.050(2) | 0.0040(16) | 0.0118(17) | −0.0055(15) |
| O1 | 0.098(3) | 0.097(3) | 0.093(3) | −0.014(2) | 0.042(2) | −0.022(2) |
| O2 | 0.0441(17) | 0.068(2) | 0.078(2) | 0.0005(18) | 0.0094(15) | −0.0147(15) |
| O3 | 0.066(2) | 0.0484(19) | 0.090(2) | 0.0103(17) | 0.0101(17) | 0.0137(15) |
| C1 | 0.052(3) | 0.038(2) | 0.048(3) | 0.003(2) | 0.001(2) | −0.0047(19) |
| C2 | 0.077(3) | 0.050(3) | 0.055(3) | 0.007(2) | 0.013(2) | −0.011(2) |
| C3 | 0.094(3) | 0.042(2) | 0.066(3) | 0.003(3) | 0.010(3) | −0.007(3) |
| C4 | 0.086(3) | 0.045(2) | 0.070(3) | −0.001(3) | 0.007(2) | 0.001(3) |
| C5 | 0.061(3) | 0.056(3) | 0.054(3) | −0.010(2) | 0.006(2) | −0.006(2) |
| C6 | 0.050(2) | 0.049(2) | 0.041(3) | −0.001(2) | −0.0008(19) | −0.011(2) |
| C7 | 0.055(2) | 0.051(2) | 0.036(2) | 0.0017(19) | 0.0025(19) | −0.0147(18) |
| C8 | 0.062(3) | 0.041(3) | 0.045(2) | 0.0068(17) | 0.003(2) | −0.0134(18) |
| C9 | 0.074(3) | 0.061(3) | 0.045(3) | 0.006(2) | −0.003(2) | −0.021(2) |
| C10 | 0.059(3) | 0.062(3) | 0.035(2) | 0.006(2) | −0.0034(19) | −0.013(2) |
| C11 | 0.074(3) | 0.077(4) | 0.055(3) | 0.010(3) | 0.011(2) | −0.005(3) |
| C12 | 0.082(4) | 0.099(4) | 0.062(4) | 0.022(3) | 0.013(3) | −0.016(3) |
| C13 | 0.098(5) | 0.085(4) | 0.068(4) | 0.032(3) | −0.013(3) | −0.017(4) |
| C14 | 0.088(4) | 0.087(4) | 0.072(4) | 0.027(3) | −0.004(3) | 0.014(3) |
| C15 | 0.065(3) | 0.092(4) | 0.060(3) | 0.020(3) | 0.004(3) | −0.002(3) |
| C16 | 0.0452(19) | 0.0411(19) | 0.055(2) | −0.001(3) | 0.0148(16) | −0.001(2) |
| C17 | 0.049(3) | 0.056(3) | 0.074(3) | 0.009(3) | 0.010(2) | −0.011(2) |
| C18 | 0.065(3) | 0.063(3) | 0.066(3) | 0.018(3) | 0.008(3) | 0.002(2) |
| C19 | 0.057(2) | 0.053(2) | 0.053(3) | −0.009(3) | 0.0125(19) | 0.011(3) |
| C20 | 0.056(3) | 0.060(3) | 0.063(3) | −0.015(3) | 0.010(2) | −0.012(2) |
| C21 | 0.065(3) | 0.055(3) | 0.055(3) | −0.003(2) | 0.016(2) | −0.016(2) |
| C22 | 0.077(3) | 0.073(4) | 0.084(4) | −0.017(3) | 0.022(3) | −0.003(3) |
| C23 | 0.076(3) | 0.080(3) | 0.063(3) | −0.011(3) | 0.000(2) | 0.023(3) |

TABLE S8
Hydrogen atomic coordinates and isotropic atomic displacement
parameters (Å²) for Z_YL_II_15A.

| | x/a | y/b | z/c | U(eq) |
|---|---|---|---|---|
| H2 | 0.3484 | 0.1994 | 0.3261 | 0.072 |
| H3 | 0.5167 | −0.0403 | 0.3004 | 0.081 |
| H4 | 0.8049 | −0.0492 | 0.2306 | 0.081 |
| H7 | 0.9005 | 0.5150 | 0.1934 | 0.057 |
| H8A | 0.7635 | 0.6317 | 0.2781 | 0.06 |
| H8B | 0.5481 | 0.6869 | 0.2268 | 0.06 |
| H9A | 0.6229 | 0.4217 | 0.1028 | 0.073 |
| H9B | 0.4074 | 0.4929 | 0.1353 | 0.073 |
| H11 | 0.9130 | 0.6019 | 0.0604 | 0.082 |
| H12 | 0.9670 | 0.8382 | 0.0092 | 0.097 |
| H13 | 0.7039 | 1.0466 | 0.0192 | 0.103 |
| H14 | 0.3832 | 1.0134 | 0.0774 | 0.1 |
| H15 | 0.3270 | 0.7762 | 0.1266 | 0.087 |
| H17 | 0.3491 | 0.3833 | 0.4514 | 0.072 |
| H18 | 0.6012 | 0.3941 | 0.5473 | 0.077 |
| H20 | 1.0087 | 0.7170 | 0.4744 | 0.071 |
| H21 | 0.7491 | 0.7175 | 0.3800 | 0.069 |
| H22 | 1.0547 | 0.0707 | 0.1597 | 0.092 |
| H23A | 1.1595 | 0.5329 | 0.5752 | 0.111 |
| H23B | 0.9423 | 0.4903 | 0.6133 | 0.111 |
| H23C | 1.0015 | 0.6678 | 0.6003 | 0.111 |

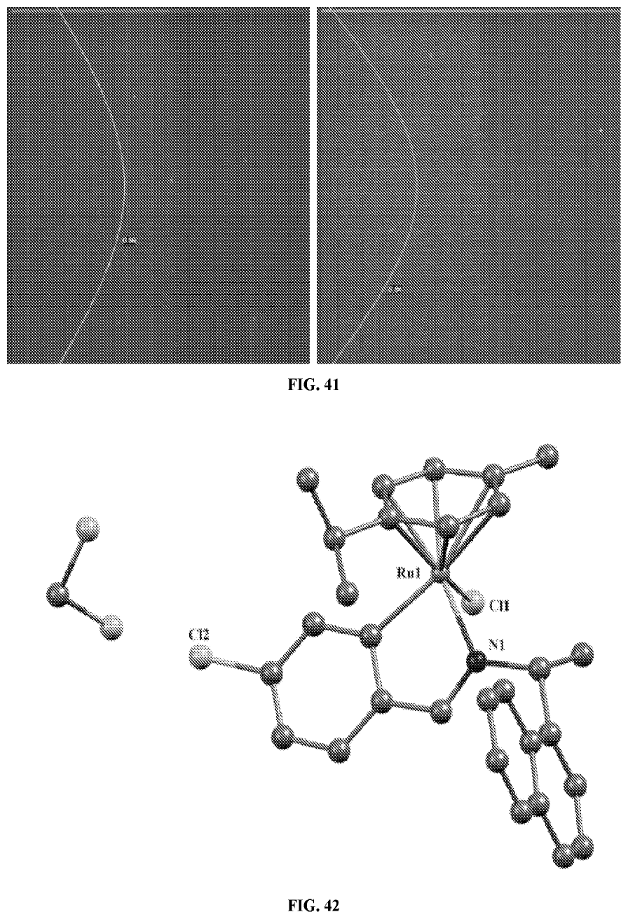

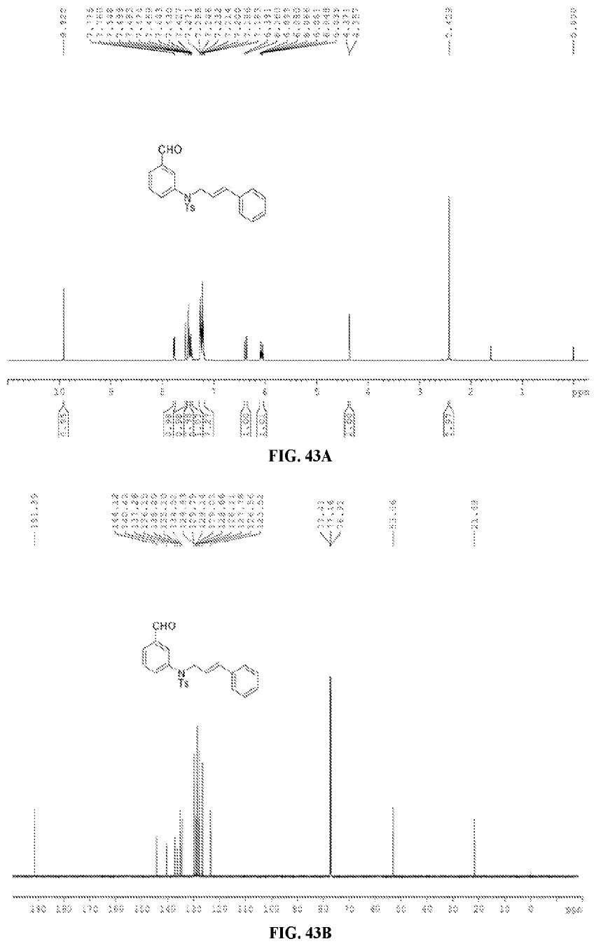

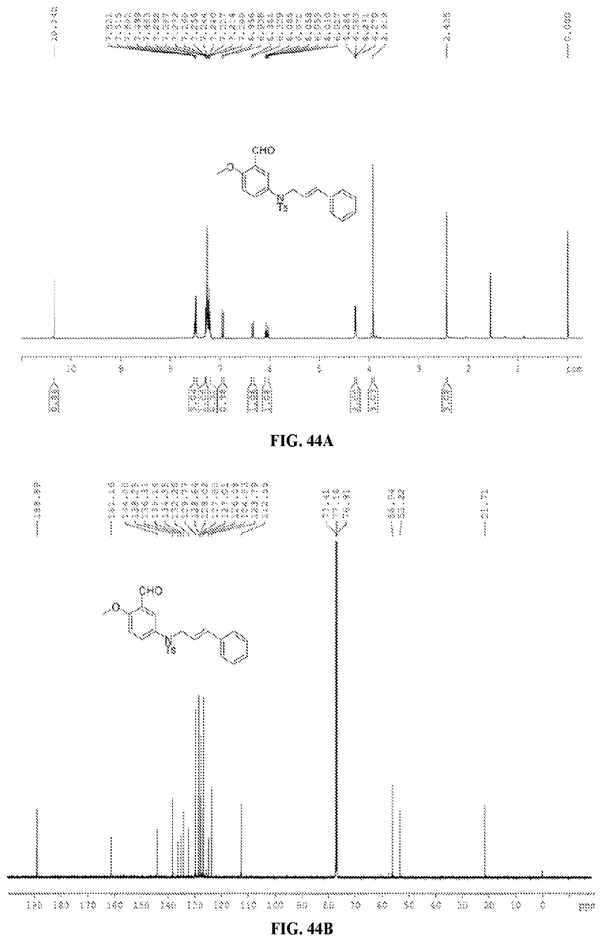

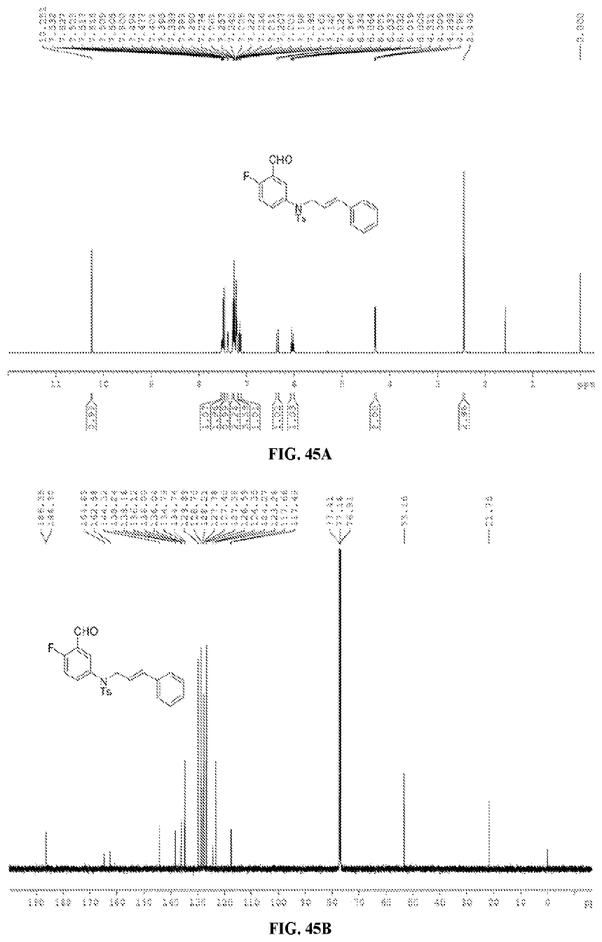

### 11. X-Ray Crystallography for 6b (FIGS. 41-42)

A yellow crystal platelet like C30H31Cl4NRu, approximate dimensions (0.034×0.067×0.345) mm³, was selected for the X-ray crystallographic analysis and mounted on a cryoloop. The X-ray diffracted intensity data was measured at low temperature (T=100 K), using a three circles goniometer Kappa geometry with a fixed Kappa angle at =54.74 deg Bruker AXS D8 Venture, equipped with a Photon 100 CMOS active pixel sensor detector. A Copper monochromatized X-ray radiation (k=1.54178 Å) was selected for the measurement. The frames were integrated with the Bruker SAINT software package¹ using a narrow-frame algorithm. The integration of the data using an orthorhombic unit cell yielded a total of 50230 reflections to a maximum 0 angle of 66.86° (0.84 Å resolution), of which 5016 were independent (average redundancy 10.014, completeness=99.9%, Rint=11.48%, Rsig=4.87%) and 4603 (91.77%) were greater than 2a (F²). The final cell constants of a=7.8236(2) Å, b=14.7618(4) Å, c=24.4735(7) Å, volume=2826.45(13) Å³, are based upon the refinement of the XYZ-centroids of 1475 reflections above 20 σ (I) with 7.224°<2θ<133.3°. Data were corrected for absorption effects using the Multi-Scan method integrated in the program (SADABS)². The ratio of minimum to maximum apparent transmission was 0.760. The calculated minimum and maximum transmission coefficients (based on crystal size) are 0.1660 and 0.7700. The structure was solved in an orthorhombic unit-cell and refined using the SHELXT-Integrated space-group and crystal-structure determination 3, using the chiral space group P 2(1) 2(1) 2(1), with Z=four for the formula unit, C30H31Cl4NRu, a molecule of solvent: dichloromethane:CH2Cl2 was found co-crystallized with the Ru complex. Refinement of the structure was carried out by least squares procedures on weighted F² values using the SHELXTL 2018/3 (Sheidrick, 2016)^{four} included in the APEX3 v2018, 7.2, AXS Bruker program s. Hydrogen atoms were localized on difference Fourier maps but then introduced in the refinement as fixed contributors in idealized geometry with an isotropic thermal parameters fixed at 20 0 higher than those carbons atoms they were connected. The final anisotropic full-matrix least-squares refinement on F² with 330 variables converged at R1=3.09%, for the observed data and wR2=6.25% for all data. The goodness-of-fit GOF was 1.019. The largest peak in the final difference electron density synthesis was 0.692 e⁻/Å³ and the largest hole was −0.391 e⁻/Å³ with an RMS deviation of 0.074 e⁻/Å³. On the basis of the final model, the calculated density was 1.524 g/cm³ and F (000), 1320 e. The Flack's parameter was refined as a 2-component inversion twin⁶

TABLE S1
Sample and crystal data for Cu_Cui_06_24_2019.

| Identification code | Cu_Cui_06_24_2019 | |
|---|---|---|
| Chemical formula | C₃₀H₃₁C₁₄NRu | |
| Formula weight | 648.43 g/mol | |
| Temperature | 100(2) K | |
| Wavelength | 1.54178 Å | |
| Crystal size | (0.034 × 0.067 × 0.345) mm³ | |
| Crystal system | orthorhombic | |
| Space group | P 2(1) 2(1) 2(1) | |
| Unit cell dimensions | a = 7.8236(2) Å | α = 90° |
| | b = 14.7618(4) Å | β = 90° |
| | c = 24.4735(7) Å | γ = 90° |
| Volume | 2826.45(13) Å³ | |
| Z | 4 | |
| Density (calculated) | 1.524 g/cm³ | |
| Absorption coefficient | 8.116 mm⁻¹ | |
| F(000) | 1320 | |

TABLE S2
Data collection and structure refinement for Cu_Cui_06_24_2019.

| | | |
|---|---|---|
| Theta range for data collection | 3.50 to 66.86° | |
| Index ranges | −8 <= h <= 9, −17 <= k <= | |
| | 17, −29 <= l <= 29 | |
| Reflections collected | 50230 | |
| Independent reflections | 5016 [R(int) = 0.1148] | |
| Coverage of independent | 99.9% | |
| reflections | | |
| Absorption correction | Multi-Scan | |
| Max. and min. transmission | 0.7700 and 0.1660 | |
| Structure solution technique | direct methods | |
| Structure solution program | XT, VERSION 2014/5 | |
| Refinement method | Full-matrix least-squares on F² | |
| Refinement program | SHELXL-2018/3 (Sheldrick, 2018) | |
| Function minimized | Σ w(Fₒ² − F_{c}²)² | |
| Data/restraints/parameters | 5016/0/330 | |
| Goodness-of-fit on F² | 1.019 | |
| Final R indices | 4603 data; I > 2σ(I) | R1 = 0.0309, wR2 = |
| | | 0.0607 |
| | all data | R1 = 0.0374, wR2 = |
| | | 0.0625 |
| Weighting scheme | w = 1/[σ²(Fₒ²) + (0.0241P)² + 1.3668P] | |
| | where P = (Fₒ² + 2F_{c}²)/3 | |
| Absolute structure parameter | 0.016(12) | |
| Largest diff. peak and hole | 0.692 and −0.391 eÅ⁻³ | |
| R.M.S. deviation from mean | 0.074 eÅ⁻³ | |

TABLE S3
Atomic coordinates and equivalent isotropic atomic
displacement parameters (Å²) for Cu_Cui_06_24_2019.
U(eq) is defined as one third of the trace of the orthogonalized Uᵢⱼ tensor.

| | x/a | y/b | z/c | U(eq) |
|---|---|---|---|---|
| Ru1 | 0.68282(5) | 0.46569(3) | 0.20179(2) | 0.00950(9) |
| Cl1 | 0.97822(15) | 0.45231(9) | 0.17348(5) | 0.0168(3) |
| Cl2 | 0.93522(19) | 0.60510(10) | 0.40966(5) | 0.0226(3) |
| N1 | 0.6699(6) | 0.5848(3) | 0.15619(16) | 0.0114(9) |
| C1 | 0.7136(7) | 0.6583(3) | 0.1816(2) | 0.0131(12) |
| C2 | 0.7661(6) | 0.6503(4) | 0.2373(2) | 0.0133(12) |
| C3 | 0.8127(8) | 0.7239(3) | 0.2693(2) | 0.0180(11) |
| C4 | 0.8612(7) | 0.7107(4) | 0.3228(2) | 0.0160(13) |
| C5 | 0.8664(6) | 0.6224(4) | 0.3423(2) | 0.0155(12) |
| C6 | 0.8231(7) | 0.5482(3) | 0.31074(19) | 0.0137(10) |
| C7 | 0.7668(6) | 0.5599(3) | 0.2570(2) | 0.0125(11) |
| C8 | 0.6160(7) | 0.5881(4) | 0.0975(2) | 0.0146(12) |
| C9 | 0.5803(7) | 0.6842(4) | 0.0782(2) | 0.0142(12) |
| C10 | 0.4215(7) | 0.7259(4) | 0.0915(2) | 0.0139(11) |
| C11 | 0.2896(7) | 0.6809(4) | 0.1209(2) | 0.0210(13) |
| C12 | 0.1366(7) | 0.7217(5) | 0.1311(3) | 0.0265(15) |
| C13 | 0.1065(8) | 0.8108(5) | 0.1137(3) | 0.0270(15) |
| C14 | 0.2307(8) | 0.8578(4) | 0.0864(2) | 0.0245(14) |
| C15 | 0.3921(7) | 0.8175(4) | 0.0751(2) | 0.0162(12) |
| C16 | 0.5202(8) | 0.8642(4) | 0.0468(2) | 0.0203(13) |
| C17 | 0.6717(8) | 0.8233(4) | 0.0339(2) | 0.0201(12) |
| C18 | 0.7009(8) | 0.7325(4) | 0.0496(2) | 0.0183(12) |
| C19 | 0.7456(7) | 0.5366(4) | 0.0632(2) | 0.0219(12) |
| C20 | 0.6145(7) | 0.3250(4) | 0.1670(2) | 0.0141(12) |
| C21 | 0.6725(8) | 0.3213(3) | 0.2222(2) | 0.0148(11) |
| C22 | 0.5954(7) | 0.3711(3) | 0.2643(2) | 0.0140(12) |
| C23 | 0.4619(7) | 0.4347(3) | 0.2522(2) | 0.0133(11) |
| C24 | 0.4102(6) | 0.4413(3) | 0.1971(2) | 0.0152(11) |
| C25 | 0.4796(7) | 0.3857(4) | 0.1554(2) | 0.0130(12) |
| C26 | 0.6955(8) | 0.2706(4) | 0.1227(2) | 0.0210(12) |
| C27 | 0.3813(7) | 0.4910(3) | 0.2971(3) | 0.0175(11) |
| C28 | 0.3027(8) | 0.5795(4) | 0.2762(2) | 0.0246(13) |
| C29 | 0.2474(7) | 0.4356(4) | 0.3271(2) | 0.0192(12) |
| C1S | 0.3971(9) | 0.6388(5) | 0.5377(3) | 0.0335(16) |
| Cl1S | 0.2346(2) | 0.56312(13) | 0.51778(7) | 0.0443(5) |
| Cl2S | 0.5602(2) | 0.64555(13) | 0.48775(7) | 0.0391(4) |

TABLE S4
Bond lengths (Å) for Cu_Cui_06_24_2019.

| | | | |
|---|---|---|---|
| Ru1-C7 | 2.048(5) | Ru1-N1 | 2.085(4) |
| Ru1-C24 | 2.166(5) | Ru1-C23 | 2.172(5) |
| Ru1-C22 | 2.181(5) | Ru1-C21 | 2.191(5) |
| Ru1-C25 | 2.283(5) | Ru1-C20 | 2.308(5) |
| Ru1-Cl1 | 2.4208(12) | Cl2-C5 | 1.754(5) |
| N1-C1 | 1.296(7) | N1-C8 | 1.497(6) |
| C1-C2 | 1.429(7) | C1-H1 | 0.95 |
| C2-C3 | 1.388(8) | C2-C7 | 1.418(7) |
| C3-C4 | 1.376(7) | C3-H3 | 0.95 |
| C4-C5 | 1.389(8) | C4-H4 | 0.95 |
| C5-C6 | 1.382(7) | C6-C7 | 1.398(7) |
| C6-H6 | 0.95 | C8-C19 | 1.520(7) |
| C8-C9 | 1.521(7) | C8-H8 | 1.0 |
| C9-C18 | 1.375(8) | C9-C10 | 1.424(8) |
| C10-C11 | 1.423(8) | C10-C15 | 1.429(8) |
| C11-C12 | 1.363(8) | C11-H11 | 0.95 |
| C12-C13 | 1.403(9) | C12-H12 | 0.95 |
| C13-C14 | 1.368(9) | C13-H13 | 0.95 |
| C14-C15 | 1.423(8) | C14-H14 | 0.95 |
| C15-C16 | 1.400(8) | C16-C17 | 1.366(9) |
| C16-H16 | 0.95 | C17-C18 | 1.412(8) |
| C17-H17 | 0.95 | C18-H18 | 0.95 |
| C19-H19A | 0.98 | C19-H19B | 0.98 |
| C19-H19C | 0.98 | C20-C25 | 1.413(8) |
| C20-C21 | 1.427(8) | C20-C26 | 1.490(8) |
| C21-C22 | 1.402(7) | C21-H21 | 0.95 |
| C22-C23 | 1.436(7) | C22-H22 | 0.95 |
| C23-C24 | 1.412(8) | C23-C27 | 1.516(8) |
| C24-C25 | 1.417(8) | C24-H24 | 0.95 |
| C25-H25 | 0.95 | C26-H26A | 0.98 |
| C26-H26B | 0.98 | C26-H26C | 0.98 |
| C27-C29 | 1.517(7) | C27-C28 | 1.532(8) |
| C27-H27 | 1.0 | C28-H28A | 0.98 |
| C28-H28B | 0.98 | C28-H28C | 0.98 |
| C29-H29A | 0.98 | C29-H29B | 0.98 |
| C29-H29C | 0.98 | C1S-Cl1S | 1.762(7) |
| C1S-Cl2S | 1.771(7) | C1S-H1S1 | 0.99 |
| C1S-H1S2 | 0.99 | | |

TABLE S5
Bond angles (°) for Cu_Cui_06_24_2019.

| | | | |
|---|---|---|---|
| C7-Ru1-N1 | 78.22(18) | C7-Ru1-C24 | 117.7(2) |
| N1-Ru1-C24 | 93.65(18) | C7-Ru1-C23 | 91.35(19) |
| N1-Ru1-C23 | 116.29(18) | C24-Ru1-C23 | 38.0(2) |
| C7-Ru1-C22 | 94.2(2) | N1-Ru1-C22 | 154.16(19) |
| C24-Ru1-C22 | 67.8(2) | C23-Ru1-C22 | 38.5(2) |
| C7-Ru1-C21 | 121.5(2) | N1-Ru1-C21 | 160.18(17) |
| C24-Ru1-C21 | 79.3(2) | C23-Ru1-C21 | 68.7(2) |
| C22-Ru1-C21 | 37.40(19) | C7-Ru1-C25 | 154.57(19) |
| N1-Ru1-C25 | 97.82(18) | C24-Ru1-C25 | 37.0(2) |
| C23-Ru1-C25 | 67.62(19) | C22-Ru1-C25 | 78.43(19) |
| C21-Ru1-C25 | 65.5(2) | C7-Ru1-C20 | 158.3(2) |
| N1-Ru1-C20 | 123.38(18) | C24-Ru1-C20 | 66.6(2) |
| C23-Ru1-C20 | 80.55(19) | C22-Ru1-C20 | 67.1(2) |
| C21-Ru1-C20 | 36.87(19) | C25-Ru1-C20 | 35.85(19) |
| C7-Ru1-Cl1 | 86.45(14) | N1-Ru1-Cl1 | 87.82(13) |
| C24-Ru1-Cl1 | 155.66(15) | C23-Ru1-Cl1 | 154.83(15) |
| C22-Ru1-Cl1 | 116.61(15) | C21-Ru1-Cl1 | 91.21(16) |
| C25-Ru1-Cl1 | 118.70(14) | C20-Ru1-Cl1 | 92.41(14) |
| C1-N1-C8 | 120.5(4) | C1-N1-Ru1 | 115.8(3) |
| C8-N1-Ru1 | 123.7(3) | N1-C1-C2 | 117.7(5) |
| N1-C1-H1 | 121.1 | C2-C1-H1 | 121.1 |
| C3-C2-C7 | 122.9(5) | C3-C2-C1 | 123.3(5) |
| C7-C2-C1 | 113.8(5) | C4-C3-C2 | 119.9(5) |
| C4-C3-H3 | 120.1 | C2-C3-H3 | 120.1 |
| C3-C4-C5 | 117.8(5) | C3-C4-H4 | 121.1 |
| C5-C4-H4 | 121.1 | C6-C5-C4 | 123.0(5) |
| C6-C5-Cl2 | 119.0(4) | C4-C5-Cl2 | 117.9(4) |
| C5-C6-C7 | 120.3(5) | C5-C6-H6 | 119.9 |
| C7-C6-H6 | 119.9 | C6-C7-C2 | 115.9(5) |
| C6-C7-Ru1 | 129.6(4) | C2-C7-Ru1 | 114.4(4) |
| N1-C8-C19 | 109.0(4) | N1-C8-C9 | 112.3(4) |
| C19-C8-C9 | 114.7(5) | N1-C8-H8 | 106.8 |
| C19-C8-H8 | 106.8 | C9-C8-H8 | 106.8 |
| C18-C9-C10 | 119.4(5) | C18-C9-C8 | 121.1(5) |
| C10-C9-C8 | 119.5(5) | C11-C10-C9 | 123.1(5) |
| C11-C10-C15 | 117.8(5) | C9-C10-C15 | 119.1(5) |
| C12-C11-C10 | 121.6(6) | C12-C11-H11 | 119.2 |
| C10-C11-H11 | 119.2 | C11-C12-C13 | 120.4(6) |
| C11-C12-H12 | 119.8 | C13-C12-H12 | 119.8 |
| C14-C13-C12 | 120.3(6) | C14-C13-H13 | 119.9 |
| C12-C13-H13 | 119.9 | C13-C14-C15 | 120.9(6) |
| C13-C14-H14 | 119.6 | C15-C14-H14 | 119.6 |
| C16-C15-C14 | 121.7(5) | C16-C15-C10 | 119.3(5) |
| C14-C15-C10 | 119.0(5) | C17-C16-C15 | 121.2(5) |
| C17-C16-H16 | 119.4 | C15-C16-H16 | 119.4 |
| C16-C17-C18 | 119.8(6) | C16-C17-H17 | 120.1 |
| C18-C17-H17 | 120.1 | C9-C18-C17 | 121.3(6) |
| C9-C18-H18 | 119.3 | C17-C18-H18 | 119.3 |
| C8-C19-H19A | 109.5 | C8-C19-H19B | 109.5 |
| H19A-C19-H19B | 109.5 | C8-C19-H19C | 109.5 |
| H19A-C19-H19C | 109.5 | H19B-C19-H19C | 109.5 |
| C25-C20-C21 | 116.8(5) | C25-C20-C26 | 120.8(5) |
| C21-C20-C26 | 122.3(5) | C25-C20-Ru1 | 71.1(3) |
| C21-C20-Ru1 | 67.1(3) | C26-C20-Ru1 | 130.9(4) |
| C22-C21-C20 | 122.6(5) | C22-C21-Ru1 | 70.9(3) |
| C20-C21-Ru1 | 76.0(3) | C22-C21-H21 | 118.7 |
| C20-C21-H21 | 118.7 | Ru1-C21-H21 | 126.3 |
| C21-C22-C23 | 120.3(5) | C21-C22-Ru1 | 71.7(3) |
| C23-C22-Ru1 | 70.4(3) | C21-C22-H22 | 119.9 |
| C23-C22-H22 | 119.9 | Ru1-C22-H22 | 130.8 |
| C24-C23-C22 | 116.8(5) | C24-C23-C27 | 122.5(5) |
| C22-C23-C27 | 120.7(5) | C24-C23-Ru1 | 70.8(3) |
| C22-C23-Ru1 | 71.1(3) | C27-C23-Ru1 | 128.9(3) |
| C23-C24-C25 | 122.5(5) | C23-C24-Ru1 | 71.2(3) |
| C25-C24-Ru1 | 76.0(3) | C23-C24-H24 | 118.7 |
| C25-C24-H24 | 118.7 | Ru1-C24-H24 | 125.9 |
| C20-C25-C24 | 120.6(5) | C20-C25-Ru1 | 73.0(3) |
| C24-C25-Ru1 | 67.0(3) | C20-C25-H25 | 119.7 |
| C24-C25-H25 | 119.7 | Ru1-C25-H25 | 133.5 |
| C20-C26-H26A | 109.5 | C20-C26-H26B | 109.5 |
| H26A-C26-H26B | 109.5 | C20-C26-H26C | 109.5 |
| H26A-C26-H26C | 109.5 | H26B-C26-H26C | 109.5 |
| C23-C27-C29 | 110.0(4) | C23-C27-C28 | 113.0(5) |
| C29-C27-C28 | 110.1(4) | C23-C27-H27 | 107.8 |
| C29-C27-H27 | 107.8 | C28-C27-H27 | 107.8 |
| C27-C28-H28A | 109.5 | C27-C28-H28B | 109.5 |
| H28A-C28-H28B | 109.5 | C27-C28-H28C | 109.5 |
| H28A-C28-H28C | 109.5 | H28B-C28-H28C | 109.5 |
| C27-C29-H29A | 109.5 | C27-C29-H29B | 109.5 |
| H29A-C29-H29B | 109.5 | C27-C29-H29C | 109.5 |
| H29A-C29-H29C | 109.5 | H29B-C29-H29C | 109.5 |
| Cl1S-C1S-Cl2S | 111.4(4) | Cl1S-C1S-H1S1 | 109.4 |
| Cl2S-C1S-H1S1 | 109.4 | Cl1S-C1S-H1S2 | 109.4 |
| Cl2S-C1S-H1S2 | 109.4 | H1S1-C1S-H1S2 | 108.0 |

TABLE S6
Torsion angles (°) for Cu_Cui_06_24_2019.

| | | | |
|---|---|---|---|
| C8-N1-C1-C2 | −179.1(5) | Ru1-N1-C1-C2 | 0.3(6) |
| N1-C1-C2-C3 | −178.7(5) | N1-C1-C2-C7 | 1.4(7) |
| C7-C2-C3-C4 | −0.4(9) | C1-C2-C3-C4 | 179.7(5) |
| C2-C3-C4-C5 | 1.9(8) | C3-C4-C5-C6 | −0.8(8) |
| C3-C4-C5-Cl2 | 177.9(4) | C4-C5-C6-C7 | −1.7(8) |
| Cl2-C5-C6-C7 | 179.6(4) | C5-C6-C7-C2 | 3.0(7) |
| C5-C6-C7-Ru1 | −176.6(4) | C3-C2-C7-C6 | −2.1(8) |
| C1-C2-C7-C6 | 177.8(5) | C3-C2-C7-Ru1 | 177.6(4) |
| C1-C2-C7-Ru1 | −2.5(6) | C1-N1-C8-C19 | 114.9(5) |
| Ru1-N1-C8-C19 | −64.5(6) | C1-N1-C8-C9 | −13.4(7) |
| Ru1-N1-C8-C9 | 167.3(3) | N1-C8-C9-C18 | 98.5(6) |
| C19-C8-C9-C18 | −26.7(7) | N1-C8-C9-C10 | −79.5(6) |
| C19-C8-C9-C10 | 155.3(5) | C18-C9-C10-C11 | 179.9(5) |
| C8-C9-C10-C11 | −2.0(8) | C18-C9-C10-C15 | −0.6(8) |
| C8-C9-C10-C15 | 177.4(5) | C9-C10-C11-C12 | −177.4(5) |
| C15-C10-C11-C12 | 3.1(8) | C10-C11-C12-C13 | −1.5(9) |
| C11-C12-C13-C14 | −0.1(9) | C12-C13-C14-C15 | 0.0(9) |
| C13-C14-C15-C16 | 179.4(6) | C13-C14-C15-C10 | 1.6(8) |
| C11-C10-C15-C16 | 179.1(5) | C9-C10-C15-C16 | −0.4(8) |
| C11-C10-C15-C14 | −3.1(8) | C9-C10-C15-C14 | 177.4(5) |
| C14-C15-C16-C17 | −176.9(5) | C10-C15-C16-C17 | 0.8(8) |
| C15-C16-C17-C18 | −0.3(8) | C10-C9-C18-C17 | 1.2(8) |
| C8-C9-C18-C17 | −176.8(5) | C16-C17-C18-C9 | −0.7(8) |
| C25-C20-C21-C22 | 3.4(8) | C26-C20-C21-C22 | −179.0(5) |
| Ru1-C20-C21-C22 | 56.0(5) | C25-C20-C21-Ru1 | −52.6(4) |
| C26-C20-C21-Ru1 | 125.0(5) | C20-C21-C22-C23 | −5.4(8) |
| Ru1-C21-C22-C23 | 52.9(4) | C20-C21-C22-Ru1 | −58.3(5) |
| C21-C22-C23-C24 | 2.2(7) | Ru1-C22-C23-C24 | 55.7(4) |
| C21-C22-C23-C27 | −178.2(5) | Ru1-C22-C23-C27 | −124.7(4) |
| C21-C22-C23-Ru1 | −53.5(4) | C22-C23-C24-C25 | 2.7(7) |
| C27-C23-C24-C25 | −176.9(5) | Ru1-C23-C24-C25 | 58.6(4) |
| C22-C23-C24-Ru1 | −55.9(4) | C27-C23-C24-Ru1 | 124.5(5) |
| C21-C20-C25-C24 | 1.6(7) | C26-C20-C25-C24 | −176.1(5) |
| Ru1-C20-C25-C24 | −49.1(4) | C21-C20-C25-Ru1 | 50.6(4) |
| C26-C20-C25-Ru1 | −127.0(5) | C23-C24-C25-C20 | −4.7(8) |
| Ru1-C24-C25-C20 | 51.7(5) | C23-C24-C25-Ru1 | −56.4(4) |
| C24-C23-C27-C29 | 98.5(6) | C22-C23-C27-C29 | −81.1(6) |
| Ru1-C23-C27-C29 | −170.8(4) | C24-C23-C27-C28 | −25.1(7) |
| C22-C23-C27-C28 | 155.3(5) | Ru1-C23-C27-C28 | 65.6(6) |

TABLE S7
Anisotropic atomic displacement parameters (Å²) for Cu_Cui_06_24_2019.
The anisotropic atomic displacement factor exponent
takes the form: −2π²[h²a*²U₁₁+ . . . +2 h k a*b*U₁₂]

| | U₁₁ | U₂₂ | U₃₃ | U₂₃ | U₁₃ | U₁₂ |
|---|---|---|---|---|---|---|
| Ru1 | 0.00801(17) | 0.00804(16) | 0.01245(17) | −0.00010(18) | −0.00034(17) | 0.00001(16) |
| Cl1 | 0.0101(6) | 0.0189(7) | 0.0214(6) | −0.0017(6) | 0.0012(5) | 0.0032(5) |
| Cl2 | 0.0315(8) | 0.0214(7) | 0.0149(7) | −0.0009(6) | −0.0061(6) | −0.0060(6) |
| N1 | 0.008(2) | 0.013(2) | 0.013(2) | −0.0036(17) | 0.0014(19) | 0.002(2) |
| C1 | 0.010(3) | 0.011(3) | 0.019(3) | 0.004(2) | 0.005(2) | −0.003(2) |
| C2 | 0.007(3) | 0.017(3) | 0.016(3) | 0.000(2) | 0.002(2) | −0.002(2) |
| C3 | 0.018(3) | 0.012(3) | 0.024(3) | −0.001(2) | 0.000(3) | 0.001(3) |
| C4 | 0.017(3) | 0.012(3) | 0.019(3) | −0.006(2) | 0.001(2) | −0.002(2) |
| C5 | 0.012(3) | 0.022(3) | 0.012(3) | −0.001(2) | −0.003(2) | −0.003(2) |
| C6 | 0.012(2) | 0.011(3) | 0.018(3) | −0.0016(19) | 0.001(2) | 0.000(2) |
| C7 | 0.007(3) | 0.013(3) | 0.017(3) | −0.001(2) | 0.004(2) | 0.0012(19) |
| C8 | 0.016(3) | 0.018(3) | 0.010(3) | 0.002(2) | −0.001(2) | 0.002(2) |
| C9 | 0.016(3) | 0.016(3) | 0.011(3) | −0.002(2) | −0.003(2) | 0.001(2) |
| C10 | 0.016(3) | 0.017(3) | 0.009(3) | −0.002(2) | −0.001(2) | 0.001(2) |
| C11 | 0.019(4) | 0.021(3) | 0.024(3) | −0.002(2) | 0.002(2) | −0.002(2) |
| C12 | 0.016(3) | 0.035(4) | 0.028(4) | −0.005(3) | 0.003(2) | 0.000(3) |
| C13 | 0.020(3) | 0.034(4) | 0.026(4) | −0.011(3) | 0.000(3) | 0.008(3) |
| C14 | 0.031(4) | 0.023(3) | 0.019(3) | −0.006(3) | −0.008(3) | 0.013(3) |
| C15 | 0.021(3) | 0.014(3) | 0.013(3) | −0.004(2) | −0.006(2) | 0.004(2) |
| C16 | 0.031(4) | 0.017(3) | 0.013(3) | 0.000(2) | −0.005(2) | 0.005(3) |
| C17 | 0.025(3) | 0.023(3) | 0.012(3) | 0.006(2) | 0.000(3) | −0.003(3) |
| C18 | 0.018(3) | 0.022(3) | 0.015(3) | −0.002(2) | 0.001(2) | 0.001(3) |
| C19 | 0.028(3) | 0.022(3) | 0.016(3) | 0.001(3) | 0.002(2) | 0.010(3) |
| C20 | 0.015(3) | 0.010(3) | 0.017(3) | −0.003(2) | 0.001(2) | −0.008(2) |
| C21 | 0.013(3) | 0.011(2) | 0.020(3) | 0.000(2) | 0.002(2) | −0.003(2) |
| C22 | 0.018(3) | 0.009(3) | 0.015(3) | 0.002(2) | 0.001(2) | −0.004(2) |
| C23 | 0.006(3) | 0.010(3) | 0.023(3) | 0.000(2) | 0.002(2) | −0.004(2) |
| C24 | 0.013(3) | 0.011(3) | 0.021(3) | 0.001(2) | 0.001(2) | −0.0023(19) |
| C25 | 0.010(3) | 0.011(3) | 0.017(3) | 0.001(2) | −0.005(2) | −0.005(2) |
| C26 | 0.028(3) | 0.017(3) | 0.018(3) | −0.007(2) | −0.001(3) | 0.002(3) |
| C27 | 0.017(3) | 0.017(3) | 0.019(3) | −0.002(3) | 0.005(3) | −0.0016(19) |
| C28 | 0.026(3) | 0.013(3) | 0.034(3) | −0.005(2) | 0.012(3) | −0.002(3) |
| C29 | 0.019(3) | 0.016(3) | 0.023(3) | −0.004(2) | 0.007(2) | −0.005(2) |
| C1S | 0.033(4) | 0.037(4) | 0.030(4) | −0.005(3) | 0.000(3) | 0.007(3) |
| Cl1S | 0.0380(10) | 0.0580(12) | 0.0369(9) | −0.0170(8) | 0.0051(7) | −0.0035(8) |
| Cl2S | 0.0377(10) | 0.0453(10) | 0.0342(9) | 0.0123(8) | −0.0014(8) | 0.0041(8) |

TABLE S8
Hydrogen atomic coordinates and isotropic
atomic displacement parameters (Å²) for
Cu_Cui_06_24_2019.

| | x/a | y/b | z/c | U(eq) |
|---|---|---|---|---|
| H1 | 0.7107 | 0.7155 | 0.1639 | 0.016 |
| H3 | 0.8111 | 0.7832 | 0.2544 | 0.022 |
| H4 | 0.8903 | 0.7604 | 0.3456 | 0.019 |
| H6 | 0.8317 | 0.4889 | 0.3256 | 0.016 |
| H8 | 0.5058 | 0.5541 | 0.0949 | 0.018 |
| H11 | 0.3087 | 0.6211 | 0.1338 | 0.025 |
| H12 | 0.0498 | 0.6896 | 0.1501 | 0.032 |
| H13 | −0.0006 | 0.8387 | 0.1210 | 0.032 |
| H14 | 0.2089 | 0.9181 | 0.0748 | 0.029 |
| H16 | 0.5015 | 0.9253 | 0.0363 | 0.024 |
| H17 | 0.7571 | 0.8558 | 0.0145 | 0.024 |
| H18 | 0.8060 | 0.7043 | 0.0402 | 0.022 |
| H19A | 0.8571 | 0.5666 | 0.0658 | 0.033 |
| H19B | 0.7081 | 0.5357 | 0.0250 | 0.033 |
| H19C | 0.7553 | 0.4743 | 0.0768 | 0.033 |
| H21 | 0.7671 | 0.2836 | 0.2308 | 0.018 |
| H22 | 0.6316 | 0.3627 | 0.3010 | 0.017 |
| H24 | 0.3257 | 0.4847 | 0.1875 | 0.018 |
| H25 | 0.4351 | 0.3893 | 0.1193 | 0.016 |
| H26A | 0.6425 | 0.2105 | 0.1213 | 0.031 |
| H26B | 0.8180 | 0.2643 | 0.1300 | 0.031 |
| H26C | 0.6791 | 0.3014 | 0.0876 | 0.031 |
| H27 | 0.4730 | 0.5069 | 0.3239 | 0.021 |
| H28A | 0.2035 | 0.5657 | 0.2532 | 0.037 |
| H28B | 0.3878 | 0.6127 | 0.2547 | 0.037 |
| H28C | 0.2665 | 0.6166 | 0.3073 | 0.037 |
| H29A | 0.2016 | 0.4710 | 0.3576 | 0.029 |
| H29B | 0.2996 | 0.3799 | 0.3411 | 0.029 |
| H29C | 0.1546 | 0.4201 | 0.3019 | 0.029 |
| H1S1 | 0.4470 | 0.6184 | 0.5728 | 0.04 |
| H1S2 | 0.3472 | 0.6997 | 0.5435 | 0.04 |

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. (a) Saint-Denis, T. G.; Zhu, R.-Y.; Chen, G.; Wu, Q.-F.; Yu, J.-Q. Enantioselective C(sp³)-H Bond Activation by Chiral Transition Metal Catalysts. Science 2018, 359, 4798; (b) Newton, C. G.; Wang, S.-G.; Oliveira, C. C.; Cramer, N. Catalytic Enantioselective Transformations Involving C—H Bond Cleavage by Transition-Metal Complexes. Chem. Rev. 2017, 117, 8908-8976; (c) Davies, H. M. L.; Morton, D. Collective Approach to Advancing C—H Functionalization. ACS Cent. Sci. 2017, 3, 936-943; (d) Doyle, M. P.; Duffy, R.; Ratnikov, M.; Zhou, L. Catalytic Carbene Insertion into C—H Bonds. Chem. Rev. 2010, 110, 704-724; (e) Gin, R.; Shi, B.-F.; Engle, K. M.; Maugel, N.; Yu, J.-Q. Transition Metal Catalyzed C—H Activation Reactions: Diastereoselectivity and Enantioselectivity. Chem. Soc. Rev. 2009, 38, 3242-3272; (f) Davies, H. M. L.; Beckwith, R. E. J. Catalytic Enantioselective C—H Activation by Means of Metal-Carbenoid-Induced C—H Insertion. Chem. Rev. 2003, 103, 28612903; (g) Zhang, X. P.; Cui, X., 7.03 Asymmetric C—H Functionalization by Transition Metal-Catalyzed Carbene Transfer Reactions. In Comprehensive Organic Synthesis II (Second Edition), Knochel, P., Ed. Elsevier: Amsterdam, 2014; pp 86-120.
2. (a) Dong, Z.; Ren, Z.; Thompson, S. J.; Xu, Y.; Dong, G. Transition-Metal-Catalyzed C—H Alkylation Using Alkenes. Chem. Rev. 2017, 117, 9333-9403; (b) Shang, R.; Ilies, L.; Nakamura, E. Iron-Catalyzed C—H Bond Activation. Chem. Rev. 2017, 117, 9086-9139; (c) Kim, D. S.; Park, W. J.; Jun, C. H. Metal-Organic Cooperative Catalysis in C—H and C—C Bond Activation. Chem. Rev. 2017, 117, 8977-9015; (d) Shi, X.-Y.; Han, W.-J.; Li, C.-J. Transition-Metal-Catalyzed Direct Addition of Aryl C—H Bonds to Unsaturated Electrophiles. Chem. Rec. 2016, 1178-1190; (e) Huang, Z.; Lim, H. N.; Mo, F.; Young, M. C.; Dong, G. Transition Metal-Catalyzed Ketone-Directed or Mediated C—H Functionalization. Chem. Soc. Rev. 2015, 44, 7764-7786; (f) Sauermann, N.; Meyer, T. H.; Qiu, Y.; Ackermann, L. Electrocatalytic C—H Activation. ACS Catal. 2018, 8, 70867103; (g) Cheng, C.; Hartwig, J. F. Catalytic Silylation of Unactivated C—H Bonds. Chem. Rev. 2015, 115, 8946-8975; (h) Mkhalid, I. A. I.; Barnard, J. H.; Marder, T. B.; Murphy, J. M.; Hartwig, J. F. C—H Activation for the Construction of C—B Bonds. Chem. Rev. 2010, 110, 890-931; (i) Lee, D. H.; Kwon, K. H.; Yi, C. S. Selective Catalytic C—H Alkylation of Alkenes with Alcohols. Science 2011, 333, 1613-1616; (j) Davies, H. M. L.; Manning, J. R. Catalytic C—H Functionalization by Metal Carbenoid and Nitrenoid Insertion. Nature 2008, 451, 417-424.
3. (a) He, J.; Wasa, M.; Chan, K. S. L.; Shao, Q.; Yu, J.-Q. Palladium-Catalyzed Transformations of Alkyl C—H Activation. Chem. Rev. 2017, 117, 8754-8786; (b) Engle, K. M.; Mei, T.-S.; Wasa, M.; Yu, J.-Q. Weak Coordination as a Powerful Means for Developing Broadly Useful C—H Functionalization Reactions. Acc. Chem. Res. 2012, 45, 788-802; (c) Lyons, T. W.; Sanford, M. S. Palladium-Catalyzed Ligand-Directed C—H Functionalization Reactions. Chem. Rev. 2010, 110, 1147-1169; (d) Ackermann, L. Carboxylate-Assisted Transition-Metal-Catalyzed C—H Bond Functionalizations: Mechanism and Scope. Chem. Rev. 2011, 111, 1315-1345; (e) Giri, R.; Shi, B.-F.; Engle, K. M.; Maugel, N.; Yu, J.-Q. Transition Metal-Catalyzed C—H Activation Reactions: Diastereoselectivity and Enantioselectivity. Chem. Soc. Rev. 2009, 38, 3242-3272.
4. (a) Shilov, A. E.; Shul'pin, G. B. Activation of C—H Bonds by Metal Complexes. Chem. Rev. 1997, 97, 2879-2932; (b) Hartwig, J. F. Carbon-Heteroatom Bond Formation Catalysed by Organometallic Complexes. Nature 2008, 455, 314; (c) Kakiuchi, F.; Murai, S. Catalytic C—H/Olefin Coupling. Acc. Chem. Res. 2002, 35, 826-834.
5. (a) Saint-Denis, T. G.; Zhu, R.-Y.; Chen, G.; Wu, Q.-F.; Yu, J.-Q. Enantioselective C(sp3)-H Bond Activation by Chiral Transition Metal Catalysts. Science 2018, 359; (b) Yang, Y.-F.; Hong, X.; Yu, J.-Q.; Houk, K. N. Experimental-Computational Synergy for Selective Pd(II)-Catalyzed C—H Activation of Aryl and Alkyl Groups. Acc. Chem. Res. 2017, 50, 28532860.
6. (a) Duarah, G.; Kaishap, P. P.; Begum, T.; Gogoi, S. Recent Advances in Ruthenium(II)-Catalyzed C—H Bond Activation and Alkyne Annulation Reactions. Adv. Synth. Catal. 2019, 361, 654-672; (b) Shan, C.; Zhu, L.; Qu, L.-B.; Bai, R.; Lan, Y. Mechanistic View of Ru-Catalyzed C—H Bond Activation and Functionalization: Computational Advances. Chem. Soc. Rev. 2018, 47, 7552-7576; (c) Nareddy, P.; Jordan, F.; Szostak, M. Recent Developments in Ruthenium-Catalyzed C—H Arylation: Array of Mechanistic Manifolds. ACS Catal. 2017, 7, 5721-5745; (d) Leitch, J. A.; Frost, C. G. Ruthenium-Catalysed σ-Activation for Remote: Meta—Selective C—H Functionalisation. Chem. Soc. Rev. 2017, 46, 7145-7153; (e) Ackermann, L. Carboxylate-Assisted Ruthenium-Catalyzed Alkyne Annulations by C—H/Het-H Bond Functionalizations. Acc. Chem. Res. 2014, 47, 281-295; (f) Arockiam, P. B.; Bruneau, C.; Dixneuf, P. H. Ruthenium(II)-Catalyzed C—H Bond Activation and Functionalization. Chem. Rev. 2012, 112, 5879-5918; (g) De Sarkar, S.; Liu, W.; Kozhushkov, S. I.; Ackermann, L. Weakly Coordinating Directing Groups for Ruthenium(II)-Catalyzed C—H Activation. Adv. Synth. Catal. 2014, 356, 1461-1479.
7. (a) Peneau, A.; Guillou, C.; Chabaud, L. Recent Advances in [Cp*MIII] (M=Co, Rh, Ir)-Catalyzed Intramolecular Annulation Through C—H Activation. Eur. J Org. Chem. 2018, 2018, 5777-5794; (b) Colby, D. A.; Tsai, A. S.; Bergman, R. G.; Ellman, J. A. Rhodium Catalyzed Chelation-Assisted C—H Bond Functionalization Reactions. Acc. Chem. Res. 2012, 45, 814-825; (c) Colby, D. A.; Bergman, R. G.; Ellman, J. A. Rhodium-Catalyzed C—C Bond Formation via Heteroatom-Directed C—H Bond Activation. Chem. Rev. 2010, 110, 624-655.
8. (a) Gandeepan, P.; Miller, T.; Zell, D.; Cera, G.; Warratz, S.; Ackermann, L. 3d Transition Metals for C—H Activation. Chem. Rev. 2018, 119, 2192-2452; (b) Pan, S.; Shibata, T. Recent Advances in Iridium-Catalyzed Alkylation of C—H and N—H Bonds. ACS Catal. 2013, 3, 704-712.
9. (a) Ye, B.; Cramer, N. Chiral Cyclopentadienyls: Enabling Ligands for Asymmetric Rh(III)-Catalyzed C—H Functionalizations. Acc. Chem. Res. 2015, 48, 1308-1318; (b) Engle, K. M.; Yu, J.-Q. Developing Ligands for Palladium(II)-Catalyzed C—H Functionalization: Intimate Dialogue between Ligand and Substrate. J. Org. Chem. 2013, 78, 8927-8955.
10. (a) Hu, L.; Shen, P.-X.; Shao, Q.; Hong, K.; Qiao, J.-X.; Yu, J.-Q. PdII-Catalyzed Enantioselective C(sp3)-H Activation/Cross-Coupling Reactions of Free Carboxylic Acids. Angew. Chem. Int. Ed. 2019, 58, 21342138; (b) Wu, Q.-F.; Wang, X.-B.; Shen, P.-X.; Yu, J.-Q. Enantioselective C—H Arylation and Vinylation of Cyclobutyl Carboxylic Amides. ACS Catal. 2018, 8, 2577-2581; (c) Shen, P.-X.; Hu, L.; Shao, Q.; Hong, K.; Yu, J.-Q. Pd(II)—Catalyzed Enantioselective C(sp3)-H Arylation of Free Carboxylic Acids. J Am. Chem. Soc. 2018, 140, 6545-6549; (d) Shao, Q.; Wu, Q.-F.; He, J.; Yu, J.-Q. Enantioselective γ-C(sp3)-H Activation of Alkyl Amines via Pd(II)/Pd(0) Catalysis. J. Am. Chem. Soc. 2018, 140, 5322-5325; (e) Park, H.; Verma, P.; Hong, K.; Yu, J.-Q. Controlling Pd(IV) Reductive Elimination Pathways Enables Pd(II)-Catalysed Enantioselective C(sp3)-H Fluorination. Nat. Chem. 2018, 10, 755-762; (f) Wu, Q.-F.; Shen, P.-X.; He, J.; Wang, X.-B.; Zhang, F.; Shao, Q.; Zhu, R.-Y.; Mapelli, C.; Qiao, J.-X.; Poss, M. A.; Yu, J.-Q. Formation of α-Chiral Centers by Asymmetric β-C(sp3)-H Arylation, Alkenylation, and Alkynylation. Science 2017, 355, 499-503; (g) Chen, G.; Gong, W.; Zhuang, Z.; Andrä, M. S.; Chen, Y.-Q.; Hong, X.; Yang, Y.-F.; Liu, T.; Houk, K. N.; Yu, J.-Q. Ligand-Accelerated Enantioselective Methylene C(sp3)-H Bond Activation. Science 2016, 353, 1023-1027.
11. (a) Zhang, F.-L.; Hong, K.; Li, T.-J.; Park, H.; Yu, J.-Q. Organic Chemistry: Functionalization of C(sp3)-H Bonds Using a Transient Directing Group. Science 2016, 351, 252-256; (b) Zhang, S.; Yao, Q.-J.; Liao, G.; Li, X.; Li, H.; Chen, H.-M.; Hong, X.; Shi, B.-F. Enantioselective Synthesis of Atropisomers Featuring Pentatomic Heteroaromatics by Pd-Catalyzed C—H Alkynylation. ACS Catal. 2019, 9, 1956-1961; (c) Liao, G.; Yao, Q.-J.; Zhang, Z.-Z.; Wu, Y.-J.; Huang, D.-Y.; Shi, B.-F. Scalable, Stereocontrolled Formal Syntheses of (+)-Isoschizandrin and (+)-Steganone: Development and Applications of Palladium(II)-Catalyzed Atroposelective C—H Alkynylation. Angew. Chem. Int. Ed. 2018, 57, 36613665; (d) Liao, G.; Li, B.; Chen, H.-M.; Yao, Q.-J.; Xia, Y.-N.; Luo, J.; Shi, B.-F. Pd-Catalyzed Atroposelective C—H Allylation through β-O Elimination: Diverse Synthesis of Axially Chiral Biaryls. Angew. Chem. Int. Ed. 2018, 57, 17151-17155; (e) Yao, Q.-J.; Zhang, S.; Zhan, B.-B.; Shi, B.-F. Atroposelective Synthesis of Axially Chiral Biaryls by Palladium-Catalyzed Asymmetric C—H Olefination Enabled by a Transient Chiral Auxiliary. Angew. Chem. Int. Ed. 2017, 56, 6617-6621.
12. Shi, H.; Herron, A. N.; Shao, Y.; Shao, Q.; Yu, J.-Q. Enantioselective Remote Meta-C—H Arylation and Alkylation via a Chiral Transient Mediator. Nature 2018, 558, 581-585.
13. Ye, B.; Cramer, N. Chiral Cyclopentadienyl Ligands as Stereocontrolling Element in Asymmetric C—H Functionalization. Science 2012, 338, 504-506.
14. Hyster, T. K.; Knorr, L.; Ward, T. R.; Rovis, T. Biotinylated Rh(III) Complexes in Engineered Streptavidin for Accelerated Asymmetric C—H Activation. Science 2012, 338, 500-503.
15. (a) Audic, B.; Wodrich, M. D.; Cramer, N. Mild Complexation Protocol for Chiral CpxRh and Ir Complexes Suitable for in Situ Catalysis. Chem. Sci. 2019, 10, 781-787; (b) Wang, S.-G.; Cramer, N. An Enantioselective Cp^{x}Rh(III)-Catalyzed C—H Functionalization/Ring-Opening Route to Chiral Cyclopentenylamines. Angew. Chem. Int. Ed. 2019, 58, 2514-2518; (c) Tian, M.; Bai, D.; Zheng, G.; Chang, J.; Li, X. Rh(III)—Catalyzed Asymmetric Synthesis of Axially Chiral Biindolyls by Merging C—H Activation and Nucleophilic Cyclization. J. Am. Chem. Soc. 2019, 141, 9527-9532; (d) Li, G.; Jiang, J.; Xie, H.; Wang, J. Introducing the Chiral Transient Directing Group Strategy to Rhodium(III)-Catalyzed Asymmetric C—H Activation. Chem. Eur. J. 2019, 25, 4688-4694; (e) Sun, Y.; Cramer, N. Tailored Trisubstituted Chiral CpxRhIII Catalysts for Kinetic Resolutions of Phosphinic Amides. Chem. Sci. 2018, 9, 2981-2985; (f) Sun, Y.; Cramer, N. Enantioselective Synthesis of Chiral-at-Sulfur 1,2-Benzothiazines by Cp^{x}Rh^{III}-Catalyzed C—H Functionalization of Sulfoximines. Angew. Chem. Int. Ed. 2018, 57, 15539-15543; (g) Shen, B.; Wan, B.; Li, X. Enantiodivergent Desymmetrization in the Rhodium(III)-Catalyzed Annulation of Sulfoximines with Diazo Compounds. Angew. Chem. Int. Ed. 2018, 57, 15534-15538; (h) Satake, S.; Kurihara, T.; Nishikawa, K.; Mochizuki, T.; Hatano, M.; Ishihara, K.; Yoshino, T.; Matsunaga, S. Pentamethylcyclopentadienyl Rhodium(III)-Chiral Disulfonate Hybrid Catalysis for Enantioselective C—H Bond Functionalization. Nat. Catal. 2018, 1, 585-591; (i) Lin, L.; Fukagawa, S.; Sekine, D.; Tomita, E.; Yoshino, T.; Matsunaga, S. Chiral Carboxylic Acid Enabled Achiral Rhodium(III)-Catalyzed Enantioselective C—H Functionalization. Angew. Chem. Int. Ed. 2018, 57, 12048-12052.
16. (a) Jang, Y. S.; Dieckmann, M.; Cramer, N. Cooperative Effects between Chiral Cp^{x}-Iridium(III) Catalysts and Chiral Carboxylic Acids in Enantioselective C—H Amidations of Phosphine Oxides. Angew. Chem. Int. Ed. 2017, 56, 15088-15092; (b) Jang, Y. S.; Wozniak, L.; Pedroni, J.; Cramer, N. Access to P- and Axially Chiral Biaryl Phosphine Oxides by Enantioselective Cp^{x}Ir^{III}-Catalyzed C—H Arylations. Angew. Chem. Int. Ed. 2018, 57, 12901-12905.
17. (a) Ozols, K.; Jang, Y. S.; Cramer, N. Chiral Cyclopentadienyl Cobalt(III) Complexes Enable Highly Enantioselective 3d-Metal-Catalyzed C—H Functionalizations. J. Am. Chem. Soc. 2019, 141, 5675-5680; (b) Fukagawa, S.; Kato, Y.; Tanaka, R.; Kojima, M.; Yoshino, T.; Matsunaga, S. Enantioselective C(sp3)-H Amidation of Thioamides Catalyzed by a Cobalt^{III}/Chiral Carboxylic Acid Hybrid System. Angew. Chem. Int. Ed. 2019, 58, 1153-1157; (c) Pesciaioli, F.; Dhawa, U.; Oliveira, J. C. A.; Yin, R.; John, M.; Ackermann, L. Enantioselective Cobalt(III)-Catalyzed C—H Activation Enabled by Chiral Carboxylic Acid Cooperation. Angew. Chem. Int. Ed. 2018, 57, 15425-15429.
18. (a) Abrams, D. J.; Provencher, P. A.; Sorensen, E. J. Recent Applications of C—H Functionalization in Complex Natural Product Synthesis. Chem. Soc. Rev. 2018, 47, 8925-8967; (b) Kakiuchi, F.; Chatani, N. Catalytic Methods for C—H Bond Functionalization: Application in Organic Synthesis. Adv. Synth. Catal. 2003, 345, 1077-1101.
19. For other Ru-catalyzed C—H Functionalization, see: (a) Kilaru, P.; Acharya, S. P.; Zhao, P. A Tethering Directing Group Strategy for Ruthenium-Catalyzed Intramolecular Alkene Hydroarylation. Chem. Commun. 2018, 54, 924-927; (b) Zhang, J.; Ugrinov, A.; Zhang, Y.; Zhao, P. Exploring Bis(cyclometalated) Ruthenium(II) Complexes as Active Catalyst Precursors: Room-Temperature Alkene-Alkyne Coupling for 1,3-Diene Synthesis. Angew. Chem. Int. Ed. 2014, 53, 8437-8440; (c) Zhang, J.; Ugrinov, A.; Zhao, P. Ruthenium(II)/N-Heterocyclic Carbene Catalyzed [3+2] Carbocyclization with Aromatic N—H Ketimines and Internal Alkynes. Angew. Chem. Int. Ed. 2013, 52, 6681-6684; (d) Yi, C. S.; Sang, Y. Y.; Guzei, I. A. Catalytic Synthesis of Tricyclic Quinoline Derivatives from the Regioselective Hydroamination and C—H Bond Activation Reaction of Benzocyclic Amines and Alkynes. J. Am. Chem. Soc. 2005, 127, 5782-5783; (e) Yi, C. S.; Yun, S. Y. Scope and Mechanistic Study of the Ruthenium-Catalyzed ortho-C—H Bond Activation and Cyclization Reactions of Arylamines with Terminal Alkynes. J. Am. Chem. Soc. 2005, 127, 17000-17006; (f) Lee, D. H.; Kwon, K. H.; Yi, C. S. Dehydrative C—H Alkylation and Alkenylation of Phenols with Alcohols: Expedient Synthesis for Substituted Phenols and Benzofurans. J. Am. Chem. Soc. 2012, 134, 7325-7328; (g) Lee, H.; Yi, C. S. Catalytic Synthesis of Substituted Indoles and Quinolines from the Dehydrative C—H Coupling of Arylamines with 1,2- and 1,3-Diols. Organometallics 2016, 35, 1973-1977.
20. Ru(0)-catalyzed C—H Functionalization has also been underdeveloped, with only 15% yield and 15% ee reported: Kakiuchi, F.; Le Gendre, P.; Yamada, A.; Ohtaki, H.; Murai, S. Atropselective Alkylation of Biaryl Compounds by Means of Transition Metal-Catalyzed C—H/Olefin Coupling. Tetrahedron: Asymmetry 2000, 11, 2647-2651.
21. Trost, B. M.; Frederiksen, M. U.; Rudd, M. T. Ruthenium-Catalyzed Reactions—A Treasure Trove of Atom-Economic Transformations. Angew. Chem. Int. Ed. 2005, 44, 6630-6666.
22. (a) Podoll, J. D.; Liu, Y.; Chang, L.; Walls, S.; Wang, W.; Wang, X. Bio-Inspired Synthesis Yields a Tricyclic Indoline that Selectively Resensitizes Methicillin-Resistant Staphylococcus Aureus (MRSA) to β-Lactam Antibiotics. Proc. Natl. Acad. Sci. U.S.A. 2013, 110, 15573-15578; (b) Gan, Z.; Reddy, P. T.; Quevillon, S.; Couve-Bonnaire, S.; Arya, P. Stereocontrolled Solid-Phase Synthesis of a 90-Membered Library of Indoline-Alkaloid-like Polycycles from an Enantioenriched Aminoindoline Scaffold. Angew. Chem. Int. Ed. 2005, 44, 1366-1368; (c) Deng, L.; Chen, M.; Dong, G. Concise Synthesis of (−)-Cycloclavine and (−)-5-epi-Cycloclavine via Asymmetric C—C Activation. J. Am. Chem. Soc. 2018, 140, 9652-9658.
23. (a) Santi, M.; Müiller, S. T. R.; Folgueiras-Amador, A. A.; Uttry, A.; Hellier, P.; Wirth, T. Enantioselective Synthesis of trans-2,3-Dihydro-1H-indoles Through C—H Insertion of α-Diazocarbonyl Compounds. Eur. J. Org. Chem. 2017, 2017, 1889-1893; (b) Davies, H. M. L.; Morton, D. Guiding Principles for Site Selective and Stereoselective Intermolecular C—H Functionalization by Donor/Acceptor Rhodium Carbenes. Chem. Soc. Rev. 2011, 40, 1857-1869; (c) Wen, X.; Wang, Y.; Zhang, X. P. Enantioselective Radical Process for Synthesis of Chiral Indolines by Metalloradical Alkylation of Diverse C(sp3)-H Bonds. Chem. Sci. 2018, 9, 5082-5086; (d) Wang, Y.; Wen, X.; Cui, X.; Zhang, X. P. Enantioselective Radical Cyclization for Construction of 5-Membered Ring Structures by Metalloradical C—H Alkylation. J. Am. Chem. Soc. 2018, 140, 4792-4796.
24. (a) Kong, C.; Jana, N.; Jones, C.; Driver, T. G. Control of the Chemoselectivity of Metal N-Aryl Nitrene Reactivity: C—H Bond Amination versus Electrocyclization. J. Am. Chem. Soc. 2016, 138, 13271-13280; (b) Villanueva, O.; Weldy, N. M.; Blakey, S. B.; MacBeth, C. E. Cobalt Catalyzed sp3 C—H Amination Utilizing Aryl Azides. Chem. Sci. 2015, 6, 6672-6675; (c) Nguyen, Q.; Sun, K.; Driver, T. G. Rh2(II)-Catalyzed Intramolecular Aliphatic C—H Bond Amination Reactions Using Aryl Azides as the N-Atom Source. J. Am. Chem. Soc. 2012, 134, 7262-7265; (d) Li, C.; Lang, K.; Lu, H.; Hu, Y.; Cui, X.; Wojtas, L.; Zhang, X. P. Catalytic Radical Process for Enantioselective Amination of C(sp3)-H Bonds. Angew. Chem. Int. Ed. 2018, 57, 16837-16841.
25. (a) Mei, T.-S.; Leow, D.; Xiao, H.; Laforteza, B. N.; Yu, J.-Q. Synthesis of Indolines via Pd(II)-Catalyzed Amination of C—H Bonds Using PhI(OAc)2 as the Bystanding Oxidant. Org. Lett. 2013, 15, 3058-3061; (b) Mei, T.-S.; Wang, X.; Yu, J.-Q. Pd(II)—Catalyzed Amination of C—H Bonds Using Single-Electron or Two-electron Oxidants. J. Am. Chem. Soc. 2009, 131, 10806-10807.
26. So far there is only one established example for the synthesis of indoline via enantioselective hydroarylation with 19% ee: (a) Ye, B.; Donets, P. A.; Cramer, N. Chiral Cp-Rhodium(III)-Catalyzed Asymmetric Hydroarylations of 1,1-Disubstituted Alkenes. Angew. Chem. Int. Ed. 2014, 53, 507-511. Alternatively, for palladium-catalyzed enantioselective synthesis of indolines via C—H Activation with aryl halides, see: (b) Anas, S.; Cordi, A.; Kagan, H. B. Enantioselective Synthesis of 2-Methyl indolines by Palladium Catalysed Asymmetric C(sp³)-H Activation/cyclisation. Chem. Commun. 2011, 47, 11483-11485. (c) Nakanishi, M.; Katayev, D.; Besnard, C.; Kundig, E. P. Fused Indolines by Palladium-Catalyzed Asymmetric C—C Coupling Involving an Unactivated Methylene Group. Angew. Chem. Int. Ed. 2011, 50, 7438-7441. (d) Saget, T.; Lemouzy, S. J.; Cramer, N. Chiral Monodentate Phosphines and Bulky Carboxylic Acids: Cooperative Effects in Palladium-Catalyzed Enantioselective C(sp³)-H Functionalization. Angew. Chem. Int. Ed. 2012, 51, 2238-2242. (e) Yang, L.; Melot, R.; Neuburger, M.; Baudoin, O. Palladium(O)-Catalyzed Asymmetric C(sp³)-H Arylation Using a Chiral Binol-Derived Phosphate and an Achiral Ligand. Chem. Sci. 2017, 8, 1344-1349.
27. (a) Thalji, R. K.; Ellman, J. A.; Bergman, R. G. Highly Efficient and Enantioselective Cyclization of Aromatic Imines via Directed C—H Bond Activation. J. Am. Chem. Soc. 2004, 126, 7192-7193; (b) Harada, H.; Thalji, R. K.; Bergman, R. G.; Ellman, J. A. Enantioselective Intramolecular Hydroarylation of Alkenes via Directed C—H Bond Activation. J. Org. Chem. 2008, 73, 6772-6779. (c) Watzke, A.; Wilson, R. M.; O'Malley, S. J.; Bergman, R. G.; Ellman, J. A. Asymmetric Intramolecular Alkylation of Chiral Aromatic Imines via Catalytic C—H Bond Activation. Synlett 2007, 2383-2389.
28. (a) Ghosh, K.; Rit, R. K.; Ramesh, E.; Sahoo, A. K. Ruthenium-Catalyzed Hydroarylation and One-Pot Twofold Unsymmetrical C—H Functionalization of Arenes. Angew. Chem. Int. Ed. 2016, 55, 7821-7825; (b) Ghosh, K.; Shankar, M.; Rit, R. K.; Dubey, G.; Bharatam, P. V.; Sahoo, A. K. Sulfoximine-Assisted One-Pot Unsymmetrical Multiple Annulation of Arenes: A Combined Experimental and Computational Study. J. Org. Chem. 2018, 83, 9667-9681; (c) Rit, R. K.; Ghosh, K.; Mandal, R.; Sahoo, A. K. Ruthenium-Catalyzed Intramolecular Hydroarylation of Arenes and Mechanistic Study: Synthesis of Dihydrobenzofurans, Indolines, and Chromans. J Org. Chem. 2016, 81, 8552-8560.
29. (a) Mantegani, S.; Arlandini, E.; Bandiera, T.; Borghi, D.; Brambilla, E.; Caccia, C.; Cervini, M. A.; Cremonesi, P.; McArthur, R. A.; Traquandi, G.; Varasi, M. D1 Agonist and/or D2 Antagonist Dopamine Receptor Properties of a Series of Ergoline Derivatives: a Structure-Activity Study. Eur. J Med. Chem. 1999, 34, 107-124; (b) Carr, M. A.; Creviston, P. E.; Hutchison, D. R.; Kennedy, J. H.; Khau, V. V.; Kress, T. J.; Leanna, M. R.; Marshall, J. D.; Martinelli, M. J.; Peterson, B. C.; Varie, D. L.; Wepsiec, J. P. Synthetic Studies toward the Partial Ergot Alkaloid LY228729, a Potent 5HT1A Receptor Agonist. J. Org. Chem. 1997, 62, 8640-8653; (c) Anderson, B. A.; Becke, L. M.; Booher, R. N.; Flaugh, M. E.; Ham, N. K.; Kress, T. J.; Varie, D. L.; Wepsiec, J. P. Application of Palladium(0)-Catalyzed Processes to the Synthesis of Oxazole-Containing Partial Ergot Alkaloids. J. Org. Chem. 1997, 62, 8634-8639; (d) Ward, J. S.; Fuller, R. W.; Merritt, L.; Snoddy, H. D.; Paschal, J. W.; Mason, N. R.; Homg, J. S. Ergolines as Selective 5-HT1 Agonists. J. Med. Chem. 1988, 31, 15121519; (e) Kurihara, T.; Terada, T.; Harusawa, S.; Yoneda, R. Synthetic Studies of (±)-Lysergic Acid and Related Compounds. Chem. Pharm. Bull. 1987, 35, 4793-4802; (f) Kurihara, T.; Terada, T.; Yoneda, R. A New Synthesis of (±)-Lysergic Acid. Chem. Pharm. Bull. 1986, 34, 442-443; (g) Armstrong, V. W.; Coulton, S.; Ramage, R. A New Synthetic Route to (±)-Lysergie Acid. Tetrahedron Lett. 1976, 17, 4311-4312; (h) Komfeld, E. C.; Fornefeld, E. J.; Kline, G. B.; Mann, M. J.; Morrison, D. E.; Jones, R. G.; Woodward, R. B. The Total Synthesis of Lysergic Acid. J. Am. Chem. Soc. 1956, 78, 3087-3114.
30. Davies, D. L.; Al-Duaij, O.; Fawcett, J.; Giardiello, M.; Hilton, S. T.; Russell, D. R. Room-Temperature Cyclometallation of Amines, Imines and Oxazolines with [MCl₂Cp*]₂ (M=Rh, Ir) and [RuCl₂(p-cymene)]₂. Dalton Trans. 2003, 4132-4138.
31. (a) Saget, T.; Lemouzy, S. J.; Cramer, N. Chiral Monodentate Phosphines and Bulky Carboxylic Acids: Cooperative Effects in Palladium-Catalyzed Enantioselective C(sp³)-H Functionalization. Angew. Chem. Int. Ed. 2012, 51, 2238-2242. (b) Brauns, M.; Cramer, N. Efficient Kinetic Resolution of Sulfur-Stereogenic Sulfoximines by Exploiting Cp^{X}Rh^{III}-Catalyzed C—H Functionalization. Angew. Chem. Int. Ed. 2019, 58, 89028906. For chiral amino acids employed for enantioselective C—H activation, see: (c) Sokolov, V. I.; Troitskaya, L. L.; Reutov, O. A. Asymmetric Cyclopalladation of Dimethylaminomethylferrocene. J. Organomet. Chem. 1979, 182, 537-546. (d) Shi, B. F.; Maugel, N.; Zhang, Y. H.; Yu, J. Q. Pd^{III}-Catalyzed Enantioselective Activation of C(sp²)-H and C(sp³)-H Bonds Using Monoprotected Amino Acids as Chiral Ligands. Angew. Chem. Int. Ed. 2008, 47, 4882-4886.
32. See Table S1 in the supporting information for the effect of the loading of the chiral amine.
33. Catalytic reactions with (E)-1as and (Z)-1as were performed in 3 hours, respectively. ¹H NMR studies of the recovered 1as did not indicate detectable E/Z isomerization in both experiments.
34. Gao, Z.; Wang, C.; Yuan, C.; Zhou, L.; Xiao, Y.; Guo, H. Chem. Commun. 2015, 51, 12653.
35. Deng, W.; Liu, L.; Zhang, C.; Liu, M.; Guo, Q.-X. Tetrahedron Lett. 2005, 46, 7295.
36. Chan, Y.-C.; Yeung, Y.-Y. Angew. Chem., Int. Ed. 2018, 57, 3483.
37. Bayeh, L.; Le, P. Q.; Tambar, U. K. Nature 2017, 547, 196.
38. Saint Program included in the package software: APEX3 v2018.1.0.
39. Bruker (2001). Program name. Bruker AXS Inc., Madison, Wis., USA.
40. SHELXT-Integrated space-group and crystal-structure determination Sheldrick, G. M. Acta Crystallogr., Sect. A 2015, A71, 3-8.
41. SHELXTL Sheldrick, G. M. Ver. 2016/6. Acta Crystallographica. Sect C Structural Chemistry 71, 3-8.
42. APEX3 v2018, 1.0, AXS Bruker program.
43. L. J. Farrugia (2012) J. Appl. Cryst. 45,849-854.
44. Farrugia, L. J. J. Appl. Cryst. 1997, 30, 565.

It will be understood that various details of the presently disclosed subject matter can be changed without departing from the scope of the subject matter disclosed herein. Furthermore, the foregoing description is for the purpose of illustration only, and not for the purpose of limitation.

1. A method of Ru(II)-catalyzed enantioselective synthesis of a cyclic compound, the method comprising:
providing a precursor compound having an unfunctionalized C—H bond; and
activating the unfunctionalized C—H bond by reacting the precursor compound in the presence of co-catalysts including:
a Ru(II) arene complex; and
a chiral transient directing group (CTDG).

2. The method of claim 1, wherein the Ru(II) arene complex comprises a structure according to Formula I: 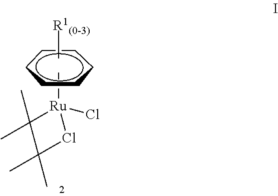
wherein R includes a branched or unbranched alkyl.

3. The method of claim 2, wherein the Ru(II) arene complex is selected from the group consisting of:

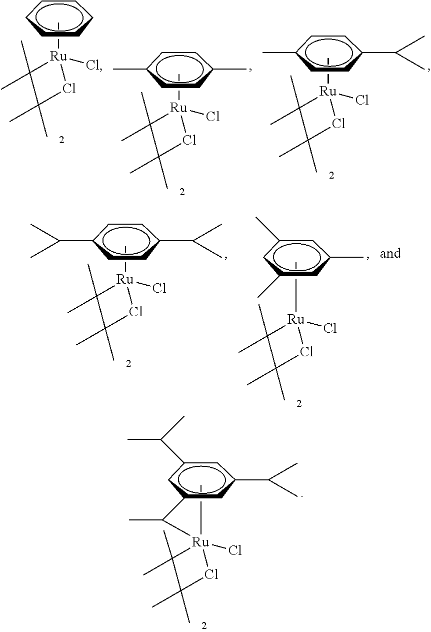

4. The method of claim 2, wherein the Ru(II) arene complex is:

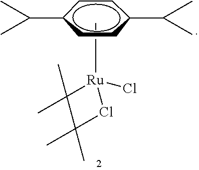

5. The method of claim 1, wherein the CTDG is an α-branched chiral amine.

6. The method of claim 5, wherein the CTDG is selected from the group consisting of:

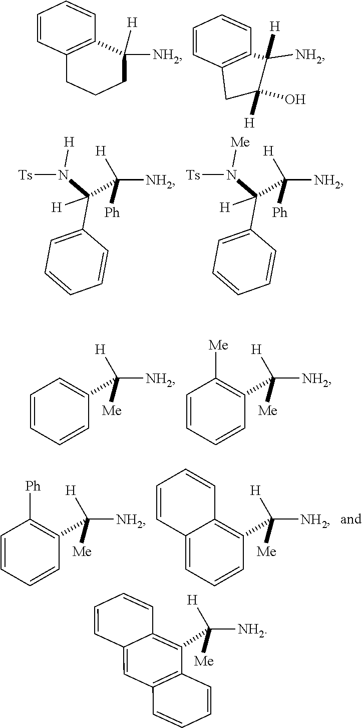

7. The method of claim 5, wherein the CTDG is:

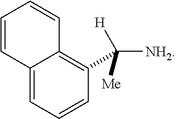

8. The method of claim 1, wherein the Ru(II) arene complex comprises a structure according to Formula I: 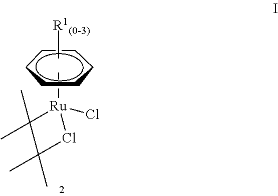
wherein R¹ includes a branched or unbranched alkyl; and
wherein the CTDG is an α-branched chiral amine.

9. The method of claim 8, wherein the Ru(II) arene complex is: 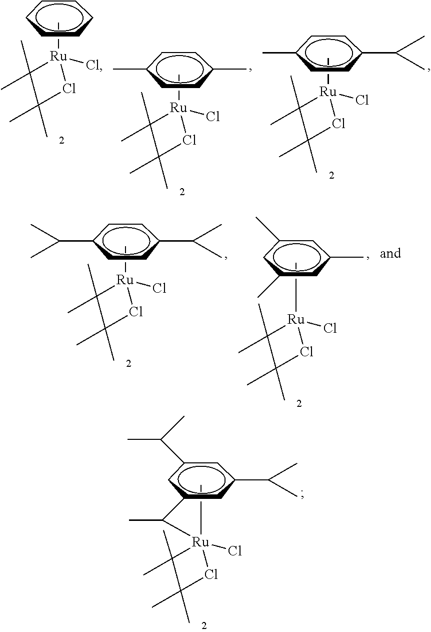
and
wherein the CTDG is selected from the group consisting of

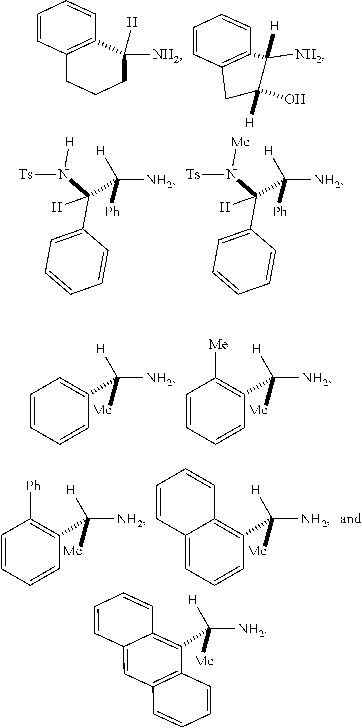

10. The method of claim 9 wherein the Ru(II) arene complex is: 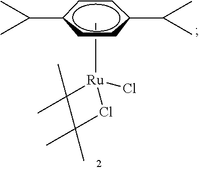
and
wherein the CTDG is:

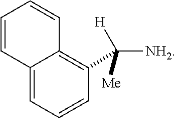

11. The method according to claim 1, wherein the precursor compound comprises a compound according to Formula II: 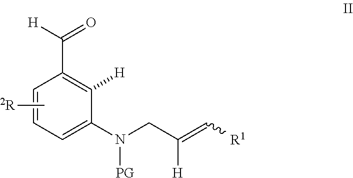
wherein R¹ is selected from the group consisting of alkyl, substituted alkyl, aryl, substituted aryl, electron withdrawing group, and combinations thereof,
wherein R² is selected from the group consisting of H, alkyl, alkoxy, CF₃, halogen, and combinations thereof, and
wherein PG is a protecting group.

12. The method of claim 11, wherein the cyclic compound is an indoline derivative.

13. The method according to claim 1, wherein the precursor compound comprises a compound according to Formula III: 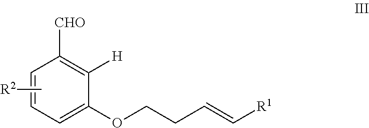
wherein R¹ is selected from the group consisting of alkyl, substituted alkyl, aryl, substituted aryl, electron withdrawing group, and combinations thereof, and
wherein R² is selected from the group consisting of H, alkyl, alkoxy, CF₃, halogen, and combinations thereof.

14. The method of claim 13, wherein the cyclic compound is a chromane derivative.

15. A cyclic compound having a structure according to Formula IV: 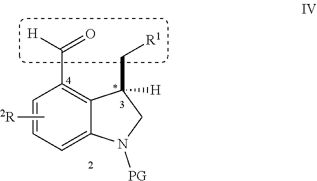
wherein R¹ is selected from the group consisting of alkyl, substituted alkyl, aryl, substituted aryl, electron withdrawing group, and combinations thereof,
wherein R² is selected from the group consisting of H, alkyl, alkoxy, CF₃, halogen, and combinations thereof, and
wherein PG is a protecting group.

16. The cyclic compound of claim 15, wherein the compound is selected from the group consisting of:

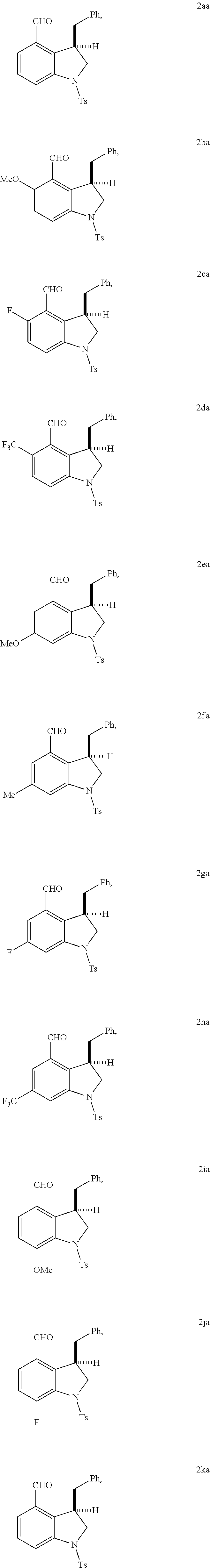

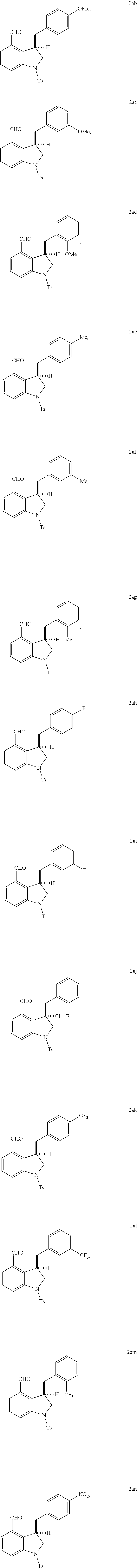

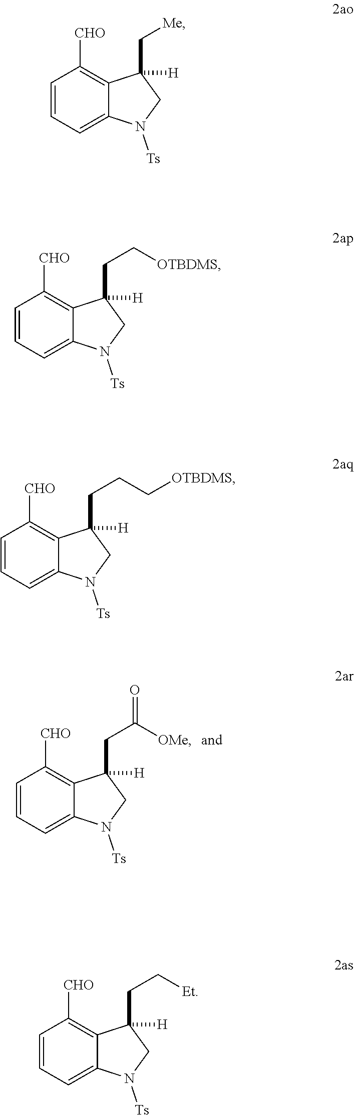 [SMILES: CC[C@H]1CN(S(=O)(=O)c2ccc(C)cc2)c2cccc(C=O)c21 ; Cc1ccc(S(=O)(=O)N2C[C@H](CCO[Si](C)(C)C(C)(C)C)c3c(C=O)cccc32)cc1 ; Cc1ccc(S(=O)(=O)N2C[C@H](CCCO[Si](C)(C)C(C)(C)C)c3c(C=O)cccc32)cc1 ; COC(=O)C[C@H]1CN(S(=O)(=O)c2ccc(C)cc2)c2cccc(C=O)c21 ; CCCC[C@H]1CN(S(=O)(=O)c2ccc(C)cc2)c2cccc(C=O)c21]

17. The cyclic compound of claim 15, wherein the compound is formed according to the method of claim 1.

18. A tricyclic compound having a structure according to Formula V: 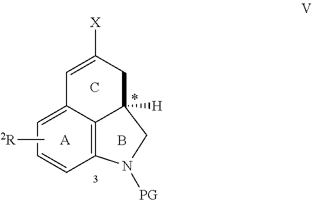
wherein X is CHO;
wherein R² is selected from the group consisting of H, alkyl, alkoxy, CF₃, halogen, and combinations thereof; and
wherein PG is a protecting group.

19. The tricyclic compound of claim 18, wherein the compound is: [chemical structure]
